SEQUENCE LISTING (52 sequences)

SEQ ID NO: 1 - protein, 214 aa, Artificial Sequence
  Ranibizumab Fab Amino Acid Sequence - light chain
DIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 231 aa, Artificial Sequence
  Ranibizumab Fab Amino Acid Sequence - heavy chain
EVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 3 - protein, 214 aa, Artificial Sequence
  Bevacizumab Fab Amino Acid Sequence - Light chain
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 231 aa, Artificial Sequence
  Bevacizumab Fab Amino Acid Sequence - Heavy chain
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 5 - protein, 26 aa, Artificial Sequence
  VEGF-A signal peptide
MNFLLSWVHWSLALLLYLHHAKWSQA

SEQ ID NO: 6 - protein, 29 aa, Artificial Sequence
  Fibulin-1 signal peptide
MERAAPSRRVPLPLLLLGGLALLAAGVDA

SEQ ID NO: 7 - protein, 19 aa, Artificial Sequence
  Vitronectin signal peptide
MAPLRPLLILALLAWVALA

SEQ ID NO: 8 - protein, 18 aa, Artificial Sequence
  Complement Factor H signal peptide
MRLLAKIICLMLWAICVA

SEQ ID NO: 9 - protein, 19 aa, Artificial Sequence
  Opticin signal peptide
MRLLAFLSLLALVLQETGT

SEQ ID NO: 10 - DNA, 728 bp, Artificial Sequence
  Bevacizumab cDNA - Light chain
gctagcgccaccatgggctggtcctgcatcatcctgttcctggtggccaccgccaccggcgtgcactccgacatccagatgacccagtccccctcctccctgtccgcctccgtgggcgaccgggtgaccatcacctgctccgcctcccaggacatctccaactacctgaactggtaccagcagaagcccggcaaggcccccaaggtgctgatctacttcacctcctccctgcactccggcgtgccctcccggttctccggctccggctccggcaccgacttcaccctgaccatctcctccctgcagcccgaggacttcgccacctactactgccagcagtactccaccgtgccctggaccttcggccagggcaccaaggtggagatcaagcggaccgtggccgccccctccgtgttcatcttccccccctccgacgagcagctgaagtccggcaccgcctccgtggtgtgcctgctgaacaacttctacccccgggaggccaaggtgcagtggaaggtggacaacgccctgcagtccggcaactcccaggagtccgtgaccgagcaggactccaaggactccacctactccctgtcctccaccctgaccctgtccaaggccgactacgagaagcacaaggtgtacgcctgcgaggtgacccaccagggcctgtcctcccccgtgaccaagtccttcaaccggggcgagtgctgagcggccgcctcgag

SEQ ID NO: 11 - DNA, 1440 bp, Artificial Sequence
  Bevacizumab cDNA - heavy chain
gctagcgccaccatgggctggtcctgcatcatcctgttcctggtggccaccgccaccggcgtgcactccgaggtgcagctggtggagtccggcggcggcctggtgcagcccggcggctccctgcggctgtcctgcgccgcctccggctacaccttcaccaactacggcatgaactgggtgcggcaggcccccggcaagggcctggagtgggtgggctggatcaacacctacaccggcgagcccacctacgccgccgacttcaagcggcggttcaccttctccctggacacctccaagtccaccgcctacctgcagatgaactccctgcgggccgaggacaccgccgtgtactactgcgccaagtacccccactactacggctcctcccactggtacttcgacgtgtggggccagggcaccctggtgaccgtgtcctccgcctccaccaagggcccctccgtgttccccctggccccctcctccaagtccacctccggcggcaccgccgccctgggctgcctggtgaaggactacttccccgagcccgtgaccgtgtcctggaactccggcgccctgacctccggcgtgcacaccttccccgccgtgctgcagtcctccggcctgtactccctgtcctccgtggtgaccgtgccctcctcctccctgggcacccagacctacatctgcaacgtgaaccacaagccctccaacaccaaggtggacaagaaggtggagcccaagtcctgcgacaagacccacacctgccccccctgccccgcccccgagctgctgggcggcccctccgtgttcctgttcccccccaagcccaaggacaccctgatgatctcccggacccccgaggtgacctgcgtggtggtggacgtgtcccacgaggaccccgaggtgaagttcaactggtacgtggacggcgtggaggtgcacaacgccaagaccaagccccgggaggagcagtacaactccacctaccgggtggtgtccgtgctgaccgtgctgcaccaggactggctgaacggcaaggagtacaagtgcaaggtgtccaacaaggccctgcccgcccccatcgagaagaccatctccaaggccaagggccagccccgggagccccaggtgtacaccctgcccccctcccgggaggagatgaccaagaaccaggtgtccctgacctgcctggtgaagggcttctacccctccgacatcgccgtggagtgggagtccaacggccagcccgagaacaactacaagaccaccccccccgtgctggactccgacggctccttcttcctgtactccaagctgaccgtggacaagtcccggtggcagcagggcaacgtgttctcctgctccgtgatgcacgaggccctgcacaaccactacacccagaagtccctgtccctgtcccccggcaagtgagcggccgcc

SEQ ID NO: 12 - DNA, 733 bp, Artificial Sequence
  Ranibizumab cDNA (Light chain comprising a signal sequence)
gagctccatggagtttttcaaaaagacggcacttgccgcactggttatgggttttagtggtgcagcattggccgatatccagctgacccagagcccgagcagcctgagcgcaagcgttggtgatcgtgttaccattacctgtagcgcaagccaggatattagcaattatctgaattggtatcagcagaaaccgggtaaagcaccgaaagttctgatttattttaccagcagcctgcatagcggtgttccgagccgttttagcggtagcggtagtggcaccgattttaccctgaccattagcagcctgcagccggaagattttgcaacctattattgtcagcagtatagcaccgttccgtggacctttggtcagggcaccaaagttgaaattaaacgtaccgttgcagcaccgagcgtttttatttttccgcctagtgatgaacagctgaaaagcggcaccgcaagcgttgtttgtctgctgaataatttttatccgcgtgaagcaaaagtgcagtggaaagttgataatgcactgcagagcggtaatagccaagaaagcgttaccgaacaggatagcaaagatagcacctatagcctgagcagcaccctgaccctgagcaaagcagattatgaaaaacacaaagtgtatgcctgcgaagttacccatcagggtctgagcagtccggttaccaaaagttttaatcgtggcgaatgctaatagaagcttggtacc

SEQ ID NO: 13 - DNA, 779 bp, Artificial Sequence
  Ranibizumab cDNA (Heavy chain comprising a signal sequence)
gagctcatatgaaatacctgctgccgaccgctgctgctggtctgctgctcctcgctgcccagccggcgatggccgaagttcagctggttgaaagcggtggtggtctggttcagcctggtggtagcctgcgtctgagctgtgcagcaagcggttatgattttacccattatggtatgaattgggttcgtcaggcaccgggtaaaggtctggaatgggttggttggattaatacctataccggtgaaccgacctatgcagcagattttaaacgtcgttttacctttagcctggataccagcaaaagcaccgcatatctgcagatgaatagcctgcgtgcagaagataccgcagtttattattgtgccaaatatccgtattactatggcaccagccactggtatttcgatgtttggggtcagggcaccctggttaccgttagcagcgcaagcaccaaaggtccgagcgtttttccgctggcaccgagcagcaaaagtaccagcggtggcacagcagcactgggttgtctggttaaagattattttccggaaccggttaccgtgagctggaatagcggtgcactgaccagcggtgttcatacctttccggcagttctgcagagcagcggtctgtatagcctgagcagcgttgttaccgttccgagcagcagcctgggcacccagacctatatttgtaatgttaatcataaaccgagcaataccaaagtggataaaaaagttgagccgaaaagctgcgataaaacccatctgtaatagggtacc

SEQ ID NO: 14 - protein, 11 aa, Artificial Sequence
  Bevacizumab and Ranibizumab Light Chain CDR1
SASQDISNYLN

SEQ ID NO: 15 - protein, 7 aa, Artificial Sequence
  Bevacizumab and Ranibizumab Light Chain CDR2
FTSSLHS

SEQ ID NO: 16 - protein, 9 aa, Artificial Sequence
  Bevacizumab and Ranibizumab Light Chain CDR3
QQYSTVPWT

SEQ ID NO: 17 - protein, 10 aa, Artificial Sequence
  Bevacizumab Heavy Chain CDR1
GYTFTNYGMN

SEQ ID NO: 18 - protein, 17 aa, Artificial Sequence
  Bevacizumab Heavy Chain CDR2
WINTYTGEPTYAADFKR

SEQ ID NO: 19 - protein, 14 aa, Artificial Sequence
  Bevacizumab Heavy Chain CDR3
YPHYYGSSHWYFDV

SEQ ID NO: 20 - protein, 10 aa, Artificial Sequence
  Ranibizumab Heavy Chain CDR1
GYDFTHYGMN

SEQ ID NO: 21 - protein, 14 aa, Artificial Sequence
  Ranibizumab Heavy Chain CDR3
YPYYYGTSHWYFDV

SEQ ID NO: 22 - protein, 18 aa, Artificial Sequence
  Albumin signal peptide
MKWVTFISLLFLFSSAYS

SEQ ID NO: 23 - protein, 18 aa, Artificial Sequence
  Chymotrypsinogen signal peptide
MAFLWLLSCWALLGTTFG

SEQ ID NO: 24 - protein, 20 aa, Artificial Sequence
  Interleukin-2 signal peptide
MYRMQLLSCIALILALVTNS

SEQ ID NO: 25 - protein, 15 aa, Artificial Sequence
  Trypsinogen-2 signal peptide
MNLLLILTFVAAAVA

SEQ ID NO: 26 - protein, 19 aa, Artificial Sequence
  F2A site
LLNFDLLKLAGDVESNPGP

SEQ ID NO: 27 - protein, 21 aa, Artificial Sequence
  Linker T2A
GSGEGRGSLLTCGDVEENPGP

SEQ ID NO: 28 - protein, 22 aa, Artificial Sequence
  Linker P2A
GSGATNFSLLKQAGDVEENPGP

SEQ ID NO: 29 - protein, 23 aa, Artificial Sequence
  Linker E2A
GSGQCTNYALLKLAGDVESNPGP

SEQ ID NO: 30 - protein, 25 aa, Artificial Sequence
  Linker F2A
GSGVKQTLNFDLLKLAGDVESNPGP

SEQ ID NO: 31 - protein, 4 aa, Artificial Sequence
  Furin linker
RKRR

SEQ ID NO: 32 - protein, 4 aa, Artificial Sequence
  Furin linker
RRRR

SEQ ID NO: 33 - protein, 4 aa, Artificial Sequence
  Furin linker
RRKR

SEQ ID NO: 34 - protein, 4 aa, Artificial Sequence
  Furin linker
RKKR

SEQ ID NO: 35 - protein, 4 aa, Artificial Sequence
  Furin linker
  MISC_FEATURE (2)..(2): X = any amino acid
  MISC_FEATURE (3)..(3): X = Lys or Arg
RXXR

SEQ ID NO: 36 - protein, 4 aa, Artificial Sequence
  Furin linker
  MISC_FEATURE (2)..(2): X = any amino acid
RXKR

SEQ ID NO: 37 - protein, 4 aa, Artificial Sequence
  Furin linker
  MISC_FEATURE (2)..(2): X = any amino acid
RXRR

SEQ ID NO: 38 - protein, 215 aa, Artificial Sequence
  Ranibizumab Fab Amino Acid Sequence - light chain
MDIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 236 aa, Artificial Sequence
  Ranibizumab Fab Amino Acid Sequence - heavy chain
MEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHLRKRR

SEQ ID NO: 40 - protein, 232 aa, Artificial Sequence
  Ranibizumab Fab Amino Acid Sequence - heavy chain
MEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHL

SEQ ID NO: 41 - protein, 736 aa, Artificial Sequence
  AAV1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 42 - protein, 735 aa, Artificial Sequence [sequence omitted]

SEQ ID NO: 43 - protein, 736 aa, Artificial Sequence
  AAV3-3
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKGAVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVRGVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTGTVNHQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 44 - protein, 734 aa, Artificial Sequence
  AAV4-4
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 45 - protein, 724 aa, Artificial Sequence
  AAV5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 46 - protein, 736 aa, Artificial Sequence
  AAV6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 47 - protein, 737 aa, Artificial Sequence
  AAV7
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 48 - protein, 738 aa, Artificial Sequence
  AAV8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 49 - protein, 736 aa, Artificial Sequence
  hu31
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGGQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVSTEGVYSEPRPIGTRYLTRNL

SEQ ID NO: 50 - protein, 736 aa, Artificial Sequence
  hu32
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 51 - protein, 736 aa, Artificial Sequence
  AAV9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 52 - protein, 20 aa, Artificial Sequence
  Mutant Interleukin-2 signal peptide
MYRMQLLLLIALSLALVTNS

(19) United States
(12) Patent Application Publication
(10) Pub. No.: US 2024/0108669 A1
(43) Pub. Date: Apr. 4, 2024

(54) ADENO-ASSOCIATED VIRUS VECTOR PHARMACEUTICAL COMPOSITION AND METHODS

(71) Applicant: REGENXBIO INC., Rockville, MD (US)

(72) Inventors: Tristan Marshall, Potomac, MD (US); Jared Bee, Gaithersburg, MD (US); Kristin O'Berry, Clarksburg, MD (US); Yu Zhang, Gaithersburg, MD (US); Roberto DePaz, Bethesda, MD (US)

(21) Appl. No.: 17/766,941

(22) PCT Filed: Oct. 6, 2020
(86) PCT No.: PCT/US2020/054400

§ 371 Date: Apr. 6, 2022

Related U.S. Application Data
(60) Provisional application No. 62/911,968, filed on Oct. 7, 2019.

(51) Int. Cl. A61K 35/761; A61K 9/08; A61K 47/02; A61K 47/10; A61K 47/26; C07K 16/22; C12N 15/86

(52) U.S. Cl. CPC A61K 35/761; A61K 9/08; A61K 47/02; A61K 47/10; A61K 47/26; C07K 16/22; C12N 15/86; C12N 2750/14143

(57) ABSTRACT

Provided herein are pharmaceutical compositions comprising a recombinant adeno-associated virus (AAV), salt excipient or buffer agent, sugar and surfactant. Also provided herein are methods for treating or preventing a disease in a subject by administering a therapeutically effective amount of the said pharmaceutical composition to the subject in need.

### CROSS-REFERENCE TO RELATED APPLICATIONS

This application claims the benefit of U.S. Provisional Application No. 62/911,968 filed Oct. 7, 2019, the content of which is incorporated herein by reference in its entirety.

### REFERENCE TO SEQUENCE LISTING SUBMITTED ELECTRONICALLY

This application incorporates by reference a Sequence Listing submitted with this application as text file entitled “Sequence_Listing_12656-124-228.TXT” created on Sep. 28, 2020 and having a size of 97,652 bytes.

### 1. BACKGROUND OF THE INVENTION

Adeno-associated virus (AAV), a member of the Parvoviridae family designated Dependovirus, is a small nonenveloped, icosahedral virus with single-stranded linear DNA genomes of approximately 4.7 kilobases (kb) to 6 kb. The properties of non-pathogenicity, broad host and cell type tropism range of infectivity, including both dividing and non-dividing cells, and ability to establish long-term transgene expression make AAV an attractive tool for gene therapy (e.g., Gongalves, 2005, Virology Journal, 2:43).

AAV product is often stored in buffers composed of various excipients to stabilize the product during manufacture, shipping, storage, and administration. AAV biotherapeutics are however distributed in −80° C. for safety against degradation and the negative effects of potential thaw of materials, even though shipment to certain territories may not provide proper cold storage at these temperatures. It is a challenge to maintain a freezer temperature at ≤−60° C. and providing a formulation that is robust to higher frozen temperatures such as up to −20° C., and stable to multiple freeze-thaw excursions, is desirable from a logistics perspective. Not all clinical sites have a −80° C. freezer and this requirement would negatively impact the ability to distribute the product to a wide range of clinical sites. Therefore, it is desirable to have a formulation that is stable for short (up to 12 months) duration at refrigerated conditions to allow the clinical site to thaw and hold the product in a refrigerator until the patient is scheduled for dosing.

It is critical to maintain various buffer properties within target specification ranges to ensure product stability, yet storage at −80° C. impacts supply chain and distribution. Crystallization of water during slow freezing can result in concentration of excipients which can impact the stability of biologics. Phase separation or pH shifts may also occur which can impact the stability of biologics. For the commercialization of any pharmaceutical product, it would be advantageous to identify formulations that offer stability for extended periods of time. It would be further advantageous to identify formulations that are stable under frozen storage at −20° C. to account for freezer temperature excursions, variability, or temporary storage (up to 18 months) in a −20° C. freezer, refrigerated conditions to allow for short-term storage (up to 12 months at 2-8° C.) at the clinic before dosing, at room temperature to allow for manufacturing and labelling, or under multiple free-thaw cycles to allow for thawing of drug substance and drug product for filling and labelling operations.

### 2. SUMMARY OF THE INVENTION

The disclosure provides a pharmaceutical composition comprising a recombinant adeno-associated virus (AAV), buffering agent, ionic salt, sucrose, and surfactant such as poloxamer 188. Sucrose is provided at a concentration that prevents crystallization of the composition and maintains a pH between 6 and 9 during frozen and liquid states.

In some embodiments, the AAV comprises components from one or more adeno-associated virus serotypes selected from the group consisting of AAV1, AAV2, AAV2tYF, AAV3, AAV4, AAV5, AAV6, AAV7, AAV8, AAV9, AAV10, AAV11, and AAVrh10, AAV.rh20, AAV.rh39, AAV.Rh74, AAV.RHM4-1, AAV.hu37, AAV.Anc80, AAV.Anc80L65, rAAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16. In some embodiments, the rAAV comprises a capsid protein of the AAV8 or AAV9 serotype.

In some embodiments, the pharmaceutical composition further comprises an amino acid.

In some embodiments, the disclosure provides a pharmaceutical composition comprises a recombinant adeno-associated virus (AAV), ionic salt excipient or buffering agent, sucrose, and poloxamer 188. In some embodiments, the ionic salt excipient or buffering agent can be one or more components from the group consisting of potassium phosphate monobasic, potassium phosphate, sodium chloride, sodium phosphate dibasic anhydrous, sodium phosphate hexahydrate, sodium phosphate monobasic monohydrate, tromethamine, tris(hydroxymethyl)aminomethane hydrochloride (Tris-HCl), amino acid, histidine, histidine hydrochloride (histidine-HCl), sodium succinate, sodium citrate, sodium acetate, and (4-(2-hydroxyethyl)-1-piperazineethanesulfonic acid) (HEPES), sodium sulfate, magnesium sulfate, magnesium chloride 6-hydrate, calcium sulfate, potassium chloride, calcium chloride, calcium citrate.

In some embodiments, the pharmaceutical composition has an ionic strength no greater than about 150 mM, about 145 mM, about 140 mM, about 135 mM, about 130 mM, about 125 mM, about 120 mM, about 115 mM, or about 110 mM. In certain embodiments, the pharmaceutical composition has a buffering agent ionic strength no greater than about 150 mM, about 145 mM, about 140 mM, about 135 mM, about 130 mM, about 125 mM, about 120 mM, about 115 mM, or about 110 mM.

In some embodiments, the pharmaceutical composition has an ionic strength no greater than 150 mM, 145 mM, 140 mM, 135 mM, 130 mM, 125 mM, 120 mM, 115 mM, or 110 mM. In certain embodiments, the pharmaceutical composition has a buffering agent ionic strength no greater than 150 mM, 145 mM, 140 mM, 135 mM, 130 mM, 125 mM, 120 mM, 115 mM, or 110 mM.

In certain embodiments, the pharmaceutical composition has a ionic strength about 60 mM to 115 mM. In certain embodiments, the pharmaceutical composition has a ionic strength about 60 mM to 100 mM. In a specific embodiment, the pharmaceutical composition has a ionic strength about 60 mM. In a specific embodiment, the pharmaceutical composition has a ionic strength about 65 mM. In a specific embodiment, the pharmaceutical composition has a ionic strength about 70 mM. In a specific embodiment, the pharmaceutical composition has a ionic strength about 75 mM. In a specific embodiment, the pharmaceutical composition has a ionic strength about 80 mM. In a specific embodiment, the pharmaceutical composition has a ionic strength about 85 mM. In a specific embodiment, the pharmaceutical composition has a ionic strength about 90 mM.

In certain embodiments, the pharmaceutical composition has a ionic strength about 30 mM to 100 mM. In a specific embodiment, the pharmaceutical composition has a ionic strength about 30 mM. In a specific embodiment, the pharmaceutical composition has a ionic strength about 35 mM. In a specific embodiment, the pharmaceutical composition has a ionic strength about 40 mM. In a specific embodiment, the pharmaceutical composition has a ionic strength about 45 mM. In a specific embodiment, the pharmaceutical composition has a ionic strength about 50 mM. In a specific embodiment, the pharmaceutical composition has a ionic strength about 55 mM. In a specific embodiment, the pharmaceutical composition has a ionic strength about 60 mM. In a specific embodiment, the pharmaceutical composition has a ionic strength about 65 mM. In a specific embodiment, the pharmaceutical composition has a ionic strength about 70 mM. In a specific embodiment, the pharmaceutical composition has a ionic strength about 75 mM. In a specific embodiment, the pharmaceutical composition has a ionic strength about 80 mM. In a specific embodiment, the pharmaceutical composition has a ionic strength about 85 mM. In a specific embodiment, the pharmaceutical composition has a ionic strength about 90 mM. In a specific embodiment, the pharmaceutical composition has a ionic strength about 95 mM. In a specific embodiment, the pharmaceutical composition has a ionic strength about 100 mM.

In certain embodiments, the pharmaceutical composition has a ionic strength about 60 mM to 115 mM. In certain embodiments, the pharmaceutical composition has a ionic strength about 65 mM to 95 mM. In certain embodiments, the pharmaceutical composition has a ionic strength about 70 mM to 90 mM. In certain embodiments, the pharmaceutical composition has a ionic strength about 75 mM to 85 mM.

In certain embodiments, the pharmaceutical composition has a ionic strength about 30 mM to 100 mM. In certain embodiments, the pharmaceutical composition has a ionic strength about 35 mM to 95 mM. In certain embodiments, the pharmaceutical composition has a ionic strength about 40 mM to 90 mM. In certain embodiments, the pharmaceutical composition has a ionic strength about 45 mM to 85 mM. In certain embodiments, the pharmaceutical composition has a ionic strength about 50 mM to 80 mM. In certain embodiments, the pharmaceutical composition has a ionic strength about 55 mM to 75 mM. In certain embodiments, the pharmaceutical composition has a ionic strength about 60 mM to 70 mM.

In certain embodiments, the pharmaceutical composition comprises potassium chloride at a concentration of 0.2 g/L.

In certain embodiments, the pharmaceutical composition comprises potassium phosphate monobasic at a concentration of 0.2 g/L.

In certain embodiments, the pharmaceutical composition comprises sodium chloride at a concentration of 5.84 g/L, and

In certain embodiments, the pharmaceutical composition comprises sodium phosphate dibasic anhydrous at a concentration of 1.15 g/L.

In certain embodiments, the pharmaceutical composition comprises sucrose at a concentration of 3% (weight/volume, 30 g/L) to 18% (weight/volume, 180 g/L). In certain embodiments, the pharmaceutical composition comprises sucrose at a concentration of 4% (weight/volume, 30 g/L) to 6% (weight/volume, 180 g/L). In certain embodiments, the pharmaceutical composition comprises sucrose at a concentration of 3% (weight/volume, 30 g/L). In certain embodiments, the pharmaceutical composition comprises sucrose at a concentration of 3% (weight/volume, 30 g/L). In certain embodiments, the pharmaceutical composition comprises sucrose at a concentration of 4% (weight/volume, 40 g/L). In certain embodiments, the pharmaceutical composition comprises sucrose at a concentration of 5% (weight/volume, 50 g/L). In certain embodiments, the pharmaceutical composition comprises sucrose at a concentration of 6% (weight/volume, 60 g/L). In certain embodiments, the pharmaceutical composition comprises sucrose at a concentration of 7% (weight/volume, 70 g/L). In certain embodiments, the pharmaceutical composition comprises sucrose at a concentration of 8% (weight/volume, 80 g/L). In certain embodiments, the pharmaceutical composition comprises sucrose at a concentration of 9% (weight/volume, 90 g/L). In certain embodiments, the pharmaceutical composition comprises sucrose at a concentration of 10% (weight/volume, 100 g/L). In certain embodiments, the pharmaceutical composition comprises sucrose at a concentration of 11% (weight/volume, 110 g/L). In certain embodiments, the pharmaceutical composition comprises sucrose at a concentration of 12% (weight/volume, 120 g/L). In certain embodiments, the pharmaceutical composition comprises sucrose at a concentration of 13% (weight/volume, 130 g/L). In certain embodiments, the pharmaceutical composition comprises sucrose at a concentration of 14% (weight/volume, 140 g/L). In certain embodiments, the pharmaceutical composition comprises sucrose at a concentration of 15% (weight/volume, 150 g/L). In certain embodiments, the pharmaceutical composition comprises sucrose at a concentration of 16% (weight/volume, 160 g/L). In certain embodiments, the pharmaceutical composition comprises sucrose at a concentration of 17% (weight/volume, 170 g/L). In certain embodiments, the pharmaceutical composition comprises sucrose at a concentration of 18% (weight/volume, 180 g/L).

In certain embodiments, the pharmaceutical composition comprises poloxamer 188 at a concentration of 0.001% (weight/volume, 0.01 g/L).

In certain embodiments, the pharmaceutical composition comprises poloxamer 188 at a concentration of 0.0005% (weight/volume, 0.005 g/L) to 0.05% (weight/volume, 0.5 g/L. In certain embodiments, the pharmaceutical composition comprises poloxamer 188 at a concentration of 0.0001% (weight/volume, 0.001 g/L) to 0.01% (weight/volume, 0.1 g/L). In certain embodiments, the pharmaceutical composition comprises poloxamer 188 at a concentration of 0.0005% (weight/volume, 0.005 g/L) to 0.001% (weight/volume, 0.01 g/L). In certain embodiments, the pharmaceutical composition comprises poloxamer 188 at a concentration of 0.001% (weight/volume, 0.01 g/L) to 0.05% (weight/volume, 0.5 g/L). In certain embodiments, the pharmaceutical composition comprises poloxamer 188 at a concentration of 0.0005% (weight/volume, 0.005 g/L). In certain embodiments, the pharmaceutical composition comprises poloxamer 188 at a concentration of 0.0006% (weight/volume, 0.006 g/L). In certain embodiments, the pharmaceutical composition comprises poloxamer 188 at a concentration of 0.0007% (weight/volume, 0.007 g/L). In certain embodiments, the pharmaceutical composition comprises poloxamer 188 at a concentration of 0.0008% (weight/volume, 0.008 g/L). In certain embodiments, the pharmaceutical composition comprises poloxamer 188 at a concentration of 0.0009% (weight/volume, 0.009 g/L). In certain embodiments, the pharmaceutical composition comprises poloxamer 188 at a concentration of 0.001% (weight/volume, 0.01 g/L). In certain embodiments, the pharmaceutical composition comprises poloxamer 188 at a concentration of 0.002% (weight/volume, 0.02 g/L). In certain embodiments, the pharmaceutical composition comprises poloxamer 188 at a concentration of 0.003% (weight/volume, 0.03 g/L). In certain embodiments, the pharmaceutical composition comprises poloxamer 188 at a concentration of 0.004% (weight/volume, 0.04 g/L). In certain embodiments, the pharmaceutical composition comprises poloxamer 188 at a concentration of 0.005% (weight/volume, 0.05 g/L). In certain embodiments, the pharmaceutical composition comprises poloxamer 188 at a concentration of 0.01% (weight/volume, 0.1 g/L). In certain embodiments, the pharmaceutical composition comprises poloxamer 188 at a concentration of 0.05% (weight/volume, 0.5 g/L).

In some embodiments, the disclosure provides a pharmaceutical composition comprises a recombinant adeno-associated virus (AAV), ionic salt excipient or buffering agent, sucrose, and surfactant. In some embodiments, the ionic salt excipient or buffering agent can be one or more components from the group consisting of potassium phosphate monobasic, potassium phosphate, sodium chloride, sodium phosphate dibasic anhydrous, sodium phosphate hexahydrate, sodium phosphate monobasic monohydrate, tromethamine, tris(hydroxymethyl)aminomethane hydrochloride (Tris-HCl), amino acid, histidine, histidine hydrochloride (histidine-HCl), sodium succinate, sodium citrate, sodium acetate, and (4-(2-hydroxyethyl)-1-piperazineethanesulfonic acid) (HEPES), sodium sulfate, magnesium sulfate, magnesium chloride 6-hydrate, calcium sulfate, potassium chloride, calcium chloride, calcium citrate. In some embodiments, the surfactant can be one or more components from the group consisting of poloxamer 188, polysorbate 20, and polysorbate 80.

In certain embodiments, the pharmaceutical composition comprises polysorbate 20 at a concentration of 0.0005% (weight/volume, 0.005 g/L) to 0.05% (weight/volume, 0.5 g/L. In certain embodiments, the pharmaceutical composition comprises polysorbate 20 at a concentration of 0.0001% (weight/volume, 0.001 g/L) to 0.01% (weight/volume, 0.1 g/L). In certain embodiments, the pharmaceutical composition comprises polysorbate 20 at a concentration of 0.0005% (weight/volume, 0.005 g/L) to 0.001% (weight/volume, 0.01 g/L). In certain embodiments, the pharmaceutical composition comprises polysorbate 20 at a concentration of 0.001% (weight/volume, 0.01 g/L) to 0.05% (weight/volume, 0.5 g/L). In certain embodiments, the pharmaceutical composition comprises polysorbate 20 at a concentration of 0.0005% (weight/volume, 0.005 g/L). In certain embodiments, the pharmaceutical composition comprises polysorbate 20 at a concentration of 0.0006% (weight/volume, 0.006 g/L). In certain embodiments, the pharmaceutical composition comprises polysorbate 20 at a concentration of 0.0007% (weight/volume, 0.007 g/L). In certain embodiments, the pharmaceutical composition comprises polysorbate 20 at a concentration of 0.0008% (weight/volume, 0.008 g/L). In certain embodiments, the pharmaceutical composition comprises polysorbate 20 at a concentration of 0.0009% (weight/volume, 0.009 g/L). In certain embodiments, the pharmaceutical composition comprises polysorbate 20 at a concentration of 0.001% (weight/volume, 0.01 g/L). In certain embodiments, the pharmaceutical composition comprises polysorbate 20 at a concentration of 0.002% (weight/volume, 0.02 g/L). In certain embodiments, the pharmaceutical composition comprises polysorbate 20 at a concentration of 0.003% (weight/volume, 0.03 g/L). In certain embodiments, the pharmaceutical composition comprises polysorbate 20 at a concentration of 0.004% (weight/volume, 0.04 g/L). In certain embodiments, the pharmaceutical composition comprises polysorbate 20 at a concentration of 0.005% (weight/volume, 0.05 g/L). In certain embodiments, the pharmaceutical composition comprises polysorbate 20 at a concentration of 0.01% (weight/volume, 0.1 g/L). In certain embodiments, the pharmaceutical composition comprises polysorbate 20 at a concentration of 0.05% (weight/volume, 0.5 g/L).

In certain embodiments, the pharmaceutical composition comprises polysorbate 80 at a concentration of 0.0005% (weight/volume, 0.005 g/L) to 0.05% (weight/volume, 0.5 g/L. In certain embodiments, the pharmaceutical composition comprises polysorbate 80 at a concentration of 0.0001% (weight/volume, 0.001 g/L) to 0.01% (weight/volume, 0.1 g/L). In certain embodiments, the pharmaceutical composition comprises polysorbate 80 at a concentration of 0.0005% (weight/volume, 0.005 g/L) to 0.001% (weight/volume, 0.01 g/L). In certain embodiments, the pharmaceutical composition comprises polysorbate 80 at a concentration of 0.001% (weight/volume, 0.01 g/L) to 0.05% (weight/volume, 0.5 g/L). In certain embodiments, the pharmaceutical composition comprises polysorbate 80 at a concentration of 0.0005% (weight/volume, 0.005 g/L). In certain embodiments, the pharmaceutical composition comprises polysorbate 80 at a concentration of 0.0006% (weight/volume, 0.006 g/L). In certain embodiments, the pharmaceutical composition comprises polysorbate 80 at a concentration of 0.0007% (weight/volume, 0.007 g/L). In certain embodiments, the pharmaceutical composition comprises polysorbate 80 at a concentration of 0.0008% (weight/volume, 0.008 g/L). In certain embodiments, the pharmaceutical composition comprises polysorbate 80 at a concentration of 0.0009% (weight/volume, 0.009 g/L). In certain embodiments, the pharmaceutical composition comprises polysorbate 80 at a concentration of 0.001% (weight/volume, 0.01 g/L). In certain embodiments, the pharmaceutical composition comprises polysorbate 80 at a concentration of 0.002% (weight/volume, 0.02 g/L). In certain embodiments, the pharmaceutical composition comprises polysorbate 80 at a concentration of 0.003% (weight/volume, 0.03 g/L). In certain embodiments, the pharmaceutical composition comprises polysorbate 80 at a concentration of 0.004% (weight/volume, 0.04 g/L). In certain embodiments, the pharmaceutical composition comprises polysorbate 80 at a concentration of 0.005% (weight/volume, 0.05 g/L). In certain embodiments, the pharmaceutical composition comprises polysorbate 80 at a concentration of 0.01% (weight/volume, 0.1 g/L). In certain embodiments, the pharmaceutical composition comprises polysorbate 80 at a concentration of 0.05% (weight/volume, 0.5 g/L).

In certain embodiments, the pH of the pharmaceutical composition is about 7.4.

In certain embodiments, the pH of the pharmaceutical composition is about 6.0 to 8.8. In certain embodiments, the pH of the pharmaceutical composition is about 6.0 to 9.0. In certain embodiments, the pH of the pharmaceutical composition is about 6.0. In certain embodiments, the pH of the pharmaceutical composition is about 6.1. In certain embodiments, the pH of the pharmaceutical composition is about 6.2. In certain embodiments, the pH of the pharmaceutical composition is about 6.3. In certain embodiments, the pH of the pharmaceutical composition is about 6.4. In certain embodiments, the pH of the pharmaceutical composition is about 6.5. In certain embodiments, the pH of the pharmaceutical composition is about 6.6. In certain embodiments, the pH of the pharmaceutical composition is about 6.7. In certain embodiments, the pH of the pharmaceutical composition is about 6.8. In certain embodiments, the pH of the pharmaceutical composition is about 6.9. In certain embodiments, the pH of the pharmaceutical composition is about 7.0. In certain embodiments, the pH of the pharmaceutical composition is about 7.1. In certain embodiments, the pH of the pharmaceutical composition is about 7.2. In certain embodiments, the pH of the pharmaceutical composition is about 7.3. In certain embodiments, the pH of the pharmaceutical composition is about 7.4. In certain embodiments, the pH of the pharmaceutical composition is about 7.5. In certain embodiments, the pH of the pharmaceutical composition is about 7.6. In certain embodiments, the pH of the pharmaceutical composition is about 7.7. In certain embodiments, the pH of the pharmaceutical composition is about 7.8. In certain embodiments, the pH of the pharmaceutical composition is about 7.9. In certain embodiments, the pH of the pharmaceutical composition is about 8.0. In certain embodiments, the pH of the pharmaceutical composition is about 8.1. In certain embodiments, the pH of the pharmaceutical composition is about 8.2. In certain embodiments, the pH of the pharmaceutical composition is about 8.3. In certain embodiments, the pH of the pharmaceutical composition is about 8.4. In certain embodiments, the pH of the pharmaceutical composition is about 8.5. In certain embodiments, the pH of the pharmaceutical composition is about 8.6. In certain embodiments, the pH of the pharmaceutical composition is about 8.7. In certain embodiments, the pH of the pharmaceutical composition is about 8.8. In certain embodiments, the pH of the pharmaceutical composition is about 8.9. In certain embodiments, the pH of the pharmaceutical composition is about 9.0.

In certain embodiments, the pH of the pharmaceutical composition is 7.4.

In certain embodiments, the pH of the pharmaceutical composition is 6.0 to 8.8. In certain embodiments, the pH of the pharmaceutical composition is 6.0 to 9.0. In certain embodiments, the pH of the pharmaceutical composition is 6.0. In certain embodiments, the pH of the pharmaceutical composition is 6.1. In certain embodiments, the pH of the pharmaceutical composition is 6.2. In certain embodiments, the pH of the pharmaceutical composition is 6.3. In certain embodiments, the pH of the pharmaceutical composition is 6.4. In certain embodiments, the pH of the pharmaceutical composition is 6.5. In certain embodiments, the pH of the pharmaceutical composition is 6.6. In certain embodiments, the pH of the pharmaceutical composition is 6.7. In certain embodiments, the pH of the pharmaceutical composition is 6.8. In certain embodiments, the pH of the pharmaceutical composition is 6.9. In certain embodiments, the pH of the pharmaceutical composition is 7.0. In certain embodiments, the pH of the pharmaceutical composition is 7.1. In certain embodiments, the pH of the pharmaceutical composition is 7.2. In certain embodiments, the pH of the pharmaceutical composition is 7.3. In certain embodiments, the pH of the pharmaceutical composition is 7.4. In certain embodiments, the pH of the pharmaceutical composition is 7.5. In certain embodiments, the pH of the pharmaceutical composition is 7.6. In certain embodiments, the pH of the pharmaceutical composition is 7.7. In certain embodiments, the pH of the pharmaceutical composition is 7.8. In certain embodiments, the pH of the pharmaceutical composition is 7.9. In certain embodiments, the pH of the pharmaceutical composition is 8.0. In certain embodiments, the pH of the pharmaceutical composition is 8.1. In certain embodiments, the pH of the pharmaceutical composition is 8.2. In certain embodiments, the pH of the pharmaceutical composition is 8.3. In certain embodiments, the pH of the pharmaceutical composition is 8.4. In certain embodiments, the pH of the pharmaceutical composition is 8.5. In certain embodiments, the pH of the pharmaceutical composition is 8.6. In certain embodiments, the pH of the pharmaceutical composition is 8.7. In certain embodiments, the pH of the pharmaceutical composition is 8.8. In certain embodiments, the pH of the pharmaceutical composition is 8.9. In certain embodiments, the pH of the pharmaceutical composition is 9.0.

As used herein and unless otherwise specified, the term “about” means within plus or minus 10% of a given value or range.

In certain embodiments, the pharmaceutical composition is in a hydrophobically-coated glass vial.

In certain embodiments, the pharmaceutical composition is in a Cyclo Olefin Polymer (COP) vial.

In certain embodiments, the pharmaceutical composition is in a Daikyo Crystal Zenith® (CZ) vial.

In certain embodiments, the pharmaceutical composition is in a TopLyo coated vial.

In certain embodiments, disclosed herein is a pharmaceutical composition consists of: (a) the recombinant AAV, (b) potassium chloride at a concentration of 0.2 g/L, (c) potassium phosphate monobasic at a concentration of 0.2 g/L, (d) sodium chloride at a concentration of 5.84 g/L, (e) sodium phosphate dibasic anhydrous at a concentration of 1.15 g/L, (f) sucrose at a concentration of 4% weight/volume (40 g/L), (g) poloxamer 188 at a concentration of 0.001% weight/volume (0.01 g/L), and (h) water, and wherein the recombinant AAV is AAV8.

In certain embodiments, the vector genome concentration (VGC) of the pharmaceutical composition is 3×10⁹ GC/mL, 1×10¹⁰ GC/mL, 1.2×10¹⁰ GC/mL, 1.6×10¹⁰ GC/mL, 4×10¹¹ GC/mL, 6×10¹⁰ GC/mL, 2×10¹¹ GC/mL, 2.4×10¹¹ GC/mL, 2.5×10¹¹ GC/mL, 3×10¹¹ GC/mL, 3.2×10¹¹ GC/mL, 6.2×10¹¹ GC/mL, 6.5×10¹¹ GC/mL, 1×10¹² GC/mL, 3×10¹² GC/mL, 2×10¹³ GC/mL, or 3×10¹³ GC/mL.

In certain embodiments, the vector genome concentration (VGC) of the pharmaceutical composition is 3×10⁹ GC/mL, 4×10⁹ GC/mL, 5×10⁹ GC/mL, 6×10⁹ GC/mL, 7×10⁹ GC/mL, 8×10⁹ GC/mL, 9×10⁹ GC/mL, 1×10¹⁰ GC/mL, 2×10¹⁰ GC/mL, 3×10¹⁰ GC/mL, 4×10¹⁰ GC/mL, 5×10¹⁰ GC/mL, 6×10¹⁰ GC/mL, 7×10¹⁰ GC/mL, 8×10¹⁰ GC/mL, 9×10¹⁰ GC/mL, 1×10¹¹ GC/mL, 2×10¹¹ GC/mL, 3×10¹¹ GC/mL, 4×10¹¹ GC/mL, 5×10¹¹ GC/mL, 6×10¹¹ GC/mL, 7×10¹¹ GC/mL, 8×10¹¹ GC/mL, 9×10¹¹ GC/mL, 1×10¹² GC/mL, 2×10¹² GC/mL, 3×10¹² GC/mL, 4×10¹² GC/mL, 5×10¹² GC/mL, 6×10¹² GC/mL, 7×10¹² GC/mL, 8×10¹² GC/mL, 9×10¹² GC/mL, 1×10¹³ GC/mL, 1×10¹³ GC/mL, 2×10¹³ GC/mL, or 3×10¹³ GC/mL.

In certain embodiments, the vector genome concentration (VGC) of the pharmaceutical composition is about 3×10⁹ GC/mL, about 1×10¹⁰ GC/mL, about 1.2×10¹⁰ GC/mL, about 1.6×10¹⁰ GC/mL, about 4×10¹¹ GC/mL, about 6×10¹⁰ GC/mL, about 2×10¹¹ GC/mL, about 2.4×10¹¹ GC/mL, about 2.5×10¹¹ GC/mL, about 3×10¹¹ GC/mL, about 3.2×10¹¹ GC/mL, about 6.2×10¹¹ GC/mL, about 6.5×10¹¹ GC/mL, about 1×10¹² GC/mL, about 3×10¹² GC/mL, about 2×10¹³ GC/mL or about 3×10¹³ GC/mL.

In certain embodiments, the vector genome concentration (VGC) of the pharmaceutical composition is about 3×10⁹ GC/mL, 4×10⁹ GC/mL, 5×10⁹ GC/mL, 6×10⁹ GC/mL, 7×10⁹ GC/mL, 8×10⁹ GC/mL, 9×10⁹ GC/mL, about 1×10¹⁰ GC/mL, about 2×10¹⁰ GC/mL, about 3×10¹⁰ GC/mL, about 4×10¹⁰ GC/mL, about 5×10¹⁰ GC/mL, about 6×10¹⁰ GC/mL, about 7×10¹⁰ GC/mL, about 8×10¹⁰ GC/mL, about 9×10¹⁰ GC/mL, about 1×10¹¹ GC/mL, about 2×10¹¹ GC/mL, about 3×10¹¹ GC/mL, about 4×10¹¹ GC/mL, about 5×10¹¹ GC/mL, about 6×10¹¹ GC/mL, about 7×10¹¹ GC/mL, about 8×10¹¹ GC/mL, about 9×10¹¹ GC/mL, about 1×10¹² GC/mL, about 2×10¹² GC/mL, about 3×10² GC/mL, about 4×10² GC/mL, about 5×10¹² GC/mL, about 6×10¹² GC/mL, about 7×10² GC/mL, about 8×10¹² GC/mL, about 9×10¹² GC/mL, about 1×10¹³ GC/mL, about 1×10¹³ GC/mL, about 2×10¹³ GC/mL, about 3×10¹³ GC/mL.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more stable to freeze/thaw cycles than the same recombinant AAV in a reference pharmaceutical composition. In certain embodiments, the stability of the recombinant AAV is determined by an assay or assays disclosed in Section 4.5 and Section 5.

In certain embodiments, the recombinant AAV in the pharmaceutical composition has at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more infectivity than the same recombinant AAV in a reference pharmaceutical composition. In certain embodiments, the virus infectivity of the recombinant AAV is determined by an assay or assays disclosed in Section 4.5 and Section 5. In certain embodiments, the infectivity is measured prior to or after freeze/thaw cycles.

In certain embodiments, the recombinant AAV in the pharmaceutical composition has at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 μless aggregation than the same recombinant AAV in a reference pharmaceutical composition. In certain embodiments, the aggregation of the recombinant AAV is determined by an assay or assays disclosed in Section 4.5 and Section 5. In certain embodiments, the aggregation is measured prior to or after freeze/thaw cycles.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more stable over a period of time, for example, about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, 12 months, about 15 months, about 18 months, about 24 months, about 2 years, about 3 years, about 4 years than the same recombinant AAV in a reference pharmaceutical composition. In certain embodiments, the stability over a period of time of the recombinant AAV is determined by an assay or assays disclosed in Section 4.5 and Section 5.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more stable over a period of time, at least for example, about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, 12 months, about 15 months, about 18 months, about 24 months, about 2 years, about 3 years, about 4 years than the same recombinant AAV in a reference pharmaceutical composition. In certain embodiments, the stability over a period of time of the recombinant AAV is determined by an assay or assays disclosed in Section 4.5 and Section 5.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 higher in vitro relative potency (IVRP) than the same recombinant AAV in a reference pharmaceutical composition. In certain embodiments, the in vitro relative potency (IVRP) of the recombinant AAV is determined by an assay or assays disclosed in Section 4.5 and Section 5. In certain embodiments, the in vitro relative potency (IVRP) is measured prior to or after freeze/thaw cycles.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 μless free DNA than the same recombinant AAV in a reference pharmaceutical composition. In certain embodiments, the free DNA of the recombinant AAV is determined by an assay or assays disclosed in Section 4.5 and Section 5. In certain embodiments, the aggregation is measured prior to or after freeze/thaw cycles.

In certain embodiments, the recombinant AAV in the pharmaceutical composition has at most 20%, 15%, 10%, 8%, 5%, 4%, 3%, 2%, or 1% change in size over a period of time, for example, about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years. In certain embodiments, the size of the recombinant AAV is determined by an assay or assays disclosed in Section 4.5 and Section 5. In certain embodiments, the size is measured prior to or after freeze/thaw cycles.

In certain embodiments, the recombinant AAV in the pharmaceutical composition has at most 20%, 15%, 10%, 8%, 5%, 4%, 3%, 2%, or 1% change in size over a period of time, for example, at least about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years. In certain embodiments, the size of the recombinant AAV is determined by an assay or assays disclosed in Section 4.5 and Section 5. In certain embodiments, the size is measured prior to or after freeze/thaw cycles.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more stable than the same recombinant AAV in a reference pharmaceutical composition when stored at −20° C. In certain embodiments, the stability of the recombinant AAV is determined by an assay or assays disclosed in Section 4.5 and Section 5.

In certain embodiments, the recombinant AAV in the pharmaceutical composition has at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more infectivity than the same recombinant AAV in a reference pharmaceutical composition when stored at −20° C. for a period of time, for example, about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years, than the same recombinant AAV in a reference pharmaceutical composition. In certain embodiments, the virus infectivity of the recombinant AAV is determined by an assay or assays disclosed in Section 4.5 and Section 5.

In certain embodiments, the recombinant AAV in the pharmaceutical composition has at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more infectivity than the same recombinant AAV in a reference pharmaceutical composition when stored at −20° C. for a period of time, for example, at least about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years, than the same recombinant AAV in a reference pharmaceutical composition. In certain embodiments, the virus infectivity of the recombinant AAV is determined by an assay or assays disclosed in Section 4.5 and Section 5.

In certain embodiments, the recombinant AAV in the pharmaceutical composition has at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 μless aggregation than the same recombinant AAV in a reference pharmaceutical composition when stored at −20° C. for a period of time, for example, about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years, than the same recombinant AAV in a reference pharmaceutical composition. In certain embodiments, the aggregation of the recombinant AAV is determined by an assay or assays disclosed in Section 4.5 and Section 5.

In certain embodiments, the recombinant AAV in the pharmaceutical composition has at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 μless aggregation than the same recombinant AAV in a reference pharmaceutical composition when stored at −20° C. for a period of time, at least for example, at least about 1 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years, than the same recombinant AAV in a reference pharmaceutical composition. In certain embodiments, the aggregation of the recombinant AAV is determined by an assay or assays disclosed in Section 4.5 and Section 5.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more stable when stored at −20° C. for a period of time, for example, about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years, than the same recombinant AAV in a reference pharmaceutical composition. In certain embodiments, the stability over a period of time of the recombinant AAV is determined by an assay or assays disclosed in Section 4.5 and Section 5.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more stable when stored at −20° C. for a period of time, for example, at least about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years, than the same recombinant AAV in a reference pharmaceutical composition. In certain embodiments, the stability over a period of time of the recombinant AAV is determined by an assay or assays disclosed in Section 4.5 and Section 5. In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more stable than the same recombinant AAV in a reference pharmaceutical composition when (i) stored at −80° C. for a first period of time; (ii) subsequently thawed; and (iii) after thawing, stored at 4° C. for a second period of time. In certain embodiments, the stability over a period of time of the recombinant AAV is determined by an assay or assays disclosed in Section 4.5 and Section 5. In certain embodiments, the first period of time is about 6 months, about 7 months, about 8 months, about 9 months, about 10 months, about 11 months, about 12 months, about 15 months, about 18 months, or about 24 months. 28. In some embodiments, the second period of time is about 1 week, about 2 weeks, about 3 weeks, about 4 weeks, about 1 month, about 2 months.

In certain embodiments, the recombinant AAV is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more stable than the same recombinant AAV in a reference pharmaceutical composition when (i) stored at −80° C. for a first period of time; (ii) subsequently thawed; and (iii) after thawing, stored at 4° C. for a second period of time. In some embodiments, the first period of time is about 6 months, about 7 months, about 8 months, about 9 months, about 10 months, about 11 months, about 12 months, about 15 months, about 18 months, or about 24 months. In some embodiments, the second period of time is about 1 week, about 2 weeks, about 3 weeks, about 4 weeks, about 1 month, about 2 months.

In some embodiments, the vector genome concentration of the recombinant AAV after being stored at −80° C. for a period of time is at least 70%, 75%, 80%, 85%, 90%, 95%, 98%, or 99% of the vector genome concentration of the recombinant AAV before being stored at −80° C. for said period of time. In some embodiments, the vector genome concentration of the recombinant AAV after being stored at −20° C. for a period of time is at least 70%, 75%, 80%, 85%, 90%, 95%, 98%, or 99% of the vector genome concentration of the recombinant AAV before being stored at −20° C. for said period of time. In some embodiments, the vector genome concentration of the recombinant AAV after being stored at 4° C. for a period of time is at least 70%, 75%, 80%, 85%, 90%, 95%, 98%, or 99% of the vector genome concentration of the recombinant AAV before being stored at 4° C. for said period of time.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 higher in vitro relative potency (IVRP) than the same recombinant AAV in a reference pharmaceutical composition when stored at −20° C. for a period of time, for example, 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years, than the same recombinant AAV in a reference pharmaceutical composition. In certain embodiments, the in vitro relative potency (IVRP) of the recombinant AAV is determined by an assay or assays disclosed in Section 4.5 and Section 5.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 higher in vitro relative potency (IVRP) than the same recombinant AAV in a reference pharmaceutical composition when stored at −20° C. for a period of time, for example, at least about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years, than the same recombinant AAV in a reference pharmaceutical composition. In certain embodiments, the in vitro relative potency (IVRP) of the recombinant AAV is determined by an assay or assays disclosed in Section 4.5 and Section 5.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 higher in vitro relative potency (IVRP) than the same recombinant AAV in a reference pharmaceutical composition when stored at −20° C. for a period of time, for example, about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years, than the same recombinant AAV in a reference pharmaceutical composition. In certain embodiments, the in vitro relative potency (IVRP) of the recombinant AAV is determined by an assay or assays disclosed in Section 4.5 and Section 5.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 higher in vitro relative potency (IVRP) than the same recombinant AAV in a reference pharmaceutical composition when stored at −20° C. for a period of time, for example, at least about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years, than the same recombinant AAV in a reference pharmaceutical composition. In certain embodiments, the in vitro relative potency (IVRP) of the recombinant AAV is determined by an assay or assays disclosed in Section 4.5 and Section 5.

In certain embodiments, the recombinant AAV in the pharmaceutical composition has at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 μless free DNA than the same recombinant AAV in a reference pharmaceutical composition when stored at −20° C. for a period of time, for example, about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years, than the same recombinant AAV in a reference pharmaceutical composition. In certain embodiments, the free DNA of the recombinant AAV is determined by an assay or assays disclosed in Section 4.5 and Section 5.

In certain embodiments, the recombinant AAV in the pharmaceutical composition has at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 μless free DNA than the same recombinant AAV in a reference pharmaceutical composition when stored at −20° C. for a period of time, for example, at least about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years, than the same recombinant AAV in a reference pharmaceutical composition. In certain embodiments, the free DNA of the recombinant AAV is determined by an assay or assays disclosed in Section 4.5 and Section 5.

In certain embodiments, the recombinant AAV in the pharmaceutical composition has at most 20%, 15%, 10%, 8%, 5%, 4%, 3%, 2%, or 1% change in size when stored at −20° C. over a period of time, for example, about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years. In certain embodiments, the size of the recombinant AAV is determined by an assay or assays disclosed in Section 4.5 and Section 5.

In certain embodiments, the recombinant AAV in the pharmaceutical composition has at most 20%, 15%, 10%, 8%, 5%, 4%, 3%, 2%, or 1% change in size when stored at −20° C. over a period of time, for example, at least about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years. In certain embodiments, the size of the recombinant AAV is determined by an assay or assays disclosed in Section 4.5 and Section 5.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more stable than the same recombinant AAV in a reference pharmaceutical composition when stored at 37° C. In certain embodiments, the stability of the recombinant AAV is determined by an assay or assays disclosed in Section 4.5 and Section 5.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more infectivity than the same recombinant AAV in a reference pharmaceutical composition when stored at 37° C. for a period of time, for example, about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years, than the same recombinant AAV in a reference pharmaceutical composition. In certain embodiments, the virus infectivity of the recombinant AAV is determined by an assay or assays disclosed in Section 4.5 and Section 5.

In certain embodiments, the recombinant AAV in the pharmaceutical composition has at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more infectivity than the same recombinant AAV in a reference pharmaceutical composition when stored at 37° C. for a period of time, for example, at least about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years, than the same recombinant AAV in a reference pharmaceutical composition. In certain embodiments, the virus infectivity of the recombinant AAV is determined by an assay or assays disclosed in Section 4.5 and Section 5.

In certain embodiments, the recombinant AAV in the pharmaceutical composition has at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 μless aggregation than the same recombinant AAV in a reference pharmaceutical composition when stored at 37° C. for a period of time, for example, about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years, than the same recombinant AAV in a reference pharmaceutical composition. In certain embodiments, the aggregation of the recombinant AAV is determined by an assay or assays disclosed in Section 4.5 and Section 5.

In certain embodiments, the recombinant AAV in the pharmaceutical composition has at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 μless aggregation than the same recombinant AAV in a reference pharmaceutical composition when stored at 37° C. for a period of time, for example, at least about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years, than the same recombinant AAV in a reference pharmaceutical composition. In certain embodiments, the aggregation of the recombinant AAV is determined by an assay or assays disclosed in Section 4.5 and Section 5.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more stable when stored at 37° C. for a period of time, for example, about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years, than the same recombinant AAV in a reference pharmaceutical composition. In certain embodiments, the stability over a period of time of the recombinant AAV is determined by an assay or assays disclosed in Section 4.5 and Section 5.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more stable when stored at 37° C. for a period of time, for example, at least about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years, than the same recombinant AAV in a reference pharmaceutical composition. In certain embodiments, the stability over a period of time of the recombinant AAV is determined by an assay or assays disclosed in Section 4.5 and Section 5. In some embodiments, the vector genome concentration of the recombinant AAV after being stored at −80° C. for a period of time is at least 70%, 75%, 80%, 85%, 90%, 95%, 98%, or 99% of the vector genome concentration of the recombinant AAV before being stored at −80° C. for said period of time. In some embodiments, the vector genome concentration of the recombinant AAV after being stored at −20° C. for a period of time is at least 70%, 75%, 80%, 85%, 90%, 95%, 98%, or 99% of the vector genome concentration of the recombinant AAV before being stored at −20° C. for said period of time. In some embodiments, the vector genome concentration of the recombinant AAV after being stored at 4° C. for a period of time is at least 70%, 75%, 80%, 85%, 90%, 95%, 98%, or 99% of the vector genome concentration of the recombinant AAV before being stored at 4° C. for said period of time. In some embodiments, the vitro potency of the recombinant AAV after being stored at −80° C. for a period of time is at least 70%, 75%, 80%, 85%, 90%, 95%, 98%, or 99% of the in vitro potency of the recombinant AAV before being stored at −80° C. for said period of time. In some embodiments, the in vitro potency of the recombinant AAV after being stored at −20° C. for a period of time is at least 70%, 75%, 80%, 85%, 90%, 95%, 98%, or 99% of the in vitro potency of the recombinant AAV before being stored at −20° C. for said period of time. In some embodiments, the in vitro potency of the recombinant AAV after being stored at −20° C. for a period of time is at least 70%, 75%, 80%, 85%, 90%, 95%, 98%, or 99% of the in vitro potency of the recombinant AAV before being stored at −20° C. for said period of time. In some embodiments, the size distribution of the recombinant AAV after being stored at −80° C. for a period of time is at least 70%, 75%, 80%, 85%, 90%, 95%, 98%, or 99% identical to the size distribution of the recombinant AAV before being stored at −80° C. for said period of time. In some embodiments, the size distribution of the recombinant AAV after being stored at −20° C. for a period of time is at least 70%, 75%, 80%, 85%, 90%, 95%, 98%, or 99% identical to the size distribution of the recombinant AAV before being stored at −20° C. for said period of time. In some embodiments, the size distribution of the recombinant AAV after being stored at 4° C. for a period of time is at least 70%, 75%, 80%, 85%, 90%, 95%, 98%, or 99% identical to the size distribution of the recombinant AAV before being stored at 4° C. for said period of time. In some embodiments, the period of time is about 1 month, about 2 months, about 3 months, about 4 months, about 5 months, about 6 months, about 7 months, about 8 months, about 9 months, about 10 months, about 11 months, about 12 months, about 15 months, about 18 months, or about 24 months.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 higher in vitro relative potency (IVRP) than the same recombinant AAV in a reference pharmaceutical composition when stored at 37° C. for a period of time, for example, about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years, than the same recombinant AAV in a reference pharmaceutical composition. In certain embodiments, the in vitro relative potency (IVRP) of the recombinant AAV is determined by an assay or assays disclosed in Section 4.5 and Section 5.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 higher in vitro relative potency (IVRP) than the same recombinant AAV in a reference pharmaceutical composition when stored at 37° C. for a period of time, for example, at least about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years, than the same recombinant AAV in a reference pharmaceutical composition. In certain embodiments, the in vitro relative potency (IVRP) of the recombinant AAV is determined by an assay or assays disclosed in Section 4.5 and Section 5.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 μless free DNA than the same recombinant AAV in a reference pharmaceutical composition when stored at 37° C. for a period of time, for example, about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years, than the same recombinant AAV in a reference pharmaceutical composition. In certain embodiments, the free DNA of the recombinant AAV is determined by an assay or assays disclosed in Section 4.5 and Section 5.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 μless free DNA than the same recombinant AAV in a reference pharmaceutical composition when stored at 37° C. for a period of time, at least for example, about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years, than the same recombinant AAV in a reference pharmaceutical composition. In certain embodiments, the free DNA of the recombinant AAV is determined by an assay or assays disclosed in Section 4.5 and Section 5.

In certain embodiments, the recombinant AAV in the pharmaceutical composition has at most 20%, 15%, 10%, 8%, 5%, 4%, 3%, 2%, or 1% change in size when stored at 37° C. over a period of time, for example, about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years. In certain embodiments, the size of the recombinant AAV is determined by an assay or assays disclosed in Section 4.5 and Section 5.

In certain embodiments, the recombinant AAV in the pharmaceutical composition has at most 20%, 15%, 10%, 8%, 5%, 4%, 3%, 2%, or 1% change in size when stored at 37° C. over a period of time, for example, at least about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years. In certain embodiments, the size of the recombinant AAV is determined by an assay or assays disclosed in Section 4.5 and Section 5.

In another aspect, provided herein is a method of treating a subject diagnosed with nAMD (wet AMD), dry AMD, retinal vein occlusion (RVO), diabetic macular edema (DME), or diabetic retinopathy (DR), the method comprising preparing a pharmaceutical composition provided herein, storing the pharmaceutical composition at −80° C. for a first period of time; (ii) thawing the pharmaceutical composition; and (iii) after thawing, storing the pharmaceutical composition at 4° C. for a second period of time. In some embodiments, the first period of time is about 6 months, about 7 months, about 8 months, about 9 months, about 10 months, about 11 months, about 12 months, about 15 months, about 18 months, or about 24 months. 61. In some embodiments, the second period of time is about 1 week, about 2 weeks, about 3 weeks, about 4 weeks, about 1 month, about 2 months.

In certain embodiments, the disclosure provides a pharmaceutical composition or formulation comprising a recombinant adeno-associated virus (AAV), potassium phosphate monobasic, sodium chloride, sodium phosphate dibasic anhydrous, sucrose, and poloxamer 188. In some embodiments, the AAV comprises components from AAV8. In some embodiments, the AAV is AAV viral vectors provided herein comprise the following elements in the following order: a) a constitutive or a hypoxia-inducible promoter sequence, and b) a sequence encoding the transgene (e.g., an anti-VEGF antigen-binding fragment moiety). In some embodiments, the transgene is a fully human post-translationally modified (HuPTM) antibody against VEGF. Antibodies include, but are not limited to, monoclonal antibodies, polyclonal antibodies, recombinantly produced antibodies, human antibodies, humanized antibodies, chimeric antibodies, synthetic antibodies, tetrameric antibodies comprising two heavy chain and two light chain molecules, antibody light chain monomers, antibody heavy chain monomers, antibody light chain dimers, antibody heavy chain dimers, antibody light chain-heavy chain pairs, intrabodies, heteroconjugate antibodies, monovalent antibodies, antigen-binding fragments of full-length antibodies, and fusion proteins of the above. Such antigen-binding fragments include, but are not limited to, single-domain antibodies (variable domain of heavy chain antibodies (VHHs) or nanobodies), Fabs, F(ab′)₂s, and scFvs (single-chain variable fragments) of full-length anti-VEGF antibodies (preferably, full-length anti-VEGF monoclonal antibodies (mAbs) (collectively referred to herein as “antigen-binding fragments”). In a preferred embodiment, the fully human post-translationally modified antibody against VEGF is a fully human post-translationally modified antigen-binding fragment of a monoclonal antibody (mAb) against VEGF (“HuPTMFabVEGFi”). In a further preferred embodiment, the HuPTMFabVEGFi is a fully human glycosylated antigen-binding fragment of an anti-VEGF mAb (“HuGlyFabVEGFi”). In an alternative embodiment, full-length mAbs can be used. In a preferred embodiment, the AAV used for delivering the transgene should have a tropism for human retinal cells or photoreceptor cells. Such AAV can include non-replicating recombinant adeno-associated virus vectors (“rAAV”), particularly those bearing an AAV8 capsid are preferred. In a specific embodiment, the viral vector or other DNA expression construct described herein is Construct I, wherein the Construct I comprises the following components: (1) AAV8 inverted terminal repeats that flank the expression cassette; (2) control elements, which include a) the CB7 promoter, comprising the CMV enhancer/chicken β-actin promoter, b) a chicken β-actin intron and c) a rabbit β-globin poly A signal; and (3) nucleic acid sequences coding for the heavy and light chains of anti-VEGF antigen-binding fragment, separated by a self-cleaving furin (F)/F2A linker, ensuring expression of equal amounts of the heavy and the light chain polypeptides. In another specific embodiment, the viral vector or other DNA expression construct described herein is Construct II, wherein the Construct II comprise the following components: (1) AAV2 inverted terminal repeats that flank the expression cassette; (2) control elements, which include a) the CB7 promoter, comprising the CMV enhancer/chicken Q-actin promoter, b) a chicken □-actin intron and c) a rabbit β-globin poly A signal; and (3) nucleic acid sequences coding for the heavy and light chains of anti-VEGF antigen-binding fragment, separated by a self-cleaving furin (F)/F2A linker, ensuring expression of equal amounts of the heavy and the light chain polypeptides. In a specific embodiment, the construct described herein is illustrated in FIG. 4. In some embodiments, the anti-hVEGF antibody comprises a heavy chain comprising the amino acid sequence of SEQ ID NO: 2 or SEQ ID NO:4, and a light chain comprising the amino acid sequence of SEQ ID NO:1 or SEQ ID NO:3.

In some embodiments, the pharmaceutical composition consists of: (a) the Construct II encoding an anti-human vascular endothelial growth factor (hVEGF) antibody, (b) potassium chloride at a concentration of 0.2 g/L, (c) potassium phosphate monobasic at a concentration of 0.2 g/L, (d) sodium chloride at a concentration of 5.84 g/L, (e) sodium phosphate dibasic anhydrous at a concentration of 1.15 g/L, (f) sucrose at a concentration of 4% weight/volume (40 g/L), (g) poloxamer 188 at a concentration of 0.001% weight/volume (0.01 g/L), and (h) water, and wherein the anti-hVEGF antibody comprises a heavy chain comprising the amino acid sequence of SEQ ID NO:2 or SEQ ID NO:4, and a light chain comprising the amino acid sequence of SEQ ID NO:1 or SEQ ID NO:3.

In some embodiments, the pharmaceutical composition is a liquid composition. In some embodiments, the pharmaceutical composition is a frozen composition. In some embodiments, the pharmaceutical composition is a lyophilized composition from a liquid composition disclosed herein. In some embodiments, the pharmaceutical composition is a reconstituted lyophilized formulation.

In some embodiments, the pharmaceutical composition is a lyophilized composition comprising a residual moisture content between about 1% and about 7%.

In certain aspects, disclosed herein is a method of treating or preventing a disease in a subject, comprising administering to the subject the pharmaceutical composition.

In certain aspects, disclosed herein is a method of treating or preventing a disease in a subject, comprising administering to the subject the pharmaceutical composition by intravenous administration, subcutaneous administration, intramuscular injection, suprachoroidal injection (for example, via a suprachoroidal drug delivery device such as a microinjector with a microneedle), subretinal injection via transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), and/or a posterior juxtascleral depot procedure (for example, via a juxtascleral drug delivery device comprising a cannula whose tip can be inserted and kept in direct apposition to the scleral surface)).

In certain aspects, the pharmaceutical composition is suitable for administrant to the eye. In certain aspects, the pharmaceutical composition is suitable for suprachoroidal injection, subretinal injection via transvitreal approach, subretinal administration via the suprachoroidal space, or a posterior juxtascleral depot procedure.

In certain embodiments, the pharmaceutical composition is suitable for intravenous administration, subcutaneous administration, intramuscular injection, suprachoroidal injection (for example, via a suprachoroidal drug delivery device such as a microinjector with a microneedle), subretinal injection via transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), and/or a posterior juxtascleral depot procedure (for example, via a juxtascleral drug delivery device comprising a cannula whose tip can be inserted and kept in direct apposition to the scleral surface)).

In certain embodiments, the pharmaceutical composition has a desired density that is suitable for intravenous administration, subcutaneous administration, intramuscular injection, suprachoroidal injection (for example, via a suprachoroidal drug delivery device such as a microinjector with a microneedle), subretinal injection via transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), and/or a posterior juxtascleral depot procedure (for example, via a juxtascleral drug delivery device comprising a cannula whose tip can be inserted and kept in direct apposition to the scleral surface)).

In certain embodiments, the pharmaceutical composition has a desired osmolality that is suitable for intravenous administration, subcutaneous administration, intramuscular injection, suprachoroidal injection (for example, via a suprachoroidal drug delivery device such as a microinjector with a microneedle), subretinal injection via transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), and/or a posterior juxtascleral depot procedure (for example, via a juxtascleral drug delivery device comprising a cannula whose tip can be inserted and kept in direct apposition to the scleral surface)). In specific embodiments, the desired osmolality for subretinal administration is 160-430 mOsm/kg H₂O. In other specific embodiments, the desired osmolality of suprachoroidal administration is less than 600 mOsm/kg H₂O.

In certain embodiments, the pharmaceutical composition has a desired viscosity that is suitable for intravenous administration, subcutaneous administration, intramuscular injection, suprachoroidal injection (for example, via a suprachoroidal drug delivery device such as a microinjector with a microneedle), subretinal injection via transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), and/or a posterior juxtascleral depot procedure (for example, via a juxtascleral drug delivery device comprising a cannula whose tip can be inserted and kept in direct apposition to the scleral surface)).

In certain embodiments, the pharmaceutical composition has a osmolality range of 200 mOsm/L to 660 mOsm/L. In certain embodiments, the pharmaceutical composition has a osmolality range of 200 mOsm/L to 660 mOsm/L. In some embodiments, the osmolality is less than 600 mOsm/kg H₂O. In certain embodiments, the pharmaceutical composition has a osmolality of about 200 mOsm/L. In certain embodiments, the pharmaceutical composition has a osmolality of about 250 mOsm/L. In certain embodiments, the pharmaceutical composition has a osmolality of about 300 mOsm/L. In certain embodiments, the pharmaceutical composition has a osmolality of about 350 mOsm/L. In certain embodiments, the pharmaceutical composition has a osmolality of about 400 mOsm/L. In certain embodiments, the pharmaceutical composition has a osmolality of about 450 mOsm/L. In certain embodiments, the pharmaceutical composition has a osmolality of about 500 mOsm/L. In certain embodiments, the pharmaceutical composition has a osmolality of about 550 mOsm/L. In certain embodiments, the pharmaceutical composition has a osmolality of about 600 mOsm/L. In certain embodiments, the pharmaceutical composition has a osmolality of about 650 mOsm/L. In certain embodiments, the pharmaceutical composition has a osmolality of about 660 mOsm/L. In certain aspects, disclosed herein are methods of treating a subject diagnosed with mucopolysaccharidosis type IVA (MIPS IVA), mucopolysaccharidosis type I (MIPS I), or mucopolysaccharidosis type II (MPS II) comprising administering to the subject the pharmaceutical composition.

In some embodiments, the vector genome concentration of the Construct II after being stored at −80° C. for a period of time is at least 70%, 75%, 80%, 85%, 90%, 95%, 98%, or 99% of the vector genome concentration of the Construct II before being stored at −80° C. for said period of time. In some embodiments, the vector genome concentration of the Construct II after being stored at −20° C. for a period of time is at least 70%, 75%, 80%, 85%, 90%, 95%, 98%, or 99% of the vector genome concentration of the Construct II before being stored at −20° C. for said period of time. In some embodiments, the vector genome concentration of the Construct II after being stored at 4° C. for a period of time is at least 70%, 75%, 80%, 85%, 90%, 95%, 98%, or 99% of the vector genome concentration of the Construct. In some embodiments, the period of time is about 1 month, about 2 months, about 3 months, about 4 months, about 5 months, about 6 months, about 7 months, about 8 months, about 9 months, about 10 months, about 11 months, about 12 months, about 15 months, about 18 months, or about 24 months.

In some embodiments, the in vitro potency of the Construct II after being stored at −80° C. for a period of time is at least 70%, 75%, 80%, 85%, 90%, 95%, 98%, or 99% of the in vitro potency of the Construct II before being stored at −80° C. for said period of time. II before being stored at 4° C. for said period of time. In some embodiments, the in vitro potency of the Construct II after being stored at −20° C. for a period of time is at least 70%, 75%, 80%, 85%, 90%, 95%, 98%, or 99% of the in vitro potency of the Construct II before being stored at −20° C. for said period of time. In some embodiments, the in vitro potency of the Construct II after being stored at 4° C. for a period of time is at least 70%, 75%, 80%, 85%, 90%, 95%, 98%, or 99% of the in vitro potency of the Construct II before being stored at 4° C. for said period of time. In some embodiments, the period of time is about 1 month, about 2 months, about 3 months, about 4 months, about 5 months, about 6 months, about 7 months, about 8 months, about 9 months, about 10 months, about 11 months, about 12 months, about 15 months, about 18 months, or about 24 months.

In some embodiments, the size distribution of the Construct II after being stored at −80° C. for a period of time is at least 70%, 75%, 80%, 85%, 90%, 95%, 98%, or 99% identical to the size distribution of the Construct II before being stored at −80° C. for said period of time. In some embodiments, the size distribution of the Construct II after being stored at −20° C. for a period of time is at least 70%, 75%, 80%, 85%, 90%, 95%, 98%, or 99% identical to the size distribution of the Construct II before being stored at −20° C. for said period of time. In some embodiments, the size distribution of the Construct II after being stored at 4° C. for a period of time is at least 70%, 75%, 80%, 85%, 90%, 95%, 98%, or 99% identical to the size distribution of the Construct II before being stored at 4° C. for said period of time. In some embodiments, the period of time is about 1 month, about 2 months, about 3 months, about 4 months, about 5 months, about 6 months, about 7 months, about 8 months, about 9 months, about 10 months. In certain embodiments, the pharmaceutical composition is capable of being stored at 4° C. for 1 weeks, about 2 weeks, about 3 weeks, about 4 weeks, about 1 month, about 2 months after having previously been stored at −80° C. for about 6 months, about 7 months, about 8 months, about 9 months, about 10 months, about 11 months, about 12 months, about 15 months, about 18 months, about 24 months.

In certain aspects, disclosed herein are methods of treating a subject diagnosed with mucopolysaccharidosis type IVA (MIPS IVA), mucopolysaccharidosis type I (MIPS I), or mucopolysaccharidosis type II (MPS II) comprising administering to the subject a therapeutically effective amount of the pharmaceutical composition by intravenous administration, subcutaneous administration, or intramuscular injection.

In certain aspects, disclosed herein are methods of treating or preventing a disease in a subject, comprising treating a subject diagnosed with nAMD (wet AMD), dry AMD, retinal vein occlusion (RVO), diabetic macular edema (DME), or diabetic retinopathy (DR) comprising administering to the subject a therapeutically effective amount of the pharmaceutical composition.

In certain aspects, disclosed herein are methods of treating a subject diagnosed with nAMD (wet AMD), dry AMD, retinal vein occlusion (RVO), diabetic macular edema (DME), or diabetic retinopathy (DR) comprising administering to the subject a therapeutically effective amount of the pharmaceutical composition by suprachoroidal injection (for example, via a suprachoroidal drug delivery device such as a microinjector with a microneedle), subretinal injection via transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), or a posterior juxtascleral depot procedure (for example, via a juxtascleral drug delivery device comprising a cannula whose tip can be inserted and kept in direct apposition to the scleral surface)).

In certain aspects, described herein are methods of treating a human subject diagnosed with nAMD (wet AMD), dry AMD, retinal vein occlusion (RVO), diabetic macular edema (DME), or diabetic retinopathy (DR) comprising delivering to the retina of said human subject a therapeutically effective amount of anti-hVEGF antigen-binding fragment produced by human retinal cells, by administering to the suprachoroidal space, subretinal space, or outer surface of the sclera in the eye of said human subject (e.g., by suprachoroidal injection (for example, via a suprachoroidal drug delivery device such as a microinjector with a microneedle), subretinal injection via transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), or a posterior juxtascleral depot procedure (for example, via a juxtascleral drug delivery device comprising a cannula whose tip can be inserted and kept in direct apposition to the scleral surface)) the pharmaceutical composition.

In certain aspects, provided herein is a method of treating a subject diagnosed with nAMD (wet AMD), dry AMD, retinal vein occlusion (RVO), diabetic macular edema (DME), or diabetic retinopathy (DR), the method comprising preparing a pharmaceutical composition provided herein, storing the pharmaceutical composition at −80° C. for a first period of time; (ii) thawing the pharmaceutical composition; and (iii) after thawing, storing the pharmaceutical composition at 4° C. for a second period of time. In some embodiments, the first period of time is about 6 months, about 7 months, about 8 months, about 9 months, about 10 months, about 11 months, about 12 months, about 15 months, about 18 months, or about 24 months. In some embodiments, the second period of time is about 1 week, about 2 weeks, about 3 weeks, about 4 weeks, about 1 month, about 2 months.

Described herein are anti-human vascular endothelial growth factor (hVEGF) antibodies, for example, anti-hVEGF antigen-binding fragments, produced by human retinal cells. Human VEGF (hVEGF) is a human protein encoded by the VEGF (VEGFA, VEGFB, VEGFC, or VEGFD) gene. An exemplary amino acid sequence of hVEGF may be found at GenBank Accession No. AAA35789.1. An exemplary nucleic acid sequence of hVEGF may be found at GenBank Accession No. M32977.1.

In certain aspects of the methods described herein, the antigen-binding fragment comprises a heavy chain comprising the amino acid sequence of SEQ ID NO. 2 or SEQ ID NO. 4, and a light chain comprising the amino acid sequence of SEQ ID NO. 1 or SEQ ID NO. 3.

In certain aspects of the methods described herein, the antigen-binding fragment comprises light chain CDRs 1-3 of SEQ ID NOs: 14-16 and heavy chain CDRs 1-3 of SEQ ID NOs:17-19 or SEQ ID NOs: 20, 18, and 21.

In a specific embodiment of the methods described herein, the antigen-binding fragment comprises light chain CDRs 1-3 of SEQ ID NOs: 14-16 and heavy chain CDRs 1-3 of SEQ ID NOs: 20, 18, and 21, wherein the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the second amino acid residue of the light chain CDR3 (i.e., the second Q in QQYSTVPWTF (SEQ ID NO. 16)) is not acetylated. In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) is not acetylated. In a preferred embodiment, the chemical modification(s) or lack of chemical modification(s) (as the case may be) described herein is determined by mass spectrometry.

In a specific embodiment of the methods described herein, the antigen-binding fragment comprises light chain CDRs 1-3 of SEQ ID NOs: 14-16 and heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) is not acetylated. In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) is not acetylated. In a preferred embodiment, the chemical modification(s) or lack of chemical modification(s) (as the case may be) described herein is determined by mass spectrometry.

In a specific embodiment of the methods described herein, the antigen-binding fragment comprises light chain CDRs 1-3 of SEQ ID NOs: 14-16 and heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein: (1) the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu); and (2) the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) is not acetylated, and the second amino acid residue of the light chain CDR3 (i.e., the second Q in QQYSTVPWTF (SEQ ID NO. 16)) is not acetylated. In a specific embodiment, the antigen-binding fragment comprises light chain CDRs 1-3 of SEQ ID NOs: 14-16 and heavy chain CDRs 1-3 of SEQ ID NOs: 20, 18, and 21, wherein: (1) the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) is not acetylated; and (2) the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) is not acetylated. In a preferred embodiment, the chemical modification(s) or lack of chemical modification(s) (as the case may be) described herein is determined by mass spectrometry.

In certain aspects, described herein are methods of treating a human subject diagnosed with nAMD (wet AMD), dry AMD, retinal vein occlusion (RVO), diabetic macular edema (DME), or diabetic retinopathy (DR), comprising: delivering to the eye of said human subject, a therapeutically effective amount of an antigen-binding fragment of a mAb against hVEGF, said antigen-binding fragment containing a α2,6-sialylated glycan. In a specific aspect, described herein are methods of treating a human subject diagnosed with nAMD (wet AMD), dry AMD, retinal vein occlusion (RVO), diabetic macular edema (DME), or diabetic retinopathy (DR), comprising: delivering to the eye of said human subject, a therapeutically effective amount of an antigen-binding fragment of a mAb against hVEGF, said antigen-binding fragment containing a α2,6-sialylated glycan, by administering to the suprachoroidal space, subretinal space, or outer surface of the sclera in the eye of said human subject (e.g., by suprachoroidal injection (for example, via a suprachoroidal drug delivery device such as a microinjector with a microneedle), subretinal injection via the transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), or a posterior juxtascleral depot procedure (for example, via a juxtascleral drug delivery device comprising a cannula whose tip can be inserted and kept in direct apposition to the scleral surface)) the pharmaceutical composition comprising an expression vector encoding the antigen-binding fragment of a mAb against hVEGF.

In certain aspects, described herein are methods of treating a human subject diagnosed with nAMD (wet AMD), dry AMD, retinal vein occlusion (RVO), diabetic macular edema (DME), or diabetic retinopathy (DR), comprising: delivering to the eye of said human subject, a therapeutically effective amount of a glycosylated antigen-binding fragment of a mAb against hVEGF, wherein said antigen-binding fragment does not contain detectable NeuGc and/or α-Gal antigen (i.e., as used herein, “detectable” means levels detectable by standard assays described infra). In a specific embodiment, described herein are methods of treating a human subject diagnosed with nAMD (wet AMD), dry AMD, retinal vein occlusion (RVO), diabetic macular edema (DME), or diabetic retinopathy (DR), comprising: delivering to the eye of said human subject, a therapeutically effective amount of a glycosylated antigen-binding fragment of a mAb against hVEGF, by administering to the suprachoroidal space, subretinal space, or outer surface of the sclera in the eye of said human subject (e.g., by suprachoroidal injection (for example, via a suprachoroidal drug delivery device such as a microinjector with a microneedle), subretinal injection via the transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), or a posterior juxtascleral depot procedure (for example, via a juxtascleral drug delivery device comprising a cannula whose tip can be inserted and kept in direct apposition to the scleral surface)) the pharmaceutical composition comprising expression vector encoding the glycosylated antigen-binding fragment of a mAb against hVEGF, wherein said antigen-binding fragment does not contain detectable NeuGc and/or α-Gal antigen.

More details on anti-hVEGF antibody or antigen-binding fragment of a mAb against hVEGF are provided in International Publication No.: WO2019/067540 (incorporated by reference in its entirety herein).

In a specific aspect, the anti-hVEGF antibody comprises a heavy chain comprising the amino acid sequence of SEQ ID NO. 2 or SEQ ID NO. 4, and a light chain comprising the amino acid sequence of SEQ ID NO. 1 or SEQ ID NO. 3. In a specific aspect, the expression vector is an AAV8 vector.

In certain aspects of the methods described herein, the antigen-binding fragment transgene encodes a leader peptide. A leader peptide may also be referred to as a signal peptide or leader sequence herein.

In certain aspects of the methods described herein, delivering to the eye comprises delivering to the retina, choroid, and/or vitreous humor of the eye. In certain aspects of the methods described herein, the antigen-binding fragment comprises a heavy chain that comprises one, two, three, or four additional amino acids at the C-terminus.

In particular embodiments, the methods encompass treating patients who have been diagnosed with nAMD (wet AMD), dry AMD, retinal vein occlusion (RVO), diabetic macular edema (DME), or diabetic retinopathy (DR), and identified as responsive to treatment with an anti-VEGF antibody. In more specific embodiments, the patients are responsive to treatment with an anti-VEGF antigen-binding fragment. In certain embodiments, the patients have been shown to be responsive to treatment with an anti-VEGF antigen-binding fragment injected intravitreally prior to treatment with gene therapy. In specific embodiments, the patients have previously been treated with LUCENTIS® (ranibizumab), EYLEA® (aflibercept), and/or AVASTIN® (bevacizumab), and have been found to be responsive to one or more of said LUCENTIS® (ranibizumab), EYLEA® (aflibercept), and/or AVASTIN® (bevacizumab).

Subjects to whom such viral vector or other DNA expression construct is delivered should be responsive to the anti-hVEGF antigen-binding fragment encoded by the transgene in the viral vector or expression construct. To determine responsiveness, the anti-VEGF antigen-binding fragment transgene product (e.g., produced in cell culture, bioreactors, etc.) may be administered directly to the subject, such as by intravitreal injection.

In certain aspects of the methods described herein, the antigen-binding fragment comprises a heavy chain that does not comprise an additional amino acid at the C-terminus.

In certain aspects of the methods described herein produces a population of antigen-binding fragment molecules, wherein the antigen-binding fragment molecules comprise a heavy chain, and wherein 0.5%, 1%, 2%, 3%, 4%, 5%, 10%, or 20%, or less of the population of antigen-binding fragment molecules comprises one, two, three, or four additional amino acids at the C-terminus of the heavy chain. In certain aspects of the methods described herein produces a population of antigen-binding fragment molecules, wherein the antigen-binding fragment molecules comprise a heavy chain, and wherein 0.5%, 1%, 2%, 3%, 4%, 5%, 10%, or 20%, or less but more than 0% of the population of antigen-binding fragment molecules comprises one, two, three, or four additional amino acids at the C-terminus of the heavy chain.

In certain aspects of the methods described herein produces a population of antigen-binding fragment molecules, wherein the antigen-binding fragment molecules comprise a heavy chain, and wherein 0.5-1%, 0.5%-2%, 0.5%-3%, 0.5%-4%, 0.5%-5%, 0.5%-10%, 0.5%-20%, 1%-2%, 1%-3%, 1%-4%, 1%-5%, 1%-10%, 1%-20%, 2%-3%, 2%-4%, 2%-5%, 2%-10%, 2%-20%, 3%-4%, 3%-5%, 3%-10%, 3%-20%, 4%-5%, 4%-10%, 4%-20%, 5%-10%, 5%-20%, or 10%-20% of the population of antigen-binding fragment molecules comprises one, two, three, or four additional amino acids at the C-terminus of the heavy chain.

The HuPTMFabVEGFi, e.g., HuGlyFabVEGFi, encoded by the transgene can include, but is not limited to an antigen-binding fragment of an antibody that binds to hVEGF, such as bevacizumab; an anti-hVEGF Fab moiety such as ranibizumab; or such bevacizumab or ranibizumab Fab moieties engineered to contain additional glycosylation sites on the Fab domain (e.g., see Courtois et al., 2016, mAbs 8: 99-112 which is incorporated by reference herein in its entirety for it description of derivatives of bevacizumab that are hyperglycosylated on the Fab domain of the full length antibody).

The recombinant vector used for delivering the transgene should have a tropism for human retinal cells or photoreceptor cells. Such vectors can include non-replicating recombinant adeno-associated virus vectors (“rAAV”), particularly those bearing an AAV8 capsid are preferred. However, other viral vectors may be used, including but not limited to lentiviral vectors, vaccinia viral vectors, or non-viral expression vectors referred to as “naked DNA” constructs. Preferably, the HuPTMFabVEGFi, e.g., HuGlyFabVEGFi, transgene should be controlled by appropriate expression control elements, for example, the CB7 promoter (a chicken 3-actin promoter and CMV enhancer), the RPE65 promoter, or opsin promoter to name a few, and can include other expression control elements that enhance expression of the transgene driven by the vector (e.g., introns such as the chicken 3-actin intron, minute virus of mice (MVM) intron, human factor IX intron (e.g., FIX truncated intron 1), β-globin splice donor/immunoglobulin heavy chain spice acceptor intron, adenovirus splice donor/immunoglobulin splice acceptor intron, SV40 μlate splice donor/splice acceptor (19S/16S) intron, and hybrid adenovirus splice donor/IgG splice acceptor intron and polyA signals such as the rabbit β-globin polyA signal, human growth hormone (hGH) polyA signal, SV40 μlate polyA signal, synthetic polyA (SPA) signal, and bovine growth hormone (bGH) polyA signal). See, e.g., Powell and Rivera-Soto, 2015, Discov. Med., 19(102):49-57.

Gene therapy constructs are designed such that both the heavy and light chains are expressed. More specifically, the heavy and light chains should be expressed at about equal amounts, in other words, the heavy and light chains are expressed at approximately a 1:1 ratio of heavy chains to light chains. The coding sequences for the heavy and light chains can be engineered in a single construct in which the heavy and light chains are separated by a cleavable linker or IRES so that separate heavy and light chain polypeptides are expressed. See, e.g., Section 5.2.4 for specific leader sequences and Section 5.2.5 for specific IRES, 2A, and other linker sequences that can be used with the methods and compositions provided herein.

In certain embodiments, gene therapy constructs are supplied as a frozen sterile, single use solution of the AAV vector active ingredient in a formulation buffer. In a specific embodiment, the pharmaceutical compositions suitable for subretinal administration comprise a suspension of the recombinant (e.g., rHuGlyFabVEGFi) vector in a formulation buffer comprising a physiologically compatible aqueous buffer, a surfactant and optional excipients.

In certain embodiments, gene therapy constructs are supplied as a frozen sterile, single use solution of the AAV vector active ingredient in a formulation buffer. In a specific embodiment, the pharmaceutical compositions suitable for suprachoroidal, subretinal, juxtascleral and/or intraretinal administration comprise a suspension of the recombinant (e.g., rHuGlyFabVEGFi) vector in a formulation buffer comprising a physiologically compatible aqueous buffer, a surfactant and optional excipients.

Therapeutically effective doses of the recombinant vector should be administered subretinally and/or intraretinally (e.g., by subretinal injection via the transvitreal approach (a surgical procedure), or subretinal administration via the suprachoroidal space) in a volume ranging from ≥0.1 mL to ≤0.5 mL, preferably in 0.1 to 0.30 mL (100-300 μl), and most preferably, in a volume of 0.25 mL (250 μl). Therapeutically effective doses of the recombinant vector may be administered in one or more injections during the same visit.

Therapeutically effective doses of the recombinant vector should be administered suprachoroidally (e.g., by suprachoroidal injection) in a volume of 100 μl or less, for example, in a volume of 50-100 μl. Therapeutically effective doses of the recombinant vector should be administered to the outer surface of the sclera (e.g., by a posterior juxtascleral depot procedure) in a volume of 500 μl or less, for example, in a volume of 10-20 μl, 20-50 μl, 50-100 μl, 100-200 l, 200-300 μl, 300-400 μl, or 400-500 μl. Subretinal injection is a surgical procedure performed by trained retinal surgeons that involves a vitrectomy with the subject under local anesthesia, and subretinal injection of the gene therapy into the retina (see, e.g., Campochiaro et al., 2017, Hum Gen Ther 28(1):99-111, which is incorporated by reference herein in its entirety). In a specific embodiment, the subretinal administration is performed via the suprachoroidal space using a suprachoroidal catheter which injects drug into the subretinal space, such as a subretinal drug delivery device that comprises a catheter which can be inserted and tunneled through the suprachoroidal space to the posterior pole, where a small needle injects into the subretinal space (see, e.g., Baldassarre et al., 2017, Subretinal Delivery of Cells via the Suprachoroidal Space: Janssen Trial. In: Schwartz et al. (eds) Cellular Therapies for Retinal Disease, Springer, Cham; International Patent Application Publication No. WO 2016/040635 A1; each of which is incorporated by reference herein in its entirety). Suprachoroidal administration procedures involve administration of a drug to the suprachoroidal space of the eye, and are normally performed using a suprachoroidal drug delivery device such as a microinjector with a microneedle (see, e.g., Hariprasad, 2016, Retinal Physician 13: 20-23; Goldstein, 2014, Retina Today 9(5): 82-87; each of which is incorporated by reference herein in its entirety). The suprachoroidal drug delivery devices that can be used to deposit the expression vector in the suprachoroidal space according to the invention described herein include, but are not limited to, suprachoroidal drug delivery devices manufactured by Clearside® Biomedical, Inc. (see, for example, Hariprasad, 2016, Retinal Physician 13: 20-23) and MedOne suprachoroidal catheters. The subretinal drug delivery devices that can be used to deposit the expression vector in the subretinal space via the suprachoroidal space according to the invention described herein include, but are not limited to, subretinal drug delivery devices manufactured by Janssen Pharmaceuticals, Inc. (see, for example, International Patent Application Publication No. WO 2016/040635 A1). In a specific embodiment, administration to the outer surface of the sclera is performed by a juxtascleral drug delivery device comprising a cannula whose tip can be inserted and kept in direct apposition to the scleral surface. See Section 5.3.2 for more details of the different modes of administration. Suprachoroidal, subretinal, juxtascleral and/or intraretinal administration should result in delivery of the soluble transgene product to the retina, the vitreous humor, and/or the aqueous humor. The expression of the transgene product (e.g., the encoded anti-VEGF antibody) by retinal cells, e.g., rod, cone, retinal pigment epithelial, horizontal, bipolar, amacrine, ganglion, and/or Müller cells, results in delivery and maintenance of the transgene product in the retina, the vitreous humor, and/or the aqueous humor. In a specific embodiment, doses that maintain a concentration of the transgene product at a Cmin of at least 0.330 μg/mL in the Vitreous humour, or 0.110 μg/mL in the Aqueous humour (the anterior chamber of the eye) for three months are desired; thereafter, Vitreous Cmin concentrations of the transgene product ranging from 1.70 to 6.60 μg/mL, and/or Aqueous Cmin concentrations ranging from 0.567 to 2.20 μg/mL should be maintained. However, because the transgene product is continuously produced, maintenance of lower concentrations can be effective. The concentration of the transgene product can be measured in patient samples of the vitreous humour and/or aqueous from the anterior chamber of the treated eye. Alternatively, vitreous humour concentrations can be estimated and/or monitored by measuring the patient's serum concentrations of the transgene product—the ratio of systemic to vitreal exposure to the transgene product is about 1:90,000. (E.g., see, vitreous humor and serum concentrations of ranibizumab reported in Xu L, et al., 2013, Invest. Opthal. Vis. Sci. 54: 1616-1624, at p. 1621 and Table 5 at p. 1623, which is incorporated by reference herein in its entirety).

In certain embodiments, dosages are measured by genome copies per ml or the number of genome copies administered to the eye of the patient (e.g., by suprachoroidal injection (for example, via a suprachoroidal drug delivery device such as a microinjector with a microneedle), subretinal injection via the transvitreal approach (a surgical procedure), or subretinal administration via the suprachoroidal space). In certain embodiments, 2.4×10¹¹ genome copies per ml to 1×10¹³ genome copies per ml are administered. In a specific embodiment, 2.4×10¹¹ genome copies per ml to 5×10¹¹ genome copies per ml are administered. In another specific embodiment, 5×10¹¹ genome copies per ml to 1×10¹² genome copies per ml are administered. In another specific embodiment, 1×10¹² genome copies per ml to 5×10¹² genome copies per ml are administered. In another specific embodiment, 5×10¹² genome copies per ml to 1×10¹³ genome copies per ml are administered. In another specific embodiment, about 2.4×10¹¹ genome copies per ml are administered. In another specific embodiment, about 5×10¹¹ genome copies per ml are administered. In another specific embodiment, about 1×10¹² genome copies per ml are administered. In another specific embodiment, about 5×10¹² genome copies per ml are administered. In another specific embodiment, about 1×10¹³ genome copies per ml are administered. In certain embodiments, 1×10⁹ to 1×10¹² genome copies are administered. In specific embodiments, 3×10⁹ to 2.5×10¹¹ genome copies are administered. In specific embodiments, 1×10⁹ to 2.5×10¹¹ genome copies are administered. In specific embodiments, 1×10⁹ to 1×10¹¹ genome copies are administered. In specific embodiments, 1×10⁹ to 5×10⁹ genome copies are administered. In specific embodiments, 6×10⁹ to 3×10¹⁰ genome copies are administered. In specific embodiments, 4×10¹⁰ to 1×10¹¹ genome copies are administered. In specific embodiments, 2×10¹¹ to 1×10¹² genome copies are administered. In a specific embodiment, about 3×10⁹ genome copies are administered (which corresponds to about 1.2×10¹⁰ genome copies per ml in a volume of 250 □l). In another specific embodiment, about 1×10¹⁰ genome copies are administered (which corresponds to about 4×10¹⁰ genome copies per ml in a volume of 250 □l). In another specific embodiment, about 6×10¹⁰ genome copies are administered (which corresponds to about 2.4×10¹¹ genome copies per ml in a volume of 250 □l). In another specific embodiment, about 1.6×10¹¹ genome copies are administered (which corresponds to about 6.2×10¹¹ genome copies per ml in a volume of 250 □l). In another specific embodiment, about 1.6×10¹¹ genome copies are administered (which corresponds to about 6.4×10¹¹ genome copies per ml in a volume of 250 □l). In another specific embodiment, about 1.55×10¹¹ genome copies are administered (which corresponds to about 6.2×10¹¹ genome copies per ml in a volume of 250 □l). In another specific embodiment, about 2.5×10¹¹ genome copies (which corresponds to about 1.0×10¹² in a volume of 250 □l) are administered.

As used herein and unless otherwise specified, the term “about” means within plus or minus 10% of a given value or range.

The invention has several advantages over standard of care treatments that involve repeated ocular injections of high dose boluses of the VEGF inhibitor that dissipate over time resulting in peak and trough levels. Sustained expression of the transgene product antibody, as opposed to injecting an antibody repeatedly, allows for a more consistent levels of antibody to be present at the site of action, and is less risky and more convenient for patients, since fewer injections need to be made, resulting in fewer doctor visits. Consistent protein production may leads to better clinical outcomes as edema rebound in the retina is less likely to occur. Furthermore, antibodies expressed from transgenes are post-translationally modified in a different manner than those that are directly injected because of the different microenvironment present during and after translation. Without being bound by any particular theory, this results in antibodies that have different diffusion, bioactivity, distribution, affinity, pharmacokinetic, and immunogenicity characteristics, such that the antibodies delivered to the site of action are “biobetters” in comparison with directly injected antibodies.

In addition, antibodies expressed from transgenes in vivo are not likely to contain degradation products associated with antibodies produced by recombinant technologies, such as protein aggregation and protein oxidation. Aggregation is an issue associated with protein production and storage due to high protein concentration, surface interaction with manufacturing equipment and containers, and purification with certain buffer systems. These conditions, which promote aggregation, do not exist in transgene expression in gene therapy. Oxidation, such as methionine, tryptophan, and histidine oxidation, is also associated with protein production and storage, and is caused by stressed cell culture conditions, metal and air contact, and impurities in buffers and excipients. The proteins expressed from transgenes in vivo may also oxidize in a stressed condition. However, humans, and many other organisms, are equipped with an antioxidation defense system, which not only reduces the oxidation stress, but sometimes also repairs and/or reverses the oxidation. Thus, proteins produced in vivo are not likely to be in an oxidized form. Both aggregation and oxidation could affect the potency, pharmacokinetics (clearance), and immunogenicity.

Without being bound by theory, the methods and compositions provided herein are based, in part, on the following principles:
(i) Human retinal cells are secretory cells that possess the cellular machinery for post-translational processing of secreted proteins—including glycosylation and tyrosine-O-sulfation, a robust process in retinal cells. (See, e.g., Wang et al., 2013, Analytical Biochem. 427: 20-28 and Adamis et al., 1993, BBRC 193: 631-638 reporting the production of glycoproteins by retinal cells; and Kanan et al., 2009, Exp. Eye Res. 89: 559-567 and Kanan & Al-Ubaidi, 2015, Exp. Eye Res. 133: 126-131 reporting the production of tyrosine-sulfated glycoproteins secreted by retinal cells, each of which is incorporated by reference in its entirety for post-translational modifications made by human retinal cells).
(ii) Contrary to the state of the art understanding, anti-VEGF antigen-binding fragments, such as ranibizumab (and the Fab domain of full length anti-VEGF mAbs such as bevacizumab) do indeed possess N-linked glycosylation sites. For example, see FIG. 1 which identifies non-consensus asparaginal (“N”) glycosylation sites in the CH domain (TVSWN¹⁶⁵SGAL) and in the C_{L} domain (QSGN¹⁵⁸SQE), as well as glutamine (“Q”) residues that are glycosylation sites in the VH domain (Q¹¹⁵GT) and V_{L} domain (TFQ¹⁰⁰GT) of ranibizumab (and corresponding sites in the Fab of bevacizumab). (See, e.g., Valliere-Douglass et al., 2009, J. Biol. Chem. 284: 32493-32506, and Valliere-Douglass et al., 2010, J. Biol. Chem. 285: 16012-16022, each of which is incorporated by reference in its entirety for the identification of N-linked glycosylation sites in antibodies).
(iii) While such non-canonical sites usually result in low level glycosylation (e.g., about 1-5%) of the antibody population, the functional benefits may be significant in immunoprivileged organs, such as the eye (See, e.g., van de Bovenkamp et al., 2016, J. Immunol. 196:1435-1441). For example, Fab glycosylation may affect the stability, half-life, and binding characteristics of an antibody. To determine the effects of Fab glycosylation on the affinity of the antibody for its target, any technique known to one of skill in the art may be used, for example, enzyme linked immunosorbent assay (ELISA), or surface plasmon resonance (SPR). To determine the effects of Fab glycosylation on the half-life of the antibody, any technique known to one of skill in the art may be used, for example, by measurement of the levels of radioactivity in the blood or organs (e.g., the eye) in a subject to whom a radiolabelled antibody has been administered. To determine the effects of Fab glycosylation on the stability, for example, levels of aggregation or protein unfolding, of the antibody, any technique known to one of skill in the art may be used, for example, differential scanning calorimetry (DSC), high performance liquid chromatography (HPLC), e.g., size exclusion high performance liquid chromatography (SEC-HPLC), capillary electrophoresis, mass spectrometry, or turbidity measurement. Provided herein, the HuPTMFabVEGFi, e.g., HuGlyFabVEGFi, transgene results in production of a Fab which is 0.5%, 1%, 2%, 3%, 4%, 5%, 6%, 7%, 8%, 9%, or 10% or more glycosylated at non-canonical sites. In certain embodiments, 0.5%, 1%, 2%, 3%, 4%, 5%, 6%, 7%, 8%, 9%, or 10% or more Fabs from a population of Fabs are glycosylated at non-canonical sites. In certain embodiments, 0.5%, 1%, 2%, 3%, 4%, 5%, 6%, 7%, 8%, 9%, or 10% or more non-canonical sites are glycosylated. In certain embodiments, the glycosylation of the Fab at these non-canonical sites is 25%, 50%, 100%, 200%, 300%, 400%, 500%, or more greater than the amount of glycosylation of these non-canonical sites in a Fab produced in HEK293 cells.
(iv) In addition to the glycosylation sites, anti-VEGF Fabs such as ranibizumab (and the Fab of bevacizumab) contain tyrosine (“Y”) sulfation sites in or near the CDRs; see FIG. 1 which identifies tyrosine-O-sulfation sites in the VH (EDTAVY⁹⁴Y⁹⁵) and V_{L} (EDFATY⁸⁶) domains of ranibizumab (and corresponding sites in the Fab of bevacizumab). (See, e.g., Yang et al., 2015, Molecules 20:2138-2164, esp. at p. 2154 which is incorporated by reference in its entirety for the analysis of amino acids surrounding tyrosine residues subjected to protein tyrosine sulfation. The “rules” can be summarized as follows: Y residues with E or D within +5 to −5 position of Y, and where position −1 of Y is a neutral or acidic charged amino acid—but not a basic amino acid, e.g., R, K, or H that abolishes sulfation). Human IgG antibodies can manifest a number of other post-translational modifications, such as N-terminal modifications, C-terminal modifications, degradation or oxidation of amino acid residues, cysteine related variants, and glycation (See, e.g., Liu et al., 2014, mAbs 6(5):1145-1154).
(v) Glycosylation of anti-VEGF Fabs, such as ranibizumab or the Fab fragment of bevacizumab by human retinal cells will result in the addition of glycans that can improve stability, half-life and reduce unwanted aggregation and/or immunogenicity of the transgene product. (See, e.g., Bovenkamp et al., 2016, J. Immunol. 196: 1435-1441 for a review of the emerging importance of Fab glycosylation). Significantly, glycans that can be added to HuPTMFabVEGFi, e.g., HuGlyFabVEGFi, provided herein, are highly processed complex-type biantennary N-glycans that contain 2,6-sialic acid (e.g., see FIG. 2 depicting the glycans that may be incorporated into HuPTMFabVEGFi, e.g., HuGlyFabVEGFi) and bisecting GlcNAc, but not NGNA (N-Glycolylneuraminic acid, Neu5Gc). Such glycans are not present in ranibizumab (which is made in E. coli and is not glycosylated at all) or in bevacizumab (which is made in CHO cells that do not have the 2,6-sialyltransferase required to make this post-translational modification, nor do CHO cells product bisecting GlcNAc, although they do add Neu5Gc (NGNA) as sialic acid not typical (and potentially immunogenic) to humans instead of Neu5Ac (NANA)). See, e.g., Dumont et al., 2015, Crit. Rev. Biotechnol. (Early Online, published online Sep. 18, 2015, pp. 1-13 at p. 5). Moreover, CHO cells can also produce an immunogenic glycan, the α-Gal antigen, which reacts with anti-α-Gal antibodies present in most individuals, and at high concentrations can trigger anaphylaxis. See, e.g., Bosques, 2010, Nat Biotech 28: 1153-1156. The human glycosylation pattern of the HuPTMFabVEGFi, e.g., HuGlyFabVEGFi, provided herein, should reduce immunogenicity of the transgene product and improve efficacy.
(vi)Tyrosine-sulfation of anti-VEGF Fabs, such as ranibizumab or the Fab fragment of bevacizumab—a robust post-translational process in human retinal cells—could result in transgene products with increased avidity for VEGF. Indeed, tyrosine-sulfation of the Fab of therapeutic antibodies against other targets has been shown to dramatically increase avidity for antigen and activity. (See, e.g., Loos et al., 2015, PNAS 112: 12675-12680, and Choe et al., 2003, Cell 114: 161-170). Such post-translational modifications are not present on ranibizumab (which is made in E. coli a host that does not possess the enzymes required for tyrosine-sulfation), and at best is under-represented in bevacizumab
a CHO cell product. Unlike human retinal cells, CHO cells are not secretory cells and have a limited capacity for post-translational tyrosine-sulfation. (See, e.g., Mikkelsen & Ezban, 1991, Biochemistry 30: 1533-1537, esp. discussion at p. 1537).

For the foregoing reasons, the production of HuPTMFabVEGFi, e.g., HuGlyFabVEGFi, should result in a “biobetter” molecule for the treatment of nAMD (wet AMD), dry AMD, retinal vein occlusion (RVO), diabetic macular edema (DME), or diabetic retinopathy (DR) accomplished via gene therapy—e.g., by administering a viral vector or other DNA expression construct encoding HuPTMFabVEGFi, e.g., HuGlyFabVEGFi, to the suprachoroidal space, subretinal space, or the outer surface of the sclera in the eye(s) of patients (human subjects) diagnosed with nAMD (wet AMD), dry AMD, retinal vein occlusion (RVO), diabetic macular edema (DME), or diabetic retinopathy (DR) (e.g., by suprachoroidal injection (for example, via a suprachoroidal drug delivery device such as a microinjector with a microneedle), subretinal injection via the transvitreal approach (a surgical procedure), subretinal administration via the suprachoroidal space, or a posterior juxtascleral depot procedure), to create a permanent depot in the eye that continuously supplies the fully-human post-translationally modified, e.g., human-glycosylated, sulfated transgene product produced by transduced retinal cells. The cDNA construct for the FabVEGFi should include a signal peptide that ensures proper co- and post-translational processing (glycosylation and protein sulfation) by the transduced retinal cells. Such signal sequences used by retinal cells may include but are not limited to:

(SEQ ID NO: 5)
MNFLLSWVHW SLALLLYLHH AKWSQA (VEGF-A signal peptide)
(SEQ ID NO: 6)
MERAAPSRRV PLPLLLLGGL ALLAAGVDA (Fibulin-1 signal peptide)
(SEQ ID NO: 7)
MAPLRPLLIL ALLAWVALA (Vitronectin signal peptide)
(SEQ ID NO: 8)
MRLLAKIICLMLWAICVA (Complement Factor H signal peptide)
(SEQ ID NO: 9)
MRLLAFLSLL ALVLQETGT (Opticin signal peptide)
(SEQ ID NO: 22)
MKWVTFISLLFLFSSAYS (Albumin signal peptide)
(SEQ ID NO: 23)
MAFLWLLSCWALLGTTFG (Chymotrypsinogen signal peptide)
(SEQ ID NO: 24)
MYRMQLLSCIALILALVTNS (Interleukin-2 signal peptide)
(SEQ ID NO: 25)
MNLLLILTFVAAAVA (Trypsinogen-2 signal peptide)
(SEQ ID NO: 52)
MYRMQLLLLIALSLALVTNS (mutant Interleukin-2 signal peptide).
See, e.g., Stern et al., 2007, Trends Cell. Mol. Biol.,
2:1-17 and Dalton & Barton, 2014, Protein Sci, 23: 517-525,
each of which is incorporated by reference herein in its
entirety for the signal peptides that can be used.

As an alternative, or an additional treatment to gene therapy, the HuPTMFabVEGFi product, e.g., HuGlyFabVEGFi glycoprotein, can be produced in human cell lines by recombinant DNA technology, and administered to patients diagnosed with nAMD (wet AMD), dry AMD, retinal vein occlusion (RVO), diabetic macular edema (DME), or diabetic retinopathy (DR) by intravitreal or subretinal injection. The HuPTMFabVEGFi product, e.g., glycoprotein, may also be administered to patients with nAMD (wet AMD), dry AMD, retinal vein occlusion (RVO), diabetic macular edema (DME), or diabetic retinopathy (DR). Human cell lines that can be used for such recombinant glycoprotein production include but are not limited to human embryonic kidney 293 cells (HEK293), fibrosarcoma HT-1080, HKB-11, CAP, HuH-7, and retinal cell lines, PER.C6, or RPE to name a few (e.g., see Dumont et al., 2015, Crit. Rev. Biotechnol. (Early Online, published online Sep. 18, 2015, pp. 1-13) “Human cell lines for biopharmaceutical manufacturing: history, status, and future perspectives” which is incorporated by reference in its entirety for a review of the human cell lines that could be used for the recombinant production of the HuPTMFabVEGFi product, e.g., HuGlyFabVEGFi glycoprotein). To ensure complete glycosylation, especially sialylation, and tyrosine-sulfation, the cell line used for production can be enhanced by engineering the host cells to co-express α-2,6-sialyltransferase (or both α-2,3- and α-2,6-sialyltransferases) and/or TPST-1 and TPST-2 enzymes responsible for tyrosine-O-sulfation in retinal cells.

Combinations of delivery of the HuPTMFabVEGFi, e.g., HuGlyFabVEGFi, to the eye/retina accompanied by delivery of other available treatments are encompassed by the methods provided herein. The additional treatments may be administered before, concurrently or subsequent to the gene therapy treatment. Available treatments for nAMD (wet AMD), dry AMD, retinal vein occlusion (RVO), diabetic macular edema (DME), or diabetic retinopathy (DR) that could be combined with the gene therapy provided herein include but are not limited to laser photocoagulation, photodynamic therapy with verteporfin, and intravitreal (IVT) injections with anti-VEGF agents, including but not limited to pegaptanib, ranibizumab, aflibercept, or bevacizumab. Additional treatments with anti-VEGF agents, such as biologics, may be referred to as “rescue” therapy.

Unlike small molecule drugs, biologics usually comprise a mixture of many variants with different modifications or forms that have a different potency, pharmacokinetics, and safety profile. It is not essential that every molecule produced either in the gene therapy or protein therapy approach be fully glycosylated and sulfated. Rather, the population of glycoproteins produced should have sufficient glycosylation (from about 1% to about 10% of the population), including 2,6-sialylation, and sulfation to demonstrate efficacy. The goal of gene therapy treatment provided herein is to slow or arrest the progression of retinal degeneration, and to slow or prevent loss of vision with minimal intervention/invasive procedures. Efficacy may be monitored by measuring BCVA (Best-Corrected Visual Acuity), intraocular pressure, slit lamp biomicroscopy, indirect ophthalmoscopy, SD-OCT (SD-Optical Coherence Tomography), electroretinography (ERG). Signs of vision loss, infection, inflammation and other safety events, including retinal detachment may also be monitored. Retinal thickness may be monitored to determine efficacy of the treatments provided herein. Without being bound by any particular theory, thickness of the retina may be used as a clinical readout, wherein the greater reduction in retinal thickness or the longer period of time before thickening of the retina, the more efficacious the treatment. Retinal thickness may be determined, for example, by SD-OCT. SD-OCT is a three-dimensional imaging technology which uses low-coherence interferometry to determine the echo time delay and magnitude of backscattered light reflected off an object of interest. OCT can be used to scan the layers of a tissue sample (e.g., the retina) with 3 to 15 m axial resolution, and SD-OCT improves axial resolution and scan speed over previous forms of the technology (Schuman, 2008, Trans. Am. Opthamol. Soc. 106:426-458). Retinal function may be determined, for example, by ERG. ERG is a non-invasive electrophysiologic test of retinal function, approved by the FDA for use in humans, which examines the light sensitive cells of the eye (the rods and cones), and their connecting ganglion cells, in particular, their response to a flash stimulation.

In preferred embodiments, the antigen-binding fragments do not contain detectable NeuGc and/or α-Gal. The phrase “detectable NeuGc and/or α-Gal” used herein means NeuGc and/or α-Gal moieties detectable by standard assay methods known in the art. For example, NeuGc may be detected by HPLC according to Hara et al., 1989, “Highly Sensitive Determination of N-Acetyl- and N-Glycolylneuraminic Acids in Human Serum and Urine and Rat Serum by Reversed-Phase Liquid Chromatography with Fluorescence Detection.” J. Chromatogr., B: Biomed. 377: 111-119, which is hereby incorporated by reference for the method of detecting NeuGc. Alternatively, NeuGc may be detected by mass spectrometry. The α-Gal may be detected using an ELISA, see, for example, Galili et al., 1998, “A sensitive assay for measuring alphα-Gal epitope expression on cells by a monoclonal anti-Gal antibody.” Transplantation. 65(8):1129-32, or by mass spectrometry, see, for example, Ayoub et al., 2013, “Correct primary structure assessment and extensive glyco-profiling of cetuximab by a combination of intact, middle-up, middle-down and bottom-up ESI and MALDI mass spectrometry techniques.” Landes Bioscience. 5(5): 699-710. See also the references cited in Platts-Mills et al., 2015, “Anaphylaxis to the Carbohydrate Side-Chain Alpha-gal” Immunol Allergy Clin North Am. 35(2): 247-260.

In certain aspects, also provided herein are anti-VEGF antigen-binding fragments (i.e., antigen-binding fragments that immunospecifically binds to VEGF) comprising light chain CDRs 1-3 of SEQ ID NOs: 14-16 and heavy chain CDRs 1-3 of SEQ ID NOs: 20, 18, and 21, wherein the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the second amino acid residue of the light chain CDR3 (i.e., the second Q in QQYSTVPWTF (SEQ ID NO. 16)) is not acetylated. In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) is not acetylated. The anti-VEGF antigen-binding fragments provided herein can be used in any method according to the invention described herein. In a preferred embodiment, the chemical modification(s) or lack of chemical modification(s) (as the case may be) described herein is determined by mass spectrometry.

In certain aspects, also provided herein are anti-VEGF antigen-binding fragments comprising light chain CDRs 1-3 of SEQ ID NOs: 14-16 and heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) is not acetylated. In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) is not acetylated. The anti-VEGF antigen-binding fragments provided herein can be used in any method according to the invention described herein. In a preferred embodiment, the chemical modification(s) or lack of chemical modification(s) (as the case may be) described herein is determined by mass spectrometry.

In certain aspects, also provided herein are anti-VEGF antigen-binding fragments comprising light chain CDRs 1-3 of SEQ ID NOs: 14-16 and heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein: (1) the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu); and (2) the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) is not acetylated, and the second amino acid residue of the light chain CDR3 (i.e., the second Q in QQYSTVPWTF (SEQ ID NO. 16)) is not acetylated. In a specific embodiment, the antigen-binding fragment comprises light chain CDRs 1-3 of SEQ ID NOs: 14-16 and heavy chain CDRs 1-3 of SEQ ID NOs: 20, 18, and 21, wherein: (1) the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) is not acetylated; and (2) the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) is not acetylated. The anti-VEGF antigen-binding fragments provided herein can be used in any method according to the invention described herein. In a preferred embodiment, the chemical modification(s) or lack of chemical modification(s) (as the case may be) described herein is determined by mass spectrometry.

Another contemplated administration route is subretinal administration via the suprachoroidal space, using a subretinal drug delivery device that has a catheter inserted and tunneled through the suprachoroidal space to inject into the subretinal space toward the posterior pole, where a small needle injects into the subretinal space. This route of administration allows the vitreous to remain intact and thus, there are fewer complication risks (less risk of gene therapy egress, and complications such as retinal detachments and macular holes), and without a vitrectomy, the resulting bleb may spread more diffusely allowing more of the surface area of the retina to be transduced with a smaller volume. The risk of induced cataract following this procedure is minimized, which is desirable for younger patients. Moreover, this procedure can deliver bleb under the fovea more safely than the standard transvitreal approach, which is desirable for patients with inherited retinal diseases effecting central vision where the target cells for transduction are in the macula. This procedure is also favorable for patients that have neutralizing antibodies (Nabs) to AAVs present in the systemic circulation which may impact other routes of delivery. Additionally, this method has shown to create blebs with less egress out the retinotomy site than the standard transvitreal approach.

Juxtascleral administration provides an additional administration route which avoids the risk of intraocular infection and retinal detachment, side effects commonly associated with injecting therapeutic agents directly into the eye.

In certain embodiments, described here is a kit comprising one or more containers and instructions for use, wherein the one or more containers comprise the pharmaceutical composition. In certain embodiments, at least one of the one or more containers is made from hydrophobically-coated glass vial. In certain embodiments, at least one of the one or more containers is made from Daikyo Crystal Zenith® (CZ) vial. In certain embodiments, at least one of the one or more containers is made from TopLyo coated vial. In certain embodiments, at least one of the one or more containers is made from Cyclo Olefin Polymer (COP).

In another aspect provided herein are single unit dosage forms comprising 3.2×10¹¹ GC/mL, 6.5×10¹¹GC/mL, 2.5×10¹²GC/mL, 3×10¹³ GC/mL of Construct II, 0.2 g/L potassium chloride, 0.2 g/L potassium phosphate monobasic, 8.01 g/L sodium chloride, 1.15 g/L sodium phosphate dibasic anhydrous, pH 7.4 and 0.001% P188 in a volume of at least about 0.5 mL, at least about 0.8 mL, about 0.6 mL, about 0.95 mL in a Cyclo Olefin Polymer (COP) vial. In some embodiments, the single unit dosage form is capable of being stored at 4° C. for 1 weeks, about 2 weeks, about 3 weeks, about 4 weeks, about 1 month, about 2 months. In some embodiments, the single unit dosage form is capable of being stored at −80° C. for about 6 months, about 7 months, about 8 months, about 9 months, about 10 months, about 11 months, about 12 months, about 15 months, about 18 months, about 24 months. In some embodiments, the single unit dosage form is capable of being stored at 4° C. for 1 weeks, about 2 weeks, about 3 weeks, about 4 weeks, about 1 month, about 2 months after having previously been stored at −80° C. for about 6 months, about 7 months, about 8 months, about 9 months, about 10 months, about 11 months, about 12 months, about 15 months, about 18 months, about 24 months. In some embodiments, the vector genome concentration of the Construct II after being stored at −80° C., −20° C. or 4° C. for a period of time is at least 70%, 75%, 80%, 85%, 90%, 95%, 98%, or 99% of the vector genome concentration of the Construct II before being stored at −80° C., −20° C. or 4° C. for said period of time. In some embodiments, the in vitro potency of the Construct II after being stored at −80° C., −20° C. or 4° C. for a period of time is at least 70%, 75%, 80%, 85%, 90%, 95%, 98%, or 99% of the in vitro potency of the Construct II before being stored at −80° C., −20° C. or 4° C. for said period of time. In some embodiments, the size distribution of the Construct II after being stored at −80° C., −20° C. or 4° C. for a period of time is at least 70%, 75%, 80%, 85%, 90%, 95%, 98%, or 99% identical to the size distribution of the Construct II before being stored at −80° C., −20° C. or 4° C. for said period of time. In some embodiments, the period of time is about 1 month, about 2 months, about 3 months, about 4 months, about 5 months, about 6 months, about 7 months, about 8 months, about 9 months, about 10 months, about 11 months, about 12 months, about 15 months, about 18 months, or about 24 months.

In another aspect provided herein are single unit dosage forms comprising 3.2×10¹¹ GC/mL, 6.5×10¹¹ GC/mL, 2.5×10¹² GC/mL, 3×10¹³ GC/mL of Construct II, 0.2 g/L potassium chloride, 0.2 g/L potassium phosphate monobasic, 8.01 g/L sodium chloride, 1.15 g/L sodium phosphate dibasic anhydrous, pH 7.4, 4% sucrose and 0.001% P188 in a volume of at least about 0.5 mL, at least about 0.8 mL, about 0.6 mL, about 0.95 mL in a COP vial. In some embodiments, the single unit dosage form is capable of being stored at 4° C. for 1 weeks, about 2 weeks, about 3 weeks, about 4 weeks, about 1 month, about 2 months. In some embodiments, the single unit dosage form is capable of being stored at −80° C. for about 6 months, about 7 months, about 8 months, about 9 months, about 10 months, about 11 months, about 12 months, about 15 months, about 18 months, about 24 months. In some embodiments, the single unit dosage form is capable of being stored at 4° C. for 1 weeks, about 2 weeks, about 3 weeks, about 4 weeks, about 1 month, about 2 months after having previously been stored at −80° C. for about 6 months, about 7 months, about 8 months, about 9 months, about 10 months, about 11 months, about 12 months, about 15 months, about 18 months, about 24 months. In some embodiments, the vector genome concentration of the Construct II after being stored at −80° C., −20° C. or 4° C. for a period of time is at least 70%, 75%, 80%, 85%, 90%, 95%, 98%, or 99% of the vector genome concentration of the Construct II before being stored at −80° C., −20° C. or 4° C. for said period of time. In some embodiments, the in vitro potency of the Construct II after being stored at −80° C.,-20° C. or 4° C. for a period of time is at least 70%, 75%, 80%, 85%, 90%, 95%, 98%, or 99% of the in vitro potency of the Construct II before being stored at −80° C., −20° C. or 4° C. for said period of time. In some embodiments, the size distribution of the Construct II after being stored at-80° C., −20° C. or 4° C. for a period of time is at least 70%, 75%, 80%, 85%, 90%, 95%, 98%, or 99% identical to the size distribution of the Construct II before being stored at −80° C., −20° C. or 4° C. for said period of time. In some embodiments, the period of time is about 1 month, about 2 months, about 3 months, about 4 months, about 5 months, about 6 months, about 7 months, about 8 months, about 9 months, about 10 months, about 11 months, about 12 months, about 15 months, about 18 months, or about 24 months.

In another aspect, provided herein is a pre-filled syringe containing a single unit dosage form provided herein. In another aspect, provided herein is a kit comprising the pre-filled syringe containing the single unit dosage form provided herein.

### 2.1 Illustrative Embodiments

1. A pharmaceutical composition comprising:
   (a) a recombinant adeno-associated virus (AAV),
   (b) potassium chloride,
   (c) potassium phosphate monobasic,
   (d) sodium chloride,
   (e) sodium phosphate dibasic anhydrous,
   (f) sucrose, and
   (e) poloxamer 188, polysorbate 20, or polysorbate 80.
2. The pharmaceutical composition of paragraph 1, wherein the recombinant AAV comprises components from one or more adeno-associated virus serotypes selected from the group consisting of AAV1, AAV2, AAV2tYF, AAV3, AAV4, AAV5, AAV6, AAV7, AAV8, AAV9, AAV10, AAV11, AAVrh10, AAV.rh20, AAV.rh39, AAV.Rh74, AAV.RHM4-1, AAV.hu37, AAV.Anc80, AAV.Anc80L65, rAAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16.
3. The pharmaceutical composition of any one of paragraphs 1-2, wherein the recombinant AAV is AAV8.
4. The pharmaceutical composition of any one of paragraphs 1-2, wherein the recombinant AAV is AAV9.
5. The pharmaceutical composition of any one of paragraphs 1-4, wherein the pharmaceutical composition further comprises one or more amino acids.
6. The pharmaceutical composition of any one of paragraphs 1-5, wherein the ionic strength of the pharmaceutical composition is in a range from about 60 mM to about 115 mM.
7. The pharmaceutical composition of paragraph 4, wherein the ionic strength of the pharmaceutical composition is in a range from about 30 mM to about 100 mM.
8. The pharmaceutical composition of any one of paragraphs 1-3, wherein the pharmaceutical composition comprises
   (a) potassium chloride at a concentration of 0.2 g/L,
   (b) potassium phosphate monobasic at a concentration of 0.2 g/L,
   (c) sodium chloride at a concentration of 5.84 g/L, and
   (d) sodium phosphate dibasic anhydrous at a concentration of 1.15 g/L.
9. The pharmaceutical composition of any one of paragraphs 1-8, wherein the pharmaceutical composition comprises sucrose at a concentration in a range from 3% (weight/volume, 30 g/L) to 18% (weight/volume, 180 g/L).
10. The pharmaceutical composition of any one of paragraphs 1-8, wherein the pharmaceutical composition comprises sucrose at a concentration of 4% (weight/volume, 40 g/L).
11. The pharmaceutical composition of any one of paragraphs 1-10, wherein the pharmaceutical composition comprises poloxamer 188, polysorbate 20, or polysorbate 80; and wherein the poloxamer 188, polysorbate 20, or polysorbate 80 is at a concentration in a range from 0.0005% (weight/volume, 0.005 g/L) to 0.05% (weight/volume, 0.5 g/L).
12. The pharmaceutical composition of any one of paragraphs 1-10, wherein the pharmaceutical composition comprises poloxamer 188, polysorbate 20, or polysorbate 80; and wherein the poloxamer 188, polysorbate 20, or polysorbate 80 is at a concentration of 0.001% (weight/volume, 0.01 g/L).
13. The pharmaceutical composition of any one of paragraphs 48-54, wherein the pH of the pharmaceutical composition is in a range from about 6.0 to about 9.0.
14. The pharmaceutical composition of any one of paragraphs 1-12, wherein the pH of the pharmaceutical composition is about 7.4.
15. The pharmaceutical composition of any one of paragraphs 1-14, wherein the osmolality of the pharmaceutical composition is in a range from about 200 mOsm/L to about 660 mOsm/L.
16. The pharmaceutical composition of any one of paragraphs 1-15, wherein the pharmaceutical composition is in a hydrophobically-coated glass vial.
17. The pharmaceutical composition of any one of paragraphs 1-15, wherein the pharmaceutical composition is in a Cyclo Olefin Polymer (COP) vial.
18. The pharmaceutical composition of any one of paragraphs 1-15, wherein the pharmaceutical composition is in a Daikyo Crystal Zenith® (CZ) vial.
19. The pharmaceutical composition of any one of paragraphs 1-15, wherein the pharmaceutical composition is in a TopLyo coated vial.
20. The pharmaceutical composition of any one of paragraphs 1-19, wherein the pharmaceutical composition consists of:
   (a) the recombinant AAV,
   (b) potassium chloride at a concentration of 0.2 g/L,
   (c) potassium phosphate monobasic at a concentration of 0.2 g/L,
   (d) sodium chloride at a concentration of 5.84 g/L,
   (e) sodium phosphate dibasic anhydrous at a concentration of 1.15 g/L,
   (f) sucrose at a concentration of 4% weight/volume (40 g/L),
   (g) poloxamer 188, polysorbate 20, or polysorbate 80 at a concentration of 0.001% weight/volume (0.01 g/L), and
   (h) water, and wherein the recombinant AAV is AAV8.
21. The pharmaceutical composition of any one of paragraphs 1-20, wherein the vector genome concentration (VGC) of the pharmaceutical composition is about 3×10⁹ GC/mL, about 1×10¹⁰ GC/mL, about 1.2×10¹⁰ GC/mL, about 1.6×10¹⁰ GC/mL, about 4×10¹⁰ GC/mL, about 6×10¹⁰ GC/mL, about 2×10¹¹ GC/mL, about 2.4×10¹¹ GC/mL, about 2.5×10¹¹ GC/mL, about 3×10¹¹ GC/mL, about 3.2×10¹¹ GC/mL, about 6.2×10¹¹ GC/mL, about 6.5×10¹¹ GC/mL, about 1×10¹² GC/mL, about 3×10¹² GC/mL, about 2×10¹³ GC/mL or about 3×10¹³ GC/mL
22. The pharmaceutical composition of any one of paragraphs 1-21, wherein the recombinant AAV is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more stable to freeze/thaw cycles than the same recombinant AAV in a reference pharmaceutical composition.
23. The pharmaceutical composition of any one of paragraphs 1-22, wherein the recombinant AAV is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more stable than the same recombinant AAV in a reference pharmaceutical composition when stored at −20° C. for a period of time, wherein the period of time is about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, or about 24 months.
24. The pharmaceutical composition of any one of paragraphs 1-23, wherein the recombinant AAV is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more stable than the same recombinant AAV in a reference pharmaceutical composition when stored at −80° C. for a period of time, wherein the period of time is about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, or about 24 months.
25. The pharmaceutical composition of any one of paragraphs 1-24, wherein the recombinant AAV is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more stable than the same recombinant AAV in a reference pharmaceutical composition when stored at room temperate for a period of time, wherein the period of time is about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, or about 24 months.
26. The pharmaceutical composition of any one of paragraphs 1-25, wherein, the recombinant AAV is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more stable than the same recombinant AAV in a reference pharmaceutical composition when (i) stored at −80° C. for a first period of time; (ii) subsequently thawed; and (iii) after thawing, stored at 4° C. for a second period of time.
27. The pharmaceutical composition of paragraph 26, wherein the first period of time is about 6 months, about 7 months, about 8 months, about 9 months, about 10 months, about 11 months, about 12 months, about 15 months, about 18 months, or about 24 months.
28. The pharmaceutical composition of paragraph 26, wherein the second period of time is about 1 week, about 2 weeks, about 3 weeks, about 4 weeks, about 1 month, about 2 months.
29. The pharmaceutical composition of any one of paragraphs 1-22, wherein the vector genome concentration of the recombinant AAV after being stored at −80° C. for a period of time is at least 70%, 75%, 80%, 85%, 90%, 95%, 98%, or 99% of the vector genome concentration of the recombinant AAV before being stored at −80° C. for said period of time.
30. The pharmaceutical composition of any one of paragraphs 1-22, wherein the vector genome concentration of the recombinant AAV after being stored at −20° C. for a period of time is at least 70%, 75%, 80%, 85%, 90%, 95%, 98%, or 99% of the vector genome concentration of the recombinant AAV before being stored at −20° C. for said period of time.
31. The pharmaceutical composition of any one of paragraphs 1-22, wherein the vector genome concentration of the recombinant AAV after being stored at 4° C. for a period of time is at least 70%, 75%, 80%, 85%, 90%, 95%, 98%, or 99% of the vector genome concentration of the recombinant AAV before being stored at 4° C. for said period of time.
32. The pharmaceutical composition of any one of paragraphs 1-22, wherein in the vitro potency of the recombinant AAV after being stored at −80° C. for a period of time is at least 70%, 75%, 80%, 85%, 90%, 95%, 98%, or 99% of the in vitro potency of the recombinant AAV before being stored at −80° C. for said period of time.
33. The pharmaceutical composition of any one of paragraphs 1-22, wherein the in vitro potency of the recombinant AAV after being stored at −20° C. for a period of time is at least 70%, 75%, 80%, 85%, 90%, 95%, 98%, or 99% of the in vitro potency of the recombinant AAV before being stored at −20° C. for said period of time.
34. The pharmaceutical composition of any one of paragraphs 1-22, wherein the in vitro potency of the recombinant AAV after being stored at 4° C. for a period of time is at least 70%, 75%, 80%, 85%, 90%, 95%, 98%, or 99% of the in vitro potency of the recombinant AAV before being stored at 4° C. for said period of time.
35. The pharmaceutical composition of any one of paragraphs 1-22, wherein the size distribution of the recombinant AAV after being stored at −80° C. for a period of time is at least 70%, 75%, 80%, 85%, 90%, 95%, 98%, or 99% identical to the size distribution of the recombinant AAV before being stored at −80° C. for said period of time.
36. The pharmaceutical composition of any one of paragraphs 1-22, wherein the size distribution of the recombinant AAV after being stored at −20° C. for a period of time is at least 70%, 75%, 80%, 85%, 90%, 95%, 98%, or 99% identical to the size distribution of the recombinant AAV before being stored at −20° C. for said period of time.
37. The pharmaceutical composition of any one of paragraphs 1-22, wherein the size distribution of the recombinant AAV after being stored at 4° C. for a period of time is at least 70%, 75%, 80%, 85%, 90%, 95%, 98%, or 99% identical to the size distribution of the recombinant AAV before being stored at 4° C. for said period of time.
38. The pharmaceutical composition of any one of paragraphs 29 to 37, wherein the period of time is about 1 month, about 2 months, about 3 months, about 4 months, about 5 months, about 6 months, about 7 months, about 8 months, about 9 months, about 10 months, about 11 months, about 12 months, about 15 months, about 18 months, or about 24 months.
39. The pharmaceutical composition of any one of paragraphs 1-21, wherein the stability of recombinant AAV is determined by the infectivity of recombinant AAV.
40. The pharmaceutical composition of any one of paragraphs 1-21, wherein the stability of recombinant AAV is determined by the levels of aggregation of recombinant AAV.
41. The pharmaceutical composition of any one of paragraphs 1-21, wherein the stability of recombinant AAV is determined by the levels of free DNA released by the recombinant AAV particles.
42. The pharmaceutical composition of any one of paragraphs 1-41, wherein the pharmaceutical composition is a liquid composition.
43. The pharmaceutical composition of any one of paragraphs 1-41, wherein the pharmaceutical composition is a frozen composition.
44. The pharmaceutical composition of any one of paragraphs 1-41, wherein the pharmaceutical composition is a lyophilized composition or a reconstituted lyophilized composition.
45. The pharmaceutical composition of any one of paragraphs 1-44, the pharmaceutical composition has a property that is suitable for intravenous administration, subcutaneous administration, intramuscular injection, suprachoroidal injection, subretinal injection via transvitreal approach, subretinal administration via the suprachoroidal space, or a posterior juxtascleral depot procedure.
46. The pharmaceutical composition of any one of paragraphs 1-44, the pharmaceutical composition has a desired density that is suitable for intravenous administration, subcutaneous administration, intramuscular injection, suprachoroidal injection, subretinal injection via transvitreal approach, subretinal administration via the suprachoroidal space, or a posterior juxtascleral depot procedure.
47. The pharmaceutical composition of any one of paragraphs 1-44, the pharmaceutical composition has a desired osmolality that is suitable for intravenous administration, subcutaneous administration, intramuscular injection, suprachoroidal injection, subretinal injection via transvitreal approach, subretinal administration via the suprachoroidal space, or a posterior juxtascleral depot procedure.
48. The pharmaceutical composition of paragraph 47, wherein the osmolality is 160-230 mOsm/kg H₂O.
49. The pharmaceutical composition of paragraph 47, wherein the osmolality is less than 600 mOsm/kg H₂O.
50. The pharmaceutical composition of any one of paragraphs 1-44, the pharmaceutical composition has a desired viscosity that is suitable for intravenous administration, subcutaneous administration, intramuscular injection, suprachoroidal injection, subretinal injection via transvitreal approach, subretinal administration via the suprachoroidal space, or a posterior juxtascleral depot procedure.
51. The pharmaceutical composition of any one of paragraphs 1-44, the pharmaceutical composition is suitable for administration to the eye.
52. The pharmaceutical composition of paragraph 45, the pharmaceutical composition is suitable for suprachoroidal injection, subretinal injection via transvitreal approach, subretinal administration via the suprachoroidal space, or a posterior juxtascleral depot procedure.
53. The pharmaceutical composition of any one of paragraphs 1-52, wherein the pharmaceutical composition is capable of being stored at 4° C. for 1 weeks, about 2 weeks, about 3 weeks, about 4 weeks, about 1 month, about 2 months after having previously been stored at −80° C. for about 6 months, about 7 months, about 8 months, about 9 months, about 10 months, about 11 months, about 12 months, about 15 months, about 18 months, about 24 months.
54. A method of treating or preventing a disease in a subject, comprising administering to the subject the pharmaceutical composition of any one of paragraphs 1-53.
55. A method of treating a subject diagnosed with nAMD (wet AMD), dry AMD, retinal vein occlusion (RVO), diabetic macular edema (DME), or diabetic retinopathy (DR) comprising administering to the subject the pharmaceutical composition of any one of any one of paragraphs 1-53.
56. A method of treating a subject diagnosed with mucopolysaccharidosis type IVA (MPS IVA), mucopolysaccharidosis type I (MPS I), mucopolysaccharidosis type II (MPS II), familial hypercholesterolemia (FH), homozygous familial hypercholesterolemia (HoFH), coronary artery disease, cerebrovascular disease, Duchenne muscular dystrophy, Limb Girdle muscular dystrophy, Becker muscular dystrophy and sporadic inclusion body myositis, or kallikrein-related disease comprising administering to the subject the pharmaceutical composition of any one of paragraphs 1-53.
57. The method of any one of paragraphs 54-56, wherein the pharmaceutical composition is administered by intravenous administration, subcutaneous administration, intramuscular injection, suprachoroidal injection, subretinal injection via transvitreal approach, subretinal administration via the suprachoroidal space, or a posterior juxtascleral depot procedure.
58. The method of any one of paragraphs 54-57, wherein the subject is a human subject.
59. A method of treating a subject diagnosed with nAMD (wet AMD), dry AMD, retinal vein occlusion (RVO), diabetic macular edema (DME), or diabetic retinopathy (DR), the method comprising preparing the pharmaceutical composition of any one of paragraphs 1 to 53, storing the pharmaceutical composition at −80° C. for a first period of time; (ii) thawing the pharmaceutical composition; and (iii) after thawing, storing the pharmaceutical composition at 4° C. for a second period of time.
60. The method of paragraph 59, wherein the first period of time is about 6 months, about 7 months, about 8 months, about 9 months, about 10 months, about 11 months, about 12 months, about 15 months, about 18 months, or about 24 months.
61. The method of paragraph 59, wherein the second period of time is about 1 week, about 2 weeks, about 3 weeks, about 4 weeks, about 1 month, about 2 months.
62. A kit comprising one or more containers and instructions for use, wherein the one or more containers comprise the pharmaceutical composition of any one of paragraphs 1-53.
63. The kit of paragraph 62, wherein at least one of the one or more containers is made from hydrophobically-coated glass vial.
64. The kit of paragraph 62, wherein at least one of the one or more containers is made from Daikyo Crystal Zenith® (CZ) vial.
65. The kit of paragraph 62, wherein at least one of the one or more containers is made from TopLyo coated vial.
66. The kit of paragraph 62, wherein at least one of the one or more containers is made from Cyclo Olefin Polymer (COP).
67. A pharmaceutical composition comprising:
(a) a Construct II encoding an anti-human vascular endothelial growth factor (hVEGF) antibody,
(b) potassium chloride,
(c) potassium phosphate monobasic,
(d) sodium chloride,
(e) sodium phosphate dibasic anhydrous,
(f) sucrose, and
(e) poloxamer 188, polysorbate 20, or polysorbate 80, and
wherein the anti-hVEGF antibody comprises a heavy chain comprising the amino acid sequence of SEQ ID NO:2 or SEQ ID NO:4, and a light chain comprising the amino acid sequence of SEQ ID NO:1 or SEQ ID NO:3.
68. The pharmaceutical composition of paragraph 67, wherein the ionic strength of the pharmaceutical composition is in a range from about 60 mM to about 100 mM.
69. The pharmaceutical composition of any one of paragraphs 67-68, wherein the pharmaceutical composition comprises:
(a) potassium chloride at a concentration of 0.2 g/L,
(b) potassium phosphate monobasic at a concentration of 0.2 g/L,
(c) sodium chloride at a concentration of 5.84 g/L, and
(d) sodium phosphate dibasic anhydrous at a concentration of 1.15 g/L.
70. The pharmaceutical composition of any one of paragraphs 67-69, wherein the pharmaceutical composition comprises sucrose at a concentration in a range from 3% (weight/volume, 30 g/L) to 18% (weight/volume, 180 g/L).
71. The pharmaceutical composition of any one of paragraphs 67-69, wherein the pharmaceutical composition comprises sucrose at a concentration of 4% (weight/volume, 40 g/L).
72. The pharmaceutical composition of any one of paragraphs 67-71, wherein the pharmaceutical composition comprises poloxamer 188, polysorbate 20, or polysorbate 80; and wherein the poloxamer 188, polysorbate 20, or polysorbate 80 is at a concentration in a range from 0.0005% (weight/volume, 0.005 g/L) to 0.05% (weight/volume, 0.5 g/L).
73. The pharmaceutical composition of any one of paragraphs 67-71, wherein the pharmaceutical composition comprises poloxamer 188, polysorbate 20, or polysorbate 80 at a concentration of 0.001% (weight/volume, 0.01 g/L).
74. The pharmaceutical composition of any one of paragraphs 67-73, wherein the pH of the pharmaceutical composition is in a range from about 6.0 to about 9.0.
75. The pharmaceutical composition of any one of paragraphs 67-73, wherein the pH of the pharmaceutical composition is about 7.4.
76. The pharmaceutical composition of any one of paragraphs 67-75, wherein the osmolality of the pharmaceutical composition is in a range from about 200 mOsm/L to about 660 mOsm/L.
77. The pharmaceutical composition of any one of paragraphs 67-76, wherein the Construct II comprises the capsid from AAV8.
78. The pharmaceutical composition of any one of paragraphs 67-77, wherein the Construct II is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more stable to freeze/thaw cycles than the same Construct II in a reference pharmaceutical composition.
78. The pharmaceutical composition of any one of paragraphs 67-77, wherein the Construct II is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more stable than the same Construct II in a reference pharmaceutical composition when stored at −20° C. for a period of time, wherein the period of time is about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, or about 24 months.
79. The pharmaceutical composition of any one of paragraphs 67-77, wherein the Construct II is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more stable than the same Construct II in a reference pharmaceutical composition when stored at −80° C. for a period of time, wherein the period of time is about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, or about 24 months.
80. The pharmaceutical composition of any one of paragraphs 67-77, wherein the Construct II is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more stable than the same Construct II in a reference pharmaceutical composition when stored at room temperate for a period of time, wherein the period of time is about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, or about 24 months.
81. The pharmaceutical composition of any one of paragraphs 67-77, wherein, the recombinant AAV is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more stable than the same Construct II in a reference pharmaceutical composition when (i) stored at −80° C. for a first period of time; (ii) subsequently thawed; and (iii) after thawing, stored at 4° C. for a second period of time.
82. The pharmaceutical composition of paragraph 81, wherein the first period of time is about 6 months, about 7 months, about 8 months, about 9 months, about 10 months, about 11 months, about 12 months, about 15 months, about 18 months, or about 24 months.
83. The pharmaceutical composition of paragraph 81, wherein the second period of time is about 1 week, about 2 weeks, about 3 weeks, about 4 weeks, about 1 month, about 2 months.
84. The pharmaceutical composition of any one of paragraphs 67-79, wherein the vector genome concentration of the Construct II after being stored at −80° C. for a period of time is at least 70%, 75%, 80%, 85%, 90%, 95%, 98%, or 99% of the vector genome concentration of the Construct II before being stored at −80° C. for said period of time.
85. The pharmaceutical composition of any one of paragraphs 67-79, wherein the vector genome concentration of the Construct II after being stored at −20° C. for a period of time is at least 70%,
75%, 80%, 85%, 90%, 95%, 98%, or 99% of the vector genome concentration of the Construct II before being stored at −20° C. for said period of time.
86. The pharmaceutical composition of any one of paragraphs 67-79, wherein the vector genome concentration of the Construct II after being stored at 4° C. for a period of time is at least 70%, 75%, 80%, 85%, 90%, 95%, 98%, or 99% of the vector genome concentration of the Construct II before being stored at 4° C. for said period of time.
87. The pharmaceutical composition of any one of paragraphs 67-79, wherein the in vitro potency of the Construct II after being stored at −80° C. for a period of time is at least 70%, 75%, 80%, 85%, 90%, 95%, 98%, or 99% of the in vitro potency of the Construct II before being stored at −80° C. for said period of time.
88. The pharmaceutical composition of any one of paragraphs 67-79, wherein the in vitro potency of the Construct II after being stored at −20° C. for a period of time is at least 70%, 75%, 80%, 85%, 90%, 95%, 98%, or 99% of the in vitro potency of the Construct II before being stored at −20° C. for said period of time.
89. The pharmaceutical composition of any one of paragraphs 67-79, wherein the in vitro potency of the Construct II after being stored at 4° C. for a period of time is at least 70%, 75%, 80%, 85%, 90%, 95%, 98%, or 99% of the in vitro potency of the Construct II before being stored at 4° C. for said period of time.
90. The pharmaceutical composition of any one of paragraphs 67-79, wherein the size distribution of the Construct II after being stored at −80° C. for a period of time is at least 70%, 75%, 80%, 85%, 90%, 95%, 98%, or 99% identical to the size distribution of the Construct II before being stored at −80° C. for said period of time.
91. The pharmaceutical composition of any one of paragraphs 67-79, wherein the size distribution of the Construct II after being stored at −20° C. for a period of time is at least 70%, 75%, 80%, 85%, 90%, 95%, 98%, or 99% identical to the size distribution of the Construct II before being stored at −20° C. for said period of time.
92. The pharmaceutical composition of any one of paragraphs 67-79, wherein the size distribution of the Construct II after being stored at 4° C. for a period of time is at least 70%, 75%, 80%, 85%, 90%, 95%, 98%, or 99% identical to the size distribution of the Construct II before being stored at 4° C. for said period of time.
93. The pharmaceutical composition of any one of paragraphs 84 to 92, wherein the period of time is about 1 month, about 2 months, about 3 months, about 4 months, about 5 months, about 6 months, about 7 months, about 8 months, about 9 months, about 10 months, about 11 months, about 12 months, about 15 months, about 18 months, or about 24 months.
94. The pharmaceutical composition of any one of paragraphs 77-83, wherein the stability of the Construct II is determined by the infectivity of recombinant AAV.
95. The pharmaceutical composition of any one of paragraphs 77-83, wherein the stability of the Construct II is determined by the levels of aggregation of recombinant AAV.
96. The pharmaceutical composition of any one of paragraphs 77-83, wherein the stability of the Construct II is determined by the levels of free DNA released by the recombinant AAV particles.
97. The pharmaceutical composition of any one of paragraphs 67-96, wherein the pharmaceutical composition is in a hydrophobically-coated glass vial.
98. The pharmaceutical composition of any one of paragraphs 67-96, wherein the pharmaceutical composition is in a Cyclo Olefin Polymer (COP) vial.
99. The pharmaceutical composition of any one of paragraphs 67-96, wherein the pharmaceutical composition is in a Daikyo Crystal Zenith® (CZ) vial.
100. The pharmaceutical composition of any one of paragraphs 67-96 wherein the pharmaceutical composition is in a TopLyo coated vial.
101. The pharmaceutical composition of any one of paragraphs 67-100, wherein the pharmaceutical composition consists of:
(a) the Construct II encoding an anti-human vascular endothelial growth factor (hVEGF) antibody,
(b) potassium chloride at a concentration of 0.2 g/L,
(c) potassium phosphate monobasic at a concentration of 0.2 g/L,
(d) sodium chloride at a concentration of 5.84 g/L,
(e) sodium phosphate dibasic anhydrous at a concentration of 1.15 g/L,
(f) sucrose at a concentration of 4% weight/volume (40 g/L),
(g) poloxamer 188, polysorbate 20, or polysorbate 80 at a concentration of 0.001%
weight/volume (0.01 g/L), and
(h) water, and
wherein the anti-hVEGF antibody comprises a heavy chain comprising the amino acid sequence of SEQ ID NO:2 or SEQ ID NO:4, and a light chain comprising the amino acid sequence of SEQ ID NO:1 or SEQ ID NO:3.
102. The pharmaceutical composition of paragraph 101, wherein the vector genome concentration (VGC) of the pharmaceutical composition is about 3×10⁹ GC/mL, about 1×10¹⁰ GC/mL, about 1.2×10¹⁰ GC/mL, about 1.6×10¹⁰ GC/mL, about 4×10¹⁰ GC/mL, about 6×10¹⁰ GC/mL, about 2×10¹¹ GC/mL, about 2.4×10¹¹ GC/mL, about 2.5×10¹¹ GC/mL, about 3×10¹¹ GC/mL, about 3.2×10¹¹ GC/mL, about 6.2×10¹¹ GC/mL, about 6.5×10¹¹ GC/mL, about 1×10¹² GC/mL, about 3×10¹² GC/mL, about 2×10¹³ GC/mL or about 3×10¹³ GC/mL.
103. The pharmaceutical composition of any one of paragraphs 67-102, the pharmaceutical composition has a property that is suitable for intravenous administration, subcutaneous administration, intramuscular injection, suprachoroidal injection, subretinal injection via transvitreal approach, subretinal administration via the suprachoroidal space, or a posterior juxtascleral depot procedure.
104. The pharmaceutical composition of any one of paragraphs 67-102, the pharmaceutical composition has a desired density that is suitable for intravenous administration, subcutaneous administration, intramuscular injection, suprachoroidal injection, subretinal injection via transvitreal approach, subretinal administration via the suprachoroidal space, or a posterior juxtascleral depot procedure.
105. The pharmaceutical composition of any one of paragraphs 67-102, the pharmaceutical composition has a desired osmolality that is suitable for intravenous administration, subcutaneous administration, intramuscular injection, suprachoroidal injection, subretinal injection via transvitreal approach, subretinal administration via the suprachoroidal space, or a posterior juxtascleral depot procedure.
106. The pharmaceutical composition of paragraph 105 wherein the osmolality is 160-230 mOsm/kg H₂O.
107. The pharmaceutical composition of paragraph 105, wherein the osmolality is less than 600 mOsm/kg H₂O.
108. The pharmaceutical composition of any one of paragraphs 67-102, the pharmaceutical composition has a desired viscosity that is suitable for intravenous administration, subcutaneous administration, intramuscular injection, suprachoroidal injection, subretinal injection via transvitreal approach, subretinal administration via the suprachoroidal space, or a posterior juxtascleral depot procedure.
109. The pharmaceutical composition of any one of paragraphs 67-108, wherein the pharmaceutical composition is a liquid composition.
110. The pharmaceutical composition of any one of paragraphs 67-108, wherein the pharmaceutical composition is a frozen composition.
111. The pharmaceutical composition of any one of paragraphs 67-108, wherein the pharmaceutical composition is a lyophilized composition or a reconstituted lyophilized composition.
112. The pharmaceutical composition of any one of paragraphs 67-111, wherein the pharmaceutical composition is capable of being stored at 4° C. for 1 weeks, about 2 weeks, about 3 weeks, about 4 weeks, about 1 month, about 2 months after having previously been stored at-80° C. for about 6 months, about 7 months, about 8 months, about 9 months, about 10 months, about 11 months, about 12 months, about 15 months, about 18 months, about 24 months.
113. A method of treating a subject diagnosed with nAMD (wet AMD), dry AMD, retinal vein occlusion (RVO), diabetic macular edema (DME), or diabetic retinopathy (DR) comprising administering to the subject the pharmaceutical composition of any one of paragraphs 67-111.
114. The method of paragraph 113, wherein the pharmaceutical composition is administered by suprachoroidal injection, subretinal injection via transvitreal approach, subretinal administration via the suprachoroidal space, or a posterior juxtascleral depot procedure.
115. The method of any one of paragraphs 113 or 114, wherein the subject is a human subject.
116. A method of treating a subject diagnosed with nAMD (wet AMD), dry AMD, retinal vein occlusion (RVO), diabetic macular edema (DME), or diabetic retinopathy (DR), the method comprising preparing the pharmaceutical composition of any one of paragraphs 67-108, storing the pharmaceutical composition at −80° C. for a first period of time; (ii) thawing the pharmaceutical composition; and (iii) after thawing, storing the pharmaceutical composition at 4° C. for a second period of time.
117. The method of paragraph 119, wherein the first period of time is about 6 months, about 7 months, about 8 months, about 9 months, about 10 months, about 11 months, about 12 months, about 15 months, about 18 months, or about 24 months.
118. The method of paragraph 119, wherein the second period of time is about 1 week, about 2 weeks, about 3 weeks, about 4 weeks, about 1 month, about 2 months.
119. A kit comprising one or more containers and instructions for use, wherein the one or more containers comprise the pharmaceutical composition of any one of paragraphs 67-102.
120. The kit of paragraph 119, wherein at least one of the one or more containers is made from Cyclo Olefin Polymer (COP).
121. A stable liquid pharmaceutical composition comprising:
(a) a recombinant adeno-associated virus (rAAV),
(b) a buffering agent comprising ionic salt and having an ionic strength between 60 mM and 150 mM,
(d) sucrose, and
(e) surfactant.
122. The composition of paragraph 121, wherein the rAAV comprises AAV1, AAV2, AAV2tYF, AAV3, AAV4, AAV5, AAV6, AAV7, AAV8, AAV9, AAV10, AAV11, AAVrh10, AAV.rh20, AAV.rh39, AAV.Rh74, AAV.RHM4-1, AAV.hu37, AAV.Anc80, AAV.Anc80L65, rAAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16.
123. The composition of paragraph 121 or 122, wherein the composition comprises 3-16% sucrose.
124. The composition of any one of paragraphs 121-123, wherein the buffering agent maintains pH between about pH 6 to about pH 9 over the temperature range of −20° C. to room temperature.
125. The composition of any one of paragraphs 121-124, wherein the composition comprises 4-6% sucrose.
126. The composition of any one of paragraphs 121-125, wherein the buffering agent has an ionic strength no greater than about 150 mM, about 145 mM, about 140 mM, about 135 mM, about 130 mM, about 125 mM, about 120 mM, about 115 mM, or about 110 mM.
127. The composition of any one of paragraphs 121-126, wherein the buffering agent has an ionic strength between about 60 mM and about 115 mM.
128. The composition of any one of paragraphs 121-127 wherein the buffering agent has an ionic strength between about 60 mM and about 110 mM.
129. The composition of any one of paragraphs 121-128, wherein the composition comprises between 60 mM and 100 mM NaCl.
130. The composition of any one of paragraphs 121-129, wherein the composition comprises 4-6% sucrose.
131. The composition of any one of paragraphs 121-130 wherein the composition is frozen to a temperature of about−20° C.
132. The composition of any one of paragraphs 121-131, wherein the frozen composition maintains pH between about pH 6 to about pH 9.
133. The composition of any one of paragraphs 121-132, wherein the buffering agent comprises one or more components selected from the group consisting of potassium phosphate monobasic, potassium phosphate, sodium chloride, sodium phosphate dibasic anhydrous, sodium phosphate hexahydrate, sodium phosphate monobasic monohydrate, sodium phosphate hexahydrate, sodium phosphate monobasic monohydrate, tromethamine, tris(hydroxymethyl)aminomethane hydrochloride (Tris-HCl), amino acid, histidine, histidine hydrochloride (histidine-HCl), sodium succinate, sodium citrate, sodium acetate, and (4-(2-hydroxyethyl)-1-piperazineethanesulfonic acid) (HEPES), sodium sulfate, magnesium sulfate, magnesium chloride 6-hydrate, calcium sulfate, potassium chloride, calcium chloride, and calcium citrate.
134. The composition of any one of paragraphs 121-132, wherein the buffering agent comprises potassium chloride, potassium phosphate monobasic, sodium chloride, sodium phosphate dibasic anhydrous.
135. The composition of any one of paragraphs 121-132, wherein the buffering agent comprises sodium chloride, and Tris hydrochloride.
136. The composition of any one of paragraphs 121-133, wherein the surfactant is poloxamer 188, polysorbate 20, or polysorbate 80.
137. The composition of any one of paragraphs 121-133, wherein the surfactant is poloxamer 188, polysorbate 20, or polysorbate 80; and wherein the poloxamer 188, polysorbate 20, or polysorbate 80 is at a concentration in a range from 0.005% (weight/volume, 0.005 g/L) to 0.05% (weight/volume, 0.5 g/L).
138. The composition of any one of paragraphs 121-133, wherein the surfactant is poloxamer 188, polysorbate 20, or polysorbate 80; and wherein the poloxamer 188, polysorbate 20, or polysorbate 80 is at a concentration of 0.001% (weight/volume, 0.01 g/L).
139. The composition of any one of paragraphs 121-138, wherein the liquid pharmaceutical composition is further lyophilized.
140. The composition of any one of paragraphs 121-138, wherein the liquid pharmaceutical composition is a reconstituted lyophilized powder.
141. The pharmaceutical composition of any one of paragraphs 121-140, wherein the vector genome concentration of the recombinant AAV after being stored at −80° C. for a period of time is at least 70%, 75%, 80%, 85%, 90%, 95%, 98%, or 99% of the vector genome concentration of the recombinant AAV before being stored at −80° C. for said period of time.
142. The pharmaceutical composition of any one of paragraphs 121-140, wherein the vector genome concentration of the recombinant AAV after being stored at −20° C. for a period of time is at least 70%, 75%, 80%, 85%, 90%, 95%, 98%, or 99% of the vector genome concentration of the recombinant AAV before being stored at −20° C. for said period of time.
143. The pharmaceutical composition of any one of paragraphs 121-140, wherein the vector genome concentration of the recombinant AAV after being stored at 4° C. for a period of time is at least 70%, 75%, 80%, 85%, 90%, 95%, 98%, or 99% of the vector genome concentration of the recombinant AAV before being stored at 4° C. for said period of time.
144. The pharmaceutical composition of any one of paragraphs 121-140, wherein the in vitro potency of the recombinant AAV after being stored at −80° C. for a period of time is at least 70%, 75%, 80%, 85%, 90%, 95%, 98%, or 99% of the in vitro potency of the recombinant AAV before being stored at −80° C. for said period of time.
145. The pharmaceutical composition of any one of paragraphs 121-140, wherein the in vitro potency of the recombinant AAV after being stored at −20° C. for a period of time is at least 70%, 75%, 80%, 85%, 90%, 95%, 98%, or 99% of the in vitro potency of the recombinant AAV before being stored at −20° C. for said period of time.
146. The pharmaceutical composition of any one of paragraphs 121-140, wherein the in vitro potency of the recombinant AAV after being stored at 4° C. for a period of time is at least 70%, 75%, 80%, 85%, 90%, 95%, 98%, or 99% of the in vitro potency of the recombinant AAV before being stored at 4° C. for said period of time.
147. The pharmaceutical composition of any one of paragraphs 121-140, wherein the size distribution of the recombinant AAV after being stored at −80° C. for a period of time is at least 70%, 75%, 80%, 85%, 90%, 95%, 98%, or 99% identical to the size distribution of the recombinant AAV before being stored at −80° C. for said period of time.
148. The pharmaceutical composition of any one of paragraphs 121-140, wherein the size distribution of the recombinant AAV after being stored at −20° C. for a period of time is at least 70%, 75%, 80%, 85%, 90%, 95%, 98%, or 99% identical to the size distribution of the recombinant AAV before being stored at −20° C. for said period of time.
149. The pharmaceutical composition of any one of paragraphs 121-140, wherein the size distribution of the recombinant AAV after being stored at 4° C. for a period of time is at least 70%, 75%, 80%, 85%, 90%, 95%, 98%, or 99% identical to the size distribution of the recombinant AAV before being stored at 4° C. for said period of time.
150. The pharmaceutical composition of any one of paragraphs 141-149, wherein the period of time is about 1 month, about 2 months, about 3 months, about 4 months, about 5 months, about 6 months, about 7 months, about 8 months, about 9 months, about 10 months, about 11 months, about 12 months, about 15 months, about 18 months, or about 24 months.
151. The pharmaceutical composition of any one of paragraphs 121-140, wherein the pharmaceutical composition is capable of being stored at 4° C. for 1 weeks, about 2 weeks, about 3 weeks, about 4 weeks, about 1 month, about 2 months after having previously been stored at-80° C. for about 6 months, about 7 months, about 8 months, about 9 months, about 10 months, about 11 months, about 12 months, about 15 months, about 18 months, about 24 months.
152. A method of treating a disease of interest comprising administering to the subject the pharmaceutical composition of any one of paragraphs 121-140, wherein the rAAV encodes a transgene that treats, or otherwise ameliorates, prevents or slows the progression of the disease of interest.
153. A method of treating a subject diagnosed with nAMD (wet AMD), dry AMD, retinal vein occlusion (RVO), diabetic macular edema (DME), or diabetic retinopathy (DR), the method comprising preparing the pharmaceutical composition of any one of paragraphs 121-140, storing the pharmaceutical composition at −80° C. for a first period of time; (ii) thawing the pharmaceutical composition; and (iii) after thawing, storing the pharmaceutical composition at 4° C. for a second period of time.
154. The method composition of paragraph 153 wherein the first period of time is about 6 months, about 7 months, about 8 months, about 9 months, about 10 months, about 11 months, about 12 months, about 15 months, about 18 months, or about 24 months.
155. The method of paragraph 153, wherein the second period of time is about 1 week, about 2 weeks, about 3 weeks, about 4 weeks, about 1 month, about 2 months.
156. A kit comprising one or more containers and instructions for use, wherein the one or more containers comprise the pharmaceutical composition of any one of paragraphs 121-140.
157. A stable liquid formulation comprising the pharmaceutical composition of any one of paragraphs 1-53, 67-111 and 121-140.
158. A single unit dosage form comprising 3.2×10¹¹ GC/mL of Construct II, 0.2 g/L potassium chloride, 0.2 g/L potassium phosphate monobasic, 8.01 g/L sodium chloride, 1.15 g/L sodium phosphate dibasic anhydrous, pH 7.4 and 0.001% P188 in a volume of about 0.95 mL in a Cyclo Olefin Polymer (COP) vial.
159. A single unit dosage form comprising 3.2×10¹¹ GC/mL of Construct II, 0.2 g/L potassium chloride, 0.2 g/L potassium phosphate monobasic, 8.01 g/L sodium chloride, 1.15 g/L sodium phosphate dibasic anhydrous, pH 7.4 and 0.001% P188 in a volume of at least 0.8 mL in a COP vial.
160. A single unit dosage form comprising 3.2×10¹¹ GC/mL of Construct II, 0.2 g/L potassium chloride, 0.2 g/L potassium phosphate monobasic, 8.01 g/L sodium chloride, 1.15 g/L sodium phosphate dibasic anhydrous, pH 7.4, 4% sucrose and 0.001% P188 in a volume of about 0.95 mL in a COP vial.
161. A single unit dosage form comprising 3.2×10¹¹ GC/mL of Construct II, 0.2 g/L potassium chloride, 0.2 g/L potassium phosphate monobasic, 8.01 g/L sodium chloride, 1.15 g/L sodium phosphate dibasic anhydrous, pH 7.4, 4% sucrose and 0.001% P188 in a volume of at least 0.8 mL in a COP vial.
162. A single unit dosage form comprising 6.5×10¹¹ GC/mL of Construct II, 0.2 g/L potassium chloride, 0.2 g/L potassium phosphate monobasic, 8.01 g/L sodium chloride, 1.15 g/L sodium phosphate dibasic anhydrous, pH 7.4 and 0.001% P188 in a volume of about 0.95 mL in a COP vial.
163. A single unit dosage form comprising 6.5×10¹¹ GC/mL of Construct II, 0.2 g/L potassium chloride, 0.2 g/L potassium phosphate monobasic, 8.01 g/L sodium chloride, 1.15 g/L sodium phosphate dibasic anhydrous, pH 7.4, and 0.001% P188 in a volume of at least 0.8 mL in a COP vial.
164. A single unit dosage form comprising 6.5×10¹¹ GC/mL of Construct II, 0.2 g/L potassium chloride, 0.2 g/L potassium phosphate monobasic, 8.01 g/L sodium chloride, 1.15 g/L sodium phosphate dibasic anhydrous, pH 7.4, 4% sucrose and 0.001% P188 in a volume of about 0.95 mL in a COP vial.
165. A single unit dosage form comprising 6.5×10¹¹ GC/mL of Construct II, 0.2 g/L potassium chloride, 0.2 g/L potassium phosphate monobasic, 8.01 g/L sodium chloride, 1.15 g/L sodium phosphate dibasic anhydrous, pH 7.4, 4% sucrose and 0.001% P188 in a volume of at least 0.8 mL in a COP vial.
166. A single unit dosage form comprising 2.5×10¹² GC/mL of Construct II, 0.2 g/L potassium chloride, 0.2 g/L potassium phosphate monobasic, 8.01 g/L sodium chloride, 1.15 g/L sodium phosphate dibasic anhydrous, pH 7.4, and 0.001% P188 in a volume of about 0.6 mL in a COP vial.
167. A single unit dosage form comprising 2.5×10¹² GC/mL of Construct II, 0.2 g/L potassium chloride, 0.2 g/L potassium phosphate monobasic, 8.01 g/L sodium chloride, 1.15 g/L sodium phosphate dibasic anhydrous, pH 7.4, and 0.001% P188 in a volume of at least 0.5 mL in a COP vial.
168. A single unit dosage form comprising 3×10¹³ GC/mL of Construct II, 0.2 g/L potassium chloride, 0.2 g/L potassium phosphate monobasic, 8.01 g/L sodium chloride, 1.15 g/L sodium phosphate dibasic anhydrous, pH 7.4, 4% sucrose and 0.001% P188 in a volume of about 0.6 mL in a COP vial.
169. A single unit dosage form comprising 3×10¹³ GC/mL of Construct II, 0.2 g/L potassium chloride, 0.2 g/L potassium phosphate monobasic, 8.01 g/L sodium chloride, 1.15 g/L sodium phosphate dibasic anhydrous, pH 7.4, 4% sucrose and 0.001% P188 in a volume of at least 0.5 mL in a COP vial.
170. The single unit dosage form of any one of paragraphs 158-169, which is capable of being stored at 4° C. for 1 weeks, about 2 weeks, about 3 weeks, about 4 weeks, about 1 month, about 2 months.
171. The single unit dosage form of any one of paragraphs 158-169, which is capable of being stored at −80° C. for about 6 months, about 7 months, about 8 months, about 9 months, about 10 months, about 11 months, about 12 months, about 15 months, about 18 months, about 24 months.
172. The single unit dosage form of any one of paragraphs 158-169, which is capable of being stored at 4° C. for 1 weeks, about 2 weeks, about 3 weeks, about 4 weeks, about 1 month, about 2 months after having previously been stored at −80° C. for about 6 months, about 7 months, about 8 months, about 9 months, about 10 months, about 11 months, about 12 months, about 15 months, about 18 months, about 24 months.
173. The single unit dosage form of any one of paragraphs 158-169, wherein the vector genome concentration of the Construct II after being stored at −80° C. for a period of time is at least 70%, 75%, 80%, 85%, 90%, 95%, 98%, or 99% of the vector genome concentration of the Construct II before being stored at −80° C. for said period of time.
174. The single unit dosage form of any one of paragraphs 158-169, wherein the vector genome concentration of the Construct II after being stored at −20° C. for a period of time is at least 70%, 75%, 80%, 85%, 90%, 95%, 98%, or 99% of the vector genome concentration of the Construct II before being stored at −20° C. for said period of time.
175. The single unit dosage form of any one of paragraphs 158-169, wherein the vector genome concentration of the Construct II after being stored at 4° C. for a period of time is at least 70%, 75%, 80%, 85%, 90%, 95%, 98%, or 99% of the vector genome concentration of the Construct II before being stored at 4° C. for said period of time.
176. The single unit dosage form of any one of paragraphs 158-169, wherein the in vitro potency of the Construct II after being stored at −80° C. for a period of time is at least 70%, 75%, 80%, 85%, 90%, 95%, 98%, or 99% of the in vitro potency of the Construct II before being stored at-80° C. for said period of time.
177. The single unit dosage form of any one of paragraphs 158-169, wherein the in vitro potency of the Construct II after being stored at −20° C. for a period of time is at least 70%, 75%, 80%, 85%, 90%, 95%, 98%, or 99% of the in vitro potency of the Construct II before being stored at-20° C. for said period of time.
178. The single unit dosage form of any one of paragraphs 158-169, wherein the in vitro potency of the Construct II after being stored at 4° C. for a period of time is at least 70%, 75%, 80%, 85%, 90%, 95%, 98%, or 99% of the in vitro potency of the Construct II before being stored at 4° C. for said period of time.
179. The single unit dosage form of any one of paragraphs 158-169, wherein the size distribution of the Construct II after being stored at −80° C. for a period of time is at least 70%, 75%, 80%, 85%, 90%, 95%, 98%, or 99% identical to the size distribution of the Construct II before being stored at −80° C. for said period of time.
180. The single unit dosage form of any one of paragraphs 158-169, wherein the size distribution of the Construct II after being stored at −20° C. for a period of time is at least 70%, 75%, 80%, 85%, 90%, 95%, 98%, or 99% identical to the size distribution of the Construct II before being stored at −20° C. for said period of time.
181. The single unit dosage form of any one of paragraphs 158-169, wherein the size distribution of the Construct 11 after being stored at 4 C for a period of time is at least 700, 750, 80%, 85%, 90, 95, 98%, or 990 identical to the size distribution of the Construct before being stored at 4C for said period of time.
182. The single unit dosage form ofany one of paragraphs 173-181, wherein the period of time is about 1 month, about 2 months, about 3 months, about 4 months, about 5 months, about 6 months, about 7 months, about 8 months, about 9 months, about 10 months, about 11 months, about 12 months, about 15 months, about 18 months, or about 24 months.
183. A pre-filled syringe containing the single unit dosage form of any one of paragraphs 158-183.
184. A kit comprising the pre-filled syringe of paragraph 183.

### 2.2 Conventions and Abbreviations

| Abbreviation | Convention |
|---|---|
| AAV | Adeno-associated virus |
| DLS | Dynamic light scattering |
| SEC | Size-exclusion chromatography |
| FDP | Final drug product |
| BDS | Bulk drug substance |
| VEGF | Vascular endothelial growth factor |
| ddPCR | Digital Droplet PCR |
| AUC | Analytical Ultracentrifugation |
| USP | United States Pharmacopeia |
| IVRP | in vitro relative potency |
| VGC | vector genome concentration |
| HRP | horseradish peroxidase |
| DSC | Differential Scanning Calorimetry |
| dPBS | Dulbecco's phosphate buffered saline |
| ELISA | Enzyme-linked immunosorbent assay |
| SF | Slow Freeze |
| ST | Slow Thaw |
| FT | Fast Thaw |
| FF | Fast Freeze |
| Tg′ | glass transition temperature |

### 3. BRIEF DESCRIPTION OF THE DRAWINGS

FIG. 1. The amino acid sequence of ranibizumab (top) showing 5 different residues in bevacizumab Fab (below). The starts of the variable and constant heavy chains (VH and CH) and light chains (V_{L} and C_{L}) are indicated by arrows (→), and the CDRs are underscored. Non-consensus glycosylation sites (“Gsite”) tyrosine-O-sulfation sites (“Ysite”) are indicated.

FIG. 2. Glycans that can be attached to HuGlyFabVEGFi. (Adapted from Bondt et al., 2014, Mol & Cell Proteomics 13.1: 3029-3039).

FIG. 3. The amino acid sequence of hyperglycosylated variants of ranibizumab (above) and bevacizumab Fab (below). The starts of the variable and constant heavy chains (VH and CH) and light chains (V_{L} and C_{L}) are indicated by arrows (→), and the CDRs are underscored. Non-consensus glycosylation sites (“Gsite”) and tyrosine-O-sulfation sites (“Ysite”) are indicated. Four hyperglycoslated variants are indicated with an asterisk (*).

FIG. 4. Schematic of AAV8-antiVEGFfab genome.

FIG. 5. A suprachoroidal drug delivery device manufactured by Clearside® Biomedical, Inc.

FIG. 6. A subretinal drug delivery device comprising a catheter that can be inserted and tunneled through the suprachoroidal space toward the posterior pole, where a small needle injects into the subretinal space, manufactured by Janssen Pharmaceuticals, Inc.

FIGS. 7A-7D. Illustration of the posterior juxtascleral depot procedure.

FIG. 8. Clustal Multiple Sequence Alignment of AAV capsids 1-9 (SEQ ID NOs: 41-51). Amino acid substitutions (shown in bold in the bottom rows) can be made to AAV9 and AAV8 capsids by “recruiting” amino acid residues from the corresponding position of other aligned AAV capsids. Sequence regions designated by “HVR”=hypervariable regions.

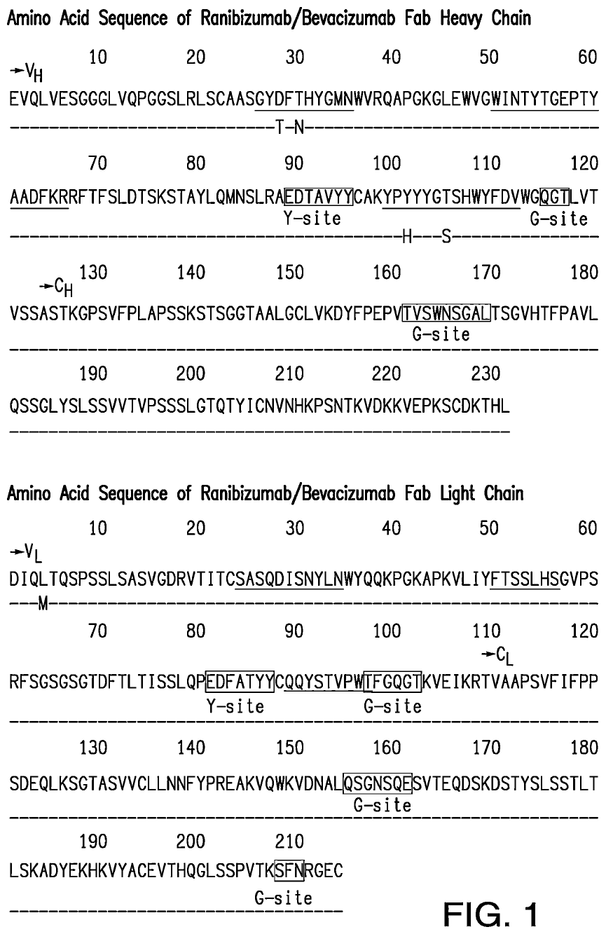

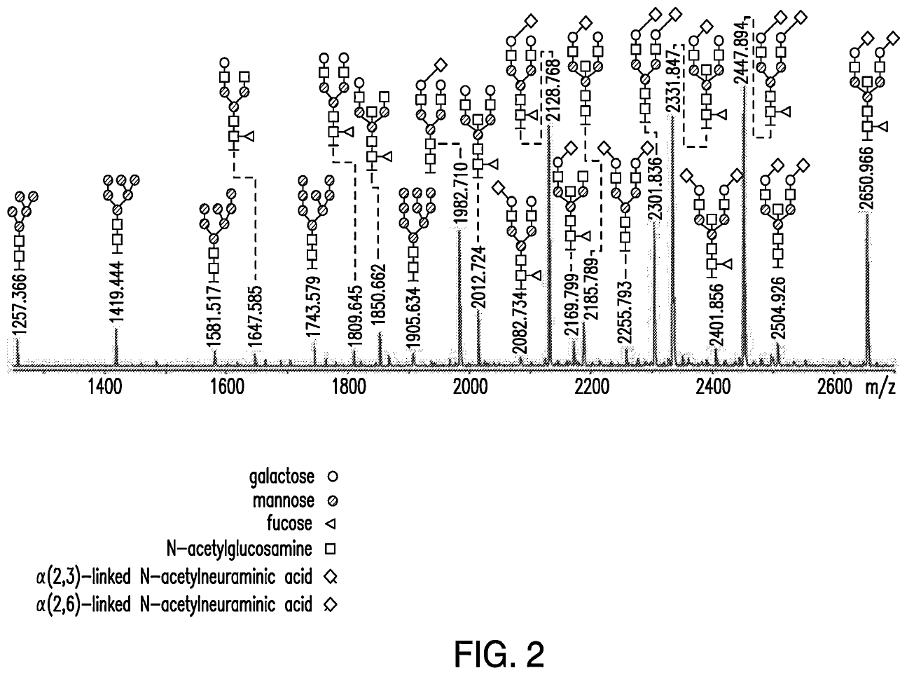

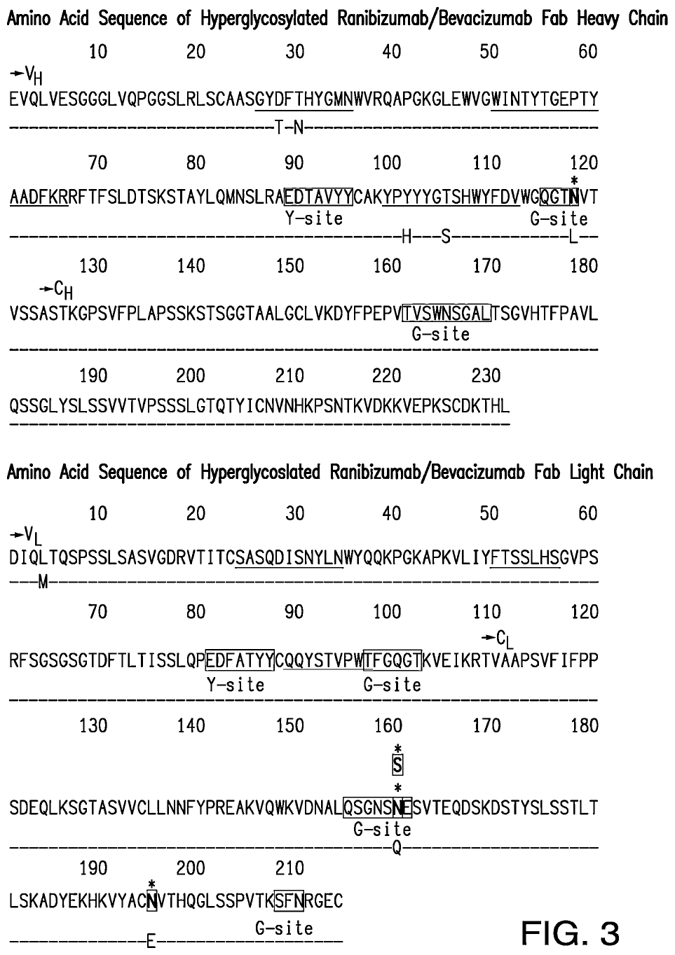

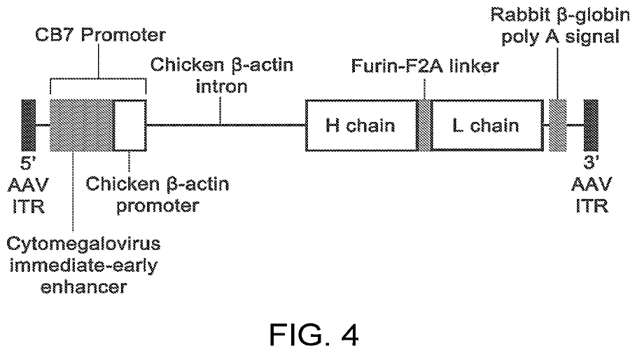

FIG. 0.9. Fast Freeze/Slow Thaw (FF/ST) Temperature Profile.

FIG. 10. Zoomed-in view of SEC result profiles for Construct II in DPBS Formulation A. pre-peaks are free DNA and aggregates. Post-peaks contain buffer and excipient species.

FIG. 11. Zoomed-in view of SEC result profiles for Construct II in Formulation B. Post-peaks contain buffer and excipient species. Post-peak due to sucrose was larger in Formulation B.

FIG. 12. Dynamic light scattering cumulants results for Construct II in Formulation A (dark grey) and Formulation B (light grey) control and after exposure to permutations of five fast and slow freeze/thaw cycles.

FIG. 13. Low temperature DSC thermogram for Construct II DPBS formulation A buffer.

FIG. 14. Low temperature DSC thermogram for Construct II in ‘modified DPBS with 4% Sucrose and 0.001% Poloxamer 188, pH 7.4’ formulation B buffer.

FIG. 15. Stability trend for Construct II potency in formulation A (dark grey circles) and formulation B (light grey squares) at 1.0×10¹² GC/mL at 37° C.

FIG. 16. Stability trend for Construct II free DNA by dye fluorescence in formulation A (dark grey circles) and formulation B (light grey squares) at 1.0×10¹² GC/mL at 37° C.

FIG. 17. Stability trend for Construct II potency in formulation B at 1.0×10¹² GC/mL at −80° C. and −20° C.

FIG. 18. Stability trend for Construct II potency in formulation B at 2.1×10¹¹ GC/mL at −80° C. and −20° C.

FIG. 19A. Temperature profile measured for 2 different fill volumes in Nalgene HDPE BDS bottles.

FIG. 19B. Temperature profiles recorded for 0.6 mL fills in 2 mL cryovials cycled between −80° C. and room temperature or −20° C.

FIG. 20A. Fast Freeze/Fast Thaw (FF/FT) Temperature Profile.

FIG. 20B. Fast Freeze/Fast Thaw (FF/FT) Temperature Profile (left axis) and Rates for the Shelf and Probes (right axis).

FIG. 21. Fast Freeze/Slow Thaw (FF/ST) Temperature Profile.

FIG. 22. Slow Freeze/Fast Thaw (SF/FT) Temperature Profile.

FIG. 23. Slow Freeze/Slow Thaw (SF/ST) Temperature Profile.

FIG. 24. Slow Freeze/Slow Thaw (SF/ST) Temperature Profile (left axis) and Rates for the Shelf and Probes (right axis).

FIG. 25. Zoomed-in view of SEC result profiles for Control formulation buffer.

FIG. 26. Zoomed-in view of SEC result profiles for Construct II in dPBS formulation buffer.

FIG. 27. Zoomed-in view of SEC result profiles for Construct II in modified dPBS with sucrose buffer.

FIG. 28. DLS diameter results for Construct II in dPBS freeze-thaw samples.

FIG. 29. DLS diameter results for Construct II in modified dPBS with sucrose freeze-thaw samples

FIG. 30. Comparison of the DLS cumulants diameter result for Construct II in dPBS compared to in modified dPBS with sucrose freeze-thaw samples.

FIG. 31. Comparison of the DLS regularization diameter result for Construct II in dPBS compared to in modified dPBS with sucrose freeze-thaw samples.

FIG. 32. Low temperature DSC thermogram for Construct II dPBS formulation buffer.

FIG. 33. Low temperature DSC thermogram for Construct II ‘modified dPBS with sucrose’ formulation buffer.

FIG. 34. Formulation B contains an amorphous excipient that inhibits crystallization/eutectic transition improving robustness to freeze/thaw stress.

FIG. 35. Free DNA increases with each freeze/thaw cycle for Formulation A.

FIG. 36. Potency of formulation a decreases with >5×freeze/thaw cycles and potency is maintained in formulation b for 30×freeze/thaw cycles. The ‘modified dPBS with 4% sucrose’ Formulation B (dark grey bars) maintained potency after 30 freeze-thaw cycles. In contrast the reference formulation (dPBS, light grey bars) potency decreased to between 66% and 72% after 15 to 30 freeze-thaw cycles. Example is for AAV8 with gene for green fluorescent protein. Freeze-thaw cycles are used to simulate transportation and storage logistics temperature changes and also as an ‘accelerated’ stress to force degradation of the AAV for formulation optimization work.

FIG. 37. Adsorption losses occur in glass vials but not detected for cop vials.

FIG. 38. Formulations A and B had similar long-term frozen stability at −80° C.; Formulation B was also stable at −20° C. The ‘modified dPBS with 4% sucrose’ formulation maintained potency for 12 months at −20° C. (circles) and −80° C. (squares). The reference formulation (dPBS) is shown for −80° C. storage as a comparator.

FIG. 39. Real-time monitoring pH and temperature of Formulation #2, Modified dPBS with 4% sucrose, in-20 AD freezer showing an approximate 3 pH unit acidification upon freezing of the formulation (top trace, axis on left). The temperature trace (bottom trace, right axis) shows the fluctuation in temperature as the auto-defrost cycle of the freezer occurs. The pH of the frozen solution, measured directly with the frozen pH electrode, shows fluctuations between about 4.3 and 5.5 when frozen that correlate with the temperature of the frozen formulation.

FIG. 40. Comparison of the pH of different buffers after being stressed by multiple defrosting cycles. The DPBS formulation shifted from 7.4 to about 4.3 when frozen. Formulations with sucrose had a lower pH shift from 7.4 to about 6.2 upon freezing. The TRIS formulation initially shifts relative to room temperature then is stable when frozen.

FIG. 41. Comparison of the pH of the buffers as the temperature decrease from 0 to-20° C. The phosphate-based Formulation #1 had a large pH shift on freezing. Formulations 2-7, showed a much lower acidification shift on freezing which is preferred for product stability. Formulation 8 shifted slightly more basic and within acceptable limits for formulation stability.

FIG. 42. Magnitude of different buffer pH shift after stabilization.

FIG. 43. Low temperature DSC thermogram for Formulation #1: dPBS formulation buffer.

FIG. 44. Low temperature DSC thermogram for Formulation #2: Modified dPBS with 4% sucrose formulation buffer.

FIG. 45. Low temperature DSC thermogram comparison of phase transition behavior of different formulations. Formulation 1 (dPBS) does not show a glass transition (trace indicated with text). Formulation 2 (Formulation B) and variations (formulation #3-7) and with TRIS buffer (formulation 8) showed similar phase transition behavior as Formulation #2, with a glass transition between −40 to −45° C.

FIG. 46. Viral particle aggregation is affected by ionic strength.

FIG. 47. Minimum ionic strength to prevent aggregation. Effective diameter of an AAV8 particle at 1.8×10¹³ GC/mL prepared at different NaCl concentrations. Ionic strength≥90 mM appears to be required to prevent particle aggregation as indicated by the diameters of the particles.

FIG. 48. Minimum ionic strength is serotype dependent. Effective diameter of an AAV8 (open square) and an AAV9 (open triangle) prepared at different NaCl concentrations. Concentration of vectors was 6×10¹¹ GC/mL.

FIG. 49. Formulation C is a variant of the ‘modified dPBS with sucrose’ with 60 mM NaCl and 6% sucrose (light grey triangles) and was stable for 2 years at −20° C. The reference formulation (dPBS) is shown for −20° C. storage as a comparator (dark grey squares) and was not stable at −20° C. Formulations B and C are shown to have comparable and superior long-term stability at −20° C.

FIG. 50. Potency Trend for Construct II in Formulation B at 2-8° C.

FIG. 51. Potency Trend for Construct II FDP Lot 200320-314-DL7 at 3.0×10¹³ GC/mL at controlled room temperature.

FIG. 52. Stability trend for Construct II potency in formulation B at 1.0×10¹² GC/mL at −80° C. and −20° C.

FIG. 53. Stability trend for Construct II potency in formulation B at 2.1×10¹¹ GC/mL at −80° C. and −20° C.

FIG. 54. Formulations A and B had similar long-term frozen stability at −80° C.; Formulation B was also stable at −20° C. The ‘modified dPBS with 4% sucrose’ formulation maintained potency for 12 months at −20° C. (circles) and −80° C. (squares). The reference formulation (dPBS) is shown for −80° C. storage as a comparator.

### 4. DETAILED DESCRIPTION OF THE INVENTION

Provided herein are pharmaceutical compositions, method of treating related to the pharmaceutical compositions and kits related to the pharmaceutical compositions. In some embodiments, compositions provided in Section 4.1 are formulated such that they have one or more functional properties described in Section 4.2. In certain embodiments, the pharmaceutical compositions provided herein has various advantages, for example, improved stability after free/thaw cycles, and improved long-term stability under various conditions. Also provided herein are assays that may be used in related studies (Section 4.5).

4.1 Formulation of Pharmaceutical Composition

The disclosure provides a pharmaceutical composition comprising a recombinant adeno-associated virus (AAV), potassium phosphate monobasic, sodium chloride, sodium phosphate dibasic anhydrous, sucrose, and surfactant.

In some embodiments, the pharmaceutical composition further comprises amino acid.

In some embodiments, the disclosure provides a pharmaceutical composition comprising a recombinant adeno-associated virus (AAV), ionic salt excipient or buffering agent, sucrose, and poloxamer 188. In some embodiments, the ionic salt excipient or buffering agent can be one or more components from the group consisting of potassium phosphate monobasic, potassium phosphate, sodium chloride, sodium phosphate dibasic anhydrous, sodium phosphate hexahydrate, sodium phosphate monobasic monohydrate, tromethamine, tris(hydroxymethyl)aminomethane hydrochloride (Tris-HCl), amino acid, histidine, histidine hydrochloride (histidine-HCl), sodium succinate, sodium citrate, sodium acetate, and (4-(2-hydroxyethyl)-1-piperazineethanesulfonic acid) (HEPES), sodium sulfate, magnesium sulfate, magnesium chloride 6-hydrate, calcium sulfate, potassium chloride, calcium chloride, calcium citrate.

In certain embodiments, the pharmaceutical composition has a ionic strength about 60 mM to 115 mM. In certain embodiments, the pharmaceutical composition has a ionic strength about 60 mM to 100 mM. In certain embodiments, the pharmaceutical composition has a ionic strength about 65 mM to 95 mM. In certain embodiments, the pharmaceutical composition has a ionic strength about 70 mM to 90 mM. In certain embodiments, the pharmaceutical composition has a ionic strength about 75 mM to 85 mM.

In certain embodiments, the pharmaceutical composition has a ionic strength about 30 mM to 100 mM. In certain embodiments, the pharmaceutical composition has a ionic strength about 35 mM to 95 mM. In certain embodiments, the pharmaceutical composition has a ionic strength about 40 mM to 90 mM. In certain embodiments, the pharmaceutical composition has a ionic strength about 45 mM to 85 mM. In certain embodiments, the pharmaceutical composition has a ionic strength about 50 mM to 80 mM. In certain embodiments, the pharmaceutical composition has a ionic strength about 55 mM to 75 mM. In certain embodiments, the pharmaceutical composition has a ionic strength about 60 mM to 70 mM.

In certain embodiments, the pharmaceutical composition has a ionic strength ranging from 60 mM to 115 mM. In certain embodiments, the pharmaceutical composition has a ionic strength ranging from 60 mM to 100 mM. In certain embodiments, the pharmaceutical composition has a ionic strength ranging from 65 mM to 95 mM. In certain embodiments, the pharmaceutical composition has a ionic strength ranging from 70 mM to 90 mM. In certain embodiments, the pharmaceutical composition has a ionic strength ranging from 75 mM to 85 mM.

In certain embodiments, the pharmaceutical composition has a ionic strength range from 30 mM to 100 mM. In certain embodiments, the pharmaceutical composition has a ionic strength range from 35 mM to 95 mM. In certain embodiments, the pharmaceutical composition has a ionic strength range from 40 mM to 90 mM. In certain embodiments, the pharmaceutical composition has a ionic strength range from 45 mM to 85 mM. In certain embodiments, the pharmaceutical composition has a ionic strength range from 50 mM to 80 mM. In certain embodiments, the pharmaceutical composition has a ionic strength range from 55 mM to 75 mM. In certain embodiments, the pharmaceutical composition has a ionic strength range from 60 mM to 70 mM.

In certain embodiments, the pharmaceutical composition comprises potassium chloride at a concentration of 0.2 g/L.

In certain embodiments, the pharmaceutical composition comprises potassium phosphate monobasic at a concentration of 0.2 g/L.

In certain embodiments, the pharmaceutical composition comprises sodium chloride at a concentration of 5.84 g/L, and

In certain embodiments, the pharmaceutical composition comprises sodium phosphate dibasic anhydrous at a concentration of 1.15 g/L.

In certain embodiments, the pharmaceutical composition comprises sucrose at a concentration of 3% (weight/volume, 30 g/L) to 18% (weight/volume, 180 g/L). In certain embodiments, the pharmaceutical composition comprises sucrose at a concentration of 4% (weight/volume, 40 g/L).

In certain embodiments, the pharmaceutical composition comprises poloxamer 188 at a concentration of 0.001% (weight/volume, 0.01 g/L).

In certain embodiments, the pharmaceutical composition comprises poloxamer 188 at a concentration of 0.0005% (weight/volume, 0.005 g/L) to 0.05% (weight/volume, 0.5 g/L). In certain embodiments, the pharmaceutical composition comprises poloxamer 188 at a concentration of 0.001% (weight/volume, 0.01 g/L).

In some embodiments, the disclosure provides a pharmaceutical composition comprises a recombinant adeno-associated virus (AAV), ionic salt excipient or buffering agent, sucrose, and surfactant. In some embodiments, the ionic salt excipient or buffering agent can be one or more components from the group consisting of potassium phosphate monobasic, potassium phosphate, sodium chloride, sodium phosphate dibasic anhydrous, sodium phosphate hexahydrate, sodium phosphate monobasic monohydrate, tromethamine, tris(hydroxymethyl)aminomethane hydrochloride (Tris-HCl), amino acid, histidine, histidine hydrochloride (histidine-HCl), sodium succinate, sodium citrate, sodium acetate, and (4-(2-hydroxyethyl)-1-piperazineethanesulfonic acid) (HEPES), sodium sulfate, magnesium sulfate, magnesium chloride 6-hydrate, calcium sulfate, potassium chloride, calcium chloride, calcium citrate. In some embodiments, the surfactant can be one or more components from the group consisting of poloxamer 188, polysorbate 20, and polysorbate 80.

In certain embodiments, the pharmaceutical composition comprises polysorbate 20 at a concentration of 0.0005% (weight/volume, 0.05 g/L) to 0.05% (weight/volume, 0.5 g/L).

In certain embodiments, the pharmaceutical composition comprises polysorbate 80 at a concentration of 0.0005% (weight/volume, 0.05 g/L) to 0.05% (weight/volume, 0.5 g/L).

In certain embodiments, the pH of the pharmaceutical composition is about 7.4.

In certain embodiments, the pH of the pharmaceutical composition is about 6.0 to 9.0.

In certain embodiments, the pH of the pharmaceutical composition is 7.4.

In certain embodiments, the pH of the pharmaceutical composition is 6.0 to 9.0.

As used herein and unless otherwise specified, the term “about” means within plus or minus 10% of a given value or range.

In certain embodiments, the pharmaceutical composition is in a hydrophobically-coated glass vial.

In certain embodiments, the pharmaceutical composition is in a Cyclo Olefin Polymer (COP) vial.

In certain embodiments, the pharmaceutical composition is in a Daikyo Crystal Zenith® (CZ) vial.

In certain embodiments, the pharmaceutical composition is in a TopLyo coated vial.

In certain embodiments, disclosed herein is a pharmaceutical composition consists of: (a) the recombinant AAV, (b) potassium chloride at a concentration of 0.2 g/L, (c) potassium phosphate monobasic at a concentration of 0.2 g/L, (d) sodium chloride at a concentration of 5.84 g/L, (e) sodium phosphate dibasic anhydrous at a concentration of 1.15 g/L, (f) sucrose at a concentration of 4% weight/volume (40 g/L), (g) poloxamer 188 at a concentration of 0.001% weight/volume (0.01 g/L), and (h) water, and wherein the recombinant AAV is AAV8.

In certain embodiments, the vector genome concentration (VGC) of the pharmaceutical composition is about 3×10⁹ GC/mL, about 1×10¹⁰ GC/mL, about 1.2×10¹⁰ GC/mL, about 1.6×10¹⁰ GC/mL, about 4×10¹⁰ GC/mL, about 6×10¹⁰ GC/mL, about 2×10¹¹ GC/mL, about 2.4×10¹¹ GC/mL, about 2.5×10¹¹ GC/mL, about 3×10¹¹ GC/mL, about 6.2×10¹¹ GC/mL, about 1×10¹² GC/mL, about 3×10¹² GC/mL, about 2×10¹³ GC/mL or about 3×10¹³ GC/mL

In certain embodiments, the disclosure provides a pharmaceutical composition or formulation comprising a recombinant adeno-associated virus (AAV), potassium phosphate monobasic, sodium chloride, sodium phosphate dibasic anhydrous, sucrose, and poloxamer 188. In some embodiments, the AAV comprises components from AAV8. In some embodiments, the AAV is AAV viral vectors provided herein comprise the following elements in the following order: a) a constitutive or a hypoxia-inducible promoter sequence, and b) a sequence encoding the transgene (e.g., an anti-VEGF antigen-binding fragment moiety). In some embodiments, the transgene is a fully human post-translationally modified (HuPTM) antibody against VEGF. Antibodies include, but are not limited to, monoclonal antibodies, polyclonal antibodies, recombinantly produced antibodies, human antibodies, humanized antibodies, chimeric antibodies, synthetic antibodies, tetrameric antibodies comprising two heavy chain and two light chain molecules, antibody light chain monomers, antibody heavy chain monomers, antibody light chain dimers, antibody heavy chain dimers, antibody light chain-heavy chain pairs, intrabodies, heteroconjugate antibodies, monovalent antibodies, antigen-binding fragments of full-length antibodies, and fusion proteins of the above. Such antigen-binding fragments include, but are not limited to, single-domain antibodies (variable domain of heavy chain antibodies (VHHs) or nanobodies), Fabs, F(ab′)₂s, and scFvs (single-chain variable fragments) of full-length anti-VEGF antibodies (preferably, full-length anti-VEGF monoclonal antibodies (mAbs) (collectively referred to herein as “antigen-binding fragments”). In a preferred embodiment, the fully human post-translationally modified antibody against VEGF is a fully human post-translationally modified antigen-binding fragment of a monoclonal antibody (mAb) against VEGF (“HuPTMFabVEGFi”). In a further preferred embodiment, the HuPTMFabVEGFi is a fully human glycosylated antigen-binding fragment of an anti-VEGF mAb (“HuGlyFabVEGFi”). In an alternative embodiment, full-length mAbs can be used. In a preferred embodiment, the AAV used for delivering the transgene should have a tropism for human retinal cells or photoreceptor cells. Such AAV can include non-replicating recombinant adeno-associated virus vectors (“rAAV”), particularly those bearing an AAV8 capsid are preferred. In a specific embodiment, the viral vector or other DNA expression construct described herein is Construct I, wherein the Construct I comprises the following components: (1) AAV8 inverted terminal repeats that flank the expression cassette; (2) control elements, which include a) the CB7 promoter, comprising the CMV enhancer/chicken β-actin promoter, b) a chicken β-actin intron and c) a rabbit β-globin poly A signal; and (3) nucleic acid sequences coding for the heavy and light chains of anti-VEGF antigen-binding fragment, separated by a self-cleaving furin (F)/F2A linker, ensuring expression of equal amounts of the heavy and the light chain polypeptides. In another specific embodiment, the viral vector or other DNA expression construct described herein is Construct II, wherein the Construct II comprise the following components: (1) AAV2 inverted terminal repeats that flank the expression cassette; (2) control elements, which include a) the CB7 promoter, comprising the CMV enhancer/chicken β-actin promoter, b) a chicken β-actin intron and c) a rabbit β-globin poly A signal; and (3) nucleic acid sequences coding for the heavy and light chains of anti-VEGF antigen-binding fragment, separated by a self-cleaving furin (F)/F2A linker, ensuring expression of equal amounts of the heavy and the light chain polypeptides. In some embodiments, the anti-hVEGF antibody comprises a heavy chain comprising the amino acid sequence of SEQ ID NO:2 or SEQ ID NO:4, and a light chain comprising the amino acid sequence of SEQ ID NO:1 or SEQ ID NO:3.

In another embodiment, the viral vector or other expression construct suitable for packaging in an AAV capsid, comprises (1) AAV inverted terminal repeats (ITRs) flank the expression cassette; (2) regulatory control elements, consisting essentially of one or more enhancers and/or promoters, d) a poly A signal, and e) optionally an intron; and (3) a transgene providing (e.g., coding for) one or more RNA or protein products of interest.

In some embodiments, the pharmaceutical composition consists of: (a) the Construct II encoding an anti-human vascular endothelial growth factor (hVEGF) antibody, (b) potassium chloride at a concentration of 0.2 g/L, (c) potassium phosphate monobasic at a concentration of 0.2 g/L, (d) sodium chloride at a concentration of 5.84 g/L, (e) sodium phosphate dibasic anhydrous at a concentration of 1.15 g/L, (f) sucrose at a concentration of 4% weight/volume (40 g/L), (g) poloxamer 188 at a concentration of 0.001% weight/volume (0.01 g/L), and (h) water, and wherein the anti-hVEGF antibody comprises a heavy chain comprising the amino acid sequence of SEQ ID NO:2 or SEQ ID NO:4, and a light chain comprising the amino acid sequence of SEQ ID NO:1 or SEQ ID NO:3.

In some embodiments, the pharmaceutical composition consists of: (a) an AAV capsid packaging vector encoding a transgene of interest, (b) potassium chloride at a concentration of 0.2 g/L, (c) potassium phosphate monobasic at a concentration of 0.2 g/L, (d) sodium chloride at a concentration of 5.84 g/L, (e) sodium phosphate dibasic anhydrous at a concentration of 1.15 g/L, (f) sucrose at a concentration of 4% weight/volume (40 g/L), (g) poloxamer 188 at a concentration of 0.001% weight/volume (0.01 g/L), and (h) water, and wherein the transgene of interest encodes an RNA of interest or a protein of interest, for example an antibody or enzyme.

In some embodiments, the pharmaceutical composition consists of: (a) the Construct II encoding an anti-human vascular endothelial growth factor (hVEGF) antibody, (b) potassium chloride at a concentration of 0.2 g/L, (c) potassium phosphate monobasic at a concentration of 0.2 g/L, (d) sodium chloride at a concentration of 5.84 g/L, (e) sodium phosphate dibasic anhydrous at a concentration of 1.15 g/L, (f) sucrose at a concentration of 4% weight/volume (40 g/L), (g) poloxamer 188 at a concentration of 0.001% weight/volume (0.01 g/L), and (h) water, and wherein the anti-hVEGF antibody comprises a heavy chain comprising the amino acid sequence of SEQ ID NO:2 or SEQ ID NO:4, and a light chain comprising the amino acid sequence of SEQ ID NO:1 or SEQ ID NO:3, wherein the pharmaceutical composition has desired viscosity, density, and/or osmolality that is suitable for intravenous administration, subcutaneous administration, intramuscular injection, suprachoroidal injection (for example, via a suprachoroidal drug delivery device such as a microinjector with a microneedle), subretinal injection via transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), and/or a posterior juxtascleral depot procedure (for example, via a juxtascleral drug delivery device comprising a cannula whose tip can be inserted and kept in direct apposition to the scleral surface)).

In some embodiments, the pharmaceutical composition consists of: (a) the Construct II encoding an anti-human vascular endothelial growth factor (hVEGF) antibody, (b) potassium chloride at a concentration of 0.2 g/L, (c) potassium phosphate monobasic at a concentration of 0.2 g/L, (d) sodium chloride at a concentration of 5.84 g/L, (e) sodium phosphate dibasic anhydrous at a concentration of 1.15 g/L, (f) sucrose at a concentration of 4% weight/volume (40 g/L), (g) poloxamer 188 at a concentration of 0.001% weight/volume (0.01 g/L), and (h) water, and wherein the anti-hVEGF antibody comprises a heavy chain comprising the amino acid sequence of SEQ ID NO:2 or SEQ ID NO:4, and a light chain comprising the amino acid sequence of SEQ ID NO:1 or SEQ ID NO:3, wherein the pharmaceutical composition has ionic strength about 60 mM to 100 mM.

In some embodiments, the pharmaceutical composition consists of: (a) the Construct II encoding an anti-human vascular endothelial growth factor (hVEGF) antibody, (b) potassium chloride at a concentration of 0.2 g/L, (c) potassium phosphate monobasic at a concentration of 0.2 g/L, (d) sodium chloride at a concentration of 5.84 g/L, (e) sodium phosphate dibasic anhydrous at a concentration of 1.15 g/L, (f) sucrose at a concentration of 4% weight/volume (40 g/L), (g) poloxamer 188 at a concentration of 0.001% weight/volume (0.01 g/L), and (h) water, and wherein the anti-hVEGF antibody comprises a heavy chain comprising the amino acid sequence of SEQ ID NO:2 or SEQ ID NO:4, and a light chain comprising the amino acid sequence of SEQ ID NO:1 or SEQ ID NO:3, wherein the pharmaceutical composition has desired viscosity, density, and/or osmolality that is suitable for intravenous administration, subcutaneous administration, intramuscular injection, suprachoroidal injection (for example, via a suprachoroidal drug delivery device such as a microinjector with a microneedle), subretinal injection via transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), and/or a posterior juxtascleral depot procedure (for example, via a juxtascleral drug delivery device comprising a cannula whose tip can be inserted and kept in direct apposition to the scleral surface)).

In some embodiments, the pharmaceutical composition is a liquid composition. In some embodiments, the pharmaceutical composition is a frozen composition. In some embodiments, the pharmaceutical composition is a lyophilized composition from a liquid composition disclosed herein. In some embodiments, the pharmaceutical composition is a reconstituted lyophilized formulation.

In some embodiments, the pharmaceutical composition is a lyophilized composition comprising a residual moisture content between about 1% and about 7%. In some embodiments, the pharmaceutical composition is a lyophilized composition comprising a residual moisture content between about 2% and about 6%. In some embodiments, the pharmaceutical composition is a lyophilized composition comprising a residual moisture content between about 3% and about 4%. In some embodiments, the pharmaceutical composition is a lyophilized composition comprising a residual moisture content about 5%.

In certain aspects, disclosed herein is a method of treating or preventing a disease in a subject, comprising administering to the subject the pharmaceutical composition. In some embodiments, a pharmaceutical composition provided herein is suitable for administration by one, two or more routes of administration (e.g., suitable for suprachoroidal and subretinal administration).

The provided methods are suitable for used in the production of pharmaceutical compositions comprising recombinant AAV encoding a transgene. In some embodiments, provided herein are rAAV viral vectors encoding an anti-VEGF Fab or anti-VEGF antibody. In some embodiments, provided herein are rAAV8-based viral vectors encoding an anti-VEGF Fab or anti-VEGF antibody. In more embodiments, provided herein are rAAV8-based viral vectors encoding ranibizumab. In some embodiments, provided herein are rAAV viral vectors encoding Iduronidase (IDUA). In some embodiments, provided herein are rAAV9-based viral vectors encoding IDUA. In some embodiments, provided herein are rAAV viral vectors encoding Iduronate 2-Sulfatase (IDS). In some embodiments, provided herein are rAAV9-based viral vectors encoding IDS. In some embodiments, provided herein are rAAV viral vectors encoding a low-density lipoprotein receptor (LDLR). In some embodiments, provided herein are rAAV8-based viral vectors encoding LDLR. In some embodiments, provided herein are rAAV viral vectors encoding tripeptidyl peptidase 1 (TPP1) protein. In some embodiments, provided herein are rAAV9-based viral vectors encoding TPP1. In some embodiments, provided herein are rAAV viral vectors encoding microdystrophin protein. In some embodiments, provided herein are rAAV8-based viral vectors encoding microdystrophin. In some embodiments, provided herein are rAAV9-based viral vectors encoding microdystrophin. In some embodiments, provided herein are rAAV viral vectors encoding anti-kallikrein (anti-pKal) protein. In some embodiments, provided herein are rAAV8-based or rAAV9-based viral vectors encoding lanadelumab Fab or full-length antibody. In some embodiments, provided herein are rAAV viral vectors encoding human-alpha-sarcoglycan-gamma-sarcoglycan. In some embodiments, provided herein are rAAV viral vectors encoding huFollistatin344. In some embodiments, provided herein are rAAV viral vectors encoding human-alpha-sarcoglycan-gamma-sarcoglycan. In some embodiments, provided herein are rAAV viral vectors encoding CLN2. In some embodiments, provided herein are rAAV viral vectors encoding CLN3. In some embodiments, provided herein are rAAV viral vectors encoding CLN6. In some embodiments, provided herein are rAAV8-based or rAAV9-based viral vectors encoding human-alpha-sarcoglycan-gamma-sarcoglycan. In some embodiments, provided herein are rAAV8-based or rAAV9-based viral vectors encoding huFollistatin344. In some embodiments, provided herein are rAAV8-based or rAAV9-based viral vectors encoding human-alpha-sarcoglycan-gamma-sarcoglycan. In some embodiments, provided herein are rAAV8-based or rAAV9-based viral vectors encoding CLN2. In some embodiments, provided herein are rAAV8-based or rAAV9-based viral vectors encoding CLN3. In some embodiments, provided herein are rAAV8-based or rAAV9-based viral vectors encoding CLN6.

In certain aspects, disclosed herein is a method of treating or preventing a disease in a subject, comprising administering to the subject the pharmaceutical composition by intravenous administration, subcutaneous administration, intramuscular injection, suprachoroidal injection (for example, via a suprachoroidal drug delivery device such as a microinjector with a microneedle), subretinal injection via transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), and/or a posterior juxtascleral depot procedure (for example, via a juxtascleral drug delivery device comprising a cannula whose tip can be inserted and kept in direct apposition to the scleral surface)).

In certain embodiments, the pharmaceutical composition provided herein is suitable for intravenous administration, subcutaneous administration, intramuscular injection, suprachoroidal injection (for example, via a suprachoroidal drug delivery device such as a microinjector with a microneedle), subretinal injection via transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), and/or a posterior juxtascleral depot procedure (for example, via a juxtascleral drug delivery device comprising a cannula whose tip can be inserted and kept in direct apposition to the scleral surface)).

In certain embodiments, the pharmaceutical composition has a desired viscosity that is suitable for intravenous administration, subcutaneous administration, intramuscular injection, suprachoroidal injection (for example, via a suprachoroidal drug delivery device such as a microinjector with a microneedle), subretinal injection via transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), and/or a posterior juxtascleral depot procedure (for example, via a juxtascleral drug delivery device comprising a cannula whose tip can be inserted and kept in direct apposition to the scleral surface)).

In certain embodiments, the pharmaceutical composition has a desired density that is suitable for intravenous administration, subcutaneous administration, intramuscular injection, suprachoroidal injection (for example, via a suprachoroidal drug delivery device such as a microinjector with a microneedle), subretinal injection via transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), and/or a posterior juxtascleral depot procedure (for example, via a juxtascleral drug delivery device comprising a cannula whose tip can be inserted and kept in direct apposition to the scleral surface)).

In certain embodiments, the pharmaceutical composition has a desired osmolality that is suitable for intravenous administration, subcutaneous administration, intramuscular injection, suprachoroidal injection (for example, via a suprachoroidal drug delivery device such as a microinjector with a microneedle), subretinal injection via transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), and/or a posterior juxtascleral depot procedure (for example, via a juxtascleral drug delivery device comprising a cannula whose tip can be inserted and kept in direct apposition to the scleral surface)). In specific embodiments, the desired osmolality for subretinal administration is 160-430 mOsm/kg H₂O. In other specific embodiments, the desired osmolality of suprachoroidal administration is less than 600 mOsm/kg H₂O.

In certain embodiments, the pharmaceutical composition has a osmolality of about 100 to 500 mOsm/kg H₂O. In certain embodiments, the pharmaceutical composition has a osmolality of about 130 to 470 mOsm/kg H₂O. In certain embodiments, the pharmaceutical composition has a osmolality of about 160 to 430 mOsm/kg H₂O. In certain embodiments, the pharmaceutical composition has a osmolality of about 200 to 400 mOsm/kg H₂O. In certain embodiments, the pharmaceutical composition has a osmolality of about 240 to 340 mOsm/kg H₂O. In certain embodiments, the pharmaceutical composition has a osmolality of about 280 to 300 mOsm/kg H₂O. In certain embodiments, the pharmaceutical composition has a osmolality of about 295 to 395 mOsm/kg H₂O. In certain embodiments, the pharmaceutical composition has a osmolality of less than 600 mOsm/kg H₂O. In certain embodiments, the pharmaceutical composition has a osmolality range of 200 mOsm/L to 660 mOsm/L. In certain embodiments, the pharmaceutical composition has a osmolality of about 200 mOsm/L. In certain embodiments, the pharmaceutical composition has a osmolality of about 250 mOsm/L. In certain embodiments, the pharmaceutical composition has a osmolality of about 300 mOsm/L. In certain embodiments, the pharmaceutical composition has a osmolality of about 350 mOsm/L. In certain embodiments, the pharmaceutical composition has a osmolality of about 400 mOsm/L. In certain embodiments, the pharmaceutical composition has a osmolality of about 450 mOsm/L. In certain embodiments, the pharmaceutical composition has a osmolality of about 500 mOsm/L. In certain embodiments, the pharmaceutical composition has a osmolality of about 550 mOsm/L. In certain embodiments, the pharmaceutical composition has a osmolality of about 600 mOsm/L. In certain embodiments, the pharmaceutical composition has a osmolality of about 650 mOsm/L. In certain embodiments, the pharmaceutical composition has a osmolality of about 660 mOsm/L. In certain aspects, disclosed herein are methods of treating a subject diagnosed with mucopolysaccharidosis type IVA (MPS IVA), mucopolysaccharidosis type I (MPS I), mucopolysaccharidosis type II (MPS II), familial hypercholesterolemia (FH), homozygous familial hypercholesterolemia (HoFH), coronary artery disease, cerebrovascular disease, Duchenne muscular dystrophy, Limb Girdle muscular dystrophy, Becker muscular dystrophy and sporadic inclusion body myositis, or kallikrein-related disease comprising administering to the subject the pharmaceutical composition.

In certain aspects, disclosed herein are methods of treating a subject diagnosed with mucopolysaccharidosis type IVA (MPS IVA), mucopolysaccharidosis type I (MPS I), mucopolysaccharidosis type II (MPS II), familial hypercholesterolemia (FH), homozygous familial hypercholesterolemia (HoFH), coronary artery disease, cerebrovascular disease, Duchenne muscular dystrophy, Limb Girdle muscular dystrophy, Becker muscular dystrophy and sporadic inclusion body myositis, or kallikrein-related disease comprising administering to the subject the pharmaceutical composition.

In certain aspects, disclosed herein are methods of treating a subject diagnosed with mucopolysaccharidosis type IVA (MPS IVA), mucopolysaccharidosis type I (MPS I), mucopolysaccharidosis type II (MPS II), familial hypercholesterolemia (FH), homozygous familial hypercholesterolemia (HoFH), coronary artery disease, cerebrovascular disease, Duchenne muscular dystrophy, Limb Girdle muscular dystrophy, Becker muscular dystrophy and sporadic inclusion body myositis, or kallikrein-related disease comprising administering to the subject a therapeutically effective amount of the pharmaceutical composition by intravenous administration, subcutaneous administration, or intramuscular injection.

In certain aspects, disclosed herein are methods of treating or preventing a disease in a subject, comprising treating a subject diagnosed with nAMD (wet AMD), dry AMD, retinal vein occlusion (RVO), diabetic macular edema (DME), diabetic retinopathy (DR), or Batten disease comprising administering to the subject a therapeutically effective amount of the pharmaceutical composition.

In certain aspects, disclosed herein are methods of treating a subject diagnosed with nAMD (wet AMD), dry AMD, retinal vein occlusion (RVO), diabetic macular edema (DME), diabetic retinopathy (DR), or Batten comprising administering to the subject a therapeutically effective amount of the pharmaceutical composition by suprachoroidal injection (for example, via a suprachoroidal drug delivery device such as a microinjector with a microneedle), subretinal injection via transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), or a posterior juxtascleral depot procedure (for example, via a juxtascleral drug delivery device comprising a cannula whose tip can be inserted and kept in direct apposition to the scleral surface)).

In certain embodiments, compositions and methods are described for the delivery of pharmaceutical composition comprising a fully human post-translationally modified (HuPTM) antibody against VEGF to the retina/vitreal humour in the eye(s) of patients (human subjects) diagnosed with nAMD (wet AMD), dry AMD, retinal vein occlusion (RVO), diabetic macular edema (DME), or diabetic retinopathy (DR). Antibodies include, but are not limited to, monoclonal antibodies, polyclonal antibodies, recombinantly produced antibodies, human antibodies, humanized antibodies, chimeric antibodies, synthetic antibodies, tetrameric antibodies comprising two heavy chain and two light chain molecules, antibody light chain monomers, antibody heavy chain monomers, antibody light chain dimers, antibody heavy chain dimers, antibody light chain-heavy chain pairs, intrabodies, heteroconjugate antibodies, monovalent antibodies, and antigen-binding fragments of full-length antibodies, and fusion proteins of the above. Such antigen-binding fragments include, but are not limited to,single-domain antibodies (variable domain of heavy chain antibodies (VHHs) or nanobodies), Fabs, F(ab′)₂s, and scFvs (single-chain variable fragments) of full-length anti-VEGF antibodies (preferably, full-length anti-VEGF monoclonal antibodies (mAbs)) (collectively referred to herein as “antigen-binding fragments”). In a preferred embodiment, the fully human post-translationally modified antibody against VEGF is a fully human post-translationally modified antigen-binding fragment of a monoclonal antibody (mAb) against VEGF (“HuPTMFabVEGFi”). In a further preferred embodiment, the HuPTMFabVEGFi is a fully human glycosylated antigen-binding fragment of an anti-VEGF mAb (“HuGlyFabVEGFi”). See, also, International Patent Application Publication No. WO/2017/180936 (International Patent Application No. PCT/US2017/027529, filed Apr. 14, 2017), International Patent Application Publication No. WO/2017/181021 (International Patent Application No. PCT/US2017/027650, filed Apr. 14, 2017), and International Patent Application Publication No. WO2019/067540 (International Patent Application No. PCT/US2018/052855, filed Sep. 26, 2018),each of which is incorporated by reference herein in its entirety, for compositions and methods that can be used according to the invention described herein. In an alternative embodiment, full-length mAbs can be used. Delivery may be accomplished via gene therapy—e.g., by administering a viral vector or other DNA expression construct encoding an anti-VEGF antigen-binding fragment or mAb (or a hyperglycosylated derivative) to the suprachoroidal space, subretinal space (from a transvitreal approach or with a catheter through the suprachoroidal space), intraretinal space, and/or outer surface of the sclera (i.e., juxtascleral administration) in the eye(s) of patients (human subjects) diagnosed with nAMD (wet AMD), dry AMD, retinal vein occlusion (RVO), diabetic macular edema (DME), or diabetic retinopathy (DR), to create a permanent depot in the eye that continuously supplies the human PTM, e.g., human-glycosylated, transgene product. See, e.g., administration modes described in Section 5.3.2.

In certain embodiments, the patients have been shown to be responsive to treatment with an anti-VEGF antigen-binding fragment injected intravitreally prior to treatment with gene therapy. In specific embodiments, the patients have previously been treated with LUCENTIS @(ranibizumab), EYLEA® (aflibercept), and/or AVASTIN® (bevacizumab), and have been found to be responsive to one or more of said LUCENTIS® (ranibizumab), EYLEA® (aflibercept), and/or AVASTIN® (bevacizumab).

Subjects to whom such viral vector or other DNA expression construct is delivered should be responsive to the anti-VEGF antigen-binding fragment encoded by the transgene in the viral vector or expression construct. To determine responsiveness, the anti-hVEGF antigen-binding fragment transgene product (e.g., produced in cell culture, bioreactors, etc.) may be administered directly to the subject, such as by intravitreal injection.

The HuPTMFabVEGFi, e.g., HuGlyFabVEGFi, encoded by the transgene can include, but is not limited to an antigen-binding fragment of an antibody that binds to hVEGF, such as bevacizumab; an anti-hVEGF Fab moiety such as ranibizumab; or such bevacizumab or ranibizumab Fab moieties engineered to contain additional glycosylation sites on the Fab domain (e.g., see Courtois et al., 2016, mAbs 8: 99-112 which is incorporated by reference herein in its entirety for it description of derivatives of bevacizumab that are hyperglycosylated on the Fab domain of the full length antibody).

The recombinant vector used for delivering the transgene should have a tropism for human retinal cells or photoreceptor cells. Such vectors can include non-replicating recombinant adeno-associated virus vectors (“rAAV”), particularly those bearing an AAV8 capsid are preferred. However, other viral vectors may be used, including but not limited to lentiviral vectors, vaccinia viral vectors, or non-viral expression vectors referred to as “naked DNA” constructs. Preferably, the HuPTMFabVEGFi, e.g., HuGlyFabVEGFi, transgene should be controlled by appropriate expression control elements, for example, the CB7 promoter (a chicken β-actin promoter and CMV enhancer), the RPE65 promoter, or opsin promoter to name a few, and can include other expression control elements that enhance expression of the transgene driven by the vector (e.g., introns such as the chicken β-actin intron, minute virus of mice (MVM) intron, human factor IX intron (e.g., FIX truncated intron 1), β-globin splice donor/immunoglobulin heavy chain spice acceptor intron, adenovirus splice donor/immunoglobulin splice acceptor intron, SV40 μlate splice donor/splice acceptor (19S/16S) intron, and hybrid adenovirus splice donor/IgG splice acceptor intron and polyA signals such as the rabbit β-globin polyA signal, human growth hormone (hGH) polyA signal, SV40 μlate polyA signal, synthetic polyA (SPA) signal, and bovine growth hormone (bGH) polyA signal). See, e.g., Powell and Rivera-Soto, 2015, Discov. Med., 19(102):49-57.

In preferred embodiments, gene therapy constructs are designed such that both the heavy and light chains are expressed. More specifically, the heavy and light chains should be expressed at about equal amounts, in other words, the heavy and light chains are expressed at approximately a 1:1 ratio of heavy chains to light chains. The coding sequences for the heavy and light chains can be engineered in a single construct in which the heavy and light chains are separated by a cleavable linker or IRES so that separate heavy and light chain polypeptides are expressed. See, e.g., Section 5.2.4 for specific leader sequences and Section 5.2.5 for specific IRES, 2A, and other linker sequences that can be used with the methods and compositions provided herein.

In certain embodiments, gene therapy constructs are supplied as a frozen sterile, single use solution of the AAV vector active ingredient in a formulation buffer. In a specific embodiment, the pharmaceutical compositions suitable for subretinal administration comprise a suspension of the recombinant (e.g., rHuGlyFabVEGFi) vector in a formulation buffer comprising a physiologically compatible aqueous buffer, a surfactant and optional excipients. In a specific embodiment, the construct is formulated in Dulbecco's phosphate buffered saline and 0.001% poloxamer 188, pH=7.4.

4.2 Functional Properties

In certain embodiments, the pharmaceutical composition described herein is suitable for intravenous administration, subcutaneous administration, intramuscular injection, suprachoroidal injection (for example, via a suprachoroidal drug delivery device such as a microinjector with a microneedle), subretinal injection via transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), and/or a posterior juxtascleral depot procedure (for example, via a juxtascleral drug delivery device comprising a cannula whose tip can be inserted and kept in direct apposition to the scleral surface)).

In certain embodiments, the pharmaceutical composition has a desired density that is suitable for intravenous administration, subcutaneous administration, intramuscular injection, suprachoroidal injection (for example, via a suprachoroidal drug delivery device such as a microinjector with a microneedle), subretinal injection via transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), and/or a posterior juxtascleral depot procedure (for example, via a juxtascleral drug delivery device comprising a cannula whose tip can be inserted and kept in direct apposition to the scleral surface)).

In certain embodiments, the pharmaceutical composition has a desired osmolality that is suitable for intravenous administration, subcutaneous administration, intramuscular injection, suprachoroidal injection (for example, via a suprachoroidal drug delivery device such as a microinjector with a microneedle), subretinal injection via transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), and/or a posterior juxtascleral depot procedure (for example, via a juxtascleral drug delivery device comprising a cannula whose tip can be inserted and kept in direct apposition to the scleral surface)).

In certain embodiments, the pharmaceutical composition has a desired viscosity that is suitable for intravenous administration, subcutaneous administration, intramuscular injection, suprachoroidal injection (for example, via a suprachoroidal drug delivery device such as a microinjector with a microneedle), subretinal injection via transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), and/or a posterior juxtascleral depot procedure (for example, via a juxtascleral drug delivery device comprising a cannula whose tip can be inserted and kept in direct apposition to the scleral surface)).

In certain embodiments, the pharmaceutical composition has a osmolality of about 100 to 500 mOsm/kg H₂O. In certain embodiments, the pharmaceutical composition has a osmolality of about 130 to 470 mOsm/kg H₂O. In certain embodiments, the pharmaceutical composition has a osmolality of about 160 to 430 mOsm/kg H₂O. In certain embodiments, the pharmaceutical composition has a osmolality of about 200 to 400 mOsm/kg H₂O. In certain embodiments, the pharmaceutical composition has a osmolality of about 280 to 300 mOsm/kg H₂O. In certain embodiments, the pharmaceutical composition has a osmolality of about 240 to 340 mOsm/kg H₂O. In certain embodiments, the pharmaceutical composition has a osmolality of about 295 to 395 mOsm/kg H₂O. In certain embodiments, the pharmaceutical composition has a osmolality of less than 600 mOsm/kg H₂O. In certain embodiments, the pharmaceutical composition has a osmolality range of 200 mOsm/L to 660 mOsm/L. In certain embodiments, the pharmaceutical composition has a osmolality of about 200 mOsm/L. In certain embodiments, the pharmaceutical composition has a osmolality of about 250 mOsm/L. In certain embodiments, the pharmaceutical composition has a osmolality of about 300 mOsm/L. In certain embodiments, the pharmaceutical composition has a osmolality of about 350 mOsm/L. In certain embodiments, the pharmaceutical composition has a osmolality of about 400 mOsm/L. In certain embodiments, the pharmaceutical composition has a osmolality of about 450 mOsm/L. In certain embodiments, the pharmaceutical composition has a osmolality of about 500 mOsm/L. In certain embodiments, the pharmaceutical composition has a osmolality of about 550 mOsm/L. In certain embodiments, the pharmaceutical composition has a osmolality of about 600 mOsm/L. In certain embodiments, the pharmaceutical composition has a osmolality of about 650 mOsm/L. In certain embodiments, the pharmaceutical composition has a osmolality of about 660 mOsm/L. In certain aspects, disclosed herein are methods of treating a subject diagnosed with mucopolysaccharidosis type IVA (MPS IVA), mucopolysaccharidosis type I (MPS I), mucopolysaccharidosis type II (MPS II), familial hypercholesterolemia (FH), homozygous familial hypercholesterolemia (HoFH), coronary artery disease, cerebrovascular disease, Duchenne muscular dystrophy, Limb Girdle muscular dystrophy, Becker muscular dystrophy and sporadic inclusion body myositis, or kallikrein-related disease comprising administering to the subject the pharmaceutical composition.

In certain embodiments, the pharmaceutical composition has a osmolality range of 200 mOsm/L to 660 mOsm/L. In certain embodiments, the pharmaceutical composition has a osmolality range of 250 mOsm/L to 600 mOsm/L. In certain embodiments, the pharmaceutical composition has a osmolality range of 300 mOsm/L to 550 mOsm/L. In certain embodiments, the pharmaceutical composition has a osmolality range of 350 mOsm/L to 500 mOsm/L. In certain embodiments, the pharmaceutical composition has a osmolality range of 400 mOsm/L to 500 mOsm/L.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more stable to freeze/thaw cycles than the same recombinant AAV in a reference pharmaceutical composition. In certain embodiments, the stability of the recombinant AAV is determined by an assay or assays disclosed in Section 4.5 and Section 5.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more infectivity than the same recombinant AAV in a reference pharmaceutical composition. In certain embodiments, the virus infectivity of the recombinant AAV is determined by an assay or assays disclosed in Section 4.5 and Section 5. In certain embodiments, the infectivity is measured prior to or after freeze/thaw cycles.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 μless aggregation than the same recombinant AAV in a reference pharmaceutical composition. In certain embodiments, the aggregation of the recombinant AAV is determined by an assay or assays disclosed in Section 4.5 and Section 5. In certain embodiments, the aggregation is measured prior to or after freeze/thaw cycles.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more stable over a period of time, for example, about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, 12 months, about 15 months, about 18 months, about 24 months, about 2 years, about 3 years, about 4 years than the same recombinant AAV in a reference pharmaceutical composition. In certain embodiments, the stability over a period of time of the recombinant AAV is determined by an assay or assays disclosed in Section 4.5 and Section 5.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more stable over a period of time, at least for example, about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, 12 months, about 15 months, about 18 months, about 24 months, about 2 years, about 3 years, about 4 years than the same recombinant AAV in a reference pharmaceutical composition. In certain embodiments, the stability over a period of time of the recombinant AAV is determined by an assay or assays disclosed in Section 4.5 and Section 5.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 higher in vitro relative potency (IVRP) than the same recombinant AAV in a reference pharmaceutical composition. In certain embodiments, the in vitro relative potency (IVRP) of the recombinant AAV is determined by an assay or assays disclosed in Section 4.5 and Section 5. In certain embodiments, the in vitro relative potency (IVRP) is measured prior to or after freeze/thaw cycles.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 μless free DNA than the same recombinant AAV in a reference pharmaceutical composition. In certain embodiments, the free DNA of the recombinant AAV is determined by an assay or assays disclosed in Section 4.5 and Section 5. In certain embodiments, the aggregation is measured prior to or after freeze/thaw cycles.

In certain embodiments, the recombinant AAV in the pharmaceutical composition has at most 20%, 15%, 10%, 8%, 5%, 4%, 3%, 2%, or 1% change in size over a period of time, for example, about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years. In certain embodiments, the size of the recombinant AAV is determined by an assay or assays disclosed in Section 4.5 and Section 5. In certain embodiments, the size is measured prior to or after freeze/thaw cycles.

In certain embodiments, the recombinant AAV in the pharmaceutical composition has at most 20%, 15%, 10%, 8%, 5%, 4%, 3%, 2%, or 1% change in size over a period of time, for example, at least about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years. In certain embodiments, the size of the recombinant AAV is determined by an assay or assays disclosed in Section 4.5 and Section 5. In certain embodiments, the size is measured prior to or after freeze/thaw cycles.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more stable than the same recombinant AAV in a reference pharmaceutical composition when stored at −20° C. In certain embodiments, the stability of the recombinant AAV is determined by an assay or assays disclosed in Section 4.5 and Section 5.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more infectivity than the same recombinant AAV in a reference pharmaceutical composition when stored at −20° C. for a period of time, for example, about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years, than the same recombinant AAV in a reference pharmaceutical composition. In certain embodiments, the virus infectivity of the recombinant AAV is determined by an assay or assays disclosed in Section 4.5 and Section 5.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more infectivity than the same recombinant AAV in a reference pharmaceutical composition when stored at −20° C. for a period of time, for example, at least about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years, than the same recombinant AAV in a reference pharmaceutical composition. In certain embodiments, the virus infectivity of the recombinant AAV is determined by an assay or assays disclosed in Section 4.5 and Section 5.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 μless aggregation than the same recombinant AAV in a reference pharmaceutical composition when stored at −20° C. for a period of time, for example, about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years, than the same recombinant AAV in a reference pharmaceutical composition. In certain embodiments, the aggregation of the recombinant AAV is determined by an assay or assays disclosed in Section 4.5 and Section 5.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 μless aggregation than the same recombinant AAV in a reference pharmaceutical composition when stored at −20° C. for a period of time, at least for example, at least about 1 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years, than the same recombinant AAV in a reference pharmaceutical composition. In certain embodiments, the aggregation of the recombinant AAV is determined by an assay or assays disclosed in Section 4.5 and Section 5.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more stable when stored at −20° C. for a period of time, for example, about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years, than the same recombinant AAV in a reference pharmaceutical composition. In certain embodiments, the stability over a period of time of the recombinant AAV is determined by an assay or assays disclosed in Section 4.5 and Section 5.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more stable when stored at −20° C. for a period of time, for example, at least about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years, than the same recombinant AAV in a reference pharmaceutical composition. In certain embodiments, the stability over a period of time of the recombinant AAV is determined by an assay or assays disclosed in Section 4.5 and Section 5.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 higher in vitro relative potency (IVRP) than the same recombinant AAV in a reference pharmaceutical composition when stored at −20° C. for a period of time, for example, 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years, than the same recombinant AAV in a reference pharmaceutical composition. In certain embodiments, the in vitro relative potency (IVRP) of the recombinant AAV is determined by an assay or assays disclosed in Section 4.5 and Section 5.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 higher in vitro relative potency (IVRP) than the same recombinant AAV in a reference pharmaceutical composition when stored at −20° C. for a period of time, for example, at least about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years, than the same recombinant AAV in a reference pharmaceutical composition. In certain embodiments, the in vitro relative potency (IVRP) of the recombinant AAV is determined by an assay or assays disclosed in Section 4.5 and Section 5.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 higher in vitro relative potency (IVRP) than the same recombinant AAV in a reference pharmaceutical composition when stored at −20° C. for a period of time, for example, about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years, than the same recombinant AAV in a reference pharmaceutical composition. In certain embodiments, the in vitro relative potency (IVRP) of the recombinant AAV is determined by an assay or assays disclosed in Section 4.5 and Section 5.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 higher in vitro relative potency (IVRP) than the same recombinant AAV in a reference pharmaceutical composition when stored at −20° C. for a period of time, for example, at least about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years, than the same recombinant AAV in a reference pharmaceutical composition. In certain embodiments, the in vitro relative potency (IVRP) of the recombinant AAV is determined by an assay or assays disclosed in Section 4.5 and Section 5.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 μless free DNA than the same recombinant AAV in a reference pharmaceutical composition when stored at −20° C. for a period of time, for example, about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years, than the same recombinant AAV in a reference pharmaceutical composition. In certain embodiments, the free DNA of the recombinant AAV is determined by an assay or assays disclosed in Section 4.5 and Section 5.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 μless free DNA than the same recombinant AAV in a reference pharmaceutical composition when stored at −20° C. for a period of time, for example, at least about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years, than the same recombinant AAV in a reference pharmaceutical composition. In certain embodiments, the free DNA of the recombinant AAV is determined by an assay or assays disclosed in Section 4.5 and Section 5.

In certain embodiments, the recombinant AAV in the pharmaceutical composition has at most 20%, 15%, 10%, 8%, 5%, 4%, 3%, 2%, or 1% change in size when stored at −20° C. over a period of time, for example, about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years. In certain embodiments, the size of the recombinant AAV is determined by an assay or assays disclosed in Section 4.5 and Section 5.

In certain embodiments, the recombinant AAV in the pharmaceutical composition has at most 20%, 15%, 10%, 8%, 5%, 4%, 3%, 2%, or 1% change in size when stored at −20° C. over a period of time, for example, at least about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years. In certain embodiments, the size of the recombinant AAV is determined by an assay or assays disclosed in Section 4.5 and Section 5.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more stable than the same recombinant AAV in a reference pharmaceutical composition when stored at 37° C. In certain embodiments, the stability of the recombinant AAV is determined by an assay or assays disclosed in Section 4.5 and Section 5.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more infectivity than the same recombinant AAV in a reference pharmaceutical composition when stored at 37° C. for a period of time, for example, about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years, than the same recombinant AAV in a reference pharmaceutical composition. In certain embodiments, the virus infectivity of the recombinant AAV is determined by an assay or assays disclosed in Section 4.5 and Section 5.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more infectivity than the same recombinant AAV in a reference pharmaceutical composition when stored at 37° C. for a period of time, for example, at least about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years, than the same recombinant AAV in a reference pharmaceutical composition. In certain embodiments, the virus infectivity of the recombinant AAV is determined by an assay or assays disclosed in Section 4.5 and Section 5.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 μless aggregation than the same recombinant AAV in a reference pharmaceutical composition when stored at 37° C. for a period of time, for example, about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years, than the same recombinant AAV in a reference pharmaceutical composition. In certain embodiments, the aggregation of the recombinant AAV is determined by an assay or assays disclosed in Section 4.5 and Section 5.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 μless aggregation than the same recombinant AAV in a reference pharmaceutical composition when stored at 37° C. for a period of time, for example, at least about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years, than the same recombinant AAV in a reference pharmaceutical composition. In certain embodiments, the aggregation of the recombinant AAV is determined by an assay or assays disclosed in Section 4.5 and Section 5.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more stable when stored at 37° C. for a period of time, for example, about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years, than the same recombinant AAV in a reference pharmaceutical composition. In certain embodiments, the stability over a period of time of the recombinant AAV is determined by an assay or assays disclosed in Section 4.5 and Section 5.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more stable when stored at 37° C. for a period of time, for example, at least about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years, than the same recombinant AAV in a reference pharmaceutical composition. In certain embodiments, the stability over a period of time of the recombinant AAV is determined by an assay or assays disclosed in Section 4.5 and Section 5.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 higher in vitro relative potency (IVRP) than the same recombinant AAV in a reference pharmaceutical composition when stored at 37° C. for a period of time, for example, about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years, than the same recombinant AAV in a reference pharmaceutical composition. In certain embodiments, the in vitro relative potency (IVRP) of the recombinant AAV is determined by an assay or assays disclosed in Section 4.5 and Section 5.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 higher in vitro relative potency (IVRP) than the same recombinant AAV in a reference pharmaceutical composition when stored at 37° C. for a period of time, for example, at least about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years, than the same recombinant AAV in a reference pharmaceutical composition. In certain embodiments, the in vitro relative potency (IVRP) of the recombinant AAV is determined by an assay or assays disclosed in Section 4.5 and Section 5.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 μless free DNA than the same recombinant AAV in a reference pharmaceutical composition when stored at 37° C. for a period of time, for example, about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years, than the same recombinant AAV in a reference pharmaceutical composition. In certain embodiments, the free DNA of the recombinant AAV is determined by an assay or assays disclosed in Section 4.5 and Section 5.

In certain embodiments, the recombinant AAV in the pharmaceutical composition is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 μless free DNA than the same recombinant AAV in a reference pharmaceutical composition when stored at 37° C. for a period of time, at least for example, about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years, than the same recombinant AAV in a reference pharmaceutical composition. In certain embodiments, the free DNA of the recombinant AAV is determined by an assay or assays disclosed in Section 4.5 and Section 5.

In certain embodiments, the recombinant AAV in the pharmaceutical composition has at most 20%, 15%, 10%, 8%, 5%, 4%, 3%, 2%, or 1% change in size when stored at 37° C. over a period of time, for example, about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years. In certain embodiments, the size of the recombinant AAV is determined by an assay or assays disclosed in Section 4.5 and Section 5.

In certain embodiments, the recombinant AAV in the pharmaceutical composition has at most 20%, 15%, 10%, 8%, 5%, 4%, 3%, 2%, or 1% change in size when stored at 37° C. over a period of time, for example, at least about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, about 24 months, about 2 years, about 3 years, and about 4 years. In certain embodiments, the size of the recombinant AAV is determined by an assay or assays disclosed in Section 4.5 and Section 5.

In certain embodiments, a pharmaceutical composition provided herein is capable of being stored for 1, 2, 3, 4, 5, 6, 7, 8, 9, 10, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 months without loss of stability as determined, e.g., by an assay or assays disclosed in Section 4.5 or 5. In certain embodiments, a pharmaceutical composition provided herein is capable of being stored for 1, 2, 3, 4, 5, 6, 7, 8, 9, 10, 12, 13, 14, 15, 16, 17, 18, 19, 20, 21, 22, 23, or 24 months at 4° C. without loss of stability. In certain embodiments, a pharmaceutical composition provided herein is capable of being stored for 1, 2, 3, 4, 5, 6, 7, 8, 9, 10, 12, 13, 14, 15, 16, 17, 18, 19, 20, 21, 22, 23,24,25,26,27,28,29,30,31,32,33,34,35,36,37,38,39,40,41,42,43,44,45,46,47, or 48 months at ≤60° C. without loss of stability. In certain embodiments, a pharmaceutical composition provided herein is capable of being stored for 1, 2, 3, 4, 5, 6, 7, 8, 9, 10, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 months at −80° C. without loss of stability. In certain embodiments, a pharmaceutical composition provided herein is capable of being stored for 1, 2, 3, 4, 5, 6, 7, 8, 9, 10, 12, 13, 14, 15, 16, 17, 18, 19, 20, 21, 22, 23, or 24 months at 4° C. after having been stored at −20° C. for 1, 2, 3, 4, 5, 6, 7, 8, 9, 10, or 12 months without loss of stability.

In certain embodiments, a pharmaceutical composition provided herein is capable of being first stored for 1, 2, 3, 4, 5, 6, 7, 8, 9, 10, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 months at −80° C., then being thawed and, after thawing, being stored at 2-10° C., 4-8° C., 2, 3, 4, 5, 6, 7, 8 or 9° C. for 1, 2, 3, 4, 5, 6, 7, 8, 9, 10, or 12 additional months without loss of stability as determined, e.g., by an assay or assays disclosed in Section 4.5 or 5. In certain embodiments, a pharmaceutical composition provided herein is capable of being first stored for 1, 2, 3, 4, 5, 6, 7, 8, 9, 10, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 months at −80° C., then being thawed and, after thawing, being stored at about 4° C. for 1, 2, 3, 4, 5, 6, 7, 8, 9, 10, or 12 additional months without loss of stability as determined, e.g., by an assay or assays disclosed in Section 4.5 or 5. In certain embodiments, a pharmaceutical composition provided herein is capable of being first stored for 1, 2, 3, 4, 5, 6, 7, 8, 9, 10, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, or 48 months at ≤60° C., then being thawed and, after thawing, being stored at about 4° C. for 1, 2, 3, 4, 5, 6, 7, 8, 9, 10, or 12 additional months without loss of stability as determined, e.g., by an assay or assays disclosed in Section 4.5 or 5.

In another aspect, provided herein are single unit dosage forms comprising a recombinant AAV provided herein (e.g., Construct II). As used herein, the term “single unit dosage form” refers to a dosage form comprising the amount of recombinant AAV (e.g., Construct II) required for one patient in one visit. In some embodiments, the patient may be administered the recombinant AAV (e.g., Construct II) to one eye. In other embodiments, the patient may be administered the recombinant AAV (e.g., Construct II) to both eyes.

In some embodiments, provided herein is a single unit dosage form comprising 3.2×10¹¹ genome copies (GC)/mL of Construct II, 0.2 g/L potassium chloride, 0.2 g/L potassium phosphate monobasic, 8.01 g/L sodium chloride, 1.15 g/L sodium phosphate dibasic anhydrous, pH 7.4 and 0.001% P188 in a volume of about 0.95 mL in a vial. In some embodiments, provided herein is a single unit dosage form comprising 3.2×10¹¹ GC/mL of Construct II, 0.2 g/L potassium chloride, 0.2 g/L potassium phosphate monobasic, 8.01 g/L sodium chloride, 1.15 g/L sodium phosphate dibasic anhydrous, pH 7.4 and 0.001% P188 in a volume of at least 0.8 mL in a vial.

In some embodiments, provided herein in is a single unit dosage form comprising 3.2×10¹¹ GC/mL of Construct II, 0.2 g/L potassium chloride, 0.2 g/L potassium phosphate monobasic, 8.01 g/L sodium chloride, 1.15 g/L sodium phosphate dibasic anhydrous, pH 7.4, 4% sucrose and 0.001% P188 in a volume of about 0.95 mL in a vial. In some embodiments, provided herein in is a single unit dosage form comprising 3.2×10¹¹ GC/mL of Construct II, 0.2 g/L potassium chloride, 0.2 g/L potassium phosphate monobasic, 8.01 g/L sodium chloride, 1.15 g/L sodium phosphate dibasic anhydrous, pH 7.4, 4% sucrose and 0.001% P188 in a volume of at least 0.8 mL in a vial.

In some embodiments, provided herein in is a single unit dosage form comprising 6.5×10¹¹ GC/mL of Construct II, 0.2 g/L potassium chloride, 0.2 g/L potassium phosphate monobasic, 8.01 g/L sodium chloride, 1.15 g/L sodium phosphate dibasic anhydrous, pH 7.4 and 0.001% P188 in a volume of about 0.95 mL in a vial. In some embodiments, provided herein in is a single unit dosage form comprising 6.5×10¹¹ GC/mL of Construct II, 0.2 g/L potassium chloride, 0.2 g/L potassium phosphate monobasic, 8.01 g/L sodium chloride, 1.15 g/L sodium phosphate dibasic anhydrous, pH 7.4, and 0.001% P188 in a volume of at least 0.8 mL in a vial.

In some embodiments, provided herein in is a single unit dosage form comprising 6.5×10¹¹ GC/mL of Construct II, 0.2 g/L potassium chloride, 0.2 g/L potassium phosphate monobasic, 8.01 g/L sodium chloride, 1.15 g/L sodium phosphate dibasic anhydrous, pH 7.4, 4% sucrose and 0.001% P188 in a volume of about 0.95 mL in a vial. In some embodiments, provided herein in is a single unit dosage form comprising 6.5×10¹¹ GC/mL of Construct II, 0.2 g/L potassium chloride, 0.2 g/L potassium phosphate monobasic, 8.01 g/L sodium chloride, 1.15 g/L sodium phosphate dibasic anhydrous, pH 7.4, 4% sucrose and 0.001% P188 in a volume of at least 0.8 mL in a vial.

In some embodiments, provided herein in is a single unit dosage form comprising 2.5×10¹² GC/mL of Construct II, 0.2 g/L potassium chloride, 0.2 g/L potassium phosphate monobasic, 8.01 g/L sodium chloride, 1.15 g/L sodium phosphate dibasic anhydrous, pH 7.4, and 0.001% P188 in a volume of about 0.6 mL in a vial. In some embodiments, provided herein in is a single unit dosage form comprising 2.5×10¹² GC/mL of Construct II, 0.2 g/L potassium chloride, 0.2 g/L potassium phosphate monobasic, 8.01 g/L sodium chloride, 1.15 g/L sodium phosphate dibasic anhydrous, pH 7.4, and 0.001% P188 in a volume of at least 0.5 mL in a vial.

In some embodiments, provided herein in is a single unit dosage form comprising 3×10¹³ GC/mL of Construct II, 0.2 g/L potassium chloride, 0.2 g/L potassium phosphate monobasic, 8.01 g/L sodium chloride, 1.15 g/L sodium phosphate dibasic anhydrous, pH 7.4, 4% sucrose and 0.001% P188 in a volume of about 0.6 mL in a vial. In some embodiments, provided herein in is a single unit dosage form comprising 3×10¹³ GC/mL of Construct II, 0.2 g/L potassium chloride, 0.2 g/L potassium phosphate monobasic, 8.01 g/L sodium chloride, 1.15 g/L sodium phosphate dibasic anhydrous, pH 7.4, 4% sucrose and 0.001% P188 in a volume of at least 0.5 mL in a vial.

In some embodiments, a single unit dosage form provided herein is contained in a hydrophobically-coated glass vial. In some embodiments, a single unit dosage form provided herein is contained in a Cyclo Olefin Polymer (COP) vial. In some embodiments, a single unit dosage form provided herein is contained in a Daikyo Crystal Zenith® (CZ) vial.

In some embodiments, the single unit dosage forms provided herein are administered to a subject via subretinal administration. In some embodiments, the single unit dosage forms provided herein are administered to a subject via suprachoroidal administration. In some embodiments, the single unit dosage forms provided herein may be suitable for both subretinal and suprachoroidal administration. Also provided herein is a pre-filled syringe containing a single unit dosage form provided herein. In some embodiments, a single unit dosage form provided herein is capable of being stored at 4° C. for 1 weeks, about 2 weeks, about 3 weeks, about 4 weeks, about 1 month, about 2 months. In some embodiments, a single unit dosage form provided herein is capable of being stored at −80° C. for about 6 months, about 7 months, about 8 months, about 9 months, about 10 months, about 11 months, about 12 months, about 15 months, about 18 months, about 24 months. In some embodiments, a single unit dosage form provided herein is capable of being stored at 4° C. for 1 weeks, about 2 weeks, about 3 weeks, about 4 weeks, about 1 month, about 2 months after having previously been stored at −80° C. for about 6 months, about 7 months, about 8 months, about 9 months, about 10 months, about 11 months, about 12 months, about 15 months, about 18 months, about 24 months.

4.3 Dosage and Mode of Administration

In certain aspects, disclosed herein is a method of treating or preventing a disease in a subject, comprising administering to the subject the pharmaceutical composition by intravenous administration, subcutaneous administration, intramuscular injection, suprachoroidal injection (for example, via a suprachoroidal drug delivery device such as a microinjector with a microneedle), subretinal injection via transvitreal approach (a surgical procedure), subretinal administration via the suprachoroidal space (for example, a surgical procedure via a subretinal drug delivery device comprising a catheter that can be inserted and tunneled through the suprachoroidal space toward the posterior pole, where a small needle injects into the subretinal space), and/or a posterior juxtascleral depot procedure (for example, via a juxtascleral drug delivery device comprising a cannula whose tip can be inserted and kept in direct apposition to the scleral surface)). In certain embodiments, the vector genome concentration (VGC) of the pharmaceutical composition is about 3×10⁹ GC/mL, about 1×10¹⁰ GC/mL, about 1.2×10¹⁰ GC/mL, about 1.6×10¹⁰ GC/mL, about 4×10¹⁰ GC/mL, about 6×10¹⁰ GC/mL, about 2×10¹¹ GC/mL, about 2.4×10¹¹ GC/mL, about 2.5×10¹¹ GC/mL, about 3×10¹¹ GC/mL, about 6.2×10¹¹ GC/mL, about 1×10¹² GC/mL, about 3×10¹² GC/mL, about 2×10¹³ GC/mL or about 3×10¹³ GC/mL

In certain embodiments, the vector genome concentration (VGC) of the pharmaceutical composition is about 3×10⁹ GC/mL, 4×10⁹ GC/mL, 5×10⁹ GC/mL, 6×10⁹ GC/mL, 7×10⁹ GC/mL, 8×10⁹ GC/mL, 9×10⁹ GC/mL, about 1×10¹⁰ GC/mL, about 2×10¹⁰ GC/mL, about 3×10¹⁰ GC/mL, about 4×10¹⁰ GC/mL, about 5×10¹⁰ GC/mL, about 6×10¹⁰ GC/mL, about 7×10¹⁰ GC/mL, about 8×10¹⁰ GC/mL, about 9×10¹⁰ GC/mL, about 1×10¹¹ GC/mL, about 2×10¹¹ GC/mL, about 3×10¹¹ GC/mL, about 4×10¹¹ GC/mL, about 5×10¹¹ GC/mL, about 6×10¹¹ GC/mL, about 7×10¹¹ GC/mL, about 8×10¹¹ GC/mL, about 9×10¹¹ GC/mL, about 1×10¹² GC/mL, about 2×10¹² GC/mL, about 3×10¹² GC/mL, about 4×10¹² GC/mL, about 5×10¹² GC/mL, about 6×10¹² GC/mL, about 7×10¹² GC/mL, about 8×10¹² GC/mL, about 9×10¹² GC/mL, about 1×10¹³ GC/mL, about 1×10¹³ GC/mL, about 2×10¹³ GC/mL, about 3×10¹³ GC/mL.

Therapeutically effective doses of the recombinant vector should be administered subretinally and/or intraretinally (e.g., by subretinal injection via the transvitreal approach (a surgical procedure), or via the suprachoroidal space) in a volume ranging from ≥0.1 mL to 0.5 mL, preferably in 0.1 to 0.30 mL (100-300 μl), and most preferably, in a volume of 0.25 mL (250 μl). Therapeutically effective doses of the recombinant vector may be administered in one or more injections during the same visit. Therapeutically effective doses of the recombinant vector should be administered suprachoroidally (e.g., by suprachoroidal injection) in a volume of 100 μl or less, for example, in a volume of 50-100 μl. Therapeutically effective doses of the recombinant vector should be administered to the outer surface of the sclera in a volume of 500 μl or less, for example, in a volume of 500 μl or less, for example, in a volume of 10-20 μl, 20-50 μl, 50-100 μl, 100-200 μl, 200-300 μl, 300-400 μl, or 400-500 μl.

In certain embodiments, the recombinant vector is administered suprachoroidally (e.g., by suprachoroidal injection). In a specific embodiment, suprachoroidal administration (e.g., an injection into the suprachoroidal space) is performed using a suprachoroidal drug delivery device. Suprachoroidal drug delivery devices are often used in suprachoroidal administration procedures, which involve administration of a drug to the suprachoroidal space of the eye (see, e.g., Hariprasad, 2016, Retinal Physician 13: 20-23; Goldstein, 2014, Retina Today 9(5): 82-87; Baldassarre et al., 2017; each of which is incorporated by reference herein in its entirety). The suprachoroidal drug delivery devices that can be used to deposit the expression vector in the subretinal space according to the invention described herein include, but are not limited to, suprachoroidal drug delivery devices manufactured by Clearside® Biomedical, Inc. (see, for example, Hariprasad, 2016, Retinal Physician 13: 20-23) and MedOne suprachoroidal catheters.

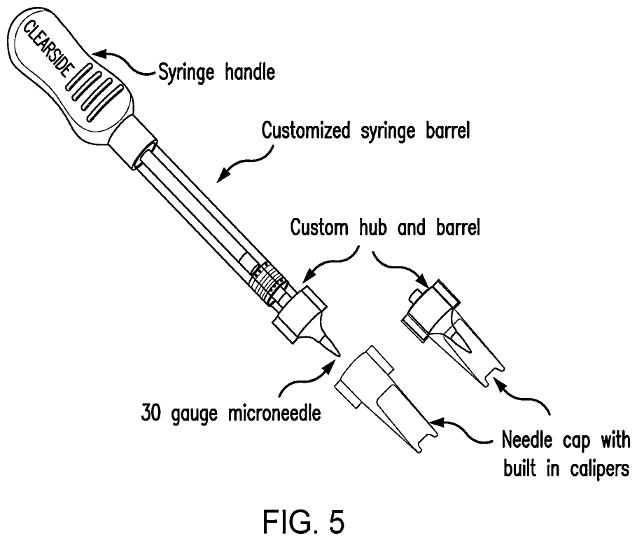

In a specific embodiment, the suprachoroidal drug delivery device is a syringe with a 1 millimeter 30 gauge needle (see FIG. 5). During an injection using this device, the needle pierces to the base of the sclera and fluid containing drug enters the suprachoroidal space, leading to expansion of the suprachoroidal space. As a result, there is tactile and visual feedback during the injection. Following the injection, the fluid flows posteriorly and absorbs dominantly in the choroid and retina. This results in the production of transgene protein from all retinal cell layers and choroidal cells. Using this type of device and procedure allows for a quick and easy in-office procedure with low risk of complications. A max volume of 100 μl can be injected into the suprachoroidal space.

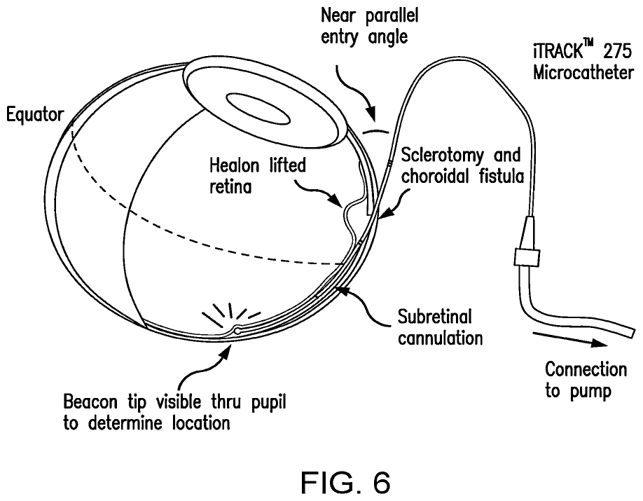

In certain embodiments, the recombinant vector is administered subretinally via the suprachoroidal space by use of a subretinal drug delivery device. In certain embodiments, the subretinal drug delivery device is a catheter which is inserted and tunneled through the suprachoroidal space around to the back of the eye during a surgical procedure to deliver drug to the subretinal space (see FIG. 6). This procedure allows the vitreous to remain intact and thus, there are fewer complication risks (less risk of gene therapy egress, and complications such as retinal detachments and macular holes), and without a vitrectomy, the resulting bleb may spread more diffusely allowing more of the surface area of the retina to be transduced with a smaller volume. The risk of induced cataract following this procedure is minimized, which is desirable for younger patients. Moreover, this procedure can deliver bleb under the fovea more safely than the standard transvitreal approach, which is desirable for patients with inherited retinal diseases effecting central vision where the target cells for transduction are in the macula. This procedure is also favorable for patients that have neutralizing antibodies (Nabs) to AAVs present in the systemic circulation which may impact other routes of delivery (such as suprachoroidal and intravitreal). Additionally, this method has shown to create blebs with less egress out the retinotomy site than the standard transvitreal approach. The subretinal drug delivery device originally manufactured by Janssen Pharmaceuticals, Inc. now by Orbit Biomedical Inc. (see, for example, Subretinal Delivery of Cells via the Suprachoroidal Space: Janssen Trial. In: Schwartz et al. (eds) Cellular Therapies for Retinal Disease, Springer, Cham; International Patent Application Publication No. WO 2016/040635 A1) can be used for such purpose.

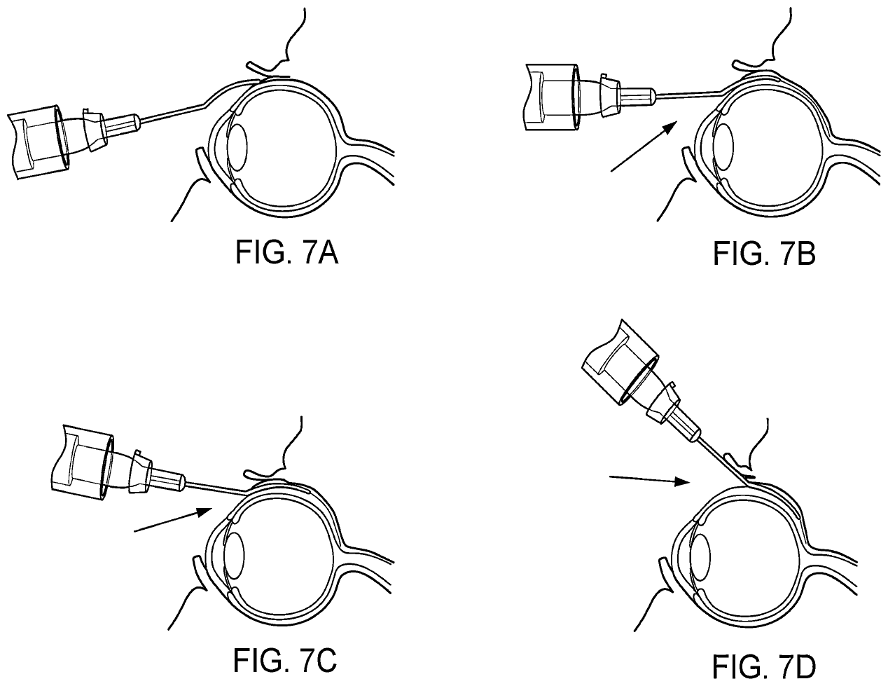

In certain embodiments, the recombinant vector is administered to the outer surface of the sclera (for example, by the use of a juxtascleral drug delivery device that comprises a cannula, whose tip can be inserted and kept in direct apposition to the scleral surface). In a specific embodiment, administration to the outer surface of the sclera is performed using a posterior juxtascleral depot procedure, which involves drug being drawn into a blunt-tipped curved cannula and then delivered in direct contact with the outer surface of the sclera without puncturing the eyeball. In particular, following the creation of a small incision to bare sclera, the cannula tip is inserted (see FIG. 7A). The curved portion of the cannula shaft is inserted, keeping the cannula tip in direct apposition to the scleral surface (see FIG. 7B-7D). After complete insertion of the cannula (FIG. 7D), the drug is slowly injected while gentle pressure is maintained along the top and sides of the cannula shaft with sterile cotton swabs. This method of delivery avoids the risk of intraocular infection and retinal detachment, side effects commonly associated with injecting therapeutic agents directly into the eye.

Doses that maintain a concentration of the transgene product at a Cmin of at least 0.330 μg/mL in the Vitreous humour, or 0.110 μg/mL in the Aqueous humour (the anterior chamber of the eye) for three months are desired; thereafter, Vitreous Cmin concentrations of the transgene product ranging from 1.70 to 6.60 μg/mL, and/or Aqueous Cmin concentrations ranging from 0.567 to 2.20 μg/mL should be maintained. However, because the transgene product is continuously produced (under the control of a constitutive promoter or induced by hypoxic conditions when using an hypoxia-inducible promoter), maintenance of lower concentrations can be effective. Vitreous humour concentrations can be measured directly in patient samples of fluid collected from the vitreous humour or the anterior chamber, or estimated and/or monitored by measuring the patient's serum concentrations of the transgene product—the ratio of systemic to vitreal exposure to the transgene product is about 1:90,000. (E.g., see, vitreous humor and serum concentrations of ranibizumab reported in Xu L, et al., 2013, Invest. Opthal. Vis. Sci. 54: 1616-1624, at p. 1621 and Table 5 at p. 1623, which is incorporated by reference herein in its entirety).

In certain embodiments, dosages are measured by genome copies per ml or the number of genome copies administered to the eye of the patient (e.g., administered suprachoroidally, subretinally, juxtasclerally and/or intraretinally (e.g., by suprachoroidal injection, subretinal injection via the transvitreal approach (a surgical procedure), subretinal administration via the suprachoroidal space, or a posterior juxtascleral depot procedure). In certain embodiments, 2.4×10¹¹ genome copies per ml to 1×10¹³ genome copies per ml are administered. In a specific embodiment, 2.4×10¹¹ genome copies per ml to 5×10¹¹ genome copies per ml are administered. In another specific embodiment, 5×10¹¹ genome copies per ml to 1×10¹² genome copies per ml are administered. In another specific embodiment, 1×10¹² genome copies per ml to 5×10¹² genome copies per ml are administered. In another specific embodiment, 5×10¹² genome copies per ml to 1×10¹³ genome copies per ml are administered. In another specific embodiment, about 2.4×10¹¹ genome copies per ml are administered. In another specific embodiment, about 5×10¹¹ genome copies per ml are administered. In another specific embodiment, about 1×10¹² genome copies per ml are administered. In another specific embodiment, about 5×10¹² genome copies per ml are administered. In another specific embodiment, about 1×10¹³ genome copies per ml are administered. In certain embodiments, 1×10⁹ to 1×10¹² genome copies are administered. In specific embodiments, 3×10⁹ to 2.5×10¹¹ genome copies are administered. In specific embodiments, 1×10⁹ to 2.5×10¹¹ genome copies are administered. In specific embodiments, 1×10⁹ to 1×10¹¹ genome copies are administered. In specific embodiments, 1×10⁹ to 5×10⁹ genome copies are administered. In specific embodiments, 6×10⁹ to 3×10¹⁰ genome copies are administered. In specific embodiments, 4×10¹⁰ to 1×10¹¹ genome copies are administered. In specific embodiments, 2×10¹¹ to 1×10¹² genome copies are administered. In a specific embodiment, about 3×10⁹ genome copies are administered (which corresponds to about 1.2×10¹⁰ genome copies per ml in a volume of 250 μl). In another specific embodiment, about 1×10¹⁰ genome copies are administered (which corresponds to about 4×10¹⁰ genome copies per ml in a volume of 250 μl). In another specific embodiment, about 6×10¹⁰ genome copies are administered (which corresponds to about 2.4×10¹¹ genome copies per ml in a volume of 250 μl). In another specific embodiment, about 1.6×10¹¹ genome copies are administered (which corresponds to about 6.2×10¹¹ genome copies per ml in a volume of 250 μl). In another specific embodiment, about 1.55×10¹¹ genome copies are administered (which corresponds to about 6.2×10¹¹ genome copies per ml in a volume of 250 μl). In another specific embodiment, about 1.6×10¹¹ genome copies are administered (which corresponds to about 6.4×10¹¹ genome copies per ml in a volume of 250 μl). In another specific embodiment, about 2.5×10¹¹ genome copies (which corresponds to about 1.0×10¹² genome copies per ml in a volume of 250 μl) are administered. In another specific embodiment, about 6.4×10¹⁰ genome copies (which corresponds to about 3.2×10¹¹ genome copies per ml in a volume of 200 μl) are administered. In another specific embodiment, about 1.3×10¹¹ genome copies (which corresponds to about 6.5×10¹¹ genome copies per ml in a volume of 200 μl) are administered.

As used herein and unless otherwise specified, the term “about” means within plus or minus 10% of a given value or range.

In another aspect, provided herein is a method of treating a subject diagnosed with nAMD (wet AMD), dry AMD, retinal vein occlusion (RVO), diabetic macular edema (DME), or diabetic retinopathy (DR), the method comprising preparing a pharmaceutical composition provided herein, storing the pharmaceutical composition at −80° C. for a first period of time, thawing the pharmaceutical composition and, after thawing, storing the pharmaceutical composition at 4° C. for a second period of time. In some embodiments, the first period of time is about 6 months, about 7 months, about 8 months, about 9 months, about 10 months, about 11 months, about 12 months, about 15 months, about 18 months, about 24 months, about 25 months, about 26 months, about 27 months, about 28 months, about 28 months, about 30 months, about 31 months, about 32 months, about 33 months, about 34 months, about 35 months, about 36 months, about 37 months, about 38 months, about 39 months, about 40 months, about 41 months, about 42 months, about 43 months, about 44 months, about 45 months, about 46 months, about 47 months, or about 48 months. In some embodiments, the second period of time is about 1 week, about 2 weeks, about 3 weeks, about 4 weeks, about 1 month, about 2 months, about 3 months, about 4 months, about 5 months, or about 6 months.

4.4 Construct I, Construct II and Other Constructs

In some embodiments, the AAV is AAV viral vectors provided herein comprise the following elements in the following order: a) a constitutive or a hypoxia-inducible promoter sequence, and b) a sequence encoding the transgene (e.g., an anti-VEGF antigen-binding fragment moiety). In some embodiments, the transgene is a fully human post-translationally modified (HuPTM) antibody against VEGF. Antibodies include, but are not limited to, monoclonal antibodies, polyclonal antibodies, recombinantly produced antibodies, human antibodies, humanized antibodies, chimeric antibodies, synthetic antibodies, tetrameric antibodies comprising two heavy chain and two light chain molecules, antibody light chain monomers, antibody heavy chain monomers, antibody light chain dimers, antibody heavy chain dimers, antibody light chain-heavy chain pairs, intrabodies, heteroconjugate antibodies, monovalent antibodies, antigen-binding fragments of full-length antibodies, and fusion proteins of the above. Such antigen-binding fragments include, but are not limited to, single-domain antibodies (variable domain of heavy chain antibodies (VHHs) or nanobodies), Fabs, F(ab′)₂s, and scFvs (single-chain variable fragments) of full-length anti-VEGF antibodies (preferably, full-length anti-VEGF monoclonal antibodies (mAbs) (collectively referred to herein as “antigen-binding fragments”). In a preferred embodiment, the fully human post-translationally modified antibody against VEGF is a fully human post-translationally modified antigen-binding fragment of a monoclonal antibody (mAb) against VEGF (“HuPTMFabVEGFi”). In a further preferred embodiment, the HuPTMFabVEGFi is a fully human glycosylated antigen-binding fragment of an anti-VEGF mAb (“HuGlyFabVEGFi”). In an alternative embodiment, full-length mAbs can be used. In a preferred embodiment, the AAV used for delivering the transgene should have a tropism for human retinal cells or photoreceptor cells. Such AAV can include non-replicating recombinant adeno-associated virus vectors (“rAAV”), particularly those bearing an AAV8 capsid are preferred. In a specific embodiment, the viral vector or other DNA expression construct described herein is Construct I, wherein the Construct I comprises the following components: (1) AAV8 inverted terminal repeats that flank the expression cassette; (2) control elements, which include a) the CB7 promoter, comprising the CMV enhancer/chicken β-actin promoter, b) a chicken β-actin intron and c) a rabbit β-globin poly A signal; and (3) nucleic acid sequences coding for the heavy and light chains of anti-VEGF antigen-binding fragment, separated by a self-cleaving furin (F)/F2A linker, ensuring expression of equal amounts of the heavy and the light chain polypeptides. In another specific embodiment, the viral vector or other DNA expression construct described herein is Construct II, wherein the Construct II comprise the following components: (1) AAV2 inverted terminal repeats that flank the expression cassette; (2) control elements, which include a) the CB7 promoter, comprising the CMV enhancer/chicken β-actin promoter, b) a chicken β-actin intron and c) a rabbit β-globin poly A signal; and (3) nucleic acid sequences coding for the heavy and light chains of anti-VEGF antigen-binding fragment, separated by a self-cleaving furin (F)/F2A linker, ensuring expression of equal amounts of the heavy and the light chain polypeptides. In some embodiments, the anti-hVEGF antibody comprises a heavy chain comprising the amino acid sequence of SEQ ID NO:2 or SEQ ID NO:4, and a light chain comprising the amino acid sequence of SEQ ID NO:1 or SEQ ID NO:3.

In some embodiments, the pharmaceutical composition consists of: (a) the Construct II encoding an anti-human vascular endothelial growth factor (hVEGF) antibody, (b) potassium chloride at a concentration of 0.2 g/L, (c) potassium phosphate monobasic at a concentration of 0.2 g/L, (d) sodium chloride at a concentration of 5.84 g/L, (e) sodium phosphate dibasic anhydrous at a concentration of 1.15 g/L, (f) sucrose at a concentration of 4% weight/volume (40 g/L), (g) poloxamer 188 at a concentration of 0.001% weight/volume (0.01 g/L), and (h) water, and wherein the anti-hVEGF antibody comprises a heavy chain comprising the amino acid sequence of SEQ ID NO:2 or SEQ ID NO:4, and a light chain comprising the amino acid sequence of SEQ ID NO:1 or SEQ ID NO:3.

In some aspects, provided herein are AAV viral vectors encoding an anti-VEGF antigen-binding fragment or a hyperglycosylated derivative of an anti-VEGF antigen-binding fragment. The viral vectors and other DNA expression constructs provided herein include any suitable method for delivery of a transgene to a target cell (e.g., retinal pigment epithelial cells). The means of delivery of a transgene include viral vectors, liposomes, other lipid-containing complexes, other macromolecular complexes, synthetic modified mRNA, unmodified mRNA, small molecules, non-biologically active molecules (e.g., gold particles), polymerized molecules (e.g., dendrimers), naked DNA, plasmids, phages, transposons, cosmids, or episomes. In some embodiments, the vector is a targeted vector, e.g., a vector targeted to retinal pigment epithelial cells.

In some aspects, the disclosure provides for a nucleic acid for use, wherein the nucleic acid encodes a HuPTMFabVEGFi, e.g., HuGlyFabVEGFi operatively linked to a promoter selected from the group consisting of: the CB7 promoter (a chicken 3-actin promoter and CMV enhancer), cytomegalovirus (CMV) promoter, Rous sarcoma virus (RSV) promoter, MMT promoter, EF-1 alpha promoter, UB6 promoter, chicken beta-actin promoter, CAG promoter, RPE65 promoter and opsin promoter. In a specific embodiment, HuPTMFabVEGFi is operatively linked to the CB7 promoter.

In certain embodiments, provided herein are recombinant vectors that comprise one or more nucleic acids (e.g. polynucleotides). The nucleic acids may comprise DNA, RNA, or a combination of DNA and RNA. In certain embodiments, the DNA comprises one or more of the sequences selected from the group consisting of promoter sequences, the sequence of the gene of interest (the transgene, e.g., an anti-VEGF antigen-binding fragment), untranslated regions, and termination sequences. In certain embodiments, viral vectors provided herein comprise a promoter operably linked to the gene of interest.

In certain embodiments, nucleic acids (e.g., polynucleotides) and nucleic acid sequences disclosed herein may be codon-optimized, for example, via any codon-optimization technique known to one of skill in the art (see, e.g., review by Quax et al., 2015, Mol Cell 59:149-161).

4.4.1 mRNA

In certain embodiments, the vectors provided herein are modified mRNA encoding for the gene of interest (e.g., the transgene, for example, an anti-VEGF antigen-binding fragment moiety). The synthesis of modified and unmodified mRNA for delivery of a transgene to retinal pigment epithelial cells is taught, for example, in Hansson et al., J. Biol. Chem., 2015, 290(9):5661-5672, which is incorporated by reference herein in its entirety. In certain embodiments, provided herein is a modified mRNA encoding for an anti-VEGF antigen-binding fragment moiety.

4.4.2 Viral Vectors

In certain embodiments, the viral vectors provided herein are AAV based viral vectors. In preferred embodiments, the viral vectors provided herein are AAV8 based viral vectors. In certain embodiments, the AAV8 based viral vectors provided herein retain tropism for retinal cells. In certain embodiments, the AAV-based vectors provided herein encode the AAV rep gene (required for replication) and/or the AAV cap gene (required for synthesis of the capsid proteins). Multiple AAV serotypes have been identified. In certain embodiments, AAV-based vectors provided herein comprise components from one or more serotypes of AAV. In certain embodiments, AAV based vectors provided herein comprise capsid components from one or more of AAV1, AAV2, AAV2tYF, AAV3, AAV4, AAV5, AAV6, AAV7, AAV8, AAV9, AAV10, AAV11, AAVrh10, AAV.rh20, AAV.rh39, AAV.Rh74, AAV.RHM4-1, AAV.hu37, AAV.Anc80, AAV.Anc80L65, rAAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In preferred embodiments, AAV based vectors provided herein comprise components from one or more of AAV8, AAV9, AAV10, AAV 11, or AAVrh10 serotypes.

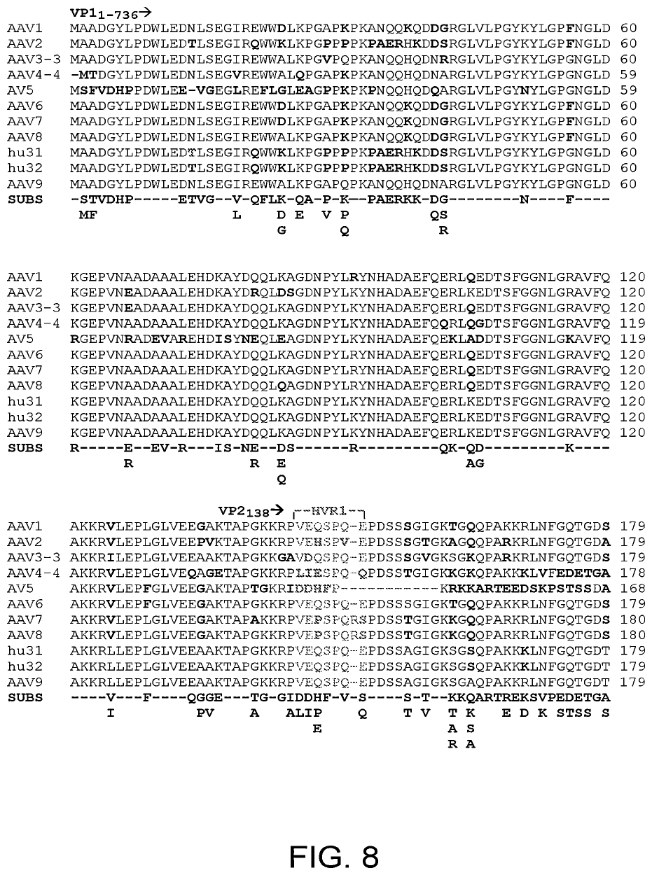

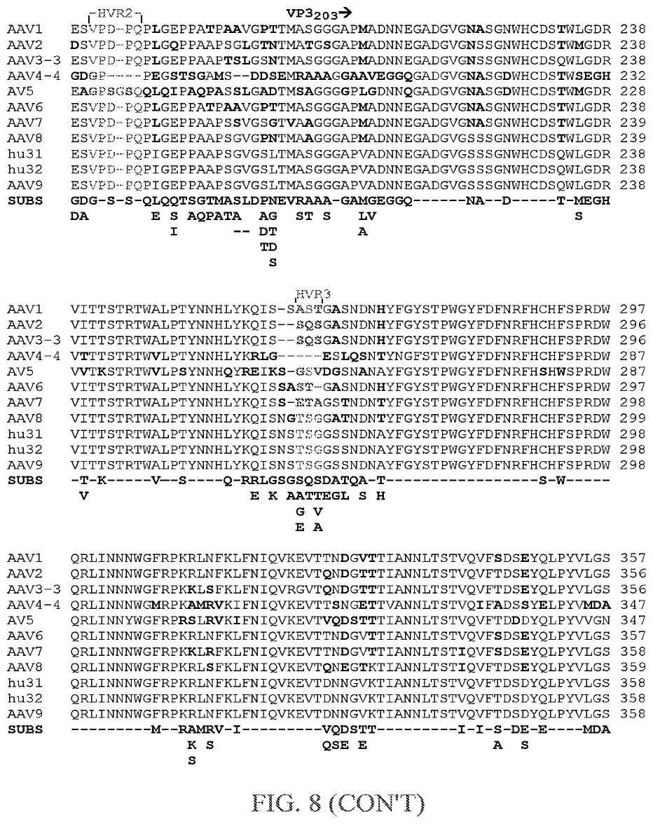

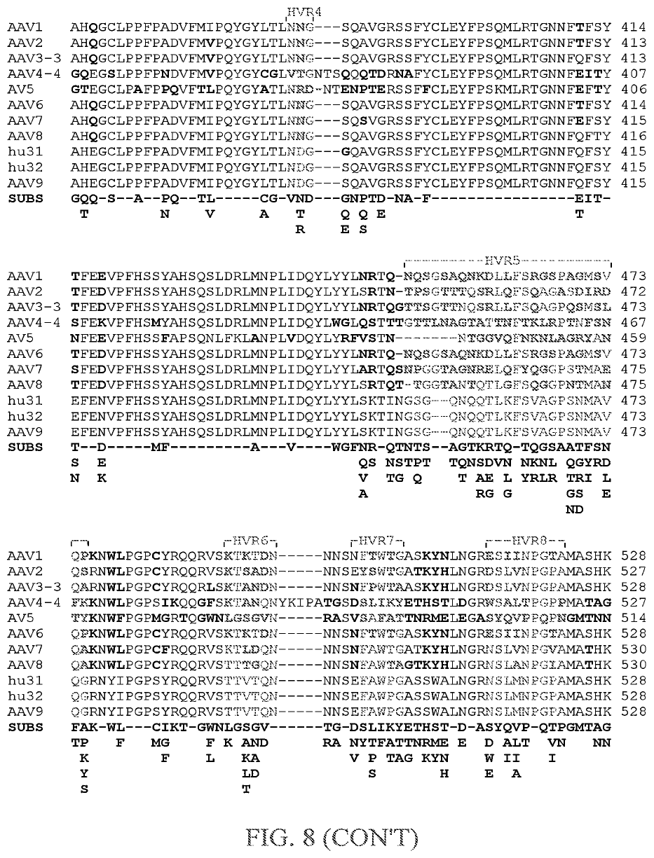

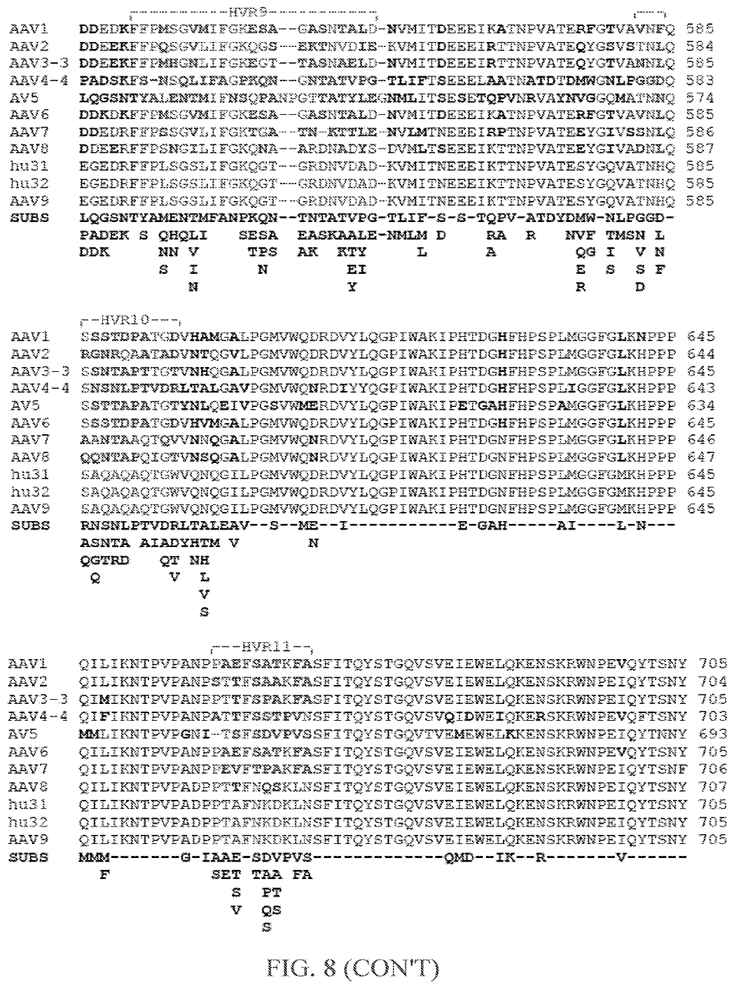

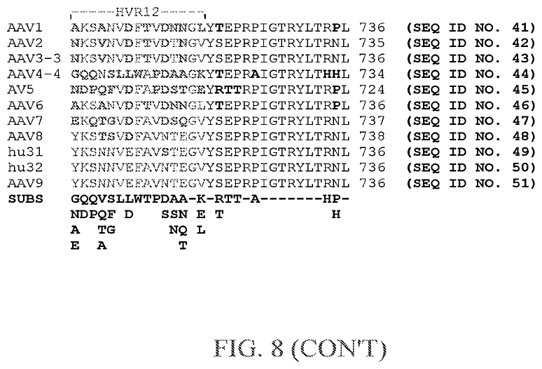

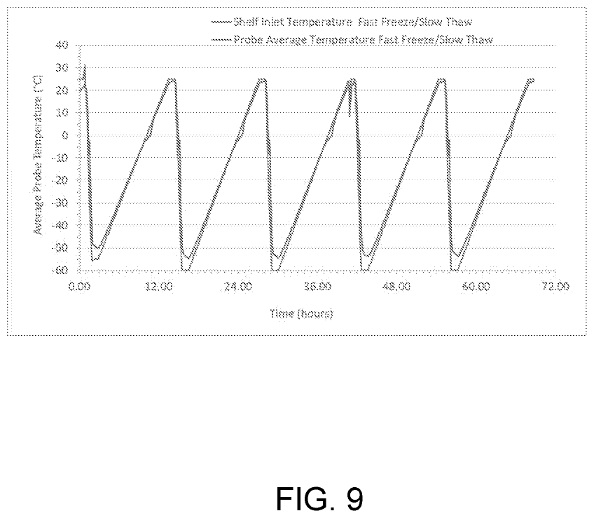

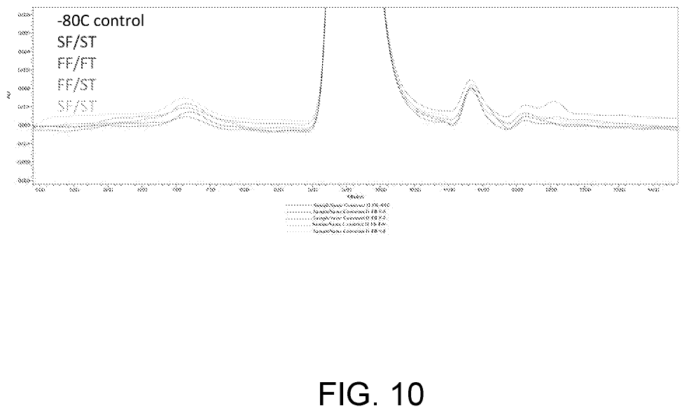

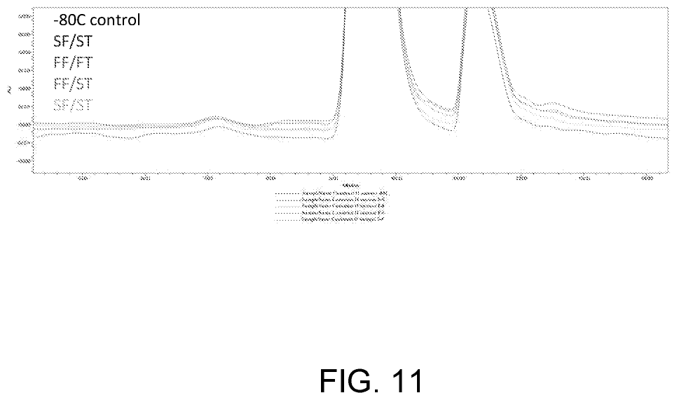

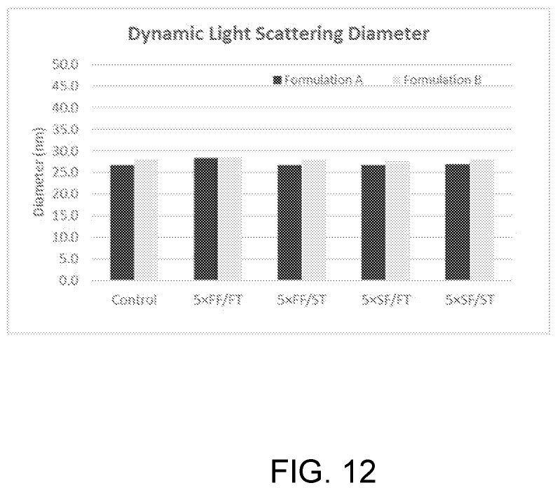

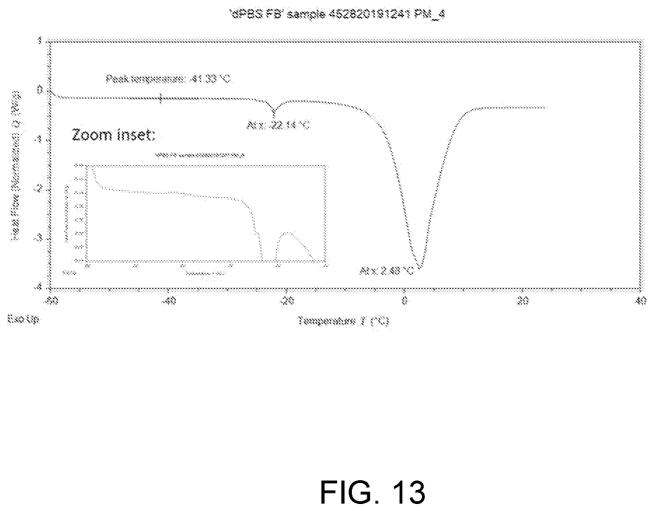

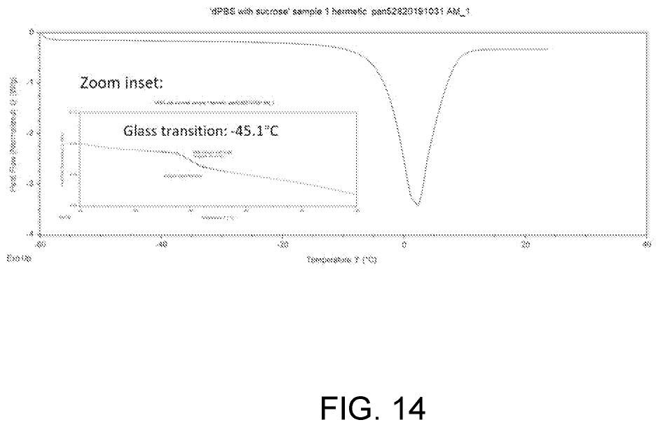

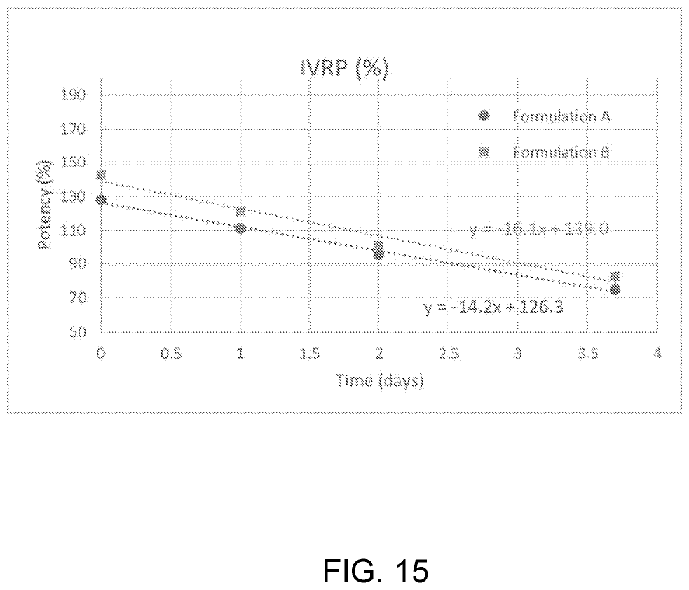

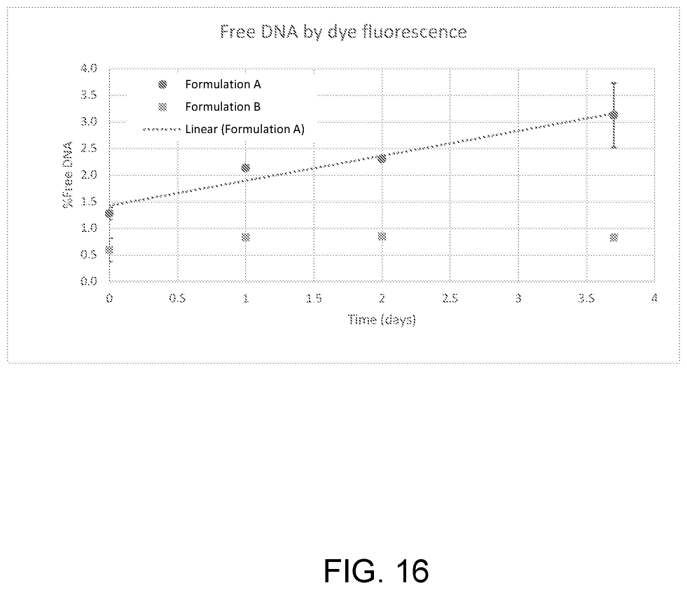

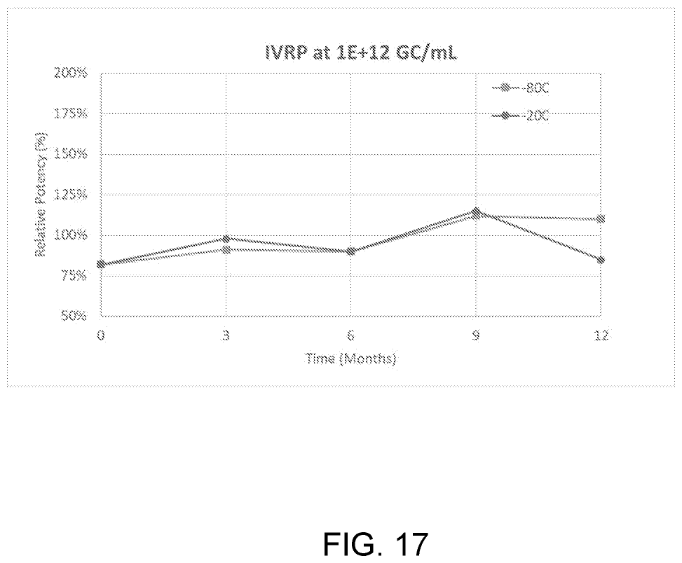

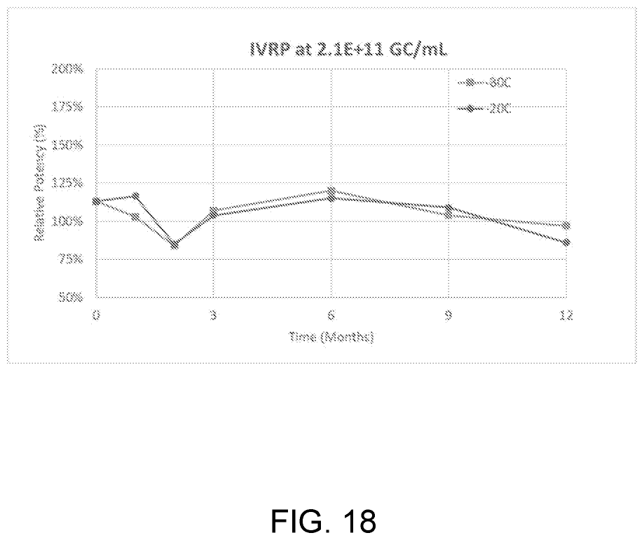

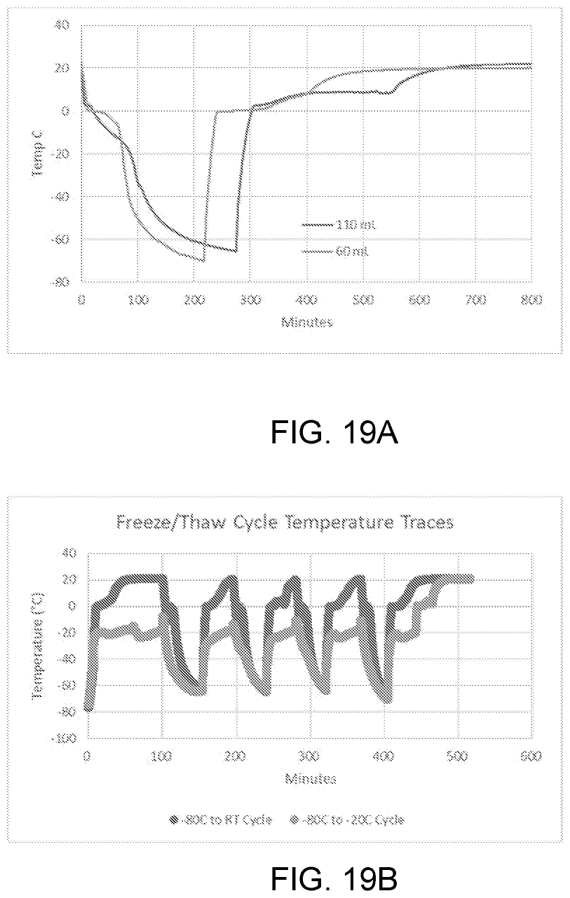

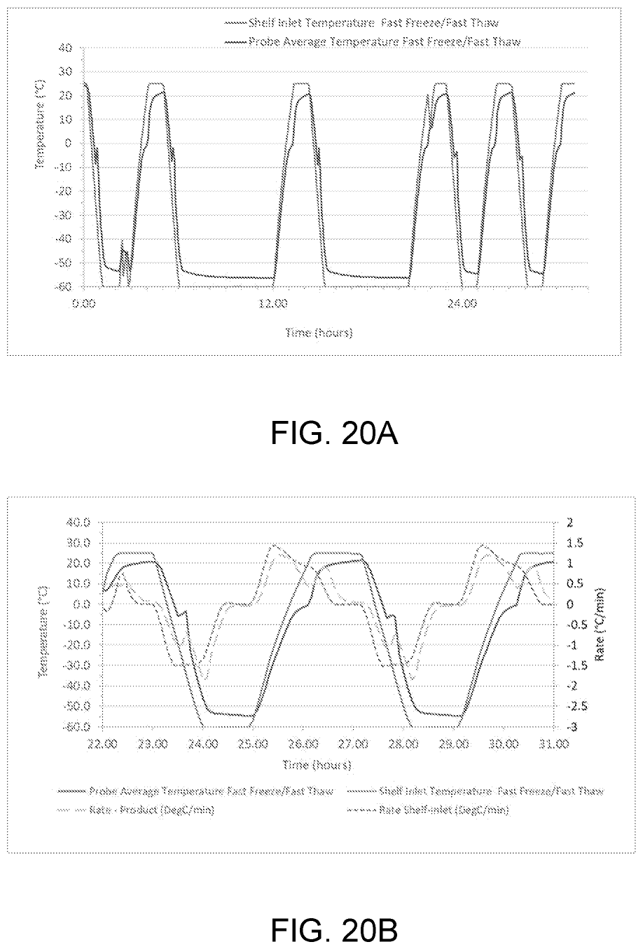

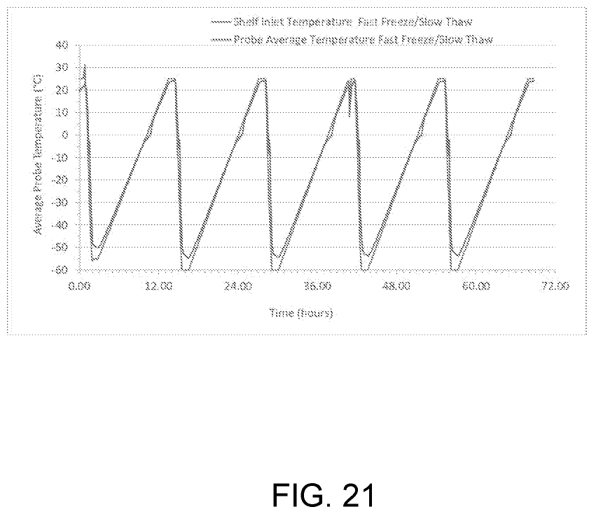

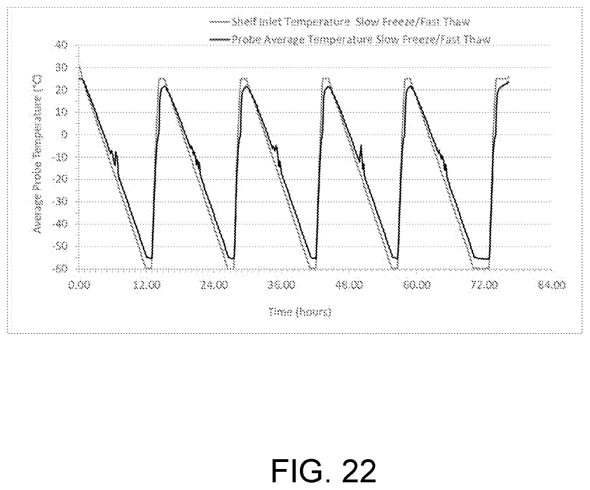

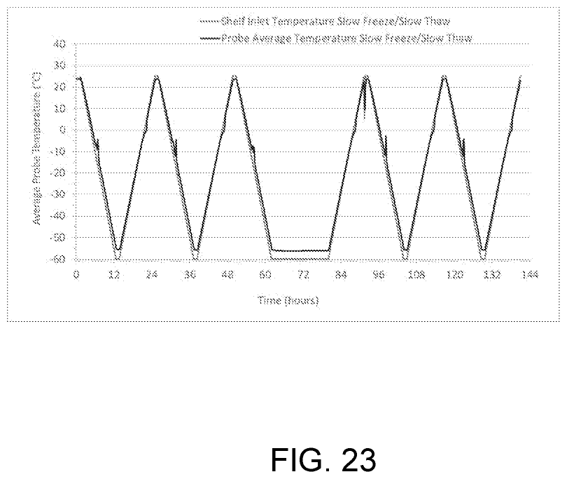

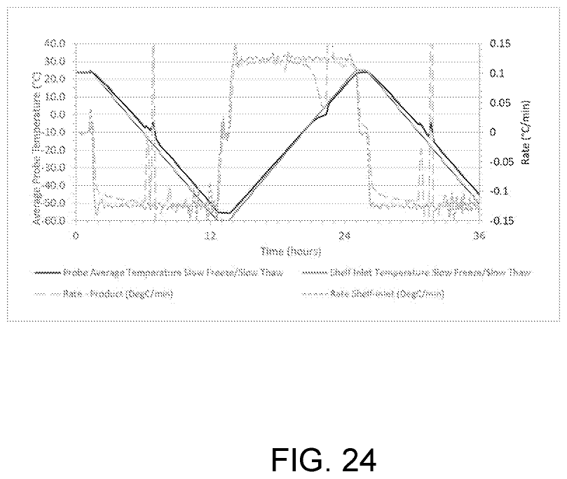

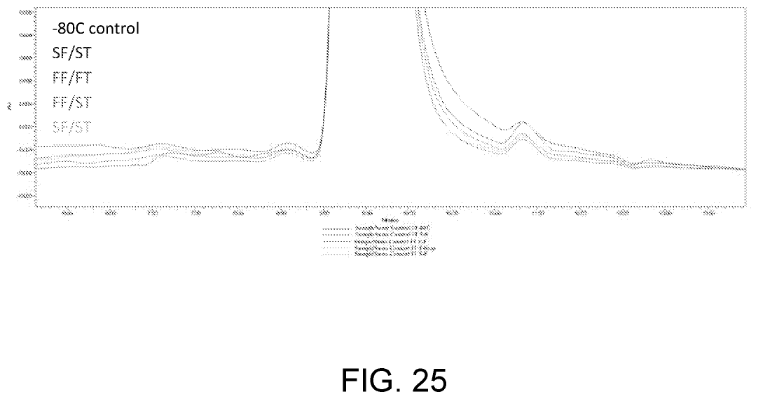

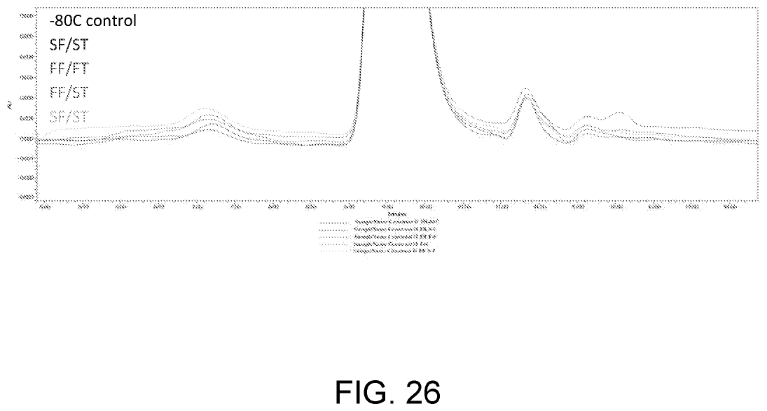

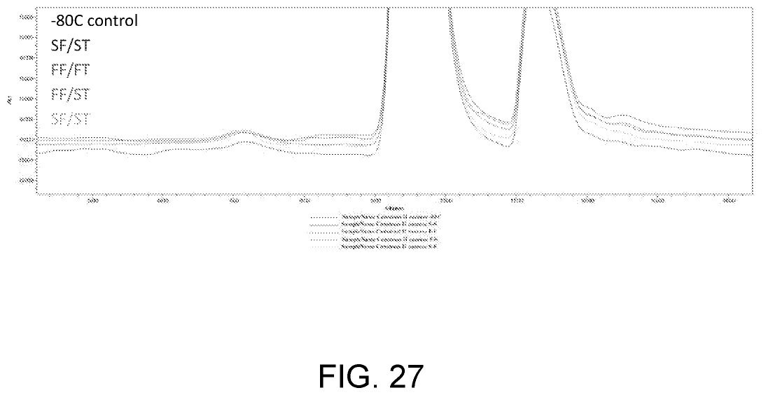

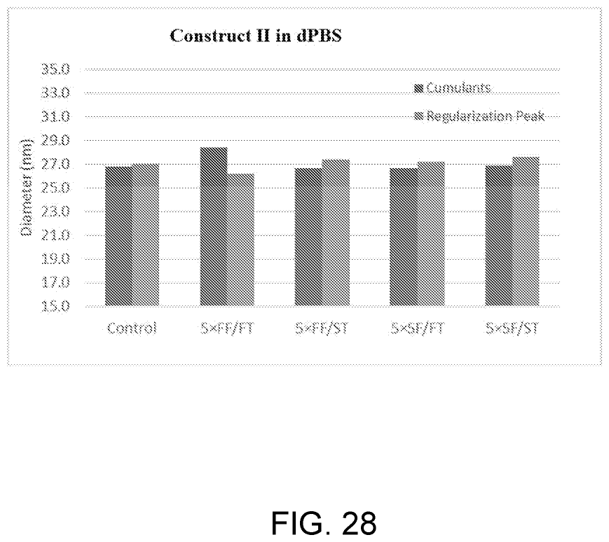

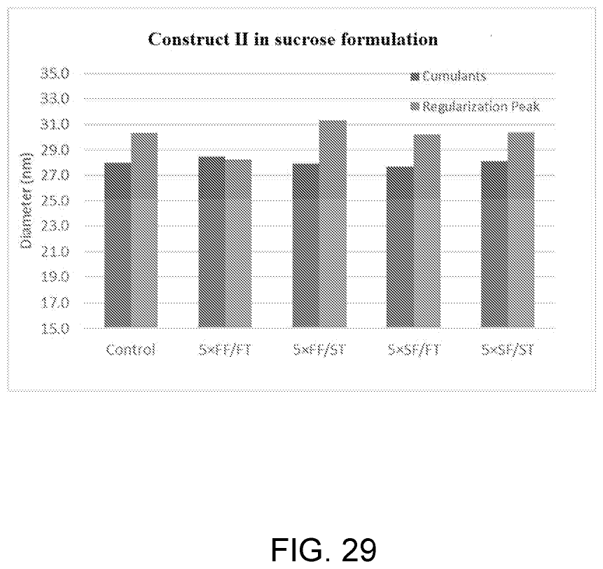

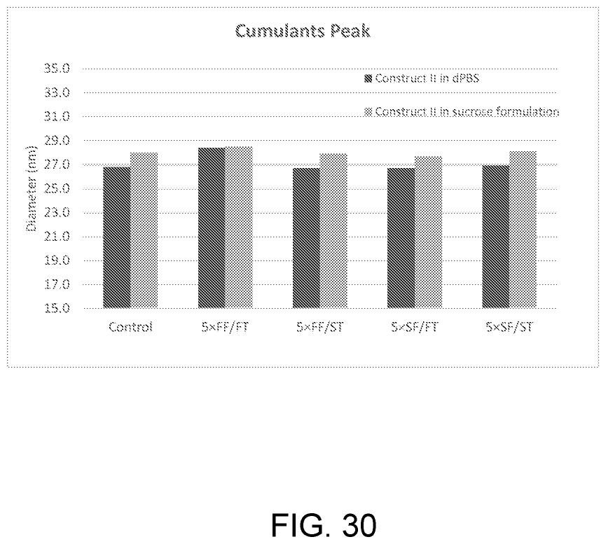

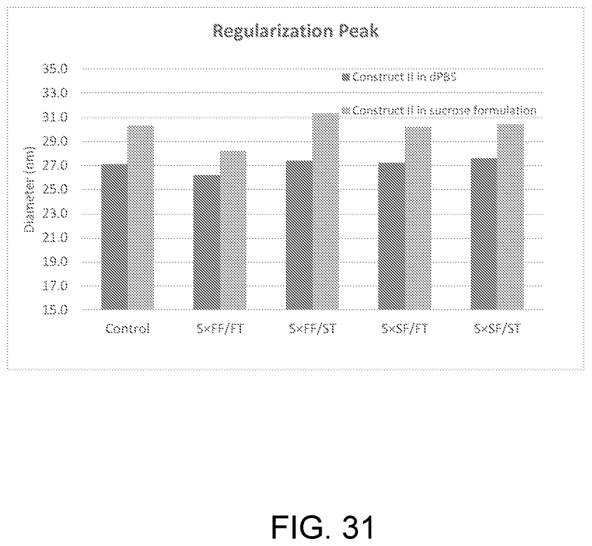

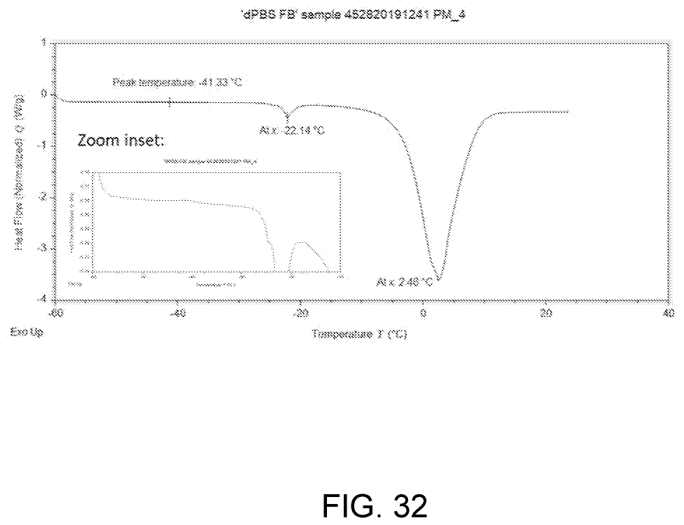

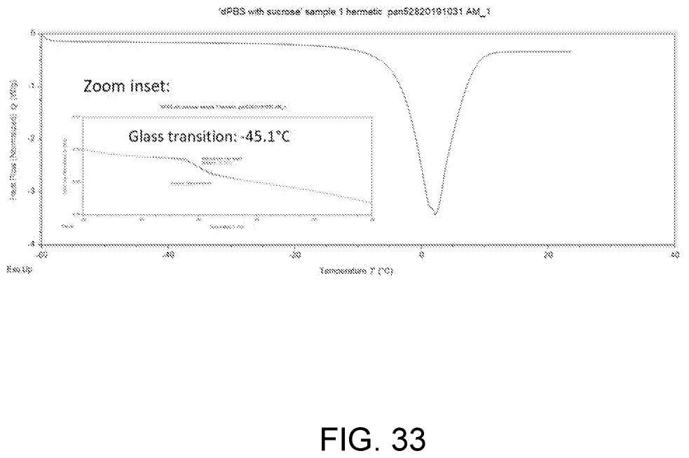

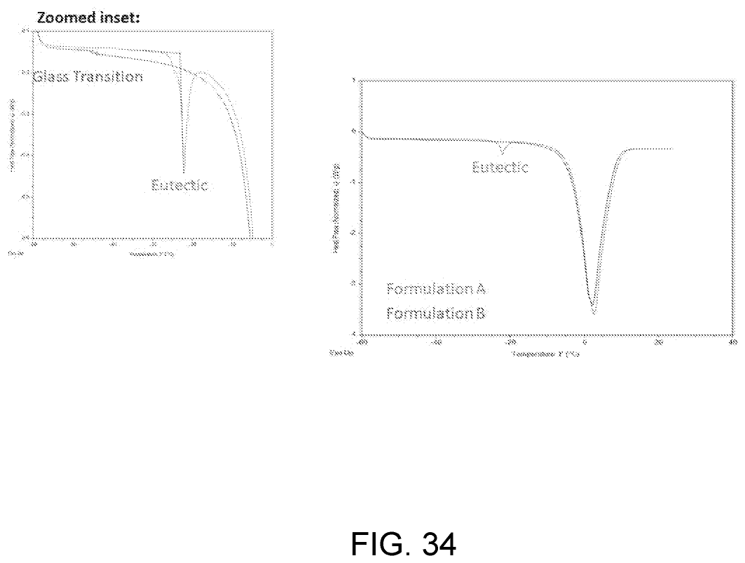

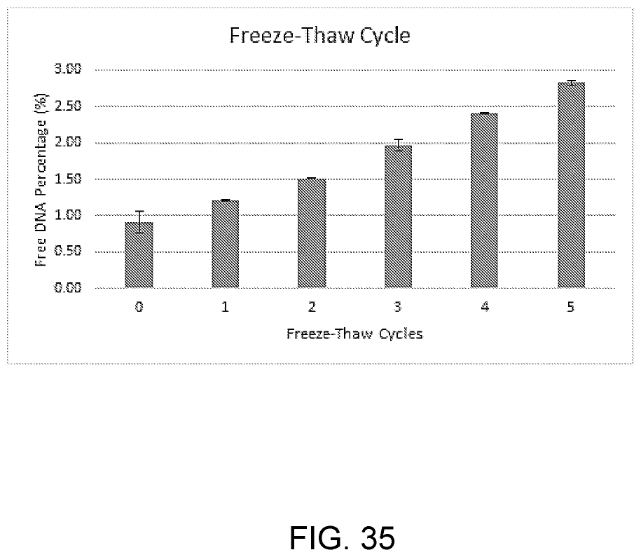

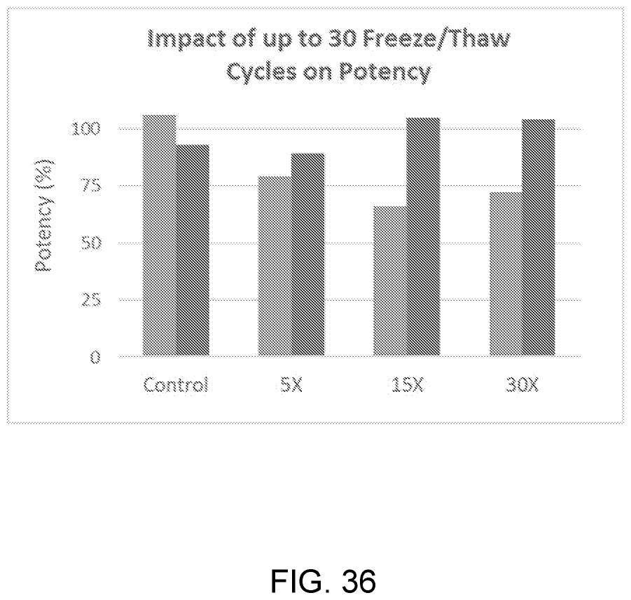

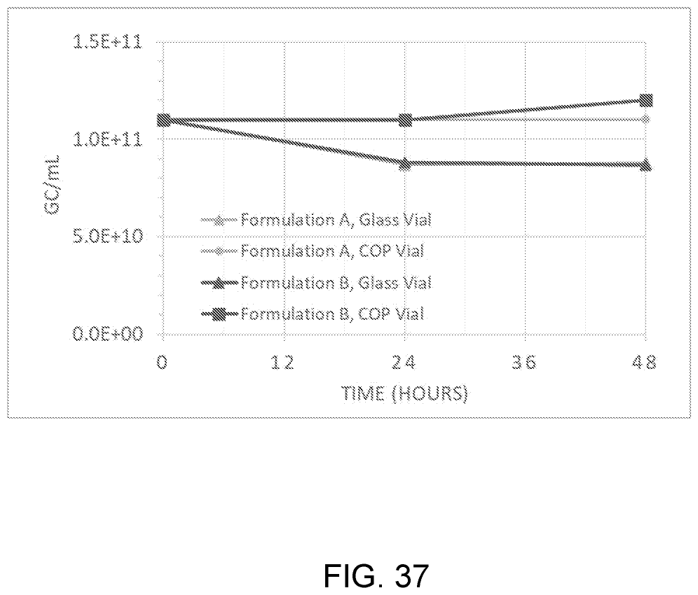

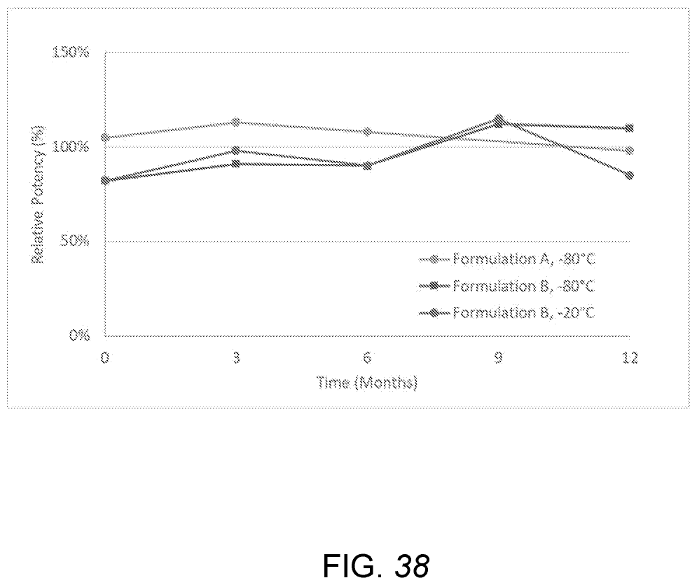

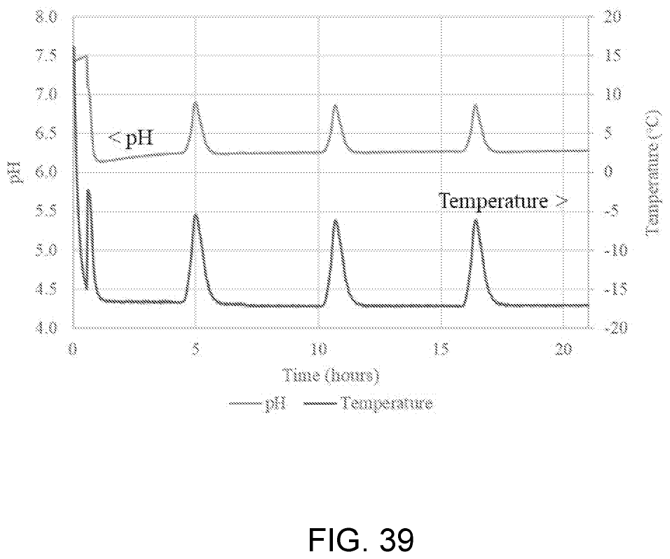

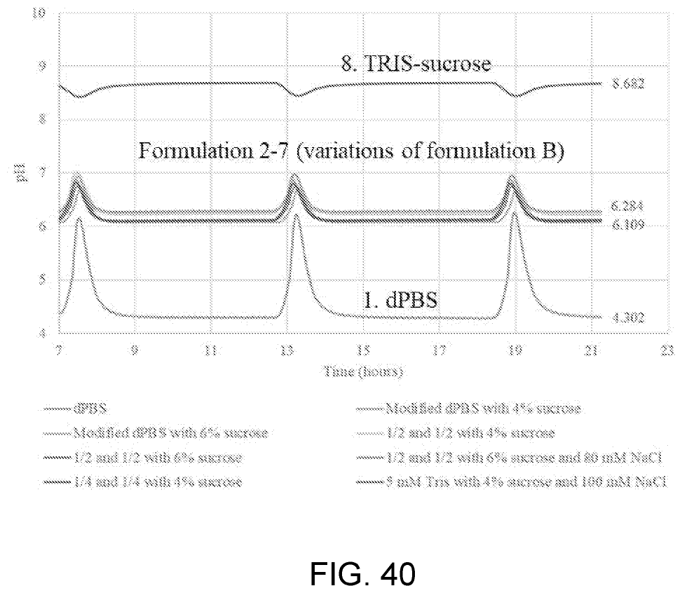

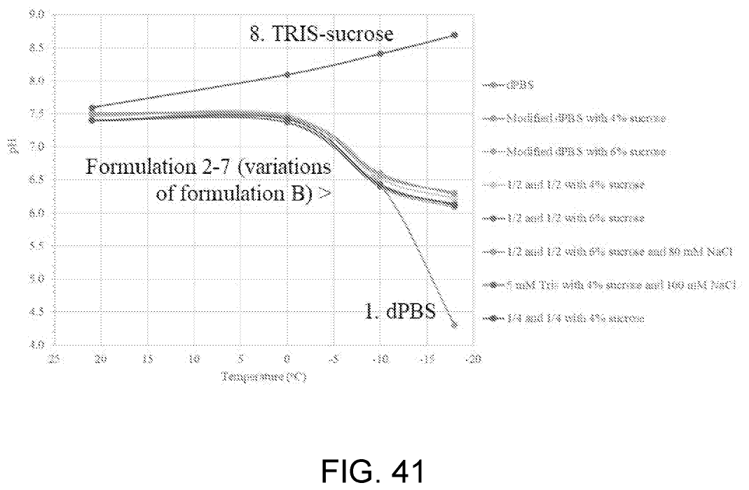

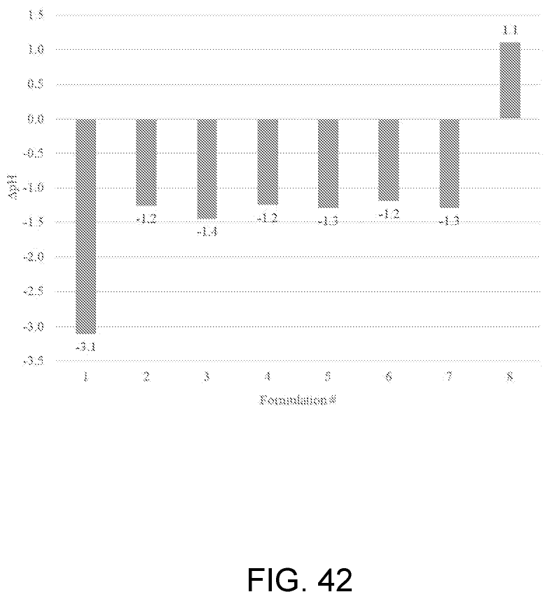

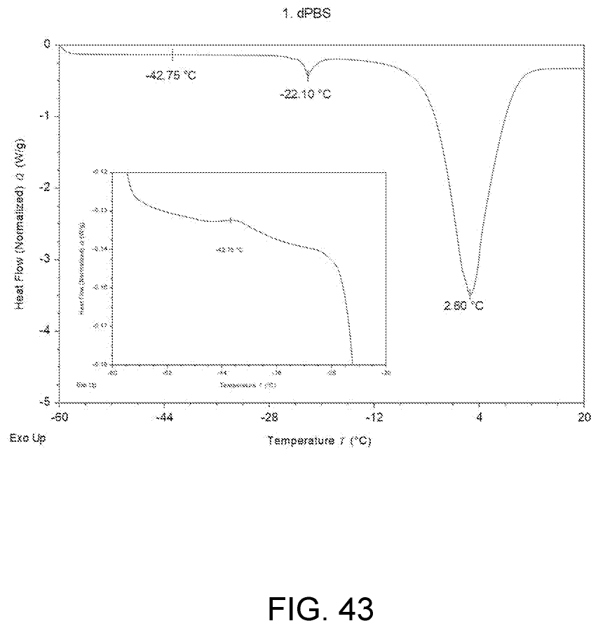

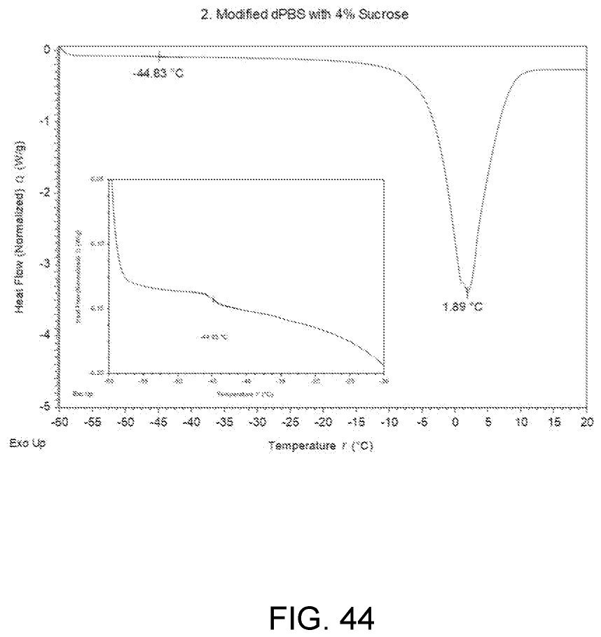

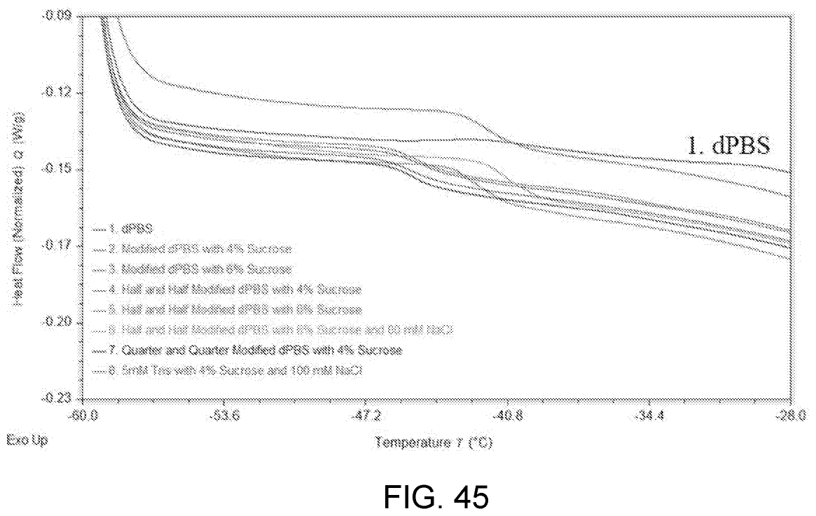

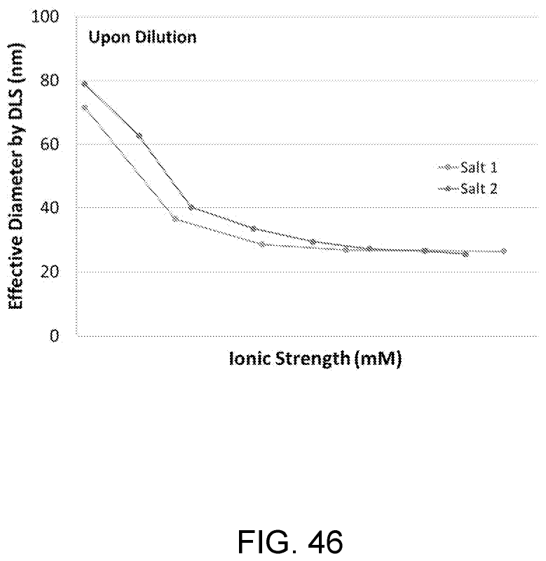

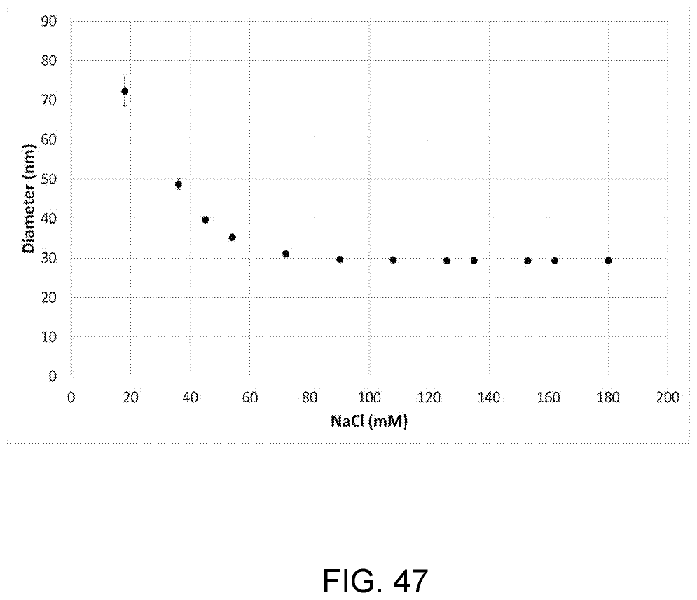

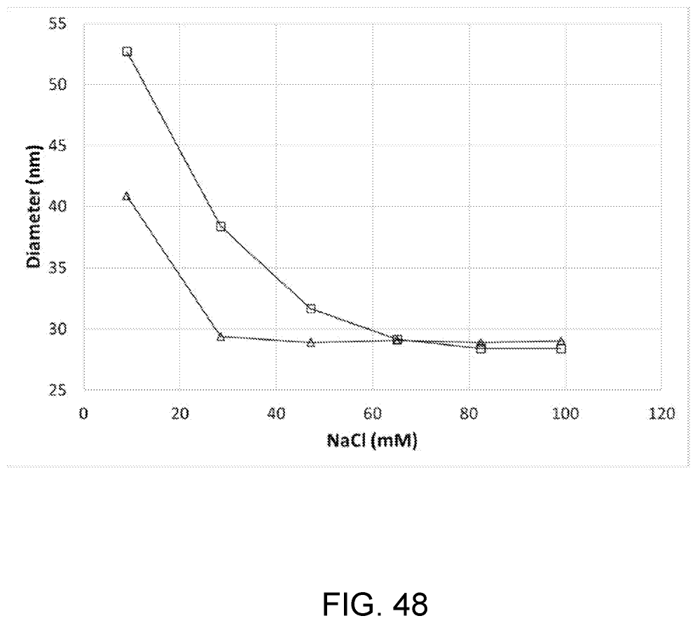

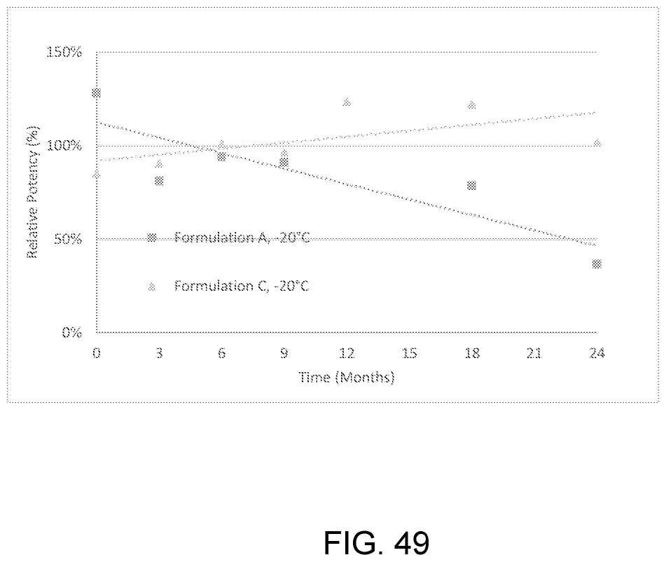

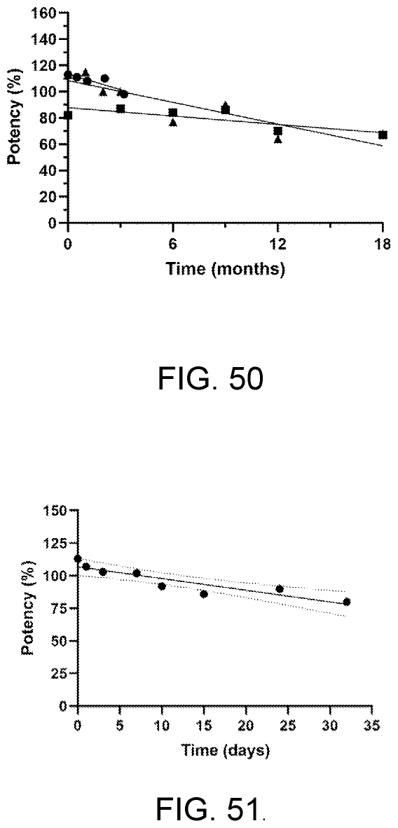

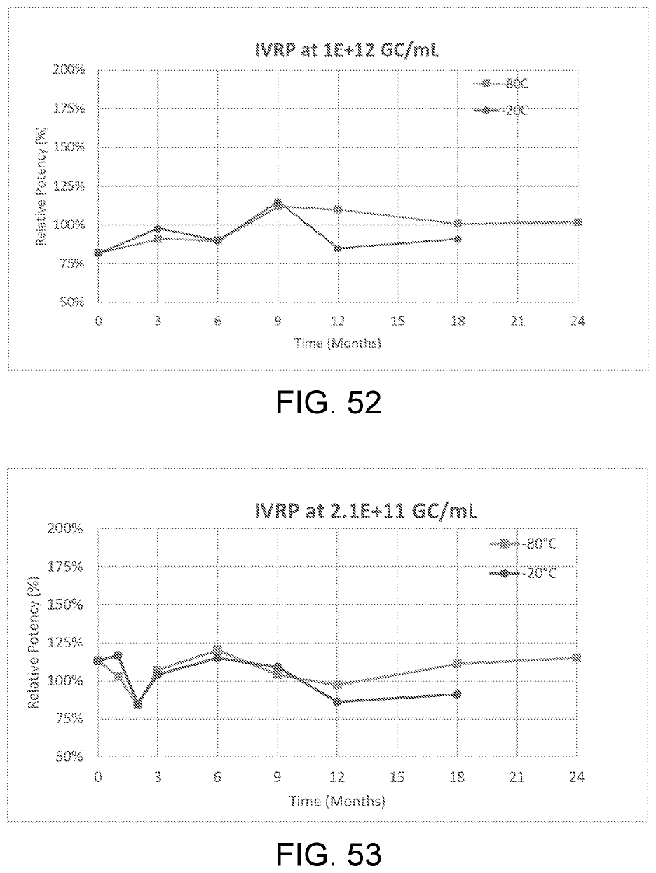

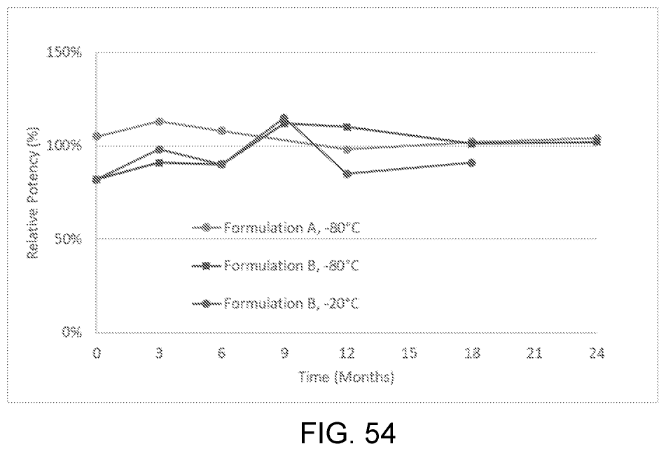

Provided in particular embodiments are AAV8 vectors comprising a viral genome comprising an expression cassette for expression of the transgene, under the control of regulatory elements and flanked by ITRs and a viral capsid that has the amino acid sequence of the AAV8 capsid protein or is at least 95%, 96%, 97%, 98%, 99% or 99.9% identical to the amino acid sequence of the AAV8 capsid protein (SEQ ID NO: 48) while retaining the biological function of the AAV8 capsid. In certain embodiments, the encoded AAV8 capsid has the sequence of SEQ ID NO: 48 with 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions and retaining the biological function of the AAV8 capsid. FIG. 8 provides a comparative alignment of the amino acid sequences of the capsid proteins of different AAV serotypes with potential amino acids that may be substituted at certain positions in the aligned sequences based upon the comparison in the row labeled SUBS. Accordingly, in specific embodiments, the AAV8 vector comprises an AAV8 capsid variant that has 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions identified in the SUBS row of FIG. 8 that are not present at that position in the native AAV8 sequence.

In certain embodiments, the AAV that is used in the methods described herein is Anc80 or Anc80L65, as described in Zinn et al., 2015, Cell Rep. 12(6): 1056-1068, which is incorporated by reference in its entirety. In certain embodiments, the AAV that is used in the methods described herein comprises one of the following amino acid insertions: LGETTRP or LALGETTRP, as described in U.S. Pat. Nos. 9,193,956; 9,458,517; and 9,587,282 and US patent application publication no. 2016/0376323, each of which is incorporated herein by reference in its entirety. In certain embodiments, the AAV that is used in the methods described herein is AAV.7m8, as described in U.S. Pat. Nos. 9,193,956; 9,458,517; and 9,587,282 and US patent application publication no. 2016/0376323, each of which is incorporated herein by reference in its entirety. In certain embodiments, the AAV that is used in the methods described herein is any AAV disclosed in U.S. Pat. No. 9,585,971, such as AAV.PHP.B. In certain embodiments, the AAV that is used in the methods described herein is an AAV disclosed in any of the following patents and patent applications, each of which is incorporated herein by reference in its entirety: U.S. Pat. Nos. 7,906,111; 8,524,446; 8,999,678; 8,628,966; 8,927,514; 8,734,809; 9,284,357; 9,409,953; 9,169,299; 9,193,956; 9,458,517; and 9,587,282 US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335.

AAV8-based viral vectors are used in certain of the methods described herein. Nucleic acid sequences of AAV based viral vectors and methods of making recombinant AAV and AAV capsids are taught, for example, in U.S. Pat. No. 7,282,199 B2, U.S. Pat. No. 7,790,449 B2, U.S. Pat. No. 8,318,480 B2, U.S. Pat. No. 8,962,332 B2 and International Patent Application No. PCT/EP2014/076466, each of which is incorporated herein by reference in its entirety. In one aspect, provided herein are AAV (e.g., AAV8)-based viral vectors encoding a transgene (e.g., an anti-VEGF antigen-binding fragment). In specific embodiments, provided herein are AAV8-based viral vectors encoding an anti-VEGF antigen-binding fragment. In more specific embodiments, provided herein are AAV8-based viral vectors encoding ranibizumab.

In certain embodiments, a single-stranded AAV (ssAAV) may be used supra. In certain embodiments, a self-complementary vector, e.g., scAAV, may be used (see, e.g., Wu, 2007, Human Gene Therapy, 18(2):171-82, McCarty et al, 2001, Gene Therapy, Vol 8, Number 16, Pages 1248-1254; and U.S. Pat. Nos. 6,596,535; 7,125,717; and 7,456,683, each of which is incorporated herein by reference in its entirety).

In certain embodiments, the viral vectors used in the methods described herein are adenovirus based viral vectors. A recombinant adenovirus vector may be used to transfer in the anti-VEGF antigen-binding fragment. The recombinant adenovirus can be a first generation vector, with an E1 deletion, with or without an E3 deletion, and with the expression cassette inserted into either deleted region. The recombinant adenovirus can be a second generation vector, which contains full or partial deletions of the E2 and E4 regions. A helper-dependent adenovirus retains only the adenovirus inverted terminal repeats and the packaging signal (phi). The transgene is inserted between the packaging signal and the 3′ITR, with or without stuffer sequences to keep the genome close to wild-type size of approx. 36 kb. An exemplary protocol for production of adenoviral vectors may be found in Alba et al., 2005, “Gutless adenovirus: last generation adenovirus for gene therapy,” Gene Therapy 12:S18-S27, which is incorporated by reference herein in its entirety.

In a specific embodiment, a vector for use in the methods described herein is one that encodes an anti-VEGF antigen-binding fragment (e.g., ranibizumab) such that, upon introduction of the vector into a relevant cell (e.g., a retinal cell in vivo or in vitro), a glycosylated and or tyrosine sulfated variant of the anti-VEGF antigen-binding fragment is expressed by the cell. In a specific embodiment, the expressed anti-VEGF antigen-binding fragment comprises a glycosylation and/or tyrosine sulfation pattern as described in Section 4.1, above. 4.4.3 Promoters and Modifiers of Gene Expression

In certain embodiments, the vectors provided herein comprise components that modulate gene delivery or gene expression (e.g., “expression control elements”). In certain embodiments, the vectors provided herein comprise components that modulate gene expression. In certain embodiments, the vectors provided herein comprise components that influence binding or targeting to cells. In certain embodiments, the vectors provided herein comprise components that influence the localization of the polynucleotide (e.g., the transgene) within the cell after uptake. In certain embodiments, the vectors provided herein comprise components that can be used as detectable or selectable markers, e.g., to detect or select for cells that have taken up the polynucleotide.

In certain embodiments, the viral vectors provided herein comprise one or more promoters. In certain embodiments, the promoter is a constitutive promoter. In certain embodiments, the promoter is an inducible promoter. Inducible promoters may be preferred so that transgene expression may be turned on and off as desired for therapeutic efficacy. Such promoters include, for example, hypoxia-induced promoters and drug inducible promoters, such as promoters induced by rapamycin and related agents. Hypoxia-inducible promoters include promoters with HIF binding sites, see, for example, Sch6del, et al., 2011, Blood 117(23):e207-e217 and Kenneth and Rocha, 2008, Biochem J. 414:19-29, each of which is incorporated by reference for teachings of hypoxia-inducible promoters. In addition, hypoxia-inducible promoters that may be used in the constructs include the erythropoietin promoter and N-WASP promoter (see, Tsuchiya, 1993, J. Biochem. 113:395 for disclosure of the erythropoietin promoter and Salvi, 2017, Biochemistry and Biophysics Reports 9:13-21 for disclosure of N-WASP promoter, both of which are incorporated by reference for the teachings of hypoxia-induced promoters). Alternatively, the constructs may contain drug inducible promoters, for example promoters inducible by administration of rapamycin and related analogs (see, for example, International Patent Application Publication Nos. WO94/18317, WO 96/20951, WO 96/41865, WO 99/10508, WO 99/10510, WO 99/36553, and WO 99/41258, and U.S. Pat. No. 7,067,526 (disclosing rapamycin analogs), which are incorporated by reference herein for their disclosure of drug inducible promoters). In certain embodiments the promoter is a hypoxia-inducible promoter. In certain embodiments, the promoter comprises a hypoxia-inducible factor (HIF) binding site. In certain embodiments, the promoter comprises a HIF-1α binding site. In certain embodiments, the promoter comprises a HIF-2a binding site. In certain embodiments, the HIF binding site comprises an RCGTG motif. For details regarding the location and sequence of HIF binding sites, see, e.g., Sch6del, et al., Blood, 2011, 117(23):e207-e217, which is incorporated by reference herein in its entirety. In certain embodiments, the promoter comprises a binding site for a hypoxia induced transcription factor other than a HIF transcription factor. In certain embodiments, the viral vectors provided herein comprise one or more IRES sites that is preferentially translated in hypoxia. For teachings regarding hypoxia-inducible gene expression and the factors involved therein, see, e.g., Kenneth and Rocha, Biochem J., 2008, 414:19-29, which is incorporated by reference herein in its entirety.

In certain embodiments, the promoter is a CB7 promoter (see Dinculescu et al., 2005, Hum Gene Ther 16: 649-663, incorporated by reference herein in its entirety). In some embodiments, the CB7 promoter includes other expression control elements that enhance expression of the transgene driven by the vector. In certain embodiments, the other expression control elements include chicken β-actin intron and/or rabbit β-globin polA signal. In certain embodiments, the promoter comprises a TATA box. In certain embodiments, the promoter comprises one or more elements. In certain embodiments, the one or more promoter elements may be inverted or moved relative to one another. In certain embodiments, the elements of the promoter are positioned to function cooperatively. In certain embodiments, the elements of the promoter are positioned to function independently. In certain embodiments, the viral vectors provided herein comprise one or more promoters selected from the group consisting of the human CMV immediate early gene promoter, the SV40 early promoter, the Rous sarcoma virus (RS) long terminal repeat, and rat insulin promoter. In certain embodiments, the vectors provided herein comprise one or more long terminal repeat (LTR) promoters selected from the group consisting of AAV, MLV, MMTV, SV40, RSV, HIV-1, and HIV-2 LTRs. In certain embodiments, the vectors provided herein comprise one or more tissue specific promoters (e.g., a retinal pigment epithelial cell-specific promoter). In certain embodiments, the viral vectors provided herein comprise a RPE65 promoter. In certain embodiments, the vectors provided herein comprise a VMD2 promoter.

In certain embodiments, the viral vectors provided herein comprise one or more regulatory elements other than a promoter. In certain embodiments, the viral vectors provided herein comprise an enhancer. In certain embodiments, the viral vectors provided herein comprise a repressor. In certain embodiments, the viral vectors provided herein comprise an intron or a chimeric intron. In certain embodiments, the viral vectors provided herein comprise a polyadenylation sequence.

4.4.4 Signal Peptides

In certain embodiments, the vectors provided herein comprise components that modulate protein delivery. In certain embodiments, the viral vectors provided herein comprise one or more signal peptides. Signal peptides may also be referred to herein as “leader sequences” or “leader peptides”. In certain embodiments, the signal peptides allow for the transgene product (e.g., the anti-VEGF antigen-binding fragment moiety) to achieve the proper packaging (e.g. glycosylation) in the cell. In certain embodiments, the signal peptides allow for the transgene product (e.g., the anti-VEGF antigen-binding fragment moiety) to achieve the proper localization in the cell. In certain embodiments, the signal peptides allow for the transgene product (e.g., the anti-VEGF antigen-binding fragment moiety) to achieve secretion from the cell. Examples of signal peptides to be used in connection with the vectors and transgenes provided herein may be found in Table 1.

TABLE 1
SIGNAL PEPTIDES FOR USE WITH THE VECTORS PROVIDED HEREIN.

| SEQ | | |
|---|---|---|
| ID NO. | Signal Peptide | Sequence |
| 5 | VEGF-A signal peptide | MNFLLSWVHW SLALLLYLHH |
| | | AKWSQA |
| 6 | Fibulin-1 signal peptide | MERAAPSRRV PLPLLLLGGL |
| | | ALLAAGVDA |
| 7 | Vitronectin signal peptide | MAPLRPLLIL ALLAWVALA |
| 8 | Complement Factor H signal peptide | MRLLAKIICLMLWAICVA |
| 9 | Opticin signal peptide | MRLLAFLSLL ALVLQETGT |
| 22 | Albumin signal peptide | MKWVTFISLLFLFSSAYS |
| 23 | Chymotrypsinogen signal peptide | MAFLWLLSCWALLGTTFG |
| 24 | Interleukin-2 signal peptide | MYRMQLLSCIALILALVTNS |
| 25 | Trypsinogen-2 signal peptide | MNLLLILTFVAAAVA |
| 52 | Mutant Interleukin-2 signal peptide | MYRMQLLLLIALSLALVINS |

4.4.5 Polycistronic Messages—IRES and F2A Linkers

Internal ribosome entry sites. A single construct can be engineered to encode both the heavy and light chains separated by a cleavable linker or IRES so that separate heavy and light chain polypeptides are expressed by the transduced cells. In certain embodiments, the viral vectors provided herein provide polycistronic (e.g., bicistronic) messages. For example, the viral construct can encode the heavy and light chains separated by an internal ribosome entry site (IRES) elements (for examples of the use of IRES elements to create bicistronic vectors see, e.g., Gurtu et al., 1996, Biochem. Biophys. Res. Comm. 229(1):295-8, which is herein incorporated by reference in its entirety). IRES elements bypass the ribosome scanning model and begin translation at internal sites. The use of IRES in AAV is described, for example, in Furling et al., 2001, Gene Ther 8(11): 854-73, which is herein incorporated by reference in its entirety. In certain embodiments, the bicistronic message is contained within a viral vector with a restraint on the size of the polynucleotide(s) therein. In certain embodiments, the bicistronic message is contained within an AAV virus-based vector (e.g., an AAV8-based vector).

Furin-F2A linkers. In other embodiments, the viral vectors provided herein encode the heavy and light chains separated by a cleavable linker such as the self-cleaving furin/F2A (F/F2A) linkers (Fang et al., 2005, Nature Biotechnology 23: 584-590, and Fang, 2007, Mol Ther 15: 1153-9, each of which is incorporated by reference herein in its entirety).

For example, a furin-F2A linker may be incorporated into an expression cassette to separate the heavy and light chain coding sequences, resulting in a construct with the structure:
Leader—Heavy chain—Furin site—F2A site—Leader—Light chain—PolyA.

The F2A site, with the amino acid sequence LLNFDLLKLAGDVESNPGP (SEQ ID NO: 26) is self-processing, resulting in “cleavage” between the final G and P amino acid residues. Additional linkers that could be used include but are not limited to:

T2A:
(SEQ ID NO: 27)
(GSG)EGRGSLTCGDVENPGP;
P2A:
(SEQ ID NO: 28)
(GSG)ATNFSLKQAGDVEENPGP;
E2A:
(SEQ ID NO: 29)
(GSG)QCTNYALLKLAGDVESNPGP;
F2A:
(SEQ ID NO: 30)
(GSG)VKQTLNFDLLKLAGDVESNPGP.

A peptide bond is skipped when the ribosome encounters the F2A sequence in the open reading frame, resulting in the termination of translation, or continued translation of the downstream sequence (the light chain). This self-processing sequence results in a string of additional amino acids at the end of the C-terminus of the heavy chain. However, such additional amino acids are then cleaved by host cell Furin at the furin sites, located immediately prior to the F2A site and after the heavy chain sequence, and further cleaved by carboxypeptidases. The resultant heavy chain may have one, two, three, or more additional amino acids included at the C-terminus, or it may not have such additional amino acids, depending on the sequence of the Furin linker used and the carboxypeptidase that cleaves the linker in vivo (See, e.g., Fang et al., 17 Apr. 2005, Nature Biotechnol. Advance Online Publication; Fang et al., 2007, Molecular Therapy 15(6):1153-1159; Luke, 2012, Innovations in Biotechnology, Ch. 8, 161-186). Furin linkers that may be used comprise a series of four basic amino acids, for example, RKRR, RRRR, RRKR, or RKKR. Once this linker is cleaved by a carboxypeptidase, additional amino acids may remain, such that an additional zero, one, two, three or four amino acids may remain on the C-terminus of the heavy chain, for example, R, RR, RK, RKR, RRR, RRK, RKK, RKRR, RRRR, RRKR, or RKKR. In certain embodiments, one the linker is cleaved by an carboxypeptidase, no additional amino acids remain. In certain embodiments, 0.5%, 1%, 2%, 3%, 4%, 5%, 10%, 15%, or 20%, or less but more than 0% of the antibody, e.g., antigen-binding fragment, population produced by the constructs for use in the methods described herein has one, two, three, or four amino acids remaining on the C-terminus of the heavy chain after cleavage. In certain embodiments, 0.5-1%, 0.5%-2%, 0.5%-3%, 0.5%-4%, 0.5%-5%, 0.5%-10%, 0.5%-20%, 1%-2%, 1%-3%, 1%-4%, 1%-5%, 1%-10%, 1%-20%, 2%-3%, 2%-4%, 2%-5%, 2%-10%, 2%-20%, 3%-4%, 3%-5%, 3%-10%, 3%-20%, 4%-5%, 4%-10%, 4%-20%, 5%-10%, 5%-20%, or 10%-20% of the antibody, e.g., antigen-binding fragment, population produced by the constructs for use in the methods described herein has one, two, three, or four amino acids remaining on the C-terminus of the heavy chain after cleavage. In certain embodiments, the furin linker has the sequence R-X-K/R-R, such that the additional amino acids on the C-terminus of the heavy chain are R, RX, RXK, RXR, RXKR, or RXRR, where X is any amino acid, for example, alanine (A). In certain embodiments, no additional amino acids may remain on the C-terminus of the heavy chain.

In certain embodiments, an expression cassette described herein is contained within a viral vector with a restraint on the size of the polynucleotide(s) therein. In certain embodiments, the expression cassette is contained within an AAV virus-based vector (e.g., an AAV8-based vector).

4.4.6 Untranslated Regions

In certain embodiments, the viral vectors provided herein comprise one or more untranslated regions (UTRs), e.g., 3′ and/or 5′ UTRs. In certain embodiments, the UTRs are optimized for the desired level of protein expression. In certain embodiments, the UTRs are optimized for the mRNA half life of the transgene. In certain embodiments, the UTRs are optimized for the stability of the mRNA of the transgene. In certain embodiments, the UTRs are optimized for the secondary structure of the mRNA of the transgene.

4.4.7 Inverted Terminal Repeats

In certain embodiments, the viral vectors provided herein comprise one or more inverted terminal repeat (ITR) sequences. ITR sequences may be used for packaging the recombinant gene expression cassette into the virion of the viral vector. In certain embodiments, the ITR is from an AAV, e.g., AAV8 or AAV2 (see, e.g., Yan et al., 2005, J. Virol., 79(1):364-379; U.S. Pat. No. 7,282,199 B2, U.S. Pat. No. 7,790,449 B2, U.S. Pat. No. 8,318,480 B2, U.S. Pat. No. 8,962,332 B2 and International Patent Application No. PCT/EP2014/076466, each of which is incorporated herein by reference in its entirety).

4.4.8 Transgenes

The HuPTMFabVEGFi, e.g., HuGlyFabVEGFi encoded by the transgene can include, but is not limited to an antigen-binding fragment of an antibody that binds to VEGF, such as bevacizumab; an anti-VEGF Fab moiety such as ranibizumab; or such bevacizumab or ranibizumab Fab moieties engineered to contain additional glycosylation sites on the Fab domain (e.g., see Courtois et al., 2016, mAbs 8: 99-112 which is incorporated by reference herein in its entirety for it description of derivatives of bevacizumab that are hyperglycosylated on the Fab domain of the full length antibody).

In certain embodiments, the vectors provided herein encode an anti-VEGF antigen-binding fragment transgene. In specific embodiments, the anti-VEGF antigen-binding fragment transgene is controlled by appropriate expression control elements for expression in retinal cells: In certain embodiments, the anti-VEGF antigen-binding fragment transgene comprises bevacizumab Fab portion of the light and heavy chain cDNA sequences (SEQ ID NOs. 10 and 11, respectively). In certain embodiments, the anti-VEGF antigen-binding fragment transgene comprises ranibizumab light and heavy chain cDNA sequences (SEQ ID NOs. 12 and 13, respectively). In certain embodiments, the anti-VEGF antigen-binding fragment transgene encodes a bevacizumab Fab, comprising a light chain and a heavy chain of SEQ ID NOs: 3 and 4, respectively. In certain embodiments, the anti-VEGF antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 3. In certain embodiments, the anti-VEGF antigen-binding fragment transgene encodes an antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 4. In certain embodiments, the anti-VEGF antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 3 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 4. In certain embodiments, the anti-VEGF antigen-binding fragment transgene encodes a hyperglycosylated ranibizumab, comprising a light chain and a heavy chain of SEQ ID NOs: 1 and 2, respectively. In certain embodiments, the anti-VEGF antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 1. In certain embodiments, the anti-VEGF antigen-binding fragment transgene encodes an antigen-binding fragment comprising a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 2. In certain embodiments, the anti-VEGF antigen-binding fragment transgene encodes an antigen-binding fragment comprising a light chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 1 and a heavy chain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 2.

In certain embodiments, the anti-VEGF antigen-binding fragment transgene encodes a hyperglycosylated bevacizumab Fab, comprising a light chain and a heavy chain of SEQ ID NOs: 3 and 4, with one or more of the following mutations: L118N (heavy chain), E195N (light chain), or Q160N or Q160S (light chain). In certain embodiments, the anti-VEGF antigen-binding fragment transgene encodes a hyperglycosylated ranibizumab, comprising a light chain and a heavy chain of SEQ ID NOs: 1 and 2, with one or more of the following mutations: L118N (heavy chain), E195N (light chain), or Q160N or Q160S (light chain). The sequences of the antigen-binding fragment transgene cDNAs may be found, for example, in Table 2. In certain embodiments, the sequence of the antigen-binding fragment transgene cDNAs is obtained by replacing the signal sequence of SEQ ID NOs: 10 and 11 or SEQ ID NOs: 12 and 13 with one or more signal sequences listed in Table 1.

In certain embodiments, the anti-VEGF antigen-binding fragment transgene encodes an antigen-binding fragment and comprises the nucleotide sequences of the six bevacizumab CDRs. In certain embodiments, the anti-VEGF antigen-binding fragment transgene encodes an antigen-binding fragment and comprises the nucleotide sequences of the six ranibizumab CDRs. In certain embodiments, the anti-VEGF antigen-binding fragment transgene encodes an antigen-binding fragment comprising a heavy chain variable region comprising heavy chain CDRs 1-3 of ranibizumab (SEQ ID NOs: 20, 18, and 21). In certain embodiments, the anti-VEGF antigen-binding fragment transgene encodes an antigen-binding fragment comprising a light chain variable region comprising light chain CDRs 1-3 of ranibizumab (SEQ ID NOs: 14-16). In certain embodiments, the anti-VEGF antigen-binding fragment transgene encodes an antigen-binding fragment comprising a heavy chain variable region comprising heavy chain CDRs 1-3 of bevacizumab (SEQ ID NOs: 17-19). In certain embodiments, the anti-VEGF antigen-binding fragment transgene encodes an antigen-binding fragment comprising a light chain variable region comprising light chain CDRs 1-3 of bevacizumab (SEQ ID NOs: 14-16). In certain embodiments, the anti-VEGF antigen-binding fragment transgene encodes an antigen-binding fragment comprising a heavy chain variable region comprising heavy chain CDRs 1-3 of ranibizumab (SEQ ID NOs: 20, 18, and 21) and a light chain variable region comprising light chain CDRs 1-3 of ranibizumab (SEQ ID NOs: 14-16). In certain embodiments, the anti-VEGF antigen-binding fragment transgene encodes an antigen-binding fragment comprising a heavy chain variable region comprising heavy chain CDRs 1-3 of bevacizumab (SEQ ID NOs: 17-19) and a light chain variable region comprising light chain CDRs 1-3 of bevacizumab (SEQ ID NOs: 14-16).

In certain embodiments, the anti-VEGF antigen-binding fragment transgene encodes an antigen-binding fragment comprising a light chain variable region comprising light chain CDRs 1-3 of SEQ ID NOs: 14-16, wherein the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a light chain variable region comprising light chain CDRs 1-3 of SEQ ID NOs: 14-16, wherein the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a light chain variable region comprising light chain CDRs 1-3 of SEQ ID NOs: 14-16, wherein the second amino acid residue of the light chain CDR3 (i.e., the second Q in QQYSTVPWTF (SEQ ID NO. 16)) is not acetylated. In a specific embodiment, the anti-VEGF antigen-binding fragment transgene encodes an antigen-binding fragment comprising a light chain variable region comprising light chain CDRs 1-3 of SEQ ID NOs: 14-16, wherein the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) is not acetylated. In a preferred embodiment, the chemical modification(s) or lack of chemical modification(s) (as the case may be) described herein is determined by mass spectrometry.

In certain embodiments, the anti-VEGF antigen-binding fragment transgene encodes an antigen-binding fragment comprising a heavy chain variable region comprising heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a heavy chain variable region comprising heavy chain CDRs 1-3 of SEQ ID NOs: 20, 18, and 21, wherein the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a heavy chain variable region comprising heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) is not acetylated. In a specific embodiment, the anti-VEGF antigen-binding fragment transgene encodes an antigen-binding fragment comprising a heavy chain variable region comprising heavy chain CDRs 1-3 of SEQ ID NOs: 20, 18, and 21, wherein the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) is not acetylated. In a preferred embodiment, the chemical modification(s) or lack of chemical modification(s) (as the case may be) described herein is determined by mass spectrometry.

In certain embodiments, the anti-VEGF antigen-binding fragment transgene encodes an antigen-binding fragment comprising a light chain variable region comprising light chain CDRs 1-3 of SEQ ID NOs: 14-16 and a heavy chain variable region comprising heavy chain CDRs 1-3 of SEQ ID NOs: 20, 18, and 21, wherein the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu), and wherein the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a light chain variable region comprising light chain CDRs 1-3 of SEQ ID NOs: 14-16 and a heavy chain variable region comprising heavy chain CDRs 1-3 of SEQ ID NOs: 20, 18, and 21, wherein: (1) the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu); and (2) the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the anti-VEGF antigen-binding fragment transgene encodes an antigen-binding fragment comprising a light chain variable region comprising light chain CDRs 1-3 of SEQ ID NOs: 14-16 and a heavy chain variable region comprising heavy chain CDRs 1-3 of SEQ ID NOs: 20, 18, and 21, wherein the second amino acid residue of the light chain CDR3 (i.e., the second Q in QQYSTVPWTF (SEQ ID NO. 16)) is not acetylated, and wherein the last amino acid residue of the heavy chain CDR1 (i.e., the N in GYDFTHYGMN (SEQ ID NO. 20)) is not acetylated. In a specific embodiment, the antigen-binding fragment comprises a heavy chain CDR1 of SEQ ID NO. 20, wherein: (1) the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) is not acetylated; and (2) the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) is not acetylated. In a preferred embodiment, the chemical modification(s) or lack of chemical modification(s) (as the case may be) described herein is determined by mass spectrometry.

In certain aspects, also provided herein are anti-VEGF antigen-binding fragments comprising light chain CDRs 1-3 of SEQ ID NOs: 14-16 and heavy chain CDRs 1-3 of SEQ ID NOs: 20, 18, and 21, and transgenes encoding such antigen-VEGF antigen-binding fragments, wherein the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the second amino acid residue of the light chain CDR3 (i.e., the second Q in QQYSTVPWTF (SEQ ID NO. 16)) is not acetylated. In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) is not acetylated. The anti-VEGF antigen-binding fragments and transgenes provided herein can be used in any method according to the invention described herein. In a preferred embodiment, the chemical modification(s) or lack of chemical modification(s) (as the case may be) described herein is determined by mass spectrometry.

In certain aspects, also provided herein are anti-VEGF antigen-binding fragments comprising light chain CDRs 1-3 of SEQ ID NOs: 14-16 and heavy chain CDRs 1-3 of SEQ ID NOs: 20, 18, and 21, and transgenes encoding such antigen-VEGF antigen-binding fragments, wherein the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) is not acetylated. In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) is not acetylated. The anti-VEGF antigen-binding fragments and transgenes provided herein can be used in any method according to the invention described herein. In a preferred embodiment, the chemical modification(s) or lack of chemical modification(s) (as the case may be) described herein is determined by mass spectrometry.

In certain aspects, also provided herein are anti-VEGF antigen-binding fragments comprising light chain CDRs 1-3 of SEQ ID NOs: 14-16 and heavy chain CDRs 1-3 of SEQ ID NOs: 20, 18, and 21, and transgenes encoding such antigen-VEGF antigen-binding fragments, wherein the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein: (1) the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) does not carry one or more of the following chemical modifications: oxidation, acetylation, deamidation, and pyroglutamation (pyro Glu); and (2) the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) does not carry one or more of the following chemical modifications: oxidation, acetylation, deamidation, and pyroglutamation (pyro Glu). In a specific embodiment, the antigen-binding fragment comprises light chain CDRs 1-3 of SEQ ID NOs: 14-16 and heavy chain CDRs 1-3 of SEQ ID NOs: 20, 18, and 21, wherein the last amino acid residue of the heavy chain CDR1 (i.e., the N in GYDFTHYGMN (SEQ ID NO. 20)) is not acetylated, and the second amino acid residue of the light chain CDR3 (i.e., the second Q in QQYSTVPWTF (SEQ ID NO. 16)) is not acetylated. In a specific embodiment, the antigen-binding fragment comprises light chain CDRs 1-3 of SEQ ID NOs: 14-16 and heavy chain CDRs 1-3 of SEQ ID NOs: 20, 18, and 21, wherein: (1) the ninth amino acid residue of the heavy chain CDR1 (i.e., the M in GYDFTHYGMN (SEQ ID NO. 20)) carries one or more of the following chemical modifications: acetylation, deamidation, and pyroglutamation (pyro Glu), the third amino acid residue of the heavy chain CDR2 (i.e., the N in WINTYTGEPTYAADFKR (SEQ ID NO. 18) carries one or more of the following chemical modifications: acetylation, deamidation, and pyroglutamation (pyro Glu), and the last amino acid residue of the heavy chain CDR1 (i.e., the N in GYDFTHYGMN (SEQ ID NO. 20)) is not acetylated; and (2) the eighth and eleventh amino acid residues of the light chain CDR1 (i.e., the two Ns in SASQDISNYLN (SEQ ID NO. 14) each carries one or more of the following chemical modifications: oxidation, acetylation, deamidation, and pyroglutamation (pyro Glu), and the second amino acid residue of the light chain CDR3 (i.e., the second Q in QQYSTVPWTF (SEQ ID NO. 16)) is not acetylated. The anti-VEGF antigen-binding fragments and transgenes provided herein can be used in any method according to the invention described herein. In a preferred embodiment, the chemical modification(s) or lack of chemical modification(s) (as the case may be) described herein is determined by mass spectrometry.

TABLE 2
EXEMPLARY TRANSGENE SEQUENCES

| VEGF antigen- | |
|---|---|
| binding fragment | |
| (SEQ ID NO.) | Sequence |
| bevacizumab cDNA | gctagcgcca ccatgggctg gtcctgcatc atcctgttcc tggtggccac |
| (Light chain) | cgccaccggc gtgcactccg acatccagat gacccagtcc ccctcctccc |
| (10) | tgtccgcctc cgtgggcgac cgggtgacca tcacctgctc cgcctcccag |
| | gacatctcca actacctgaa ctggtaccag cagaagcccg gcaaggcccc |
| | caaggtgctg atctacttca cctcctccct gcactccggc gtgccctccc |
| | ggttctccgg ctccggctcc ggcaccgact tcaccctgac catctcctcc |
| | ctgcagcccg aggacttcgc cacctactac tgccagcagt actccaccgt |
| | gccctggacc ttcggccagg gcaccaaggt ggagatcaag cggaccgtgg |
| | ccgccccctc cgtgttcatc ttccccccct ccgacgagca gctgaagtcc |
| | ggcaccgcct ccgtggtgtg cctgctgaac aacttctacc cccgggaggc |
| | caaggtgcag tggaaggtgg acaacgccct gcagtccggc aactcccagg |
| | agtccgtgac cgagcaggac tccaaggact ccacctactc cctgtcctcc |
| | accctgaccc tgtccaaggc cgactacgag aagcacaagg tgtacgcctg |
| | cgaggtgacc caccagggcc tgtcctcccc cgtgaccaag tccttcaacc |
| | ggggcgagtg ctgagcggcc gcctcgag |
| bevacizumab cDNA | gctagcgcca ccatgggctg gtcctgcatc atcctgttcc tggtggccac |
| (Heavy chain) | cgccaccggc gtgcactccg aggtgcagct ggtggagtcc ggcggcggcc |
| (11) | tggtgcagcc cggcggctcc ctgcggctgt cctgcgccgc ctccggctac |
| | accttcacca actacggcat gaactgggtg cggcaggccc ccggcaaggg |
| | cctggagtgg gtgggctgga tcaacaccta caccggcgag cccacctacg |
| | ccgccgactt caagcggcgg ttcaccttct ccctggacac ctccaagtcc |
| | accgcctacc tgcagatgaa ctccctgcgg gccgaggaca ccgccgtgta |
| | ctactgcgcc aagtaccccc actactacgg ctcctcccac tggtacttcg |
| | acgtgtgggg ccagggcacc ctggtgaccg tgtcctccgc ctccaccaag |
| | ggcccctccg tgttccccct ggccccctcc tccaagtcca cctccggcgg |
| | caccgccgcc ctgggctgcc tggtgaagga ctacttcccc gagcccgtga |
| | ccgtgtcctg gaactccggc gccctgacct ccggcgtgca caccttcccc |
| | gccgtgctgc agtcctccgg cctgtactcc ctgtcctccg tggtgaccgt |
| | gccctcctcc tccctgggca cccagaccta catctgcaac gtgaaccaca |
| | agccctccaa caccaaggtg gacaagaagg tggagcccaa gtcctgcgac |
| | aagacccaca cctgcccccc ctgccccgcc cccgagctgc tgggcggccc |
| | ctccgtgttc ctgttccccc ccaagcccaa ggacaccctg atgatctccc |
| | ggacccccga ggtgacctgc gtggtggtgg acgtgtccca cgaggacccc |
| | gaggtgaagt tcaactggta cgtggacggc gtggaggtgc acaacgccaa |
| | gaccaagccc cgggaggagc agtacaactc cacctaccgg gtggtgtccg |
| | tgctgaccgt gctgcaccag gactggctga acggcaagga gtacaagtgc |
| | aaggtgtcca acaaggccct gcccgccccc atcgagaaga ccatctccaa |
| | ggccaagggc cagccccggg agccccaggt gtacaccctg cccccctccc |
| | gggaggagat gaccaagaac caggtgtccc tgacctgcct ggtgaagggc |
| | ttctacccct ccgacatcgc cgtggagtgg gagtccaacg gccagcccga |
| | gaacaactac aagaccaccc cccccgtgct ggactccgac ggctccttct |
| | tcctgtactc caagctgacc gtggacaagt cccggtggcagcagggcaac |
| | gtgttctcct gctccgtgat gcacgaggcc ctgcacaacc actacaccca |
| | gaagtccctg tccctgtccc ccggcaagtg agcggccgcc |
| bevacizumab Fab | DIQMTQSPSSLSASVGDRVTITCSASQDISNYLNWYQQKPGKAPKVLIYFTSSLH |
| Amino Acid | SGVPSRFSGSGSGTDFTLTISSLQPEDFATYYCQQYSTVPWTFGQGTKVEIKRTV |
| Sequence (Light | AAPSVFIFPPSDEQLKSGTASVVCLLNNFYPREAKVQWKVDNALQSGNSQESVTE |
| chain) | QDSKDSTYSLSSTLTLSKADYEKHKVYACEVTHQGLSSPVTKSENRGEC |
| (3) | |
| bevacizumab Fab | EVQLVESGGGLVQPGGSLRLSCAASGYTFTNYGMNWVRQAPGKGLEWVGWINTYT |
| Amino Acid | GEPTYAADFKRRFTFSLDTSKSTAYLQMNSLRAEDTAVYYCAKYPHYYGSSHWYF |
| Sequence (Heavy | DVWGQGTLVTVSSASTKGPSVFPLAPSSKSTSGGTAALGCLVKDYFPEPVTVSWN |
| chain) | SGALTSGVHTFPAVLQSSGLYSLSSVVTVPSSSLGTQTYICNVNHKPSNTKVDKK |
| (4) | VEPKSCDKTHL |
| ranibizumab cDNA | gagctccatg gagtttttca aaaagacggc acttgccgca ctggttatgg |
| (Light chain | gttttagtgg tgcagcattg gccgatatcc agctgaccca gagcccgagc |
| comprising a | agcctgagcg caagcgttgg tgatcgtgtt accattacct gtagcgcaag |
| signal sequence) | ccaggatatt agcaattatc tgaattggta tcagcagaaa ccgggtaaag |
| (12) | caccgaaagt tctgatttat tttaccagca gcctgcatag cggtgttccg |
| | agccgtttta gcggtagcgg tagtggcacc gattttaccc tgaccattag |
| | cagcctgcag ccggaagatt ttgcaaccta ttattgtcag cagtatagca |
| | ccgttccgtg gacctttggt cagggcacca aagttgaaat taaacgtacc |
| | gttgcagcac cgagcgtttt tatttttccg cctagtgatg aacagctgaa |
| | aagcggcacc gcaagcgttg tttgtctgct gaataatttt tatccgcgtg |
| | aagcaaaagt gcagtggaaa gttgataatg cactgcagag cggtaatagc |
| | caagaaagcg ttaccgaaca ggatagcaaa gatagcacct atagcctgag |
| | cagcaccctg accctgagca aagcagatta tgaaaaacac aaagtgtatg |
| | cctgcgaagt tacccatcag ggtctgagca gtccggttac caaaagtttt |
| | aatcgtggcg aatgctaata gaagcttggt acc |
| ranibizumab cDNA | gagctcatat gaaatacctg ctgccgaccg ctgctgctgg tctgctgctc |
| (Heavy chain | ctcgctgccc agccggcgat ggccgaagtt cagctggttg aaagcggtgg |
| comprising a | tggtctggtt cagcctggtg gtagcctgcg tctgagctgt gcagcaagcg |
| signal sequence) | gttatgattt tacccattat ggtatgaatt gggttcgtca ggcaccgggt |
| (13) | aaaggtctgg aatgggttgg ttggattaat acctataccg gtgaaccgac |
| | ctatgcagca gattttaaac gtcgttttac ctttagcctg gataccagca |
| | aaagcaccgc atatctgcag atgaatagcc tgcgtgcaga agataccgca |
| | gtttattatt gtgccaaata tccgtattac tatggcacca gccactggta |
| | tttcgatgtt tggggtcagg gcaccctggt taccgttagc agcgcaagca |
| | ccaaaggtcc gagcgttttt ccgctggcac cgagcagcaa aagtaccagc |
| | ggtggcacag cagcactggg ttgtctggtt aaagattatt ttccggaacc |
| | ggttaccgtg agctggaata gcggtgcact gaccagcggt gttcatacct |
| | ttccggcagt tctgcagagc agcggtctgt atagcctgag cagcgttgtt |
| | accgttccga gcagcagcct gggcacccag acctatattt gtaatgttaa |
| | tcataaaccg agcaatacca aagtggataa aaaagttgag ccgaaaagct |
| | gcgataaaac ccatctgtaa tagggtacc |
| ranibizumab Fab | DIQLTQSPSSLSASVGDRVTITCSASQDISNYLNWYQQKPGKAPKVLIYFTSSLH |
| Amino Acid | |
| Sequence (Light | SGVPSRFSGSGSGTDFTLTISSLQPEDFATYYCQQYSTVPWTFGQGTKVEIKRTV |
| chain) | AAPSVFIFPPSDEQLKSGTASVVCLLNNFYPREAKVQWKVDNALQSGNSQESVTE |
| (1) | QDSKDSTYSLSSTLTLSKADYEKHKVYACEVTHQGLSSPVTKSENRGEC |
| ranibizumab Fab | EVQLVESGGGLVQPGGSLRLSCAASGYDFTHYGMNWVRQAPGKGLEWVGWINTYT |
| Amino Acid | GEPTYAADFKRRFTFSLDTSKSTAYLQMNSLRAEDTAVYYCAKYPYYYGTSHWYF |
| Sequence (Heavy | DVWGQGTLVTVSSASTKGPSVFPLAPSSKSTSGGTAALGCLVKDYFPEPVTVSWN |
| chain) | SGALTSGVHTFPAVLQSSGLYSLSSVVTVPSSSLGTQTYICNVNHKPSNTKVDKK |
| (2) | VEPKSCDKTHL |
| bevacizumab Light | SASQDISNYLN |
| Chain CDRs | FTSSLHS |
| (14, 15, and 16) | QQYSTVPWT |
| bevacizumab Heavy | GYTFTNYGMN |
| Chain CDRs | WINTYTGEPTYAADFKR |
| (17, 18, and 19) | YPHYYGSSHWYFDV |
| ranibizumab Light | SASQDISNYLN |
| Chain CDRs | FTSSLHS |
| (14, 15, and 16) | QQYSTVPWT |
| ranibizumab Heavy | GYDFTHYGMN |
| Chain CDRs | WINTYTGEPTYAADFKR |
| (20, 18, and 21) | YPYYYGTSHWYFDV |

4.4.9 Manufacture and Testing of Vectors

The viral vectors provided herein may be manufactured using host cells. The viral vectors provided herein may be manufactured using mammalian host cells, for example, A549, WEHI, 1OT½, BHK, MDCK, COS1, COS7, BSC 1, BSC 40, BMT 10, VERO, W138, HeLa, 293, Saos, C2C12, L, HT1080, HepG2, primary fibroblast, hepatocyte, and myoblast cells. The viral vectors provided herein may be manufactured using host cells from human, monkey, mouse, rat, rabbit, or hamster.

The host cells are stably transformed with the sequences encoding the transgene and associated elements (i.e., the vector genome), and the means of producing viruses in the host cells, for example, the replication and capsid genes (e.g., the rep and cap genes of AAV). For a method of producing recombinant AAV vectors with AAV8 capsids, see Section IV of the Detailed Description of U.S. Pat. No. 7,282,199 B2, which is incorporated herein by reference in its entirety. Genome copy titers of said vectors may be determined, for example, by TAQMAN® analysis. Virions may be recovered, for example, by CsCl₂ sedimentation.

In vitro assays, e.g., cell culture assays, can be used to measure transgene expression from a vector described herein, thus indicating, e.g., potency of the vector. For example, the PER.C6® Cell Line (Lonza), a cell line derived from human embryonic retinal cells, or retinal pigment epithelial cells, e.g., the retinal pigment epithelial cell line hTERT RPE-1 (available from ATCC®), can be used to assess transgene expression. Once expressed, characteristics of the expressed product (i.e., HuGlyFabVEGFi) can be determined, including determination of the glycosylation and tyrosine sulfation patterns associated with the HuGlyFabVEGFi. Glycosylation patterns and methods of determining the same are discussed in Section 5.1.1, while tyrosine sulfation patterns and methods of determining the same are discussed in Section 5.1.2. In addition, benefits resulting from glycosylation/sulfation of the cell-expressed HuGlyFabVEGFi can be determined using assays known in the art, e.g., the methods described in Sections 5.1.1 and 5.1.2.

4.4.10 Target Patient Populations

The subjects treated in accordance with the methods described herein can be any mammals such as rodents, domestic animals such as dogs or cats, or primates, e.g. non-human primates. In a preferred embodiment, the subject is a human. In certain embodiments, the methods provided herein are for the administration to patients diagnosed with an ocular disease, in particular an ocular disease caused by increased neovascularization. In certain embodiments, the methods provided herein are for the administration to patients diagnosed with nAMD (wet AMD), dry AMD, retinal vein occlusion (RVO), diabetic macular edema (DME), or diabetic retinopathy (DR).

4.4.11 Sampling and Monitoring of Efficacy

Effects of the methods of treatment provided herein on visual deficits may be measured by BCVA (Best-Corrected Visual Acuity), intraocular pressure, slit lamp biomicroscopy, and/or indirect ophthalmoscopy. BCVA may be monitored using the Early Treatment Diabetic Retinopathy Study (ETDRS) score.

Effects of the methods of treatment provided herein on physical changes to eye/retina may be measured by SD-OCT (SD-Optical Coherence Tomography).

Efficacy may be monitored as measured by electroretinography (ERG).

Effects of the methods of treatment provided herein may be monitored by measuring signs of vision loss, infection, inflammation and other safety events, including retinal detachment.

Retinal thickness (e.g., central retinal thickness) or foveal thickness may be monitored to determine efficacy of the treatments provided herein. Without being bound by any particular theory, thickness of the retina may be used as a clinical readout, wherein the greater reduction in retinal thickness or the longer period of time before thickening of the retina, the more efficacious the treatment. Retinal function may be determined, for example, by ERG. ERG is a non-invasive electrophysiologic test of retinal function, approved by the FDA for use in humans, which examines the light sensitive cells of the eye (the rods and cones), and their connecting ganglion cells, in particular, their response to a flash stimulation. Retinal thickness and/or foveal thickness may be determined, for example, by SD-OCT. SD-OCT is a three-dimensional imaging technology which uses low-coherence interferometry to determine the echo time delay and magnitude of backscattered light reflected off an object of interest. OCT can be used to scan the layers of a tissue sample (e.g., the retina) with 3 to 15 m axial resolution, and SD-OCT improves axial resolution and scan speed over previous forms of the technology (Schuman, 2008, Trans. Am. Opthamol. Soc. 106:426-458).

Transgene product concentrations may be measured in the aqueous humor by any suitable method known in the art, e.g. by ELISA or Western Blot.

The potential for the vector transgene to spread to unintended recipients from shedding (release of vectors that did not infect the target cells and were cleared from the body via feces or bodily fluids), mobilization (transgene replication and transfer out of the target cell), or germ line transmission (genetic transmission to offspring through semen) may be evaluated by any suitable method known in the art. For example, vector shedding in biological fluids (e.g., urine, tears or serum) may be assayed by quantitative polymerase chain reaction measuring vector DNA. In some embodiments, no vector gene copies are detectable in a biological fluid (e.g., urine, tears or serum) at any time point after administration of the vector. In some embodiments, less than 210 gene copies/5 μL are detectable in the serum 14 weeks after administration of the vector.

The number of anti-VEGF injections (e.g., ranibizumab injections or Aflibercept injections) may be monitored to determine the efficacy and duration of a treatment provided herein. In some embodiments, the amount of anti-VEGF injections required by a subject treated in accordance with a method described herein over a certain amount of time (e.g., a month) is decreased by 10-20%, 20-30%, 30-40%, 40-50%, 50-60%, 60-70%, 70-80%, 80-90% or more than 90% compared to standard of care. In some embodiments, the amount of anti-VEGF injections required by a subject over a certain amount of time (e.g., a month) treated in accordance with a method described herein is decreased by 10-20%, 20-30%, 30-40%, 40-50%, 50-60%, 60-70%, 70-80%, 80-90% or more than 90% compared to what was required by the same subject prior to starting treatment according to the method described herein. Incidence of new retinal pigmentation and incidence of new geographic atrophy may be monitored to assess the safety of a treatment described herein.

TABLE 3
TABLE OF SEQUENCES

| SEQ | | |
|---|---|---|
| ID | | |
| NO: | Description | Sequence |
| 1 | Ranibizumab | DIQLTQSPSSLSASVGDRVTITCSASQDISNYLNWYQQKPGKAPKVLIYFTSSLH |
| | Fab Amino Acid | SGVPSRFSGSGSGTDFTLTISSLQPEDFATYYCQQYSTVPWTFGQGTKVEIKRTV |
| | Sequence (Light | AAPSVFIFPPSDEQLKSGTASVVCLLNNFYPREAKVQWKVDNALQSGNSQESVTE |
| | chain) | QDSKDSTYSLSSTLTLSKADYEKHKVYACEVTHQGLSSPVTKSENRGEC |
| 2 | Ranibizumab | EVQLVESGGGLVQPGGSLRLSCAASGYDFTHYGMNWVRQAPGKGLEWVGWINTYT |
| | Fab Amino Acid | GEPTYAADFKRRFTFSLDTSKSTAYLQMNSLRAEDTAVYYCAKYPYYYGTSHWYF |
| | Sequence | DVWGQGTLVTVSSASTKGPSVFPLAPSSKSTSGGTAALGCLVKDYFPEPVTVSWN |
| | (Heavy chain) | SGALTSGVHTFPAVLQSSGLYSLSSVVTVPSSSLGTQTYICNVNHKPSNTKVDKK |
| | | VEPKSCDKTHL |
| 3 | Bevacizumab | DIQMTQSPSSLSASVGDRVTITCSASQDISNYLNWYQQKPGKAPKVLIYFTSSLH |
| | Fab Amino Acid | SGVPSRFSGSGSGTDFTLTISSLQPEDFATYYCQQYSTVPWTFGQGTKVEIKRTV |
| | Sequence (Light | AAPSVFIFPPSDEQLKSGTASVVCLLNNFYPREAKVQWKVDNALQSGNSQESVTE |
| | chain) | QDSKDSTYSLSSTLTLSKADYEKHKVYACEVTHQGLSSPVTKSENRGEC |
| 4 | Bevacizumab | EVQLVESGGGLVQPGGSLRLSCAASGYTFTNYGMNWVRQAPGKGLEWVGWINTYT |
| | Fab Amino Acid | GEPTYAADFKRRFTFSLDTSKSTAYLQMNSLRAEDTAVYYCAKYPHYYGSSHWYF |
| | Sequence | DVWGQGTLVTVSSASTKGPSVFPLAPSSKSTSGGTAALGCLVKDYFPEPVTVSWN |
| | (Heavy chain) | SGALTSGVHTFPAVLQSSGLYSLSSVVTVPSSSLGTQTYICNVNHKPSNTKVDKK |
| | | VEPKSCDKTHL |
| 5 | VEGF-A signal | MNFLLSWVHW SLALLLYLHH AKWSQA |
| | peptide | |
| 6 | Fibulin-1 signal | MERAAPSRRV PLPLLLLGGL ALLAAGVDA |
| | peptide | |
| 7 | Vitronectin | MAPLRPLLIL ALLAWVALA |
| | signal peptide | |
| 8 | Complement | MRLLAKIICLMLWAICVA |
| | Factor H signal | |
| | peptide | |
| 9 | Opticin signal | MRLLAFLSLL ALVLQETGT |
| | peptide | |
| 10 | Bevacizumab | gctagcgcca ccatgggctg gtcctgcatc atcctgttcc tggtggccac |
| | CDNA | cgccaccggc gtgcactccg acatccagat gacccagtcc ccctcctccc |
| | (Light chain) | tgtccgcctc cgtgggcgac cgggtgacca tcacctgctc cgcctcccag |
| | | gacatctcca actacctgaa ctggtaccag cagaagcccg gcaaggcccc |
| | | caaggtgctg atctacttca cctcctccct gcactccggc gtgccctccc |
| | | ggttctccgg ctccggctcc ggcaccgact tcaccctgac catctcctcc |
| | | ctgcagcccg aggacttcgc cacctactac tgccagcagt actccaccgt |
| | | gccctggacc ttcggccagg gcaccaaggt ggagatcaag cggaccgtgg |
| | | ccgccccctc cgtgttcatc ttccccccct ccgacgagca gctgaagtcc |
| | | ggcaccgcct ccgtggtgtg cctgctgaac aacttctacc cccgggaggc |
| | | caaggtgcag tggaaggtgg acaacgccct gcagtccggc aactcccagg |
| | | agtccgtgac cgagcaggac tccaaggact ccacctactc cctgtcctcc |
| | | accctgaccc tgtccaaggc cgactacgag aagcacaagg tgtacgcctg |
| | | cgaggtgacc caccagggcc tgtcctcccc cgtgaccaag tccttcaacc |
| | | ggggcgagtg ctgagcggcc gcctcgag |
| 11 | Bevacizumab | gctagcgcca ccatgggctg gtcctgcatc atcctgttcc tggtggccac |
| | CDNA (Heavy | cgccaccggc gtgcactccg aggtgcagct ggtggagtcc ggcggcggcc |
| | chain) | tggtgcagcc cggcggctcc ctgcggctgt cctgcgccgc ctccggctac |
| | | accttcacca actacggcat gaactgggtg cggcaggccc ccggcaaggg |
| | | cctggagtgg gtgggctgga tcaacaccta caccggcgag cccacctacg |
| | | ccgccgactt caagcggcgg ttcaccttct ccctggacac ctccaagtcc |
| | | accgcctacc tgcagatgaa ctccctgcgg gccgaggaca ccgccgtgta |
| | | ctactgcgcc aagtaccccc actactacgg ctcctcccac tggtacttcg |
| | | acgtgtgggg ccagggcacc ctggtgaccg tgtcctccgc ctccaccaag |
| | | ggcccctccg tgttccccct ggccccctcc tccaagtcca cctccggcgg |
| | | caccgccgcc ctgggctgcc tggtgaagga ctacttcccc gagcccgtga |
| | | ccgtgtcctg gaactccggc gccctgacct ccggcgtgca caccttcccc |
| | | gccgtgctgc agtcctccgg cctgtactcc ctgtcctccg tggtgaccgt |
| | | gccctcctcc tccctgggca cccagaccta catctgcaac gtgaaccaca |
| | | agccctccaa caccaaggtg gacaagaagg tggagcccaa gtcctgcgac |
| | | aagacccaca cctgcccccc ctgccccgcc cccgagctgc tgggcggccc |
| | | ctccgtgttc ctgttccccc ccaagcccaa ggacaccctg atgatctccc |
| | | ggacccccga ggtgacctgc gtggtggtgg acgtgtccca cgaggacccc |
| | | gaggtgaagt tcaactggta cgtggacggc gtggaggtgc acaacgccaa |
| | | gaccaagccc cgggaggage agtacaactc cacctaccgg gtggtgtccg |
| | | tgctgaccgt gctgcaccag gactggctga acggcaagga gtacaagtgc |
| | | aaggtgtcca acaaggccct gcccgccccc atcgagaaga ccatctccaa |
| | | ggccaagggc cagccccggg agccccaggt gtacaccctg cccccctccc |
| | | gggaggagat gaccaagaac caggtgtccc tgacctgcct ggtgaagggc |
| | | ttctacccct ccgacatcgc cgtggagtgg gagtccaacg gccagcccga |
| | | gaacaactac aagaccaccc cccccgtgct ggactccgac ggctccttct |
| | | tcctgtactc caagctgacc gtggacaagt cccggtggca gcagggcaac |
| | | gtgttctcct gctccgtgat gcacgaggcc ctgcacaacc actacaccca |
| | | gaagtccctg tccctgtccc ccggcaagtg agcggccgcc |
| 12 | Ranibizumab | gagctccatg gagtttttca aaaagacggc acttgccgca ctggttatgg |
| | CDNA (Light | gttttagtgg tgcagcattg gccgatatcc agctgaccca gagcccgagc |
| | chain | agcctgagcg caagcgttgg tgatcgtgtt accattacct gtagcgcaag |
| | comprising a | ccaggatatt agcaattatc tgaattggta tagcagaaa ccgggtaaag |
| | signal sequence) | caccgaaagt tctgatttat tttaccagca gcctgcatag cggtgttccg |
| | | agccgtttta gcggtagcgg tagtggcacc gattttaccc tgaccattag |
| | | cagcctgcag ccggaagatt ttgcaaccta ttattgtcag cagtatagca |
| | | ccgttccgtg gacctttggt cagggcacca aagttgaaat taaacgtacc |
| | | gttgcagcac cgagcgtttt tatttttccg cctagtgatg aacagctgaa |
| | | aagcggcacc gcaagcgttg tttgtctgct gaataatttt tatccgcgtg |
| | | aagcaaaagt gcagtggaaa gttgataatg cactgcagag cggtaatagc |
| | | caagaaagcg ttaccgaaca ggatagcaaa gatagcacct atagcctgag |
| | | cagcaccctg accctgagca aagcagatta tgaaaaacac aaagtgtatg |
| | | cctgcgaagt tacccatcag ggtctgagca gtccggttac caaaagtttt |
| | | aatcgtggcg aatgctaata gaagcttggt acc |
| 13 | Ranibizumab | gagctcatat gaaatacctg ctgccgaccg ctgctgctgg tctgctgctc |
| | cDNA (Heavy | ctcgctgccc agccggcgat ggccgaagtt cagctggttg aaagcggtgg |
| | chain | tggtctggtt cagcctggtg gtagcctgcg tctgagctgt gcagcaagcg |
| | comprising a | gttatgattt tacccattat ggtatgaatt gggttcgtca ggcaccgggt |
| | signal sequence) | aaaggtctgg aatgggttgg ttggattaat acctataccg gtgaaccgac |
| | | ctatgcagca gattttaaac gtcgttttac ctttagcctg gataccagca |
| | | aaagcaccgc atatctgcag atgaatagcc tgcgtgcaga agataccgca |
| | | gtttattatt gtgccaaata tccgtattac tatggcacca gccactggta |
| | | tttcgatgtt tggggtcagg gcaccctggt taccgttagc agcgcaagca |
| | | ccaaaggtcc gagcgttttt ccgctggcac cgagcagcaa aagtaccagc |
| | | ggtggcacag cagcactggg ttgtctggtt aaagattatt ttccggaacc |
| | | ggttaccgtg agctggaata gcggtgcact gaccagcggt gttcatacct |
| | | ttccggcagt tctgcagagc agcggtctgt atagcctgag cagcgttgtt |
| | | accgttccga gcagcagcct gggcacccag acctatattt gtaatgttaa |
| | | tcataaaccg agcaatacca aagtggataa aaaagttgag ccgaaaagct |
| | | gcgataaaac ccatctgtaa tagggtacc |
| 14 | Bevacizumab | SASQDISNYLN |
| | and | |
| | Ranibizumab | |
| | Light Chain | |
| | CDR1 | |
| 15 | Bevacizumab | FTSSLHS |
| | and | |
| | Ranibizumab | |
| | Light Chain | |
| | CDR2 | |
| 16 | Bevacizumab | QQYSTVPWT |
| | and | |
| | Ranibizumab | |
| | Light Chain | |
| | CDR3 | |
| 17 | Bevacizumab | GYTFTNYGMN |
| | Heavy Chain | |
| | CDR1 | |
| 18 | Bevacizumab | WINTYTGEPTYAADFKR |
| | and | |
| | Ranibizumab | |
| | Heavy Chain | |
| | CDR2 | |
| 19 | Bevacizumab | YPHYYGSSHWYFDV |
| | Heavy Chain | |
| | CDR3 | |
| 20 | Ranibizumab | GYDFTHYGMN |
| | Heavy Chain | |
| | CDR1 | |
| 21 | Ranibizumab | YPYYYGTSHWYFDV |
| | Heavy Chain | |
| | CDR3 | |
| 22 | Albumin signal | MKWVTFISLLFLESSAYS |
| | peptide | |
| 23 | Chymotrypsinog | MAFLWLLSCWALLGTTFG |
| | en signal peptide | |
| 24 | Interleukin-2 | MYRMQLLSCIALILALVINS |
| | signal peptide | |
| 25 | Trypsinogen-2 | MNLLLILTFVAAAVA |
| | signal peptide | |
| 26 | F2A site | LLNFDLLKLAGDVESNPGP |
| 27 | T2A site | (GSG)EGRGSLLTCGDVEENPGP |
| 28 | P2A site | (GSG)ATNFSLLKQAGDVEENPGP |
| 29 | E2A site | (GSG)QCTNYALLKLAGDVESNPGP |
| 30 | F2A site | (GSG)VKQTLNFDLLKLAGDVESNPGP |
| 31 | Furin linker | RKRR |
| 32 | Furin linker | RRRR |
| 33 | Furin linker | RRKR |
| 34 | Furin linker | RKKR |
| 35 | Furin linker | R-X-K/R-R |
| 36 | Furin linker | RXKR |
| 37 | Furin linker | RXRR |
| 38 | Ranibizumab | MDIQLTQSPSSLSASVGDRVTITCSASQDISNYLNWYQQKPGKAPKVLIYFTSSL |
| | Fab amino acid | HSGVPSRFSGSGSGTDFTLTISSLQPEDFATYYCQQYSTVPWTFGQGTKVEIKRT |
| | sequence (Light | VAAPSVFIFPPSDEQLKSGTASVVCLLNNFYPREAKVQWKVDNALQSGNSQESVT |
| | chain) | EQDSKDSTYSLSSTLTLSKADYEKHKVYACEVTHQGLSSPVTKSENRGEC |
| 39 | Ranibizumab | MEVQLVESGGGLVQPGGSLRLSCAASGYDFTHYGMNWVRQAPGKGLEWVGWINTY |
| | Fab amino acid | TGEPTYAADFKRRFTFSLDTSKSTAYLQMNSLRAEDTAVYYCAKYPYYYGTSHWY |
| | sequence (Heavy | FDVWGQGTLVTVSSASTKGPSVFPLAPSSKSTSGGTAALGCLVKDYFPEPVTVSW |
| | chain) | NSGALTSGVHTFPAVLQSSGLYSLSSVVTVPSSSLGTQTYICNVNHKPSNTKVDK |
| | | KVEPKSCDKTHLRKRR |
| 40 | Ranibizumab | MEVQLVESGGGLVQPGGSLRLSCAASGYDFTHYGMNWVRQAPGKGLEWVGWINTY |
| | Fab amino acid | TGEPTYAADFKRRFTFSLDTSKSTAYLQMNSLRAEDTAVYYCAKYPYYYGTSHWY |
| | sequence (Heavy | FDVWGQGTLVTVSSASTKGPSVFPLAPSSKSTSGGTAALGCLVKDYFPEPVTVSW |
| | chain) | NSGALTSGVHTFPAVLQSSGLYSLSSVVTVPSSSLGTQTYICNVNHKPSNTKVDK |
| | | KVEPKSCDKTHL |
| 41 | AAV1 | MAADGYLPDWLEDNLSEGIREWWDLKPGAPKPKANQQKQDDGRGLVLPGYKYLGP |
| | | FNGLDKGEPVNAADAAALEHDKAYDQQLKAGDNPYLRYNHADAEFQERLQEDTSF |
| | | GGNLGRAVFQAKKRVLEPLGLVEEGAKTAPGKKRPVEQSPQEPDSSSGIGKTGQQ |
| | | PAKKRLNFGQTGDSESVPDPQPLGEPPATPAAVGPTTMASGGGAPMADNNEGADG |
| | | VGNASGNWHCDSTWLGDRVITTSTRTWALPTYNNHLYKQISSASTGASNDNHYFG |
| | | YSTPWGYFDFNRFHCHFSPRDWQRLINNNWGFRPKRLNFKLENIQVKEVTTNDGV |
| | | TTIANNLTSTVQVFSDSEYQLPYVLGSAHQGCLPPFPADVFMIPQYGYLTLNNGS |
| | | QAVGRSSFYCLEYFPSQMLRTGNNFTFSYTFEEVPFHSSYAHSQSLDRLMNPLID |
| | | QYLYYLNRTQNQSGSAQNKDLLFSRGSPAGMSVQPKNWLPGPCYRQQRVSKTKTD |
| | | NNNSNFTWTGASKYNLNGRESIINPGTAMASHKDDEDKFFPMSGVMIFGKESAGA |
| | | SNTALDNVMITDEEEIKATNPVATERFGTVAVNFQSSSTDPATGDVHAMGALPGM |
| | | VWQDRDVYLQGPIWAKIPHTDGHFHPSPLMGGFGLKNPPPQILIKNTPVPANPPA |
| | | EFSATKFASFITQYSTGQVSVEIEWELQKENSKRWNPEVQYTSNYAKSANVDFTV |
| | | DNNGLYTEPRPIGTRYLTRPL |
| 42 | AAV2 | MAADGYLPDWLEDTLSEGIRQWWKLKPGPPPPKPAERHKDDSRGLVLPGYKYLGP |
| | | FNGLDKGEPVNEADAAALEHDKAYDRQLDSGDNPYLKYNHADAEFQERLKEDTSF |
| | | GGNLGRAVFQAKKRVLEPLGLVEEPVKTAPGKKRPVEHSPVEPDSSSGTGKAGQQ |
| | | PARKRLNFGQTGDADSVPDPQPLGQPPAAPSGLGTNTMATGSGAPMADNNEGADG |
| | | VGNSSGNWHCDSTWMGDRVITTSTRTWALPTYNNHLYKQISSQSGASNDNHYFGY |
| | | STPWGYFDFNRFHCHFSPRDWQRLINNNWGFRPKRLNFKLFNIQVKEVTQNDGTT |
| | | TIANNLTSTVQVFTDSEYQLPYVLGSAHQGCLPPFPADVFMVPQYGYLTLNNGSQ |
| | | AVGRSSFYCLEYFPSQMLRTGNNFTFSYTFEDVPFHSSYAHSQSLDRLMNPLIDQ |
| | | YLYYLSRTNTPSGTTTQSRLQFSQAGASDIRDQSRNWLPGPCYRQQRVSKTSADN |
| | | NNSEYSWTGATKYHLNGRDSLVNPGPAMASHKDDEEKFFPQSGVLIFGKQGSEKT |
| | | NVDIEKVMITDEEEIRTTNPVATEQYGSVSTNLQRGNRQAATADVNTQGVLPGMV |
| | | WQDRDVYLQGPIWAKIPHTDGHFHPSPLMGGFGLKHPPPQILIKNTPVPANPSTT |
| | | FSAAKFASFITQYSTGQVSVEIEWELQKENSKRWNPEIQYTSNYNKSVNVDFTVD |
| | | TNGVYSEPRPIGTRYLTRNL |
| 43 | AAV3-3 | MAADGYLPDWLEDNLSEGIREWWALKPGVPQPKANQQHQDNRRGLVLPGYKYLGP |
| | | GNGLDKGEPVNEADAAALEHDKAYDQQLKAGDNPYLKYNHADAEFQERLQEDTSF |
| | | GGNLGRAVFQAKKRILEPLGLVEEAAKTAPGKKGAVDQSPQEPDSSSGVGKSGKQ |
| | | PARKRLNFGQTGDSESVPDPQPLGEPPAAPTSLGSNTMASGGGAPMADNNEGADG |
| | | VGNSSGNWHCDSQWLGDRVITTSTRTWALPTYNNHLYKQISSQSGASNDNHYFGY |
| | | STPWGYFDFNRFHCHFSPRDWQRLINNNWGFRPKKLSFKLFNIQVRGVTQNDGTT |
| | | TIANNLTSTVQVFTDSEYQLPYVLGSAHQGCLPPFPADVFMVPQYGYLTLNNGSQ |
| | | AVGRSSFYCLEYFPSQMLRTGNNFQFSYTFEDVPFHSSYAHSQSLDRLMNPLIDQ |
| | | YLYYLNRTQGTTSGTTNQSRLLFSQAGPQSMSLQARNWLPGPCYRQQRLSKTAND |
| | | NNNSNFPWTAASKYHLNGRDSLVNPGPAMASHKDDEEKFFPMHGNLIFGKEGTTA |
| | | SNAELDNVMITDEEEIRTTNPVATEQYGTVANNLQSSNTAPTTGTVNHQGALPGM |
| | | VWQDRDVYLQGPIWAKIPHTDGHFHPSPLMGGFGLKHPPPQIMIKNTPVPANPPT |
| | | TFSPAKFASFITQYSTGQVSVEIEWELQKENSKRWNPEIQYTSNYNKSVNVDFTV |
| | | DTNGVYSEPRPIGTRYLTRNL |
| 44 | AAV4-4 | MTDGYLPDWLEDNLSEGVREWWALQPGAPKPKANQQHQDNARGLVLPGYKYLGPG |
| | | NGLDKGEPVNAADAAALEHDKAYDQQLKAGDNPYLKYNHADAEFQQRLQGDTSFG |
| | | GNLGRAVFQAKKRVLEPLGLVEQAGETAPGKKRPLIESPQQPDSSTGIGKKGKQP |
| | | AKKKLVFEDETGAGDGPPEGSTSGAMSDDSEMRAAAGGAAVEGGQGADGVGNASG |
| | | DWHCDSTWSEGHVTTTSTRTWVLPTYNNHLYKRLGESLQSNTYNGFSTPWGYFDF |
| | | NRFHCHFSPRDWQRLINNNWGMRPKAMRVKIFNIQVKEVTTSNGETTVANNLTST |
| | | VQIFADSSYELPYVMDAGQEGSLPPFPNDVFMVPQYGYCGLVTGNTSQQQTDRNA |
| | | FYCLEYFPSQMLRTGNNFEITYSFEKVPFHSMYAHSQSLDRLMNPLIDQYLWGLQ |
| | | STTTGTTLNAGTATTNFTKLRPTNFSNFKKNWLPGPSIKQQGFSKTANQNYKIPA |
| | | TGSDSLIKYETHSTLDGRWSALTPGPPMATAGPADSKFSNSQLIFAGPKQNGNTA |
| | | TVPGTLIFTSEEELAATNATDTDMWGNLPGGDQSNSNLPTVDRLTALGAVPGMVW |
| | | QNRDIYYQGPIWAKIPHTDGHFHPSPLIGGFGLKHPPPQIFIKNTPVPANPATTE |
| | | SSTPVNSFITQYSTGQVSVQIDWEIQKERSKRWNPEVQFTSNYGQQNSLLWAPDA |
| | | AGKYTEPRAIGTRYLTHHL |
| 45 | AAV5 | MSFVDHPPDWLEEVGEGLREFLGLEAGPPKPKPNQQHQDQARGLVLPGYNYLGPG |
| | | NGLDRGEPVNRADEVAREHDISYNEQLEAGDNPYLKYNHADAEFQEKLADDTSFG |
| | | GNLGKAVFQAKKRVLEPFGLVEEGAKTAPTGKRIDDHFPKRKKARTEEDSKPSTS |
| | | SDAEAGPSGSQQLQIPAQPASSLGADTMSAGGGGPLGDNNQGADGVGNASGDWHC |
| | | DSTWMGDRVVTKSTRTWVLPSYNNHQYREIKSGSVDGSNANAYFGYSTPWGYFDF |
| | | NRFHSHWSPRDWQRLINNYWGFRPRSLRVKIFNIQVKEVTVQDSTTTIANNLTST |
| | | VQVFTDDDYQLPYVVGNGTEGCLPAFPPQVFTLPQYGYATLNRDNTENPTERSSF |
| | | FCLEYFPSKMLRTGNNFEFTYNFEEVPFHSSFAPSQNLFKLANPLVDQYLYRFVS |
| | | TNNTGGVQFNKNLAGRYANTYKNWFPGPMGRTQGWNLGSGVNRASVSAFATTNRM |
| | | ELEGASYQVPPQPNGMTNNLQGSNTYALENTMIFNSQPANPGTTATYLEGNMLIT |
| | | SESETQPVNRVAYNVGGQMATNNQSSTTAPATGTYNLQEIVPGSVWMERDVYLQG |
| | | PIWAKIPETGAHFHPSPAMGGFGLKHPPPMMLIKNTPVPGNITSFSDVPVSSFIT |
| | | QYSTGQVTVEMEWELKKENSKRWNPEIQYTNNYNDPQFVDFAPDSTGEYRTTRPI |
| | | GTRYLTRPL |
| 46 | AAV6 | MAADGYLPDWLEDNLSEGIREWWDLKPGAPKPKANQQKQDDGRGLVLPGYKYLGP |
| | | FNGLDKGEPVNAADAAALEHDKAYDQQLKAGDNPYLRYNHADAEFQERLQEDTSF |
| | | GGNLGRAVFQAKKRVLEPFGLVEEGAKTAPGKKRPVEQSPQEPDSSSGIGKTGQQ |
| | | PAKKRLNFGQTGDSESVPDPQPLGEPPATPAAVGPTTMASGGGAPMADNNEGADG |
| | | VGNASGNWHCDSTWLGDRVITTSTRTWALPTYNNHLYKQISSASTGASNDNHYFG |
| | | YSTPWGYFDFNRFHCHFSPRDWQRLINNNWGFRPKRLNFKLFNIQVKEVTTNDGV |
| | | TTIANNLTSTVQVFSDSEYQLPYVLGSAHQGCLPPFPADVFMIPQYGYLTLNNGS |
| | | QAVGRSSFYCLEYFPSQMLRTGNNFTFSYTFEDVPFHSSYAHSQSLDRLMNPLID |
| | | QYLYYLNRTQNQSGSAQNKDLLESRGSPAGMSVQPKNWLPGPCYRQQRVSKTKTD |
| | | NNNSNFTWTGASKYNLNGRESIINPGTAMASHKDDKDKFFPMSGVMIFGKESAGA |
| | | SNTALDNVMITDEEEIKATNPVATERFGTVAVNLQSSSTDPATGDVHVMGALPGM |
| | | VWQDRDVYLQGPIWAKIPHTDGHFHPSPLMGGFGLKHPPPQILIKNTPVPANPPA |
| | | EFSATKFASFITQYSTGQVSVEIEWELQKENSKRWNPEVQYTSNYAKSANVDFTV |
| | | DNNGLYTEPRPIGTRYLTRPL |
| 47 | AAV7 | MAADGYLPDWLEDNLSEGIREWWDLKPGAPKPKANQQKQDNGRGLVLPGYKYLGP |
| | | FNGLDKGEPVNAADAAALEHDKAYDQQLKAGDNPYLRYNHADAEFQERLQEDTSF |
| | | GGNLGRAVFQAKKRVLEPLGLVEEGAKTAPAKKRPVEPSPQRSPDSSTGIGKKGQ |
| | | QPARKRLNFGQTGDSESVPDPQPLGEPPAAPSSVGSGTVAAGGGAPMADNNEGAD |
| | | GVGNASGNWHCDSTWLGDRVITTSTRTWALPTYNNHLYKQISSETAGSTNDNTYF |
| | | GYSTPWGYFDFNRFHCHFSPRDWQRLINNNWGFRPKKLRFKLFNIQVKEVTTNDG |
| | | VTTIANNLTSTIQVFSDSEYQLPYVLGSAHQGCLPPFPADVFMIPQYGYLTLNNG |
| | | SQSVGRSSFYCLEYFPSQMLRTGNNFEFSYSFEDVPFHSSYAHSQSLDRLMNPLI |
| | | DQYLYYLARTQSNPGGTAGNRELQFYQGGPSTMAEQAKNWLPGPCFRQQRVSKTL |
| | | DQNNNSNFAWTGATKYHLNGRNSLVNPGVAMATHKDDEDRFFPSSGVLIFGKTGA |
| | | TNKTTLENVLMTNEEEIRPTNPVATEEYGIVSSNLQAANTAAQTQVVNNQGALPG |
| | | MVWQNRDVYLQGPIWAKIPHTDGNFHPSPLMGGFGLKHPPPQILIKNTPVPANPP |
| | | EVFTPAKFASFITQYSTGQVSVEIEWELQKENSKRWNPEIQYTSNFEKQTGVDFA |
| | | VDSQGVYSEPRPIGTRYLTRNL |
| 48 | AAV8 | MAADGYLPDWLEDNLSEGIREWWALKPGAPKPKANQQKQDDGRGLVLPGYKYLGP |
| | | FNGLDKGEPVNAADAAALEHDKAYDQQLQAGDNPYLRYNHADAEFQERLQEDTSF |
| | | GGNLGRAVFQAKKRVLEPLGLVEEGAKTAPGKKRPVEPSPQRSPDSSTGIGKKGQ |
| | | QPARKRLNFGQTGDSESVPDPQPLGEPPAAPSGVGPNTMAAGGGAPMADNNEGAD |
| | | GVGSSSGNWHCDSTWLGDRVITTSTRTWALPTYNNHLYKQISNGTSGGATNDNTY |
| | | FGYSTPWGYFDFNRFHCHFSPRDWQRLINNNWGFRPKRLSFKLFNIQVKEVTQNE |
| | | GTKTIANNLTSTIQVFTDSEYQLPYVLGSAHQGCLPPFPADVFMIPQYGYLTLNN |
| | | GSQAVGRSSFYCLEYFPSQMLRTGNNFQFTYTFEDVPFHSSYAHSQSLDRLMNPL |
| | | IDQYLYYLSRTQTTGGTANTQTLGFSQGGPNTMANQAKNWLPGPCYRQQRVSTTT |
| | | GQNNNSNFAWTAGTKYHLNGRNSLANPGIAMATHKDDEERFFPSNGILIFGKQNA |
| | | ARDNADYSDVMLTSEEEIKTTNPVATEEYGIVADNLQQQNTAPQIGTVNSQGALP |
| | | GMVWQNRDVYLQGPIWAKIPHTDGNFHPSPLMGGFGLKHPPPQILIKNTPVPADP |
| | | PTTFNQSKLNSFITQYSTGQVSVEIEWELQKENSKRWNPEIQYTSNYYKSTSVDF |
| | | AVNTEGVYSEPRPIGTRYLTRNL |
| 49 | hu31 | MAADGYLPDWLEDTLSEGIRQWWKLKPGPPPPKPAERHKDDSRGLVLPGYKYLGP |
| | | GNGLDKGEPVNAADAAALEHDKAYDQQLKAGDNPYLKYNHADAEFQERLKEDTSF |
| | | GGNLGRAVFQAKKRLLEPLGLVEEAAKTAPGKKRPVEQSPQEPDSSAGIGKSGSQ |
| | | PAKKKLNFGQTGDTESVPDPQPIGEPPAAPSGVGSLTMASGGGAPVADNNEGADG |
| | | VGSSSGNWHCDSQWLGDRVITTSTRTWALPTYNNHLYKQISNSTSGGSSNDNAYF |
| | | GYSTPWGYFDFNRFHCHFSPRDWQRLINNNWGFRPKRLNFKLFNIQVKEVTDNNG |
| | | VKTIANNLTSTVQVFTDSDYQLPYVLGSAHEGCLPPFPADVFMIPQYGYLTLNDG |
| | | GQAVGRSSFYCLEYFPSQMLRTGNNFQFSYEFENVPFHSSYAHSQSLDRLMNPLI |
| | | DQYLYYLSKTINGSGQNQQTLKFSVAGPSNMAVQGRNYIPGPSYRQQRVSTTVTQ |
| | | NNNSEFAWPGASSWALNGRNSLMNPGPAMASHKEGEDRFFPLSGSLIFGKQGTGR |
| | | DNVDADKVMITNEEEIKTTNPVATESYGQVATNHQSAQAQAQTGWVQNQGILPGM |
| | | VWQDRDVYLQGPIWAKIPHTDGNFHPSPLMGGFGMKHPPPQILIKNTPVPADPPT |
| | | AFNKDKLNSFITQYSTGQVSVEIEWELQKENSKRWNPEIQYTSNYYKSNNVEFAV |
| | | STEGVYSEPRPIGTRYLTRNL |
| 50 | hu32 | MAADGYLPDWLEDTLSEGIRQWWKLKPGPPPPKPAERHKDDSRGLVLPGYKYLGP |
| | | GNGLDKGEPVNAADAAALEHDKAYDQQLKAGDNPYLKYNHADAEFQERLKEDTSF |
| | | GGNLGRAVFQAKKRLLEPLGLVEEAAKTAPGKKRPVEQSPQEPDSSAGIGKSGSQ |
| | | PAKKKLNFGQTGDTESVPDPQPIGEPPAAPSGVGSLTMASGGGAPVADNNEGADG |
| | | VGSSSGNWHCDSQWLGDRVITTSTRTWALPTYNNHLYKQISNSTSGGSSNDNAYF |
| | | GYSTPWGYFDFNRFHCHFSPRDWQRLINNNWGFRPKRLNFKLFNIQVKEVTDNNG |
| | | VKTIANNLTSTVQVFTDSDYQLPYVLGSAHEGCLPPFPADVFMIPQYGYLTLNDG |
| | | SQAVGRSSFYCLEYFPSQMLRTGNNFQFSYEFENVPFHSSYAHSQSLDRLMNPLI |
| | | DQYLYYLSKTINGSGQNQQTLKFSVAGPSNMAVQGRNYIPGPSYRQQRVSTTVTQ |
| | | NNNSEFAWPGASSWALNGRNSLMNPGPAMASHKEGEDRFFPLSGSLIFGKQGTGR |
| | | DNVDADKVMITNEEEIKTTNPVATESYGQVATNHQSAQAQAQTGWVQNQGILPGM |
| | | VWQDRDVYLQGPIWAKIPHTDGNFHPSPLMGGFGMKHPPPQILIKNTPVPADPPT |
| | | AFNKDKLNSFITQYSTGQVSVEIEWELQKENSKRWNPEIQYTSNYYKSNNVEFAV |
| | | NTEGVYSEPRPIGTRYLTRNL |
| 51 | AAV9 | MAADGYLPDWLEDNLSEGIREWWALKPGAPQPKANQQHQDNARGLVLPGYKYLGP |
| | | GNGLDKGEPVNAADAAALEHDKAYDQQLKAGDNPYLKYNHADAEFQERLKEDTSF |
| | | GGNLGRAVFQAKKRLLEPLGLVEEAAKTAPGKKRPVEQSPQEPDSSAGIGKSGAQ |
| | | PAKKRLNFGQTGDTESVPDPQPIGEPPAAPSGVGSLTMASGGGAPVADNNEGADG |
| | | VGSSSGNWHCDSQWLGDRVITTSTRTWALPTYNNHLYKQISNSTSGGSSNDNAYF |
| | | GYSTPWGYFDFNRFHCHFSPRDWQRLINNNWGFRPKRLNFKLFNIQVKEVTDNNG |
| | | VKTIANNLTSTVQVFTDSDYQLPYVLGSAHEGCLPPFPADVFMIPQYGYLTLNDG |
| | | SQAVGRSSFYCLEYFPSQMLRTGNNFQFSYEFENVPFHSSYAHSQSLDRLMNPLI |
| | | DQYLYYLSKTINGSGQNQQTLKFSVAGPSNMAVQGRNYIPGPSYRQQRVSTTVTQ |
| | | NNNSEFAWPGASSWALNGRNSLMNPGPAMASHKEGEDRFFPLSGSLIFGKQGTGR |
| | | DNVDADKVMITNEEEIKTTNPVATESYGQVATNHQSAQAQAQTGWVQNQGILPGM |
| | | VWQDRDVYLQGPIWAKIPHTDGNFHPSPLMGGFGMKHPPPQILIKNTPVPADPPT |
| | | AFNKDKLNSFITQYSTGQVSVEIEWELQKENSKRWNPEIQYTSNYYKSNNVEFAV |
| | | NTEGVYSEPRPIGTRYLTRNL |
| 52 | Mutant | MYRMQLLLLIALSLALVINS |
| | Interleukin-2 | |
| | signal peptide | |

4.5 Assays

The skilled artesian may use the assays as described herein and/or techniques known in the art to study the composition and methods described herein, for example to test the formulations provided herein. The examples provided in Section 5 also demonstrate in more detail how such assays can be used to test the formulations provided herein.

As described in Li et al., 2019 Cell & Gene Therapy Insights, 5(4):537-547 (incorporated by references herein in its entirety), exemplary assays include but are not limited the following: (1) Digital Droplet PCR (ddPCR) for Genome Copy Determinations; (2) Genome Content and % Full Capsid Analysis of AAV by Spectrophotometry; (3) Size Exclusion Chromatography to Determine DNA Distribution and Purity in Capsid; (4) Assessing Capsid Viral Protein Purity Using Capillary Electrophoresis; (5) In Vitro Potency Methods-Relative Infectivity as a Reliable Method for Quantifying Differences in the Infectivity of AAV Vectors in vitro; and (6) Analytical Ultracentrifugation (AUC) to Determine Capsid Empty/Full Ratios and Size Distributions.

In addition, related conventional methods include methods provided in the United States Pharmacopeia (USP) published in 2019 and previous versions thereof (incorporated by reference herein in their entirety), for example USP<791> for pH measurements, USP<785> for osmolality measurements, USP<787> for particular matter (impurity) measurements, and USP<785> for endotoxin (safety) measurements, and USP<71> for sterility measurements.

As detailed in Section 5, the following assays are also provided herein. 4.5.1 Freeze/Thaw Cycles Assay

Controlled freeze/thaw cycles can be run in the lyophilizer according to Table 12. Vials can be well-spaced on the shelves and 4 vials of buffer can be thermocoupled. 4.5.2 Temperature Stress Assay

A temperature stress development stability study can be conducted at 1.0×10¹² GC/mL over 4 days at 37° C. to evaluate the relative stability of formulations provided herein.

Assays can be used to assess stability include but are not limited to in vitro relative potency (IVRP), vector genome concentration (VGC by ddPCR), free DNA by dye fluorescence, dynamic light scattering, appearance, and pH.

4.5.3 Long-Term Stability Assay

Long-term development stability studies can be carried out for 12 months to demonstrate maintenance of in-vitro relative potency and other quality at −80° C. (≤−60° C.) and-20° C. (−25° C. to −15° C.) in the formulations provided herein.

4.5.4 In Vitro Relative Potency (IVRP) Assay

In various embodiments, the compositions provided herein are more stable than a reference pharmaceutical composition as determined by the following assay. A percentage or fold difference in stability refers to in vitro relative potency as determined by this assay.

To relate the ddPCR GC titer to gene expression, an in vitro bioassay may be performed by transducing HEK293 cells and assaying the cell culture supernatant for anti-VEGF Fab protein levels. HEK293 cells are plated onto three poly-D-lysine-coated 96-well tissue culture plates overnight. The cells are then pre-infected with wild-type human Ad5 virus followed by transduction with three independently prepared serial dilutions of Construct II reference standard and test article, with each preparation plated onto separate plates at different positions. On the third day following transduction, the cell culture media is collected from the plates and measured for VEGF-binding Fab protein levels via ELISA. For the ELISA, 96-well ELISA plates coated with VEGF are blocked and then incubated with the collected cell culture media to capture anti-VEGF Fab produced by HEK293 cells. Fab-specific anti-human IgG antibody is used to detect the VEGF-captured Fab protein. After washing, horseradish peroxidase (HRP) substrate solution is added, allowed to develop, stopped with stop buffer, and the plates are read in a plate reader. The absorbance or OD of the HRP product is plotted versus log dilution, and the relative potency of each test article is calculated relative to the reference standard on the same plate fitted with a four-parameter logistic regression model after passing the parallelism similarity test, using the formula: EC50 reference÷EC50 test article. The potency of the test article is reported as a percentage of the reference standard potency, calculated from the weighted average of the three plates.

To relate the ddPCR GC titer to functional gene expression, an in vitro bioassay may be performed by transducing HEK293 cells and assaying for transgene (e.g. enzyme) activity. HEK293 cells are plated onto three 96-well tissue culture plates overnight. The cells are then pre-infected with wild-type human adenovirus serotype 5 virus followed by transduction with three independently prepared serial dilutions of enzyme reference standard and test article, with each preparation plated onto separate plates at different positions. On the second day following transduction, the cells are lysed, treated with low pH to activate the enzyme, and assayed for enzyme activity using a peptide substrate that yields increased fluorescence signal upon cleavage by transgene (enzyme). The fluorescence or RFU is plotted versus log dilution, and the relative potency of each test article is calculated relative to the reference standard on the same plate fitted with a four-parameter logistic regression model after passing the parallelism similarity test, using the formula: EC50 reference÷EC50 test article. The potency of the test article is reported as a percentage of the reference standard potency, calculated from the weighted average of the three plates.

In some embodiments, the in vitro potency of a recombinant AAV provided herein after being stored at ≤−60° C. (e.g., about −80° C.), at −30° C. to −15° C. (e.g., about −20° C.), or at 2° C. to 10° C. (e.g., about 4° C.) for a period of time is at least 70%, 75%, 80%, 85%, 90%, 95%, 98%, or 99% of the in vitro potency of the recombinant AAV before being stored at ≤−60° C. (e.g., about −80° C.), at −30° C. to −15° C. (e.g., about −20° C.), or at 2° C. to 10° C. (e.g., about 4° C.) for said period of time. In some embodiments, the period of time is about 1 month, about 2 months, about 3 months, about 4 months, about 5 months, about 6 months, about 7 months, about 8 months, about 9 months, about 10 months, about 11 months, about 12 months, about 15 months, about 18 months, or about 24 months.

4.5.5 Vector Genome Concentration Assay

In various embodiments, the compositions provided herein are more stable than a reference pharmaceutical composition as determined by the following assay. A percentage or fold difference in stability refers to vector genome concentration as determined by this assay. Vector genome concentration GC can also be evaluated using ddPCR. In some embodiments, the vector genome concentration of a recombinant AAV provided herein after being stored at ≤−60° C. (e.g., about −80° C.), at −30° C. to −15° C. (e.g., about −20° C.), or at 2° C. to 10° C. (e.g., about 4° C.) for a period of time is at least 70%, 75%, 80%, 85%, 90%, 95%, 98%, or 99% of the vector genome concentration of the recombinant AAV before being stored at ≤−60° C. (e.g., about-80° C.), at −30° C. to −15° C. (e.g., about −20° C.), or at 2° C. to 10° C. (e.g., about 4° C.) for said period of time. In some embodiments, the period of time is about 1 month, about 2 months, about 3 months, about 4 months, about 5 months, about 6 months, about 7 months, about 8 months, about 9 months, about 10 months, about 11 months, about 12 months, about 15 months, about 18 months, or about 24 months.

4.5.6 Free DNA Analysis Using Dye Fluorescence Assay

In various embodiments, the compositions provided herein are more stable than a reference pharmaceutical composition as determined by the following assay. A percentage or fold difference in stability refers to the amount of free DNA as determined by this assay.

Free DNA can be determined by fluorescence of SYBR® Gold nucleic acid gel stain (‘SYBR Gold dye’) that is bound to DNA. The fluorescence can be measured using a microplate reader and quantitated with a DNA standard. The results in ng/μL can be reported.

Two approaches can be used to estimate the total DNA in order to convert the measured free DNA in ng/μL to a percentage of free DNA. In the first approach the GC/mL (OD) determined by UV-visible spectroscopy was used to estimate the total DNA in the sample, where M is the molecular weight of the DNA and 1×10⁶ is a unit conversion factor:

Total DNA (ng/μL) estimated=1×10⁶×GC/mL (OD)×M (g/mol)/6.02×10²³

In the second approach, the sample can be heated to 85° C. for 20 min with 0.05% poloxamer 188 and the actual DNA measured in the heated sample by the SYBR Gold dye assay can be used as the total. This therefore has the assumption that all the DNA was recovered and quantitated. For example, the determination of total DNA by the SYBR gold dye (relative to the UV reading) can be found to be 131% for the Construct II dPBS formulation and 152% for the Construct II modified dPBS with sucrose formulation (This variation in the conversion of ng/μL to percentage of free DNA can be captured as a range in the reported results). For trending, either the raw ng/μL can be used or the percentage determined by a consistent method can be used.

In some embodiments, the amount of free DNA in a composition provided herein after being stored at ≤−60° C. (e.g., about −80° C.), at −30° C. to −15° C. (e.g., about −20° C.), or at 2° C. to 10° C. (e.g., about 4° C.) for a period of time is at least 70%, 75%, 80%, 85%, 90%, 95%, 98%, or 99% identical to the amount of free DNA in said composition before being stored at ≤−60° C. (e.g., about −80° C.), at −30° C. to −15° C. (e.g., about −20° C.), or at 2° C. to 10° C. (e.g., about 4° C.) for said period of time. In some embodiments, the period of time is about 1 month, about 2 months, about 3 months, about 4 months, about 5 months, about 6 months, about 7 months, about 8 months, about 9 months, about 10 months, about 11 months, about 12 months, about 15 months, about 18 months, or about 24 months.

4.5.7 Size Exclusion Chromatography (SEC)

In various embodiments, the compositions provided herein are more stable than a reference pharmaceutical composition as determined by the following assay. A percentage or fold difference in stability refers to size distribution as determined by this assay.

SEC can be performed using a Sepax SRT SEC-1000 Peek column (PN 215950P-4630, SN: 8A11982, LN: BTO90, 5 μm 1000A, 4.6×300 mm) on Waters Acquity Arc Equipment ID 0447 (C3PO), with a 25 mm pathlength flowcell. The mobile phase can be, for example, 20 mM sodium phosphate, 300 mM NaCl, 0.005% poloxamer 188, pH 6.5, with a flow rate of 0.35 mL/minute for 20 minutes, with the column at ambient temperature. Data collection can be performed with 2 point/second sampling rate and 1.2 nm resolution with 25 point mean smoothing at 214, 260, and 280 nm. The ideal target load can be 1.5¹¹ GC. The samples can be injected with 50 μL, about ⅓ of the ideal target or injected with 5 μL.

In some embodiments, the size distribution of a recombinant AAV provided herein as determined by SEC after being stored at ≤−60° C. (e.g., about −80° C.), at −30° C. to −15° C. (e.g., about −20° C.), or at 2° C. to 10° C. (e.g., about 4° C.) for a period of time is at least 70%, 75%, 80%, 85%, 90%, 95%, 98%, or 99% identical to the size distribution as determined by SEC of the recombinant AAV before being stored at ≤−60° C. (e.g., about −80° C.), at −30° C. to −15° C. (e.g., about −20° C.), or at 2° C. to 10° C. (e.g., about 4° C.) for said period of time. In some embodiments, the period of time is about 1 month, about 2 months, about 3 months, about 4 months, about 5 months, about 6 months, about 7 months, about 8 months, about 9 months, about 10 months, about 11 months, about 12 months, about 15 months, about 18 months, or about 24 months.

4.5.8 Dynamic Light Scattering (DLS) Assay

In various embodiments, the compositions provided herein are more stable than a reference pharmaceutical composition as determined by the following assay. A percentage or fold difference in stability refers to size distribution as determined by this assay.

Dynamic light scattering (DLS) can be performed on a Wyatt DynaProIII using Corning 3540 384 well plates with a 30 μL sample volume. Ten acquisitions each for 10 s can be collected per replicate and there were three replicate measurements per sample. The solvent can be set according to the solvent used in the samples, for example ‘PBS’ for Construct II in dPBS and ‘4% sucrose’ for the Construct II in modified dPBS with sucrose samples. Results not meeting data quality criteria (baseline, SOS, noise, fit) can be ‘marked’ and excluded from the analysis. The low delay time cutoff can be changed from 1.4 μs to 10 μs for the modified dPBS with sucrose samples to eliminate the impact of the sucrose excipient peak at about 1 nm on causing artifactually low cumulants analysis diameter results.

In some embodiments, the size distribution of a recombinant AAV provided herein as determined by DLS after being stored at ≤−60° C. (e.g., about −80° C.), at −30° C. to −15° C. (e.g., about −20° C.), or at 2° C. to 10° C. (e.g., about 4° C.) for a period of time is at least 70%, 75%, 80%, 85%, 90%, 95%, 98%, or 99% identical to the size distribution as determined by DLS of the recombinant AAV before being stored at ≤−60° C. (e.g., about −80° C.), at −30° C. to −15° C. (e.g., about −20° C.), or at 2° C. to 10° C. (e.g., about 4° C.) for said period of time. In some embodiments, the period of time is about 1 month, about 2 months, about 3 months, about 4 months, about 5 months, about 6 months, about 7 months, about 8 months, about 9 months, about 10 months, about 11 months, about 12 months, about 15 months, about 18 months, or about 24 months.

4.5.9 Differential Scanning Calorimetry

Low temperature Differential Scanning Calorimetry (low-temp DSC) can be run using a TA Instruments DSC250. About 20 μL of sample can be loaded into a Tzero pan and crimped with a Tzero Hermetic lid. Samples can be equilibrated at 25° C. for 2 min, then cooled at 5° C./min to −60° C., equilibrated for 2 min, then heated at 5° C./min to 25° C. Heat flow data can be collected in conventional mode.

4.5.10 Real-time Buffer pH Tracking

The pH of different formulation buffers was monitored with INLAB COOL PRO-ISM low temperature pH probe, which can detect pH down to −30° C. One milliliter buffer was placed in 15 mL Falcon tube and then the pH probe was submerged in the buffer. A piece of parafilm was used to seal the gap between Falcon tube and pH probe to avoid contamination and evaporation. The probe along with the Falcon tube was placed in −20 AD freezer. The pH and temperature of the buffer were recorded every 2.5 min for around 20 hour or until the pH versus temperature behavior achieved repeating pattern. The temperature change caused by the automatic defrosting process created a stress condition for buffer pH stability.

4.5.11 Osmolality

In various embodiments, the compositions provided herein are more stable than a reference pharmaceutical composition as determined by the following assay. A percentage or fold difference in stability refers to osmolality as determined by this assay.

The osmometer uses the technique of freezing-point depression to measure osmolality. Calibration of the instrument can be performed using 50 mOsm/kg, 850 mOsm/kg, and 2000 mOsm/kg NIST traceable standards. The reference solution of 290 mOsm/kg can be used to determine the system suitability of the osmometer.

In some embodiments, the osmolality of a recombinant AAV after being stored at ≤−60° C. (e.g., about −80° C.), at −30° C. to −15° C. (e.g., about −20° C.), or at 2° C. to 10° C. (e.g., about 4° C.) for a period of time is at least 70%, 75%, 80%, 85%, 90%, 95%, 98%, or 99% of the osmolality of the recombinant AAV before being stored a at ≤−60° C. (e.g., about −80° C.), at-30° C. to −15° C. (e.g., about −20° C.), or at 2° C. to 10° C. (e.g., about 4° C.) for said period of time. In some embodiments, the period of time is about 1 month, about 2 months, about 3 months, about 4 months, about 5 months, about 6 months, about 7 months, about 8 months, about 9 months, about 10 months, about 11 months, about 12 months, about 15 months, about 18 months, or about 24 months.

4.5.12 Density Measurement

In various embodiments, the compositions provided herein are more stable than a reference pharmaceutical composition as determined by the following assay. A percentage or fold difference in stability refers to density as determined by this assay.

The density can be measured with Anton Paar DMA500 densitometer, using water as reference. The densitometer can be washed with water and then methanol, followed by air-drying between samples.

In some embodiments, the density of a recombinant AAV after being stored at ≤−60° C. (e.g., about −80° C.), at −30° C. to −15° C. (e.g., about −20° C.), or at 2° C. to 10° C. (e.g., about 4° C.) for a period of time is at least 70%, 75%, 80%, 85%, 90%, 95%, 98%, or 99% of the density of the recombinant AAV before being stored at ≤−60° C. (e.g., about −80° C.), at −30° C. to −15° C. (e.g., about −20° C.), or at 2° C. to 10° C. (e.g., about 4° C.) for said period of time. In some embodiments, the period of time is about 1 month, about 2 months, about 3 months, about 4 months, about 5 months, about 6 months, about 7 months, about 8 months, about 9 months, about 10 months, about 11 months, about 12 months, about 15 months, about 18 months, or about 24 months.

4.5.13 Viscosity Measurement

In various embodiments, the compositions provided herein are more stable than a reference pharmaceutical composition as determined by the following assay. A percentage or fold difference in stability refers to viscosity as determined by this assay.

Viscosity can be measured using methods known in the art, for example methods provide in the United States Pharmacopeia (USP) published in 2019 and previous versions thereof (incorporated by reference herein in their entirety).

In some embodiments, the viscosity of a recombinant AAV after being stored at ≤−60° C. (e.g., about −80° C.), at −30° C. to −15° C. (e.g., about −20° C.), or at 2° C. to 10° C. (e.g., about 4° C.) for a period of time is at least 70%, 75%, 80%, 85%, 90%, 95%, 98%, or 99% of the viscosity of the recombinant AAV before being stored at ≤−60° C. (e.g., about −80° C.), at −30° C. to −15° C. (e.g., about −20° C.), or at 2° C. to 10° C. (e.g., about 4° C.) for said period of time. In some embodiments, the period of time is about 1 month, about 2 months, about 3 months, about 4 months, about 5 months, about 6 months, about 7 months, about 8 months, about 9 months, about 10 months, about 11 months, about 12 months, about 15 months, about 18 months, or about 24 months.

4.5.14 Virus Infectivity Assay

In various embodiments, the compositions provided herein are more stable than a reference pharmaceutical composition as determined by the following assay. A percentage or fold difference in stability refers to virus infectivity as determined by this assay.

TCID₅₀ infectious titer assay as described in Frangois, et al. Molecular Therapy Methods & Clinical Development (2018) Vol. 10, pp. 223-236 (incorporated by reference herein in its entirety) can be used. Relative infectivity assay as described in Provisional Application 62/745,859 filed Oct. 15, 2018) can be used.

In some embodiments, the viral infectivity of a recombinant AAV after being stored at ≤−60° C. (e.g., about −80° C.), at −30° C. to −15° C. (e.g., about −20° C.), or at 2° C. to 10° C. (e.g., about 4° C.) for a period of time is at least 70%, 75%, 80%, 85%, 90%, 95%, 98%, or 99% of the viral infectivity of the recombinant AAV before being stored at ≤−60° C. (e.g., about −80° C.), at-30° C. to −15° C. (e.g., about −20° C.), or at 2° C. to 10° C. (e.g., about 4° C.) for said period of time. In some embodiments, the period of time is about 1 month, about 2 months, about 3 months, about 4 months, about 5 months, about 6 months, about 7 months, about 8 months, about 9 months, about 10 months, about 11 months, about 12 months, about 15 months, about 18 months, or about 24 months.

4.5.15 Crystallization and Glass Transition Temperatures

Exemplary methods are described in Croyle et al., 2001, Gene Ther. 8(17):1281-90 (incorporated by reference in its entirety herein).

4.5.16 Reference Compositions

The stability of a composition provided herein may be evaluated by comparing the composition to a reference pharmaceutical composition. In some embodiments, the reference pharmaceutical composition is a pharmaceutical composition comprising the same recombinant AAV in the same concentration as the composition being evaluated in phosphate-buffered saline. In some embodiments, the reference pharmaceutical composition is a pharmaceutical composition comprising the same recombinant AAV in the same concentration as the composition being evaluated, but does not comprise sucrose. In some embodiments, the reference pharmaceutical composition is the same as the composition being evaluated before the composition has been stored at ≤−60° (e.g., about −80° C.) for 6-12 months. In some embodiments, the reference pharmaceutical composition is the same as the composition being evaluated before the composition has been stored at 2° C. to 10° C. (e.g., about 4° C.) for 1-6 months. In some embodiments, the reference pharmaceutical composition is the same as the composition being evaluated before the composition has been stored at about ≤−60° (e.g., about-80° C.) for 6-12 months and subsequently stored at 2° C. to 10° C. (e.g., about 4° C.) for 1-6 months.

In some embodiments, a given property (e.g., a property determined by an assay described in this section, i.e., section 4.5) of a recombinant AAV after being stored at ≤−60° C. (e.g., about −80° C.), at −30° C. to −15° C. (e.g., about −20° C.), or at 2° C. to 10° C. (e.g., about 4° C.) for a period of time is at least 70%, 75%, 80%, 85%, 90%, 95%, 98%, or 99% identical to the property of the recombinant AAV as determined by the same assay before being stored at ≤−60° C. (e.g., about −80° C.), at −30° C. to −15° C. (e.g., about −20° C.), or at 2° C. to 10° C. (e.g., about 4° C.) for said period of time. In some embodiments, the period of time is about 1 month, about 2 months, about 3 months, about 4 months, about 5 months, about 6 months, about 7 months, about 8 months, about 9 months, about 10 months, about 11 months, about 12 months, about 15 months, about 18 months, or about 24 months.

### 5. EXAMPLES

The examples in this section (i.e., section 5) are offered by way of illustration, and not by way of limitation.

### 5.1 Example 1: Components in Formulation a and Formulation B

This example shows the components in Formulation A (Dulbecco's phosphate buffered saline with 0.001% poloxamer 188, pH 7.4), stored at ≤−60° C., and Formulation B (‘modified Dulbecco’s phosphate buffered saline with 4% sucrose and 0.001% poloxamer 188, pH 7.4’), stored between−15° C. and −25° C. The comparison and impact analysis for the two Formulations is provided in Table 4. Formulation B has improved storage feasibility, without impact on the AAV product observed to date after 1 year of storage.

TABLE 4
FORMULATIONS A AND B.

| Process Site/Stage | Formulation A | Formulation B |
|---|---|---|
| Formulation Buffer | DPBS with 0.001% Poloxamer | ‘modified DPBS with 4% Sucrose and 0.001% |
| | 188, pH 7.4. | Poloxamer 188, pH 7.4.’ |
| | Composition: | The ‘modified DPBS with 4% Sucrose and |
| | 0.2 mg/mL potassium chloride, | 0.001% Poloxamer 188, pH 7.4.’formulation has |
| | 0.2 mg/mL potassium phosphate | 4% w/v of sucrose and a lower sodium chloride |
| | monobasic, 8.1 mg/mL sodium | level (reduced from 137 mM to 100 mM) to |
| | chloride, 1.15 mg/mL sodium | compensate tonicity. The other formulation |
| | phosphate dibasic anyhydrous, | excipients and levels are identical. |
| | 0.001% (0.01 mg/mL) | Composition: 0.2 mg/mL potassium chloride, |
| | poloxamer 188, pH 7.4 | 0.2 mg/mL potassium phosphate monobasic, |
| | | 5.84 mg/mL sodium chloride, 1.15 mg/mL |
| | | sodium phosphate dibasic anyhydrous, |
| | | 40.0 mg/mL (4% w/v) sucrose, 0.001% |
| | | (0.01 mg/mL) poloxamer 188, pH 7.4 |
| FDP Storage | ≤−60° C. | −15° C. to −25° C. |
| Temperature | | |

Formulation B (Modified DPBS with Sucrose) includes 0.2 mg/mL potassium chloride, 0.2 mg/mL potassium phosphate monobasic, 5.84 mg/mL sodium chloride, 1.15 mg/mL sodium phosphate dibasic anyhydrous, 40.0 mg/mL (40 w/v) sucrose, 0.001 (0.01 mg/mL) poloxamer 188, pH 7.4 (Table 5). In molar units, Formulation B includes 2.70 mM potassium chloride, 1.47 mM potassium phosphate monobasic, 100 mM sodium chloride, 8.1 mM sodium phosphate dibasic anyhydrous, 117 mM sucrose, 0.00100 (0.01 mg/mL) poloxamer 188, pH 7.4. The density of Formulation B may be 1.0188 g/mL; The osmolality of Formulation B may be approximately 345 (331-354).

TABLE 5
FORMULATION B WITH CONSTRUCT II AS ACTIVE PHARMACEUTICAL INGREDIENT (API).

| | | | | | | | Vendor | | |
|---|---|---|---|---|---|---|---|---|---|
| | | | | | | Mass | and | | Molecular |
| | | Quality | Concentration | Concentration | | Fraction | Part | Chemical | Weight |
| Ingredient | Function | Standard | (mg/mL) | (mM or %) | | (g/kg)^{b} | Number | Formula | (g/mol) |
| Construct II | API | Internal | Varies based on | — | | — | — | — | — |
| | | | dose level | | | | | | |
| Sodium | Buffering | USP, | 5.84 | 100 | mM | 5.736 | Avantor, | NaCl | 58.440 |
| Chloride | Agent | Ph. Eur, | | | | | 3627 | | |
| | | BP, JPE | | | | | | | |
| Potassium | | USP, BP, | 0.201 | 2.70 | mM | 0.198 | Avantor, | KCl | 74.5513 |
| Chloride | | Ph. Eur, | | | | | 3045 | | |
| | | JPE | | | | | | | |
| Sodium | | USP, | 1.15 | 8.10 | mM | 1.129 | Avantor, | Na₂HPO₄ | 141.960 |
| Phosphate | | Ph. Eur, | | | | | 3804 | | |
| Dibasic | | JPE | | | | | | | |
| Anhydrous | | | | | | | | | |
| Potassium | | NF, BP, | 0.200 | 1.47 | mM | 0.196 | Avantor, | KH₂PO₄ | 136.086 |
| Phosphate | | Ph. Eur | | | | | 3248 | | |
| Monobasic | | | | | | | | | |
| Sucrose | Cryoprotectant | USP, NF, | 40.0 | 117 | mM | 39.26 | Pfanstiehl, | C12H22O11 | 342.3 |
| | | Ph. Eur, | | | | | S-124-2- | | |
| | | BP, JPE | | | | | MC | | |
| Poloxamer | Surfactant^{a} | NF, | 0.010 | 0.001% | | 0.1 mL/ | BASF, | HO(C₃H₆O)ₐ | 7680 to 9510 |
| 188 | | Ph. Eur, | | | | kg of | 50424596 | (C₂H₄O)_{b}(C₃H₆O)ₐH | |
| | | JPE | | | | 10% | | | |
| | | | | | | stock | | | |

| | | | | | | | | |
|---|---|---|---|---|---|---|---|---|
| Water | Aqueous | WFI | Approximately | Approximately | QS to 1 | Varies | H₂O | 18.0153 |
| | Vehicle | | 971 mg/mL | 54M | kg | | | |

(need
approx.
953 g/kg)

^{a}Spike 0.1 mL/L = 0.1 mL/kg of 10% stock P188. NF grade Pluronic ® F-68 (poloxamer 188) from Spectrum and
Kolliphor ® P188 BIO from BASF may be used.
^{b}Volume of 1 kg of solution is approximately 982 mL (1 kg/1.0188 kg/L = 982 mL)

### 5.2 Example 2: Comparison of Formulation a and Formulation B in Release Characterization

This example shows the comparison of Formulation A and Formulation B in release characterization.

5.2.1 Release and Characterization Analytical Methods and Specification

This section shows comparison of lot release results from the certificate of analysis for one lot of final drug product (FDP) in the Formulation A and one lot of FDP in the new ‘modified DPBS with 4% Sucrose and 0.001% Poloxamer 188, pH 7.4’ (formulation B). In addition to the release tests the routine characterization test result for dynamic light scattering (DLS) may be compared. The panel of proposed tests to and the acceptance criteria to support comparability are shown in Table 6.

TABLE 6
ACCEPTANCE CRITERIA FOR RELEASE
AND CHARACTERIZATION METHODS
MAY BE USED TO PERFORM ANALYTICAL
COMPARABILITY OF THE FDP

| Test | Analytical Method | Acceptance Criteria |
|---|---|---|
| Appearance | Visual Inspection | Colorless, clear to opalescent |
| | | solution, free of foreign |
| | | particulates |
| pH | USP<791> | 6.9-7.9 |
| Osmolality | USP<785> | Meets lot release specification.¹ |
| Vector Genome | Transgene ddPCR | Meets lot release specification.² |
| Concentration | | |
| (Content) | | |
| Vector Genome | Transgene ddPCR | Confirmed as Construct II Genome |
| Concentration | | |
| (Identity) | | |
| Capsid Identity | ELISA | Confirmed as AAV8 Serotype |
| (Identity) | | |
| In Vitro | HEK293 | 50-200% Relative Potency |
| Potency | Transduction/Anti- | |
| (Potency) | VEGF ELISA | |
| Viral Capsid | SDS-CGE | ≥90% Purity (% Purity: Sum of |
| Purity (Purity) | | VP1, VP2, and VP3) |
| Particulate | USP<787> HIAC | ≤50 particles/mL |
| Matter | | (Size ≥10 μm) ≤5 particles/mL |
| (Impurity) | | (Size ≥25 μm) ≤2 particles/mL |
| | | (Size ≥50 μm) |
| Endotoxin | USP<85> | ≤0.1 EU/mL (Action |
| (Safety) | | Limit) ≤0.2 EU/mL |
| Sterility | USP<71> | No Growth |
| (Safety) | | |
| Size | Dynamic Light | Main Peak diameter consistent |
| Distribution | Scattering | with intact AAV |
| (Purity) | | |

5.2.2 Background on FDP Formulation Change

The ‘modified DPBS with 4% Sucrose and 0.00100 Poloxamer 188, pH 7.4’ (formulation B) was developed to improve the long-term frozen storage stability and robustness of the FDP stability to freeze/thaw cycles. The formulation change involved addition of 4% w/v of the cryoprotective excipient sucrose and a reduction in the sodium chloride level from 137 mM to 100 mM to maintain appropriate tonicity. The other formulation excipients and levels were identical. The generation of FDP in the ‘modified DPBS with 4% Sucrose and 0.00100 Poloxamer 188, pH 7.4’ FDP formulation was achieved by addition of a more concentrated sucrose spike solution to the BDS in DPBS (formulation A) to adjust the composition to the final composition.

5.2.3 Assessment of Changes to FDP Formulation Process

Spiking, mixing, and filtration steps involved similar process steps, handling, and contact surfaces for both processes. The spiking process resulted in a dilution of 1.37-fold. Then there was a subsequent dilution to target concentration.

5.2.4 Assessment of Impacts of FDP Formulation Change to Clinical Safety and Efficacy

FDP formulation B might have no difference in safety or efficacy when compared to the DPBS formulation A. The product quality profile and release specifications were the same, except for the general attribute, osmolality.

The osmolality for the DPBS formulation A was 240-340 mOsm/kg and for the formulation B the osmolality was 295-395 mOsm/kg. These were due to the adjustment of the level of sucrose and sodium chloride in the formulation B. There might be no impact of on the resorption time for blebs with these slightly higher osmolality values based on the literature (see, e.g., Negi and Marmour, 1984 Invest Ophthalmol Vis Sci. 25(5):616-20).

5.2.5 Assessment of the Impact of Changing from Formulation a to Formulation B to Analytical Release Methods

Assessment of analytical methods indicated they are fit-for-purpose. Minor modifications to their procedures can accommodate the new FDP (Table 7.

TABLE 7
ASSESSMENT OF NEW FDP COMPOSITION IMPACT
TO ANALYTICAL RELEASE METHODS

| | Analytical | |
|---|---|---|
| Test | Method | Assessment of Impact of FDP Change |
| Appearance | Visual Inspection | No impact. New FDP formulation (formulation B) |
| | | is expected to be a colorless, clear to opalescent |
| | | solution, free of foreign particulates. |
| pH | USP<791> | No impact. New FDP formulation has same |
| | | buffering species and levels. |
| Osmolality | USP<785> | No impact. Freezing-point depression method |
| | | gave expected result for new FDP. |
| Vector Genome | Transgene ddPCR | No Impact. Development samples were on-target |
| Concentration | | and met assay suitability criteria for new FDP. |
| (Content) | | |
| Vector Genome | Transgene ddPCR | No Impact. |
| Concentration | | |
| (Identity) | | |
| Capsid Identity | ELISA | No impact expected. |
| (Identity) | | |
| In Vitro | HEK293 | No Impact. Development samples were on-target |
| Potency | Transduction/Anti- | and met assay suitability criteria for new FDP. |
| (Potency) | VEGF ELISA | |
| Viral Capsid | SDS-CGE | No impact expected. |
| Purity (Purity) | | |
| Particulate | USP<787> HIAC | No impact expected. Light obscuration depends on |
| Matter | | clarity of solution which is similar for new FDP. |
| (Impurity) | | |
| Endotoxin | USP<85> | No impact. Samples tested met assay suitability |
| (Safety) | | criteria for new FDP. |
| Sterility | USP<71> | No impact. New FDP buffer was shown not to |
| (Safety) | | inhibit growth. |
| Size | Dynamic Light | Change in solution viscosity can be adjusted in |
| Distribution | Scattering | method settings. Development data shows a slight |
| (Purity) | (data from routine | increase (1 to 3 nm) in apparent size related to the |
| | characterization | impact of sucrose on the hydration and therefore |
| | testing performed | hydrodynamic behavior of the AAV capsids. |
| | either at release or | |
| | T0 stability) | |

### 5.3 Example 3: Comparison of Formulation a and Formulation B in Stability

This example shows the comparison of Formulation A and Formulation B in their stability.

The new Formulation B protects against disruption of capsids and release of small amounts of free DNA upon freeze/thaw cycles and temperature stress. Long-term stability studies currently 12 months demonstrated that the in-vitro relative potency and other quality attributes are maintained at −80° C. (≤−60° C.) and −20° C. (−25° C. to −15° C.) in the FDP formulation B.

The available freeze/thaw data, temperature stress data, and long-term stability data indicated similar or improved stability in the new formulation.

FDP lots in the new FDP formulation B can be set down on long-term stability at −80° C. (≤−60° C.) and −20° C. (−25° C. to −15° C.) and the stability trends data can be monitored as part of the stability program to ensure that the expiration date for the new FDP is compliant with regulations.

5.3.1 Freeze/Thaw Study for Construct II in DPBS and in the New FDP Formulation B

A study was conducted to assess the impact of freeze/thaw cycles on Construct II in DPBS and in the new FDP formulation B. Freeze/thaw rates were selected to bracket the expected rates that could occur during manufacturing for bottles of BDS or in the supply chain or clinic for vials of DP. Multiple cycles were applied to stress the samples beyond what might occur in the clinic.

The results of the study demonstrated that Formulation B was more robust than Formulation A when exposed to up to five freeze/thaw cycles from <−60° C. to 25° C. with either slow (0.12° C./min or over about 11 hours) and/or fast (1° C./min or over about 1 hour). All permutations of slow and fast rates were assessed for freezing and thawing respectively (i.e. FF/FT=fast freeze/fast thaw; FF/ST=fast freeze/slow thaw; SF/FT=slow freeze/fast thaw; SF/ST=slow freeze/slow thaw).

Freezing and thawing rates can impact the stability of biologics (Cao et al., 2003, Biotechnol. Bioeng. 82(6):684-90)). Crystallization of water during freezing can result in concentration of excipients which can impact the stability of biologics. Phase separation or pH shifts may also occur with an impact the stability of biologics. Fast freezing can lead to smaller ice crystals and a larger ice-water interface area which could impart interfacial stresses. Fast freezing could also entrap air bubbles in the ice leading to air-water interfacial stress during thawing. Slow thawing can result in re-crystallization of ice which can impact the stability of biologics in solution due to interfacial stress.

In this study, samples were analyzed by in vitro relative potency, size-exclusion chromatography purity (SEC), free DNA levels by fluorescent dye, and size distribution by dynamic light scattering. Phase changes of the formulations upon freezing and thawing were assessed by calorimetry.

There was little differentiation in results for different rates of fast and slow rates of freezing and thawing in this study. An overall summary of the freeze-thaw studies results is provided in Table 8. A representative example of a temperature profile (Fast Freeze/Slow Thaw) applied in this study is shown in FIG. 9 (other permutations of profiles not shown). Potency results were similar to the control for all permutations of fast and slow rates of freezing and thawing. There was no change in the size distribution within method variability upon freeze/thaw cycling of either Formulation A or Formulation B (see FIG. 12). The cumulants DLS diameter in formulation B of about 28 nm was slightly higher than in DPBS formulation A at 27 nm. This very slight apparent size increase is related to the impact of sucrose on the hydration and therefore hydrodynamic behavior of the AAV capsids. For Construct II in the DPBS formulation A, there was an increase in free DNA by dye fluorescence for all the freeze-thaw stress conditions (from 1.7% up to 6.9%). The free DNA quantitation by SEC was generally consistent with the dye fluorescence. By SEC the first pre-peak contains free DNA, the second small pre-peak contains aggregates, and the post-peak contains buffer species (see FIG. 10 and FIG. 11). The slightly lower free DNA by SEC might be due to very small or very large DNA fragments either co-eluting with the main peak or with the buffer, or not entering the column. The increases were of similar magnitude and not clearly differentiated for the different permutations of fast or very slow freeze-thaw rates. No increase in free DNA or aggregates were observed for Formulation B.

A small exotherm was observed in Formulation A at about −41° C. due to crystallization of amorphous sodium chloride that had not crystallized fully during cooling (FIG. 13). A eutectic melt with an unresolved low temperature shoulder was also observed with a peak at −22.1° C. for Formulation A. The same eutectic and re-crystallization events have been reported in the literature for sodium chloride (Milton et al., 2007, Journal of Pharmaceutical Sciences, 96(7)). The lack of a eutectic melt for formulation B (FIG. 14) was consistent with the inhibition of crystallization. Maintenance of an amorphous viscous state by the cryoprotective sucrose excipient in formulation B might provide protection against capsid disruption and release of free DNA upon freeze/thaw cycles. Therefore, the change to formulation B resulted in similar or improved stability compared to formulation A.

TABLE 8
IMPACT OF FREEZE-THAW RATES ON CONSTRUCT II
IN DPBS AND IN THE NEW FDP FORMULATION B

| | Construct II in DPBS with 0.001% | | | | Construct II in ‘modified DPBS with 4% | | | |
|---|---|---|---|---|---|---|---|---|
| | poloxamer 188, pH 7.4 | | | | Sucrose and 0.001% Poloxamer 188, pH 7.4’ | | | |
| | (Formulation A) | | | | (Formulation B) | | | |
| | | SE- | DLS | In vitro | | SE- | DLS | In vitro |
| Freeze | Free | HPLC^{b} | Cumulants | Relative | Free | HPLC^{b} | Cumulants | Relative |
| and Thaw | DNA | Pre-peak | Diameter | Potency | DNA | Pre-peak | Diameter | Potency |
| Rates^{c} | (%)^{a} | (%) | (nm) | (%) | (%)¹ | (%) | (nm) | (%) |
| Control | 1.7 | 2.1 | 26.8 | 88 | 2.5 | 2.3 | 28.0 | 87 |
| 5 × FF/FT | 6.9 | 4.5 | 28.4 | 88 | 2.7 | 1.6 | 28.5 | 89 |
| 5 × FF/ST | 5.9 | 4.5 | 26.7 | 87 | 2.5 | 1.2 | 27.9 | 88 |
| 5 × SF/FT | 6.6 | 3.6 | 26.7 | 88 | 2.6 | 1.1 | 27.7 | 92 |
| 5 × SF/ST | 6.8 | 3.4 | 26.9 | 86 | 2.7 | 1.5 | 28.1 | 88 |

^{a}Percent free DNA is based on the measured level compared to the total calculated from GC/mL (OD 260 nm).
^{b}SEC results calculated based on the 260 nm wavelength channel.
^{c}The actual product temperature ‘fast’ rate was about an hour for freezing and 1.5 hours for thawing. The ‘slow’ rate was about 11 hours for both freezing and thawing.

5.3.2 Temperature Stress Stability of Construct II in Formulation a and the New FDP Formulation B

A temperature stress development stability study conducted at 1.0×10¹² GC/mL over 4 days at 37° C. was used to evaluate the relative stability of formulation A and formulation B. Assays used to assess stability included in vitro relative potency (IVRP), vector genome concentration (VGC by ddPCR), free DNA by dye fluorescence, dynamic light scattering, appearance, and pH. Results for formulation A are shown in Table 9 and results for formulation B are shown in Table 10. A comparable decrease in potency within the assay variability of about 14 to 16% per day was observed for both formulations (see FIG. 15). There was also a small decrease in vector genome copies that was comparable for both formulation A and formulation B. The cumulants DLS diameter in Formulation B of about 29-34 nm was slightly higher than in DPBS formulation A at 28-30 nm. This very slight (1 to 3 nm) apparent size increase was related to the impact of sucrose on the hydration and therefore hydrodynamic behavior of the AAV capsids. There was no trend in DLS diameter within method variability for either Formulation A or Formulation B during the stress study. Formulation B was slightly more stable with respect to capsid disruption and had lower levels of free DNA. Free DNA increased from about 1 to 3% for Formulation A and remained below 1% for Formulation B (see FIG. 16). Overall the temperature stress stability of Formulation A and Formulation B were similar, with Formulation B being slightly more stable with respect to capsid disruption and release of free DNA. Therefore the change to Formulation B may result in similar or improved stability compared to Formulation A.

TABLE 9
TEMPERATURE STRESS STABILITY OF CONSTRUCT II
IN FORMULATION A 1.0 × 10¹² GC/ML HELD AT 37° C.

| | Time Point | | | |
|---|---|---|---|---|
| Test | 0 days | 1 day | 2 days | 4 days |
| IVRP (%) | 128 | 111 | 96 | 75 |
| VGC (GC/mL) | 1 × 10¹² | 9.70 × 10¹¹ | 9.50 × 10¹¹ | 8.60 × 10¹¹ |
| Free DNA (%) | 1.27 | 2.13 | 2.30 | 3.12 |
| DLS Diameter | 28.3 | 28.7 | 28.4 | 30.2 |
| (nm) | | | | |
| pH | 7.4 | 7.4 | 7.4 | 7.6 |
| Appearance | Clear, | Clear, | Clear, | Clear, |
| | colorless, | colorless, | colorless, | colorless, |
| | particle | particle | particle | particle |
| | free | free | free | free |

TABLE 10
TEMPERATURE STRESS STABILITY OF CONSTRUCT II
IN FORMULATION B 1.0 × 10¹² GC/ML HELD AT 37° C.

| | Time Point | | | |
|---|---|---|---|---|
| Test | 0 days | 1 day | 2 days | 4 days |
| IVRP (%) | 143 | 121 | 101 | 83 |
| VGC (GC/mL) | 11 × 10¹² | 1 × 10¹² | 9.20 × 10¹¹ | 8.80 × 10¹¹ |
| Free DNA (%) | 0.59 | 0.83 | 0.84 | 0.83 |
| DLS Diameter | 28.7 | 34.2 | 32.8 | 29.5 |
| (nm) | | | | |
| pH | 7.2 | 7.4 | 7.3 | 7.3 |
| Appearance | Clear, | Clear, | Clear, | Clear, |
| | colorless, | colorless, | colorless, | colorless, |
| | particle | particle | particle | particle |
| | free | free | free | free |

5.3.3 Long-Term Stability of Construct II in the New FDP Formulation B

Long-term development stability studies at 12 months demonstrated that the in-vitro relative potency and other quality attributes were maintained at −80° C. (≤−60° C.) and −20° C. (−25° C. to −15° C.) in the FDP formulation B. The study was conducted at both 1.0×10¹² GC/mL and at 2.1×10¹¹ GC/mL. Assays used to assess stability included in vitro relative potency (IVRP), vector genome concentration (VGC by ddPCR), free DNA by dye fluorescence (relative to-80° C. for time points up until 9 months, and absolute percentage at 12 months), size exclusion chromatography (SEC) for 1. Ox 10¹² GC/mL only, dynamic light scattering (DLS), pH, and appearance.

There was no trend on stability for all results at both −80° C. and −20° C. for both concentrations over 12 months. All results are similar for the −80° C. and −20° C. within method variability and similar to the initial time point results. The long-term stability data for 1.0×10¹² GC/mL held at −80° C. (Table 11) and −20° C. (Table 12) and for 2.1×10¹¹ GC/mL held at-80° C. (Table 13) and −20° C. (Table 14) demonstrate that Construct II is stable in formulation B for at least 12 months. IVRP potency trend graphs are shown in FIG. 17 and FIG. 18. The IVRP potency shows no consistent trend compared to method variability over the 12 months of long-term stability data.

TABLE 11
LONG-TERM STABILITY OF CONSTRUCT II IN FORMULATION B
AT 1.0 × 10¹² GC/ML AT −80° C.

| | Time Point | | | | |
|---|---|---|---|---|---|
| Test | 0 months | 3 months | 6 months | 9 months | 12 months |
| IVRP (%) | 82% | 91% | 90% | 112% | 110% |
| VGC (GC/mL) | 1 × 10¹² | 9.4 × 10¹¹ | 9.6 × 10¹¹ | 9.80 × 10¹¹ | 9.7 × 10¹¹ |
| Free DNA (relative | 1.00 | 1.00 | 1.00 | NA | 1.00 |
| to −80° C. control) | | | | | (1.1% |
| | | | | | absolute) |
| SEC (% purity) | 99.1 | 98.9 | 99.1 | Not tested | pending |
| DLS Diameter (nm) | NA | 28.4 | 28.1 | 27.9 | 28.7 |
| pH | 7.3 | Not tested | Not tested | Not tested | 7.3 |
| Appearance | Clear, | Clear, | Clear, | Clear, | Clear, |
| | colorless, | colorless, | colorless, | colorless, | colorless, |
| | particle free | particle free | particle free | particle free | particle free |

TABLE 12
LONG-TERM STABILITY OF CONSTRUCT II IN FORMULATION B
AT 1.0 × 10¹² GC/ML AT −20° C.

| | Time Point | | | | |
|---|---|---|---|---|---|
| Test | 0 months | 3 months | 6 months | 9 months | 12 months |
| IVRP (%) | 82% | 98% | 90% | 115% | 85% |
| VGC (GC/mL) | 1 × 10¹² | 9 × 10¹¹ | 9.6 × 10¹¹ | 9.9 × 10¹¹ | 9.5 × 10¹¹ |
| Free DNA (relative | 1.00 | 0.96 | 0.97 | Not tested | 1.37 |
| to −80° C. control) | | | | | (1.5% |
| | | | | | absolute) |
| SEC (% purity) | 99.1 | 98.8 | 99.3 | Not tested | pending |
| DLS Diameter (nm) | NA | 29.8 | 28.2 | 28 | 29.1 |
| pH | 7.3 | Not tested | Not tested | Not tested | 7.3 |
| Appearance | Clear, | Clear, | Clear, | Clear, | Clear, |
| | colorless, | colorless, | colorless, | colorless, | colorless, |
| | particle free | particle free | particle free | particle free | particle free |

TABLE 13
LONG-TERM STABILITY OF CONSTRUCT II IN FORMULATION B
AT 2.1 × 10¹¹ GC/ML AT −80° C.

| | Time Point | | | | |
|---|---|---|---|---|---|
| Test | 0 months | 3 months | 6 months | 9 months | 12 months |
| IVRP (%) | 113% | 107% | 120% | 104% | 97% |
| VGC (GC/mL) | 2.1 × 10¹¹ | 2.1 × 10¹¹ | 2.2 × 10¹¹ | 2.3 × 10¹¹ | 2.3 × 10¹¹ |
| Free DNA (relative | 1.00 | 1.00 | 1.00 | Not tested | 1.00 |
| to −80° C. control) | | | | | (1.4% |
| | | | | | absolute) |
| DLS Diameter (nm) | NA | 25.3 | 26.0 | 28.9 | 28.1 |
| pH | 7.3 | Not tested | Not tested | Not tested | 7.2 |
| Appearance | Clear, | Clear, | Clear, | Clear, | Clear, |
| | colorless, | colorless, | colorless, | colorless, | colorless, |
| | particle free | particle free | particle free | particle free | particle free |

TABLE 14
LONG-TERM STABILITY OF CONSTRUCT II IN FORMULATION B
AT 2.1 × 10¹¹ GC/ML AT −20° C.

| | Time Point | | | | |
|---|---|---|---|---|---|
| Test | 0 months | 3 months | 6 months | 9 months | 12 months |
| IVRP (%) | 113% | 104% | 115% | 109% | 86% |
| VGC (GC/mL) | 2.1 × 10¹¹ | 2.1 × 10¹¹ | 2.1 × 10¹¹ | 2.2 × 10¹¹ | 2.3 × 10¹¹ |
| Free DNA (relative | 1.00 | 0.99 | 1.10 | Not tested | 1.22 |
| to −80° C. control) | | | | | (1.8% |
| | | | | | absolute) |
| DLS Diameter(nm) | NA | 26.1 | 25.8 | 28.9 | 30.3 |
| pH | 7.3 | Not tested | Not tested | Not tested | 7.4 |
| Appearance | Clear, | Clear, | Clear, | Clear, | Clear, |
| | colorless, | colorless, | colorless, | colorless, | colorless, |
| | particle free | particle free | particle free | particle free | particle free |

### 5.4 Example 4: Comparison of Formulation a and Formulation B in Stability to Freeze and Thaw Cycles

5.4.1 Introduction

Freezing and thawing rates can impact the stability of biologics (Cao et al., 2003, Biotechnol. Bioeng. 82(6):684-90). Crystallization of water during slow freezing can result in concentration of excipients which can impact the stability of biologics. Phase separation or pH shifts may also occur with an impact the stability of biologics. Fast freezing can lead to smaller ice crystals and a larger ice-water interface area which could impart interfacial stresses. Fast freezing could also entrap air bubbles in the ice leading to air-water interfacial stress during thawing. Slow thawing can result in re-crystallization of ice which can impact the stability of biologics in solution due to interfacial stress.

Freeze-thaw stress can potentially disrupt AAV capsids resulting in release of small amounts of free DNA. During clinical trials, FDP can be shipped between the multiple vendors used for fill finish, storage, clinical packaging and labelling, and can be ultimately delivered to clinical sites. Un-planned temperature excursions encountered during shipment or product handling could lead to product warming or even thawing and re-freezing. The relative impacts of the rates of freezing and thawing could be used to assess excursions as well as guide freezing and thawing instructions at CMOs and at the clinic. The impact of freezing and thawing may also depend on the AAV type and its formulation. These factors were assessed in this example.

Larger volumes (60-110 mL) in BDS bottles were found to freeze at an overall average rate of about 0.5° C./min and 0.3° C./min for 60 and 110 mL of water, respectively. Smaller 0.6 mL fills in CZ vials took about 30-40 minutes to freeze from either room temperature or −20° C. (rates of about 2.0° C./min) while thawing took 30 minutes from room temperature and 10 minutes from −20° C. (rates of about 2.4° C./min and 4.5° C./min, respectively). In a prior study, it was shown that 250 mL Nalgene HDPE (BDS) bottles filled with 60 and 110 mL of water, took 163 and 266 minutes to freeze below −65° C., respectively. The corresponding freeze rates for these volumes were 0.53° C./min and 0.33° C./min for 60 and 110 mL of water, respectively. During thawing, a rapid rise in temperature was observed until the melting point was achieved—at which point the temperature increased slowly toward room temperature. Thawing took 273 and 337 minutes for 60 and 110 mL bottles, respectively (equivalent to an overall averaged rate of 0.32° C. to 0.25° C./min). The temperature profiles for freezing and thawing in bottles is shown in FIG. 19A.

In a separate prior study, freeze/thaw temperature profiles of 0.6 mL water fill into 2 mL Nalgene cryovials was explored. Temperature cycling occurred between a −80° C. freezer and either a −20° C. freezer or benchtop (room temperature). Data is shown in FIG. 19B. On average, freezing from room temperature to −60° C. took about 40 minutes while freezing from −20° C. to-60° C. took about 30 minutes. This corresponds to rates of about 2.0° C./min for both studies. Thawing from −60° C. to −20° C. occurred relatively rapid and took about 10 minutes while thawing to room temperature took around 30 minutes. This corresponds to rates of around 4.5° C./min for the −20° C. study and 2.4° C./min for the room temperature study.

In this example, the impact of multiple cycles of freeze/thaw at slow and fast freezing and thawing rates was evaluated. The impact of freeze/thaw rates of about 0.13° C./min (11 hour freeze or thaw) and 1.5° C./min (1 hour freeze or thaw) on the product quality of representative AAV8 (Construct II) was assessed. This was performed to further characterize the potential for variability in real-life excursions of temperature on the quality of AAV8. The slow rate was selected to be slower than expected for BDS slow freezing and thawing. For the fast rate, the maximum achievable rate that of about 1° C./min (about 10× faster than the slow rate) was studied as representative of fast thawing and freezing. Multiple cycles were applied to stress the samples beyond what might occur in the clinic. Samples were analyzed by in vitro relative potency, size-exclusion chromatography purity, free DNA levels (using a new SYBR Gold dye-based assay that was found to be sensitive to freeze-thaw stress), and size distribution by dynamic light scattering.

5.4.2 Summary of Freeze-Thaw Studies Results

Overall, the data showed that there was minimal impact to the quality attributes of Construct II in the dPBS and ‘modified dPBS with sucrose’ for five freeze/thaw cycles with rates ranging from as slow as 0.12° C./min (or over about 11 hours) to as fast as 1° C./min (or over about 1 hour).

The freeze/thaw rates were selected to bracket the expected rates that could occur in the clinic for bottles of BDS or vials of DP. Multiple cycles were applied to stress the samples beyond what might occur in the clinic.

An overall summary of the freeze-thaw studies results is provided in Table 15.

TABLE 15
SUMMARY OF FINDINGS

| | | | | | CONSTRUCT II in modified dPBS | | | |
|---|---|---|---|---|---|---|---|---|
| | CONSTRUCT II in dPBS | | | | with sucrose formulation | | | |
| | | SE- | | | | SE- | | |
| Freeze | | HPLC^{b} | DLS | In vitro | | HPLC^{b} | DLS | In vitro |
| and | Free | Pre- | Cumulants | Relative | Free | Pre- | Cumulants | Relative |
| Thaw | DNA | peak | Diameter | Potency | DNA | peak | Diameter | Potency |
| Rates^{c} | (%)^{a} | (%) | (nm) | %) | (%)^{a} | (%) | (nm) | (%) |
| Control | 1.7 | 2.1 | 26.8 | 88 | 2.5 | 2.3 | 28.0 | 87 |
| 5 × FF/FT | 6.9 | 4.5 | 28.4 | 88 | 2.7 | 1.6 | 28.5 | 89 |
| 5 × FF/ST | 5.9 | 4.5 | 26.7 | 87 | 2.5 | 1.2 | 27.9 | 88 |
| 5 × SF/FT | 6.6 | 3.6 | 26.7 | 88 | 2.6 | 1.1 | 27.7 | 92 |
| 5 × SF/ST | 6.8 | 3.4 | 26.9 | 86 | 2.7 | 1.5 | 28.1 | 88 |

^{a}Percent free DNA is based on the measured level compared to the total calculated from GC/mL (OD 260 nm). See results section for more details on the ng/μL levels and comparison to total results after capsid disruption by heat.
^{b}SEC results calculated based on the 260 nm wavelength channel.
^{c}The actual product temperature ‘fast’ rate was about an hour for freezing and 1.5 hours for thawing. The ‘slow’ rate was about 11 hours for both freezing and thawing.

5.4.3 Materials

Vials: CZ 2 mL vials, 13 mm, 19550057 (West, Daikyo)

Stoppers: 13 mm Serum NovaPure RP S2-F451 4432/50 Gray (West)

Construct II: Formulated at about 1×10¹² GC/mL in Dulbecco's phosphate-buffered saline (dPBS) buffer (0.2 g/L potassium chloride, 0.2 g/L potassium phosphate monobasic, 8.01 g/L sodium chloride, 1.15 g/L sodium phosphate dibasic anyhydrous, pH 7.4) with 0.001% poloxamer 188 and vialed at 0.5 mL in CZ vials (In this example, when dPBS is mentioned this implicitly describes the dPBS buffer that also contains 0.001% poloxamer 188).

Construct II: Formulated at about 1×10¹² GC/mL in ‘modified dPBS with sucrose’ (0.2 g/L potassium chloride, 0.2 g/L potassium phosphate monobasic, 5.84 g/L sodium chloride, 1.15 g/L sodium phosphate dibasic anyhydrous, 4% sucrose, 0.001% poloxamer 188, pH 7.4) and vialed at 0.5 mL in CZ vials. 5.4.4 Equipment

Genesis 25EL Lyophilizer (SP Scientific) Asset Tag 0941 (FFF) with temperature probe thermocouples.

Cytation 5 μlate reader (BioTek, Winooski, VT), Asset Tag 0867 (FFF instrument)

Cary 60 UV-Visible Spectrophotometer (Agilent, Santa Clara, CA), Asset Tag 0999 (FFF)

Thermal mixer/Heat block (Thermo Scientific), S/N: 01014318110768

Waters Acquity Arc Equipment ID 0447 (C3PO) and Sepax SRT SEC-1000 Peek column (PN 215950P-4630, SN: 8A11982, LN: BTO90, 5 μm 1000A, 4.6×300 mm)

TA instruments differential scanning calorimeter, DSC250/RCS90, Serial number DSC2A-00980, Asset Tag 0866.

5.4.5 Methods

(a) Controlled Freeze-Thaw Cycles in the Lyophilizer

Controlled freeze/thaw cycles were run in the lyophilizer according to Table 16. Vials were well-spaced on the shelves and 4 vials of buffer were thermocoupled.

TABLE 16
CONTROLLED FREEZE AND THAW RATE SETTINGS

| Condition^{a,b} | Freeze Rate | Thaw Rate |
|---|---|---|
| Frozen Control | NA | NA |
| 5 × Fast Freeze/ | 25° C. to −60° C. at | −60° C. to 25° C. at |
| Fast Thaw | 1.5° C./min | 1.5° C./min |
| (FF/FT) | (1 hour) | (1 hour) |
| 5 × Fast Freeze/ | 25° C. to −60° C. at | −60° C. to 25° C. at |
| Slow Thaw | 1.5° C./min^{c} | 0.13° C./min |
| (FF/ST) | (1 hour) | (11 hours) |
| 5 × Slow Freeze/ | 25° C. to −60° C. at | −60° C. to 25° C. at |
| Fast Thaw | 0.13° C./min | 1.5° C./min |
| (SF/FT) | (11.3 hours) | (1 hour) |
| 5 × Slow Freeze/ | 25° C. to −60° C. at | −60° C. to 25° C. at |
| Slow Thaw | 0.13° C./min | 0.13° C./min |
| (SF/ST) | (11 hours) | (11 hours) |

^{a}Shelves were programmed to hold at −60° C. and 25° C. for at least 1 hour between freeze and thaw cycles (there was a longer frozen hold was for some runs for laboratory scheduling purposes).
^{b}All samples and the frozen control were subjected to an uncontrolled freeze to −80° C. in the freezer and a thaw on the bench at room temperature at the end of the study before analysis.
^{c}The first cycle of the FF/ST was run from 25° C. to −55° C. in an attempt to reduce the load on the condenser. The subsequent 4 cycles were set to −60° C.

(b) In-Vitro Relative Potency

IVRP of Construct II was Performed:

To relate the ddPCR GC titer to gene expression, an in vitro bioassay was performed by transducing HEK293 cells and assaying the cell culture supernatant for anti-VEGF Fab protein levels. HEK293 cells were plated onto three poly-D-lysine-coated 96-well tissue culture plates overnight. The cells were then pre-infected with wild-type human Ad5 virus followed by transduction with three independently prepared serial dilutions of Construct II reference standard and test article, with each preparation plated onto separate plates at different positions. On the third day following transduction, the cell culture media was collected from the plates and measured for VEGF-binding Fab protein levels via ELISA. For the ELISA, 96-well ELISA plates coated with VEGF were blocked and then incubated with the collected cell culture media to capture anti-VEGF Fab produced by HEK293 cells. Fab-specific anti-human IgG antibody was used to detect the VEGF-captured Fab protein. After washing, horseradish peroxidase (RP) substrate solution was added, allowed to develop, stopped with stop buffer, and the plates were read in a plate reader. The absorbance or OD of the HRP product was plotted versus log dilution, and the relative potency of each test article was calculated relative to the reference standard on the same plate fitted with a four-parameter logistic regression model after passing the parallelism similarity test, using the formula: EC50 reference÷EC50 test article. The potency of the test article was reported as a percentage of the reference standard potency, calculated from the weighted average of the three plates.

(c) Free-DNA Analysis

Free DNA was determined by fluorescence of SYBR® Gold nucleic acid gel stain (‘SYBR Gold dye’) that is bound to DNA. The fluorescence was measured using a microplate reader and quantitated with a DNA standard. The results in ng/μL were reported.

Two approaches were used to estimate the total DNA in order to convert the measured free DNA in ng/μL to a percentage of free DNA. In the first approach the GC/mL (OD) determined by UV-visible spectroscopy was used to estimate the total DNA in the sample, where M is the molecular weight of the DNA and 1E6 is a unit conversion factor:

Total DNA (ng/μL) estimated=1E6×GC/mL (OD)×M (g/mol)/6.02×10²³

In the second approach the sample was heated to 85° C. for 20 min with 0.05% poloxamer 188 and the actual DNA measured in the heated sample by the SYBR Gold dye assay was used as the total. This therefore has the assumption that all the DNA was recovered and quantitated. The determination of total DNA by the SYBR gold dye (relative to the UV reading) was found to be 131% for the Construct II dPBS formulation, and higher for the Construct II modified dPBS with sucrose formulation (152%). This variation in the conversion of ng/μL to percentage of free DNA was captured as a range in the reported results. For trending, either the raw ng/μL can be used or the percentage determined by a consistent method can be used.

(d) Size-Exclusion HPLC Analysis

SEC was performed using a Sepax SRT SEC-1000 Peek column (PN 215950P-4630, SN: 8A11982, LN: BTO90, 5 μm 1000A, 4.6×300 mm) on Waters Acquity Arc Equipment ID 0447 (C3PO), with a 25 mm pathlength flowcell. The mobile phase was (20 mM sodium phosphate, 300 mM NaCl, 0.005% poloxamer 188, pH 6.5−VA 15Apr19), with a flow rate of 0.35 mL/minute for 20 minutes, with the column at ambient temperature. Data collection was performed with 2 point/second sampling rate and 1.2 nm resolution with 25 point mean smoothing at 214, 260, and 280 nm. The ideal target load was 1.5×10¹¹ GC. The Construct II samples were injected with 50 μL, about ⅓ of the ideal target and the Constuct II were injected with 5 μL.

(e) Dynamic Light Scattering

Dynamic light scattering (DLS) was performed on a Wyatt DynaProIII using Corning 3540 384 well plates with a 30 μL sample volume. Ten acquisitions each for 10 s were collected per replicate and there were three replicate measurements per sample. The solvent was set to ‘PBS’ Construct II in dPBS and was set to ‘4% sucrose’ for the Construct II in modified dPBS with sucrose samples. Results not meeting data quality criteria (baseline, SOS, noise, fit) were ‘marked’ and excluded from the analysis. The low delay time cutoff was changed from 1.4 μs to 10 μs for the modified dPBS with sucrose samples to eliminate the impact of the sucrose excipient peak at about 1 nm on causing artifactually low cumulants analysis diameter results.

(f) Differential Scanning Calorimetry

Low temperature Differential Scanning Calorimetry (low-temp DSC) was run using a TA Instruments DSC250. About 20 μL of sample was loaded into a Tzero pan and crimped with a Tzero Hermetic lid. Samples were equilibrated at 25° C. for 2 min, then cooled at 5° C./min to-60° C., equilibrated for 2 min, then heated at 5° C./min to 25° C. Heat flow data was collected in conventional mode.

5.4.6 Results

(a) Freeze-Thaw Study Temperature Profiles

The product temperature did not match the shelf exactly due to heat transfer limitations and phase transitions of the buffer during freezing and melting. The average rates determined using duration between when the product was near 25° C. and −60° C. for a representative portion of the cycle to calculate the overall average rates are summarized in Table 17.

As expected, there is a characteristic upward spike in temperature where the product was slightly warmer than the shelf temperature at approximately −10° C. as energy is released during freezing. Similarly, the product was at a slightly lower temperature relative to the shelf during melting of ice at around 0° C. The sections below show the probe temperature data. In addition, rates for the shelf and probes (i.e. 5 point slope of temperature and time) are shown for the FT/FT and the SF/SF as representative of the actual rates achieved for slow and fast rates.

The fast freeze average rate was limited to about 1° C./min and the fast thaw average rate was limited to about 0.8 to 1° C./min.

The actual product temperature ‘fast’ rate was about an hour for freezing and 1.5 hours for thawing. The ‘slow’ rate was about 0.12° C./min taking about 11 hours for both freezing and thawing.

TABLE 17
ACTUAL PRODUCT TEMPERATURE AND RATES
DURING FREEZE AND THAW CYCLES

| Condition^{1, 2} | Freeze Rate | Thaw Rate |
|---|---|---|
| 5 × Fast | Target: 25° C. to −60° C. | Target: −60° C. to 25° C. at |
| Freeze/Fast | at 1.5° C./min (1 hour) | 1.5° C./min (1 hour) |
| Thaw (FF/FT) | Actual: 20° C. to −53° C. | Actual: −53° C. to 20° C. at |
| | at 1° C./min (~1 hour) | 1° C./min (~1.5 hour) |
| 5 × Fast | Target: 25° C. to −60° C. | Target: −60° C. to 25° C. at |
| Freeze/Slow | at 1.5° C./min (1 hour) | 0.13° C./min (11 hours) |
| Thaw (FF/ST) | Actual: 23° C. to −55° C. | Actual: −55° C. to 23° C. at |
| | at 1.2° C./min (~1 hour) | 0.12° C./min (~11 hours) |
| 5 × Slow | Target: 25° C. to −60° C. | Target: −60° C. to 25° C. at |
| Freeze/Fast | at 0.13° C./min (11.3 hours) | 1.5° C./min (1 hour) |
| Thaw (SF/FT) | Actual: 23° C. to −55° C. | Actual: −55° C. to 23° C. at |
| | at 0.12° C./min (~11 hours) | 0.8° C./min (~1.5 hours) |
| 5 × Slow | Target: 25° C. to −60° C. | Target: −60° C. to 25° C. at |
| Freeze/Slow | at 0.13° C./min (11 hours) | 0.13° C./min (11 hours) |
| Thaw (SF/ST) | Actual: 24° C. to −56° C. | Actual: −56° C. to 24° C. at |
| | at 0.12° C./min (~11 hours) | 0.12° C./min (~11 hours) |

(b) Fast Freeze/Fast Thaw (FF/FT)

FIG. 20A shows the shelf and probe temperature profile for the FF/FT. There was a longer frozen hold was for some cycles for laboratory scheduling purposes.

The fast freeze average rate was limited to about 1° C./min and the fast thaw average rate was limited to about 0.8 to 1° C./min.

The temperature spike during the frozen portion of the first cycle appears to be an instrument spike. The spike near room temperature on the third cycle was due to a manual reset of the system to continue the cycles and associated temporary (for a few minutes) decrease in shelf temperature setting to a default closer to 10° C.

FIG. 20B shows a zoom in of both the shelf and product temperatures and their rates (averaged over 25 min). The actual rates show that the average product and shelf temperatures were impacted by the very rapid freezing and melting and associated limitations of heat transfer during these processes at the fast rates programmed. The melting of the product extracted heat from the shelf and resulted in a reduction in the shelf temperature rate during the melting temperature range.

(c) Fast Freeze/Slow Thaw (FF/ST)

FIG. 21 shows the shelf and probe temperature profile for the FF/ST. The first cycles of the FF/ST were run from 25° C. to −55° C. and back to 25° C. in an attempt to reduce the load on the condenser. The subsequent 4 cycles were set to −60° C. The time to freeze and thaw were not updated which increased the target rates to 1.6° C./min (from 1.5° C./min) and to 0.13° C./min (from 0.125° C./min). This difference is negligible at less than 10% from the original target rates. The spike near room temperature on the third cycle was due to a manual reset of the system to continue the cycles and associated temporary (for a few minutes) decrease in shelf temperature setting to a default closer to 10° C.

(d) Slow Freeze/Fast Thaw (SF/FT)

FIG. 22 shows the shelf and probe temperature profile for the SF/FT. There was a longer frozen hold was for the last cycle for laboratory scheduling purposes.

(e) Slow Freeze/Slow Thaw (SF/ST)

FIG. 23 shows the shelf and probe temperature profile for the FF/FT. There was a longer frozen hold for the third cycles for laboratory scheduling purposes. The spike near room temperature on the third cycle was due to a manual reset of the system to continue the cycles and associated temporary (for a few minutes) decrease in shelf temperature setting to a default closer to 10° C.

FIG. 24 shows a zoom in of both the shelf and product temperatures and their rates (averaged over 25 min). The rates show that the average product temperatures were impacted by the freezing and melting processes but that the shelf temperature rates remained stable at these slower rates (as compared with the FT/FT).

5.4.7 In-vitro Relative Potency

The in-vitro relative potency (IVRP) results were similar to the control and within method variability for all permutations of fast and slow rates of freezing and thawing for Construct II in both the dPBS and modified dPBS with sucrose formulations (Table 18).

TABLE 18
IVRP RESULTS

| | In-vitro Relative Potency (%) | | |
|---|---|---|---|
| | | | Construct II in |
| Freeze and Thaw | Control | Construct II | modified dPBS with |
| Rates | formulation | in dPBS | sucrose formulation |
| Control | 93 | 88 | 87 |
| 5 × FF/FT | 87 | 88 | 89 |
| 5 × FF/ST | 90 | 87 | 88 |
| 5 × SF/FT | 97 | 88 | 92 |
| 5 × SF/ST | 94 | 86 | 88 |

5.4.8 Free-DNA Results by SYBR Dye Binding and SEC

An overall result summary for free DNA is provided in Table 19. A range is provided for free DNA by SYBR Gold binding which represents the percentage based on either the GC/mL (GD) value for 10000 or the heat-stressed result for 1000% basis.

TABLE 19
FREE DNA BY SYBR GOLD AND PRE-PEAK BY SEC RESULTS

| | | | Construct II in modified dPBS with | |
|---|---|---|---|---|
| Freeze and | Construct II in dPBS | | sucrose formulation | |
| Thaw | Free | SE-HPLC^{b} | Free | SE-HPLC^{b} |
| Rates | DNA^{a} | Pre-peak (%) | DNA^{a} | Pre-peak (%) |
| Frozen | 0.046 ng/μL | 2.1% | 0.056 ng/μL | 2.3% |
| Control | 1.4% to 1.7% | Relative 1.0 | 1.7% to 2.5% | Relative 1.0 |
| | Relative 1.0 | | Relative 1.0 | |
| 5 × FF/FT | 0.181 ng/μL | 4.5% | 0.057 ng/μL | 1.6% |
| | 5.4% to 6.9% | Relative 2.2 | 1.8% to 2.7% | Relative 0.7 |
| | Relative 3.9 | | Relative 1.0 | |
| 5 × FF/ST | 0.156 ng/μL | 4.5% | 0.057 ng/μL | 1.2% |
| | 4.7% to 5.9% | Relative 2.2 | 1.7% to 2.5% | Relative 0.5 |
| | Relative 3.4 | | Relative 1.0 | |
| 5 × SF/FT | 0.173 ng/μL | 3.6% | 0.058 ng/μL | 1.1% |
| | 5.2% to 6.6% | Relative 1.7 | 1.7% to 2.6% | Relative 0.5 |
| | Relative 3.7 | | Relative 1.0 | |
| 5 × SF/ST | 0.178 ng/μL | 3.4% | 0.060 ng/μL | 1.5% |
| | 5.3% to 6.8% | Relative 1.7 | 1.8% to 2.7% | Relative 0.6 |
| | Relative 3.9 | | Relative 1.1 | |

^{a}Percent free DNA range is calculated as the percentage of the total detected in an 85° C. 20 min heat-stressed sample and the percentage calculated from GC/mL (OD 260 nm). ‘Relative’ is the ratio of the free DNA in the freeze-thaw samples compared to the frozen controls using the GC/mL (OD) values.
^{b}SEC results calculated based on the 260 nm wavelength channel.

A zoomed-in view of SEC result profiles are shown in FIG. 25, FIG. 26, and FIG. 27. The 260 nm UV channel was used to determine percent pre-peak which represents free DNA (earlier peaks) and some protein (closed pre-peak). A spectral analysis of the peaks and their elution positions indicate that the pre-peaks were predominantly free DNA. Peaks after the main peak are related to excipients.

5.4.9 Dynamic Light Scattering Results

DLS results are shown in for Construct II in dPBS in FIG. 28, and Construct II in modified dPBS with sucrose in FIG. 29. The diameter results for cumulants fitting and the main peak by regularization fitting are shown in Table 20.

The cumulants diameters ranged from 27.1 to 27.5 nm (control=27.5) and the regularization fit results ranged from 28.2 to 28.7 nm (control=28.4). The range in the cumulants data was 0.4 nm and the range in the regularization data was 0.5 nm. The standard deviation of the mean diameter for replicate measurements was about 0.2 for cumulants fitting and up to 0.8 nm for regularization fitting.

There was no change in the size distribution within method variability for Construct II in dPBS after any freeze-thaw condition. The cumulants diameters ranged from 26.7 to 28.4 nm (control=26.8) and the regularization from 26.2 to 27.6 nm (control=27.1). The range in the cumulants data was 1.7 nm and the range in the regularization data was 1.4 nm. The standard deviation of the mean diameter for replicate measurements was about 0.1 for cumulants fitting and up to 0.4 nm for regularization fitting.

There was no change in the size distribution within method variability for Construct II in modified dPBS with sucrose after any freeze-thaw condition. The cumulants diameters ranged from 27.7 to 28.5 nm (control=28.0) and the regularization from 28.2 to 31.3 nm (control=30.3). The range in the cumulants data was 0.8 nm and the range in the regularization data was 3.1 nm. In the modified dPBS with sucrose the standard deviation of the mean diameter for replicate measurements was about 0.2 nm for cumulants fitting and up to 1.3 nm for regularization fitting.

The cumulants diameter in sucrose of about 28 nm was slightly higher than in dPBS at 27 nm (FIG. 30). The regularization fit diameter of 30 nm was also slightly higher than in dPBS at about 27 nm as is shown in FIG. 31. This very slight apparent size increase might be related to the impact of sucrose on the hydration and therefore hydrodynamic behavior of the capsids.

TABLE 20
DLS DIAMETER RESULTS

| | | | Construct II in modified dPBS | |
|---|---|---|---|---|
| Freeze | Construct II in dPBS | | with sucrose formulation | |
| and Thaw | | Regularization | | Regularization |
| Rates | Cumulants | Peak | Cumulants | Peak |
| Control | 26.8 | 27.1 | 28.0 | 30.3 |
| 5 × FF/FT | 28.4 | 26.2 | 28.5 | 28.2^{a} |
| 5 × FF/ST | 26.7 | 27.4 | 27.9 | 31.3 |
| 5 × SF/FT | 26.7 | 27.2 | 27.7 | 30.2^{b} |
| 5 × SF/ST | 26.9 | 27.6 | 28.1 | 30.4 |
| Range in | 1.7 | 1.4 | 0.8 | 3.1 |
| data (nm) | | | | |

^{a}A sucrose excipient peak at 1.2 nm representing 2.4% intensity was detected.
^{b}A second peak with a diameter of 101 nm was detected in 1 replicate at 2% intensity.

5.4.10 Thermal Properties of Formulations

The low temperature DSC thermogram for the dPBS formulation buffer shown in FIG. 32 has a small exotherm at about −41° C. due to crystallization of amorphous sodium chloride that had not crystallized fully during cooling. Recrystallization of excipients has been implicated in glass vial breakage. This is not expected to be an issue for CZ vials (COP material). In addition, the literature reference for this scenario indicates vial breakage may not be a concern even for glass vials (Milton et al., 2007, Journal of Pharmaceutical Sciences, 96(7)). A eutectic melt with an unresolved low temperature shoulder was observed with a peak at −22.1° C. followed by a large endothermic peak due to melting of ice. The eutectic melt is consistent with a sodium chloride and water eutectic. The same eutectic and re-crystallization events have been reported in the literature for sodium chloride (Milton et al., 2007, Journal of Pharmaceutical Sciences, 96(7)).

The low temperature DSC thermogram for the ‘modified dPBS with sucrose’ buffer is shown FIG. 33 has a glass transition at −45.1° C. followed by a large endothermic peak due to melting of ice. No other transitions were observed. The glass transition is consistent with the presence of the glass-forming excipient, sucrose. The lack of a eutectic melt is consistent with the inhibition of crystallization and maintenance of an amorphous viscous state by the sucrose excipient. These results are consistent with the inhibition of crystallization and lack of a eutectic melt that was reported for lower (3%) sucrose with a higher (0.25 M) sodium chloride content (Milton et al., 2007, Journal of Pharmaceutical Sciences, 96(7)).

5.4.11 Conclusions

These results of this study demonstrated that up to five freeze/thaw cycles with either slow (0.12° C./min or over about 11 hours) and/or fast (1° C./min or over about 1 hour) rates were acceptable allowable excursions for Construct II in the dPBS and ‘modified dPBS with sucrose’ formulations.

The freeze/thaw rates were selected to bracket the expected rates that could occur for bottles of BDS or vials of DP. Multiple cycles were applied to stress the samples beyond what might occur in the clinic to support multiple excursions.

The potency results were similar to the control and within method variability for all permutations of fast and slow rates of freezing and thawing for Construct II in both the dPBS and modified dPBS with sucrose formulations

There was no change in the size distribution within method variability for Construct II in dPBS, or Construct II in modified dPBS with sucrose formulations after any freeze-thaw condition.

Freeze-thaw stress was shown to disrupt a small number of capsids of Construct II (AAV8) resulting in release of small amounts of free DNA when formulated without a cryoprotective excipient.

For Construct II in the dPBS formulation, there was an increase in free DNA for all the freeze-thaw stress conditions (from 1.7% up to 6.9%). The increases were of similar magnitude and not clearly differentiated for the different permutations of fast or very slow freeze-thaw rates.

The most stable formulation was the modified dPBS with sucrose formulation where no increase in free DNA was observed. The lack of a eutectic melt for this formulation is consistent with the inhibition of crystallization and maintenance of an amorphous viscous state by the cryoprotective sucrose excipient.

### 5.5 Example 5: Comparison of Formulation a and Formulation B in Scanning Calorimetry Profiles

This example shows comparison of Formulation A and Formulation B in calorimetry profiles.

The study was carried out by procedures the same as or similar to methods shown in Section 4.5.9, Section 5.4.5(f) and/or other relevant sections provided herein. As shown in FIG. 34, Formulation B contained an amorphous excipient that inhibited crystallization/eutectic transition improving robustness to freeze/thaw stress.

### 5.6 Example 6: Free DNA Increases with Each Freeze/Thaw Cycle for Formulation A

This example shows that free DNA increases with each freeze/thaw cycle for Formulation A.

The study was carried out by procedures the same as or similar to methods shown in Section 4.5.1, Section 5.4.5(c) and/or other relevant sections provided herein. As shown in FIG. 35, small and consistent increase in free DNA was detected for each freeze/thaw cycle for formulation A.

### 5.7 Example 7: Comparison of Formulation a and Formulation B in Potency after 30 Freeze and Thaw Cycles

This example shows the comparison of Formulation A and Formulation B in potency after 30 freeze and thaw cycles.

The study was carried out by procedures similar to the procedures shown in Section 4.5.1, Section 5.4.5(c) and/or other relevant sections provided herein.

The example was carried out for AAV8 with gene for green fluorescent protein. Freeze-thaw cycles were used to simulate transportation and storage logistics temperature changes and also as an ‘accelerated’ stress to force degradation of the AAV for formulation optimization work. As shown in FIG. 36, the ‘modified dPBS with 4% sucrose’ formulation B (dark grey bars) maintained potency after 30 freeze-thaw cycles. In contrast the reference formulation (dPBS, light grey bars) potency decreased to between 66% and 72% after 15 to 30 freeze-thaw cycles.

Shifts to acidic pH resulting in chemical and physical degradation, crystallization of salts (exposure to surfaces resulting in potency loss), and low viscosity (lower viscosity results in higher molecular mobility and reaction rates) can be the cause of potency loss for the reference DPBS formulation. These degradation pathways may be mitigated in the ‘modified dPBS with 4% sucrose’ formulation.

### 5.8 Example 8: Comparison of Formulation a and Formulation B in Adsorption Loss

This example shows methods to compare Formulation A and Formulation B in adsorption loss.

Contact materials must be considered in AAV Drug Product Design and for administration components. As shown in FIG. 37, amorphous excipient in Formulation B did not change the adsorption properties of AAV to Glass Vials. Adsorption losses occurred in glass vials but not detected for COP vials.

### 5.9 Example 9: Comparison of Formulation a and Formulation B in Long Term Stability

This example shows the comparison of Formulation A and Formulation B in long term stability.

The study was carried out by procedures the same as or similar to the procedures shown in Section 4.5.3 and/or other relevant sections provided herein. As shown in FIGS. 38 and 54, Formulation A and B had similar long-term frozen stability at −80° C., and Formulation B was also stable at −20° C. The ‘modified dPBS with 4% sucrose’ formulation B maintained potency for 12 months at −20° C. and −80° C. The reference formulation A (dPBS) is shown for −80° C. storage as a comparator.

### 5.10 Example 10: Comparison of Formulations in Physicochemical Properties

This example shows the characterization of physicochemical properties of different Construct II Formulation Buffer Candidates, including pH, glass transition temperature (Tg′), osmolality and density. CONSTRUCT II is currently designed to be stored in frozen formulation. The impact of freezing and freezer temperature fluctuation on buffer pH was investigated by real-time tracking of pH and temperature with low temperature pH probe in −20° C. automatic defrosting (−20° C. AD) freezer. Phase changes of the formulations upon freezing and thawing were assessed by calorimetry. The results of this study show that different magnitudes of pH shift happened depending on formulation buffer composition, which is a key factor to consider given the criticality of stable pH for drug product long term storage. In addition, the glass transition temperature of different formulation buffers also varied depending on formulation buffer composition. Lower Tg′ requires lower storage temperature to fully solidify the solution to minimize molecule mobility for potential physical and/or chemical degradation. The osmolality and density of all buffers were within the acceptable range for Construct II indication. This characterization study provides information for Construct II formulation buffer screening based on physicochemical properties.

5.10.1 Introduction

Biologics are often stored in buffers composed of various excipients to stabilize the drug product during storage. It is critical to maintain buffer pH and osmolality within target specification range to ensure product stability. Construct II drug product is targeted to be stored in frozen state between −80 to −20° C. Crystallization of water during slow freezing can result in concentration of excipients which can impact the stability of biologics. Phase separation or pH shifts may also occur which can impact the stability of biologics.

In this study, the impact of freezing and temperature excursion on formulation buffer pH stability was investigated. Construct II is currently stored in dPBS and ‘Modified dPBS with sucrose formulation. The impact of buffer component and concentration on stabilizing pH against freezing stress was illustrated. In addition, a Tris buffered saline (TBS) based formulation was also evaluated as a potential alternative formulation for AAV.

5.10.2 Summary of Freeze-Thaw Studies Results

Overall, the data showed that PBS-based formulation buffer showed an acceptable pH shift in response to freezing and temperature fluctuation. Adding 400 and 600 sucrose can mitigate the magnitude of pH shift for PBS-based buffer with ionic strength up to 150 mM. Tris-based formulation buffer showed one pH unit shift upon freezing. The impact of buffer component and concentration on stabilizing pH against freezing stress was investigated. All formulations tested are listed in Table 21.

TABLE 21
SUMMARY OF DIFFERENT FORMULATION BUFFER COMPOSITION

| | | Formulation # | | | | | | | |
|---|---|---|---|---|---|---|---|---|---|
| | | 1 | 2 | 3 | 4 | 5 | 6 | 7 | 8 |
| | | Also Known As | | | | | | | |
| | | | | | | | | | 5 mM |
| | | | | | | | ½ and | | Tris |
| | | | | | | | ½ | | with |
| | | | | | | | with | | 4% |
| | | | | | ½ and | ½ and | 6% | ¼ and | sucrose |
| | | | Modified | Modified | ½ | ½ | sucrose | ¼ | and |
| | | | dPBS | dPBS | with | with | and 80 | with | 100 |
| | Excipient | | with 4% | with 6% | 4% | 6% | mM | 4% | mM |
| | Function | dPBS | sucrose | sucrose | sucrose | sucrose | NaCl | sucrose | NaCl |
| Sodium | Buffering | 137 | 100 | 100 | 100 | 100 | 80 | 100 | 100 |
| Chloride | Agent | | | | | | | | |
| (mM) | | | | | | | | | |
| Potassium | | 3 | 3 | 3 | 3 | 3 | 3 | 3 | None |
| Chloride | | | | | | | | | |
| (mM) | | | | | | | | | |
| Tromethamine | | None | None | None | None | None | None | None | 0.9 |
| (mM) | | | | | | | | | |
| TRIS | | None | None | None | None | None | None | None | 4.1 |
| Hydrochloride | | | | | | | | | |
| (mM) | | | | | | | | | |
| Sodium | | 8.1 | 8.1 | 8.1 | 4.05 | 4.05 | 4.05 | 2.03 | None |
| Phosphate | | | | | | | | | |
| Dibasic | | | | | | | | | |
| Anhydrous | | | | | | | | | |
| (mM) | | | | | | | | | |
| Potassium | | 1.5 | 1.5 | 1.5 | 0.74 | 0.74 | 0.74 | 0.37 | None |
| Phosphate | | | | | | | | | |
| Monobasic | | | | | | | | | |
| (mM) | | | | | | | | | |
| Sucrose (% | Cryoprotectant | None | 4 | 6 | 4 | 6 | 6 | 4 | 4 |
| w/w) | | | | | | | | | |
| Poloxamer 188 | Surfactant¹ | 0.001 | 0.001 | 0.001 | 0.001 | 0.001 | 0.001 | 0.001 | 0.001 |
| (%) | | | | | | | | | |
| Water | Aqueous | QS to 1 kg | | | | | | | |
| | Vehicle | | | | | | | | |

Spike 0.1 mL/L = 0.1 mL/kg of 10% stock P188. Previously NF grade Pluronic ® F-68 (poloxamer 188) from Spectrum was used. Kolliphor ® P188 BIO (BASF) may also be used.

5.10.3 Definitions and Abbreviations

dPBS: Dulbecco's phosphate-buffered saline (has slightly lower phosphate level than regular PBS). Note, in this report where dPBS is mentioned this implicitly describes the dPBS buffer that also contains 0.00100 poloxamer 188.

glass transition temperature (Tg′)

−20 TC automatic defrosting freezer: −20 TC AD. The freezer prevents frosting from happening by increasing temperature from −20 to −6 TC and then decreasing back to −20 every 4 hours. One defrosting cycle (−20° C.→6° C.→20° C.) takes around one hour.

5.10.4 Materials

TABLE 22
SUMMARY OF EXCIPIENT INFORMATION

| | Vendor and Part | Chemical | Molecular |
|---|---|---|---|
| Chemical Name | Number | Formula | Weight (g/mol) |
| Sodium Chloride | Avantor, 3627 | NaCl | 58.44 |
| Potassium Chloride | Avantor, 3045 | KCl | 74.5513 |
| Tromethamine | Avantor, 4102 | C₄H₁₁NO₃ | 121.14 |
| TRIS Hydrochloride | Avantor, 4106 | NH₂C(CH₂OH)₃•HCl | 157.6 |
| Sodium Phosphate | Avantor, 3804 | Na₂HPO₄ | 141.96 |
| Dibasic Anhydrous | | | |
| Potassium Phosphate | Avantor, 3248 | KH₂PO₄ | 136.086 |
| Monobasic | | | |
| Sucrose | Pfanstiehl, S-124- | C₁₂H₂₂O₁₁ | 342.3 |
| | 2-MC | | |
| Poloxamer 188 | BASF, 50424596 | HO(C₃H₆O)ₐ(C₂H₄O)_{b}(C₃H₆O)ₐH | 7680 to 9510 |
| Water | RMBIO, WFI- | H₂O | 18.0153 |
| | SUP-1X6 | | |

5.10.5 Equipment
pH meter: Mettler Toledo SevenExcellence, Asset Tag 1000
Low temperature pH probe: INLAB COOL PRO-ISM. This pH probe can detect pH down to −30° C.
Balance: Mettler Toledo, XSR4002S, Asset Tag 0971 (for weighing larger masses)
Balance: Mettler Toledo, XSR105, Asset Tag 1020 (for weighing smaller masses)
−20° C. AD freezer: VWR 10819-664, Asset Tag 1213
TA instruments differential scanning calorimeter, DSC250/RCS90, Serial number DSC2A-00980, Asset Tag 0866.
Densitometer: Anton Paar DMA500 densitometer, Asset Tag 1087
Osmometer: Advanced Instruments, Asset Tag 0472

5.10.6 Methods

(a) Real-Time Buffer pH Tracking

The pH of different formulation buffers was monitored with INLAB COOL PRO-ISM low temperature pH probe, which can detect pH down to −30° C. One milliliter buffer was placed in 15 mL Falcon tube and then the pH probe was submerged in the buffer. A piece of parafilm was used to seal the gap between Falcon tube and pH probe to avoid contamination and evaporation. The probe along with the Falcon tube was placed in −20 AD freezer. The pH and temperature of the buffer were recorded every 2.5 min for around 20 hr or until the pH versus temperature behavior achieved repeating pattern. The temperature change caused by the automatic defrosting process created a stress condition for buffer pH stability.

(b) Differential Scanning Calorimetry

Low temperature Differential Scanning Calorimetry (low-temp DSC) was run using a TA Instruments DSC250. About 20 μL of sample was loaded into a Tzero pan and crimped with a Tzero Hermetic lid. Samples were equilibrated at 25° C. for 2 min, then cooled at 5° C./min to-60° C., equilibrated for 2 min, then heated at 5° C./min to 25° C. Heat flow data was collected in conventional mode.

(c) Osmolality

The osmometer uses the technique of freezing-point depression to measure osmolality. Calibration of the instrument was performed using 50 mOsm/kg, 850 mOsm/kg, and 2000 mOsm/kg NIST traceable standards. The reference solution of 290 mOsm/kg was used to determine the system suitability of the osmometer.

(d) Density

The density was measured with Anton Paar DMA500 densitometer, using water as reference. The densitometer was washed with water and then methanol, followed by air-drying between samples.

5.10.7 Results

(a) Real-Time Buffer pH Tracking

The pH of formulation buffer is critical in maintaining biologic drug product stability during long term storage. Some excipient components might precipitate when temperature decrease to below eutectic point, causing buffer pH shift and potentially impact drug product stability. Using a low temperature pH probe, the pH change along with temperature of the eight formulation buffers were monitored. An example of monitoring the pH and temperature of Formulation #2, Modified dPBS with 4% sucrose is shown in FIG. 39. The pH of all formulations after reaching stabilization is in FIG. 40.

The PBS-based Formulation #1-7 showed a tendency of decreasing pH as the temperature decreased from 0 to −18° C. (FIG. 41). Formulation #1 (dPBS) showed three pH units decrease from 7.4 to 4.2, the largest magnitude of pH shift among PBS-based Formulation #1-7 (FIG. 42). Sodium phosphate dibasic in phosphate-based formulation precipitates upon solution freezing, and a three pH units change is consistent with previous report (Zbacnik, 2017, Journal of Pharmaceutical Sciences, 106(3):713-733). However, the pH of Formulation #2-7 only decreased one pH unit when 4% to 6% sucrose was added to the solution. Previous research has shown that trehalose and mannitol can attenuate the magnitude of pH shift by inhibiting buffer salt crystallization under certain conditions, rendering the frozen solution in amorphous state and suppressing eutectic melting (Thorat and Suryanarayanan, 2019, Pharmaceutical Research 26(7):98) To illustrate the mechanism of sucrose attenuating pH shift of PBS-based formulation, the phase transition behavior of all formulations was characterized by DSC.

The pH of Tris-based Formulation #8 increased from 7.6 to 8.7, which is consistent with the pKa of Tris change (−0.03/° C.) in response to decreasing temperature (Zbacnik, 2017, Journal of Pharmaceutical Sciences, 106(3):713-733). Sucrose functions as cryoprotectant in this formulation since Tris salt does not crystallize as solution freezes.

(b) Thermal Analysis

The low temperature DSC thermogram for Formulation #1 (dPBS) shown in FIG. 43 has a eutectic melt with a peak at −22.1° C. followed by a large endothermic peak due to melting of ice. No other transitions were observed. The eutectic melt is consistent with a sodium chloride and water eutectic. The low temperature DSC thermogram for the Formulation #1 shown in FIG. 43 has a small exotherm at about −43° C. due to crystallization of amorphous sodium chloride that had not crystallized fully during cooling.

The low temperature DSC thermogram for the Formulation #2 (Modified dPBS with sucrose) is shown FIG. 44 has a glass transition at −44.83° C. followed by a large endothermic peak due to melting of ice. No other transitions were observed. The glass transition is consistent with the presence of the glass-forming excipient, sucrose. The lack of a eutectic melt is consistent with the inhibition of crystallization and maintenance of an amorphous viscous state by the sucrose excipient. These results are consistent with the inhibition of crystallization and lack of a eutectic melt that was reported for lower (3%) sucrose with a higher (0.25 M) sodium chloride content (Milton et al., 2007, Journal of Pharmaceutical Sciences, 96(7)).

Formulation #2-7 have the same components but different concentration for sodium phosphate dibasic anhydrous, potassium phosphate monobasic, or sucrose. Hence Formulation #3-7 showed similar phase transition behavior as Formulation #2, with a glass transition between −40 to −45° C. followed by a large endothermic peak due to melting of ice (FIG. 45). The phase transition temperature of all 8 formulations are listed in Table 21. Higher glass transition temperature at −41 to −42° C. were observed in formulations containing 6% sucrose, further proving the effect of sucrose on inhibiting buffer salt crystallization.

(c) Osmolality and Density

Osmolality is one the key factors determining formulation tolerability upon injection. Hypertonicity can cause local discomfort, irritation, and sensation of heat and pain etc. It is recommended that the upper osmolality limit should be generally controlled under 600 mOsm/kg for drug products intended for intramuscular or subcutaneous injection (Wang, 2015, Int J Pharm, 490(1-2):308-15). The osmolality of the 8 formulations in Table 23 range from 276 to 404 mOsm/kg, all within the safe limits for intramuscular or subcutaneous injection. The osmolality of human tears is around 318 mOsm/Kg (Hill et al., 1983, Investigative Ophthalmology & Visual Science, Vol. 24:1624-1626). The ‘Compounding Guide for Ophthalmic Preparations’ reported most patients can tolerate solutions with an osmolality range of 200 to 600 mOsm/L (McElhiney, 2013, Compounding Guide for Ophthalmic Preparations, 1 ed.). The density of all 8 formulations are in Table 23. Even though the density of Formulation 1 compared to Formulations 2-8 may not appear to be significantly different from each other, the density may have an impact on the calculation of sucrose preparations.

TABLE 23
SUMMARY OF THERMAL ANALYSIS AND PHYSIOCHEMICAL PARAMETERS

| Formulation | | Osmolality | Density | Tg′ | Crystallization |
|---|---|---|---|---|---|
| # | Also Known As | (mOsm/kg) | (g/mL) | (° C.) | (° C.) |
| 1 | dPBS | 276 | 1.0049 | Not Detected | −42.75 |
| 2 | Modified dPBS with 4% | 341 | 1.0188 | −44.83 | Not Detected |
| | sucrose | | | | |
| 3 | Modified dPBS with 6% | 402 | 1.0257 | −41.59 | |
| | sucrose | | | | |
| 4 | ½ and ½ with 4% sucrose | 328 | 1.0181 | −44.84 | |
| 5 | ½ and ½ with 6% sucrose | 404 | 1.0264 | −42.15 | |
| 6 | ½ and ½ with 6% sucrose | 364 | 1.0255 | −40.76 | |
| | and 80 mM NaCl | | | | |
| 7 | ¼ and ¼ with 4% sucrose | 328 | 1.0182 | −45.03 | |
| 8 | 5 mM Tris with 4% sucrose | 327 | 1.018 | −45.25 | |
| | and 100 mM NaCl | | | | |

5.10.8 Conclusions

These results of this study demonstrate that dPBS formulation undergone three pH units shift in frozen solution. ‘Modified dPBS formulation with 4 to 6% sucrose can attenuate pH shift to only one pH unit by inhibiting salt crystallization and maintenance of an amorphous viscous state by the cryoprotective sucrose excipient, as demonstrated by the thermal analysis with DSC. This suggest that ‘Modified dPBS formulation with sucrose’ is superior to dPBS formulation in maintain solution pH in frozen formulation. TBS-based formulation demonstrated great pH stability upon frozen and might worth further exploration for use as alternative formulation buffer for AAV frozen formulation. The osmolality of all 8 formulations are within acceptable range for ophthalmic and/or parenteral use.

### 5.11 Example 11: Aggregation of AAV is a Formulation Challenge

AAV particle aggregation has been described, with a solution ionic strength of at least 200 mM reported to be required to prevent this aggregation. U.S. Pat. No. 9,051,542. However, higher ionic strength is discouraged for prevention of crystallization (e.g., Bhatangar et al., 2007, Blood, 110(9):3233-44).

A minimum ionic strength is required to prevent aggregation or self-association of AAV particles. (FIG. 46). It was found that the minimum ionic strength required to prevent particle aggregation or self-association is AAV serotype dependent. AAV8 aggregation could be prevented at ionic strengths lower than 200 mM (FIG. 47), and lower ionic strength is required for AAV9 compared to AAV8 (FIG. 48). The ability to formulate with less salt is advantageous for frozen and dried formulations. However, the serotype specific differences in particle aggregation indicate that different formulations may be needed for different serotypes, at least with respect to level of ionic strength.

### 5.12 Example 12: Comparison of Formulation a and Formulation C In-Vitro Potency

This example shows the comparison of Formulation A and Formulation C in long term stability.

The study has been carried out by the same or similar procedures shown in Section 4.5.3 and/or other relevant sections provided herein. Formulation C is a variant of the ‘modified dPBS with sucrose’ with 60 mM NaCl and 6% sucrose. Formulation C includes 0.2 mg/mL potassium chloride, 0.2 mg/mL potassium phosphate monobasic, 3.50 mg/mL sodium chloride, 1.15 mg/mL sodium phosphate dibasic anyhydrous, 60.0 mg/mL (6% w/v) sucrose, 0.001% (0.01 mg/mL) poloxamer 188, pH 7.4.

As shown in FIG. 49, Formulation C was stable for 2 years at −20° C. The reference formulation A (dPBS) is shown for −20° C. storage as a comparator and was not stable at −20° C. Formulations B and C may have comparable and superior long-term stability at −20° C.

### 5.13 Example 13: Therapeutic Use of Construct II

This examples shows the therapeutic use of Construct II.

5.13.1 Nonclinical Studies

A tabular summary of the pharmacology and toxicology studies supporting the therapeutic use of Construct II in Formulation A is provided in Table 24.

TABLE 24
NONCLINICAL STUDIES.
Table 22: Nonclinical Studies.

| | | | | | | Gender and | |
|---|---|---|---|---|---|---|---|
| | | Route of | Doses | | | No. per | |
| Type of Study | Species | Administration | (GC/eye) | Duration | | group | Noteworthy findings |
| In vitro, binding | NA | In vitro | NA | NA | | NA | The transgene product (anti-VEGF Fab) was produced in mammalian cells |
| affinity | | | | | | | (Expi293), isolated and the binding affinity determined. The equilibrium |
| | | | | | | | binding affinity constant for binding of the Construct II transgene product to |
| | | | | | | | recombinant human VEGF is 333 pM. |
| In vivo, | Transgenic | Subretinal | 3 × 10⁶ to | 7 | days | (n = 10-17/ | Aim of this study was to determine the in vivo efficacy of a single dose of |
| pharmacology | Rho/VEGF | | 1 × | | | group | Construct II in juvenile transgenic Rho/VEGF mice, a model of human |
| | mice | | 10¹⁰ | | | | VEGF overexpression. The total area of retinal neovascularization was |
| | | | | | | | significantly reduced in Construct II-treated eyes. A dose of 1 × 10⁷ GC |
| | | | | | | | Construct II/eye was determined to be the MED. |
| In vivo, | Transgenic | Subretinal | 1 × 10⁸ | 14 | days | (n = 10/group) | Aim of this study was to determine the MED in a murine model of |
| pharmacology | Tet/opsin/ | | to 1 × | | | | neovascularization. Dose-dependent reduction in the incidence and degree of |
| | VEGF mice | | 10¹⁰ | | | | retinal detachment. A dose of 3 × 10⁸ GC Construct II/eye was determined to |
| | | | | | | | be the MED in this model of very high VEGF expression. Anti-VEGF Fab |
| | | | | | | | expression was detected in all eyes injected at ≥3 × 10⁹ GC/eye, and in some |
| | | | | | | | eyes in groups injected at lower doses. These results correlate anti-VEGF |
| | | | | | | | FAb expression with the prevention of retinal detachment in Tet/opsin/VEGF |
| | | | | | | | mice. |
| In vivo, | Cynomolgus | Subretinal | 1.00 × | 7 | Days | 2M/2F | The aim of this study was to assess the transgene product expression and |
| pharmacokinetics | monkey | | 10¹² | | | | biodistribution of Construct II. In addition, limited endpoints for toxicity |
| | | | | | | | were included in this study. Transgene product was expressed in all Construct |
| | | | | | | | II injected eyes, but not detected in blood. No adverse findings related to |
| | | | | | | | Construct II or the Construct II transgene product were observed. Assessment |
| | | | | | | | of biodistribution was not possible due to short duration of study. |
| In vivo, | Cynomolgus | Subretinal | 1.00 × | 10 | months | 2M/2F | The aim of this study was to select the AAV8 vector for development and |
| pharmacokinetics | monkey | | 10¹¹ | (study | | | included a limited number of endpoints to assess transgene product and |
| | | | | ongoing) | | | toxicity. Construct II-treated animals rapidly expressed transgene product in |
| | | | | | | | anterior chamber fluid. Onset of expression of the transgene product was |
| | | | | | | | rapid being first detected at day 15 in all eyes. |
| | | | | | | | 10 months: ONL thinning observed in 1/4 animals with no microscopic |
| | | | | | | | correlate. In 2 animals euthanized after 10 months, mild retinal atrophy in |
| | | | | | | | one animal (1/4 eyes) that was regional and involved the outer nuclear and |
| | | | | | | | rods/cones layers was observed. |
| In vivo, | Cynomolgus | Subretinal | 1.00 × | 7 | days | 1M/2F | Aim of study was to evaluate retinal distribution of AAV8 vector mRNA and |
| pharmacokinetics | monkey | | 10¹² | 10 | months | | distribution of anti-VEGF Fab throughout the eye. Levels of mRNA in |
| | | | | | | | different parts of retina were assessed by RT-qPCR and by ISH. |
| | | | | | | | Concentrations of anti-VEGF Fab were determined in retinal section, anterior |
| | | | | | | | chamber fluid, and vitreous humor by enzyme-linked immunosorbent assay. |
| | | | | | | | Anti-VEGF Fab transgene mRNA was expressed throughout the retina, at |
| | | | | | | | highest levels near injection site. Anti-VEGF Fab was distributed throughout |
| | | | | | | | all regions of the retina, along with vitreous and anterior chamber. |
| In vivo, | Cynomolgus | Subretinal | 1.00 × | 14 | days | 1M/2F (14 | Aim of this study was to evaluate the biodistribution of Construct II. The |
| biodistribution | monkey | | 10¹¹ | 3 | months | days) | majority of Construct II DNA was principally confined within retinal sections |
| | | | | | | 3 M (90 | in and adjacent to the injection site. Lower amounts were detected in the |
| | | | | | | days) | retinal sections distal to the injection site, in the lens and cornea, and in one |
| | | | | | | | animal in the right optic nerve. Construct II vector DNA was detected |
| | | | | | | | transiently at Study day 14 in the anterior and vitreal chamber fluids of |
| | | | | | | | Construct II treated right eyes. Construct II DNA was not detectable above |
| | | | | | | | background. In non-ocular tissues, Construct II DNA was detected slightly |
| | | | | | | | above background in one Study day 90 animal in one lacrimal gland and one |
| | | | | | | | submandibular lymph node sample. |
| In vitro, Tissue | Human | In vitro | NA | NA | | NA | No specific positive staining was observed with Construct II transgene |
| cross-reactivity | tissue | | | | | | product-Biotin or Ranibizumab-Biotin in any of the tissues examined. All |
| | | | | | | | other observed staining was variable and considered to be non-specific. |
| | | | | | | | Under the conditions of this study, antigen-specific binding of Construct II |
| | | | | | | | transgene product-Biotin and Ranibizumab-Biotin was demonstrated in the |
| | | | | | | | positive control materials (human glioblastoma and VEGF protein spots). |
| In vivo, | Cynomolgus | subretinal | 0, 1.00 × | 7 | Days, | 1M/2F or | Construct II transgene product was detected in anterior chamber fluid in all |
| Toxicology | monkeys | | 10¹⁰ or | 3 | Months | 2F/1M/group/ | treated animals. Construct II DNA was readily detectable in most samples |
| | | | 1.00 × | 12 | Months | timepoint | collected from animals administered CONSTRUCT II. The presence of |
| | | | 10¹² | | | | Construct II DNA was dose-dependent, transient, and decreased over time. |
| | | | (right | | | | Key findings: |
| | | | eye) | | | | 1.00 × 10¹² GC/eye: Time-dependent decrease in expressed protein levels. |
| | | | | | | | Sustained IgG responses to the Construct II transgene product in serum (1 of |
| | | | | | | | 6 animals) and anterior chamber fluid (3 of 6 animals). Higher NAb response |
| | | | | | | | to AAV8 capsid. Sustained T-lymphocyte response to Construct II AAV8 |
| | | | | | | | capsid in 3 of 6 animals. Retinal effects described as ONL and total retina |
| | | | | | | | thinning (SD-OCT) from 3 months onwards, decreases in photoreceptor |
| | | | | | | | function and post-receptor function after 3, 6, 9 and 12 months (ERG) and |
| | | | | | | | microscopic changes described as inflammation, atrophy (retinal and optic |
| | | | | | | | nerve) and, retinal detachment from 3 months onwards. |
| | | | | | | | 1.00 × 10¹⁰ GC/eye: NOAEL |

NA: not applicable;
GLP: Good Laboratory Practice;
GC: genome copy;
VEGF: Vascular Endothelial Growth Factor;
Fab: antigen-binding fragment;
MED: Minimal Effective Dose;
M: Male;
F: Female;
GLP: Good Laboratory Practice;
GC: genome copy;
VEGF: Vascular Endothelial Growth Factor;
Fab: antigen-binding fragment;
ONL: outer nuclear layer;
INL: inner nuclear layer;
RT-qPCR: quantitative reverse transcription-polymerase chain reaction;
ISH: in situ hybridization

The pharmacodynamics (anti-VEGF Fab), immunogenicity, biodistribution and toxicity of Construct II prepared using the new manufacturing process can be evaluated in a 3 month toxicity study in cynomolgus monkeys. This GLP study was initiated to support a potential alternative route of administration (suprachoroidal) and includes a group administered Construct II by subretinal injection. The study can include 4 groups of animals given test article manufactured with the modified BDS manufacturing process in each eye and one cohort of animals given vehicle control in each eye (total of 9 males and 7 females). The study can evaluate 3 doses of test article administered via two 50 μL suprachoroidal injections in each eye [3×10¹⁰ GC/eye (3×10¹¹ GC/mL); 3×10¹¹ GC/eye (3×10¹² GC/mL); 3×10² GC/eye (3×10¹³ GC/mL)] and 1 dose of test article administered via a single 100 μL subretinal delivery in each eye [3×10¹¹ GC/eye (3×10¹² GC/mL)]. At 3 months post dose, animals can be euthanized for full terminal evaluation

This study can assess for ocular toxicities with ophthalmic exams, intraocular pressure measurements, optical coherence tomography, fundus ocular photography and full-field electroretinography. The study can also evaluate transgene product concentration in aqueous humor and serum, biodistribution, immunogenicity, clinical pathology, organ weights and histopathology. The results may support the clinical use of FDP manufactured with the modified BDS manufacturing process via evaluation of safety and transgene product expression.

5.13.2 Clinical Studies

Construct II in Formulation A is currently being evaluated in a phase ½a, first-in-human, open-label, single ascending dose study with five dose cohorts in adult subjects with nAMD who are assessed over 2 years. The primary objective is to evaluate the safety and tolerability of Construct II in treated subjects through 24 weeks after single dose administration.

Subjects were treated across five dose cohorts, with 6 (Cohorts 1-3) or 12 (Cohorts 4 and 5) subjects per cohort: 3×10⁹ GC/eye (Cohort 1), 1×10¹⁰ GC/eye (Cohort 2), 6×10¹⁰ GC/eye (Cohort 3), 1.6×10¹¹ GC/eye (Cohort 4), and 2.5×10¹¹ GC/eye (Cohort 5).

Safety is the primary focus for the initial 24 weeks after Construct II administration (primary study period). The safety and tolerability and clinical effects of Construct II can be monitored through assessment of ocular and non-ocular AEs and serious adverse events (SAEs), clinical laboratory testing (chemistry, hematology, coagulation, urinalysis), immunogenicity, ocular examinations and imaging (BCVA, IOP, slit lamp biomicroscopy, indirect ophthalmoscopy, SD-OCT, fluorescein angiography, fundus autofluorescence, and color fundus photography), and vital signs.

Following completion of the primary study period, subjects can continue to be assessed for safety until 104 weeks following treatment with Construct II (Week 106). At the end of the study, subjects can be invited to participate in a long-term follow-up (LTFU) study for safety follow-up through five years' cumulative duration in the phase ½a study plus LTFU study post Construct II administration.

Forty-two subjects have been exposed to Construct II in the phase ½a trial in nAMD, and interim data were evaluated (through at least 4 weeks of safety follow-up post-Construct II administration from the last enrolled subject in Cohort 5). Construct II subretinal administration appears to be well tolerated at all dose levels tested, with no Construct II related AEs or SAEs reported. No thinning of the retina was observed, nor has there been any sign of ocular manifestations such as peripheral vision loss, decreased visual acuity or photopsia that were considered Construct II-related AEs.

To support the comparability of the of the FDP manufactured with the modified BDS manufacturing process, following evaluation of analytical comparability and nonclinical tolerability, the new process material (for example, Formulation B) may be introduced into an additional separate cohort in the ongoing phase ½a study. The cohort may comprise of 6 subjects given a single subretinal administration of the highest tested dose with an acceptable margin of safety as determined by a minimum exposure of 3 months (currently anticipated to be Cohort 5 (2.5×10¹¹ GC/eye; 1×10¹¹ GC/mL). Safety, tolerability and clinical effect of the new manufacturing process material can be evaluated as described for the previous phase ½a cohorts, and if the benefit:risk profile is comparable to previous clinical material, the new FDP material can be considered for use in the confirmatory phase 3 study.

### 5.14 Example 14: Refrigerated Short-Term Stability

The logistics of transportation of frozen drug product to clinical sites and temporary storage at the clinic until the patient is scheduled to receive their dose can be a challenge for local clinics (non-hospital clinics). Many clinical sites do not have a −80° C. (≤−60° C.) freezer for temporary storage of the drug product. Some clinical sites may have a −20° C. freezer and the preceding examples (e.g. Example 1, 3, and 9) show that formulation B is stable for at 18 months at −20° C. based on real-time stability data and 30 months when extrapolated. Other clinics do not have a reliable freezer but may have a 2-8° C. refrigerator. Therefore, allowing for thawing of the drug product in a refrigerator, followed by short-term (up to between 9 and 12 months) storage in the refrigerator is logistically desirable.

Refrigerated short-term development stability in modified DPBS with sucrose at 2-8° C. data show that the in vitro potency and other quality attributes are maintained for at least 9 months based on studies at 3.0×10¹³ GC/mL, 1.0×10¹² GC/mL, and 2.1×10¹¹ GC/mL. The data are shown in Table 25, Table 26, and Table 27.

There was no trend in vector genome concentration, appearance, purity by SDS-CGE, size distribution by DLS, subvisible particles by HIAC, free DNA by SYBR gold, pH or osmolality at 2-8° C. There was a decreasing trend in potency after extended time at 2-8° C. A trend analysis was performed using the simple linear regression fit in Prism 8 software (GraphPad LLC, San Diego, CA). The potency data trends are shown in FIG. 50. The solid black lines shows the regression fits to the data: 3.0×10¹³ GC/mL (circles), 1.0×10¹² GC/mL (squares), and 2.1×10¹¹ GC/mL (triangles).

The rate of potency loss at 2-8° C. was similar for the three studies, covering two orders of magnitude in concentration. The analysis indicated that the differences in the slopes was not significant and the data could be fit with a pooled slope of about −2.0% per month. The data indicates the potency will remain within an acceptable range (>75%) for between 9 months and 12 months at 2-8° C.

TABLE 25
SHORT-TERM STABILITY AT 2-8° C. FOR CONSTRUCT II FDP IN MODIFIED
DPBS WITH SUCROSE AT 3.0 × 10¹³ GC/ML (INVERTED VIALS)

| | Time point (days) | | | | |
|---|---|---|---|---|---|
| Test | 0 | 15 | 32 | 63 | 96 |
| In Vitro Potency (%) | 113 | 111 | 108 | 110 | 98 |
| Vector Genome Concentration | 3.09 × 10¹³ | 3.32 × 10¹³ | 3.10 × 10¹³ | 3.26 × 10¹³ | 3.31 × 10¹³ |
| by ddPCR (GC/mL) | | | | | |
| Appearance | Clear, | Clear, | Clear, | Clear, | Clear, |
| | Colorless, | Colorless, | Colorless, | Colorless, | Colorless, |
| | Particle Free | Particle Free | Particle Free | Particle Free | Particle Free |
| Viral Capsid Purity by SDS- | 100 | 100 | 100 | 100 | 100 |
| CGE (%) | | | | | |
| Size Distribution by DLS | Peak 1: | Peak 1: | Peak 1: | Peak 1: | Peak 1: |
| Diameter (nm) | 29.6 | 29.7 | 30.3 | 29.2 | 29.0 |
| % PD | 14.1 | 12.8 | 15.1 | 15.1 | 12.6 |
| % Mass | 100 | 100 | 100 | 100 | 100 |
| % Intensity | 100 | 100 | 100 | 100 | 100 |
| Subvisible particles | ≥2 μm = 22 | Not tested | ≥2 μm = 50 | Not tested | ≥2 μm = 128 |
| by HIAC | ≥10 μm = 2 | | ≥10 μm = 2 | | ≥10 μm = 3 |
| | ≥25 μm = 0 | | ≥25 μm = 0 | | ≥25 μm = 0 |
| | ≥50 μm = 0 | | ≥50 μm = 0 | | ≥50 μm = 0 |
| Free DNA (%) by SYBR Gold | 1.0 | 1.1 | 1.2 | 0.7 | 0.8 |
| pH | 7.5 | NT | 7.3 | 7.5 | 7.4 |
| Osmolality (mOsm/kg) | 347 | NT | 347 | 343 | 345 |

NT = Not tested

TABLE 26
SHORT-TERM STABILITY AT 2-8° C. FOR CONSTRUCT II FDP IN
MODIFIED DPBS WITH SUCROSE AT 1.0 × 10¹² GC/ML

| | Time point (months) | | | | | |
|---|---|---|---|---|---|---|
| Test | 0 | 3 | 6 | 9 | 12 | 18 |
| In Vitro Potency (%) | 82 | 87 | 84 | 86 | 70 | 67 |
| Vector Genome | 1.0 × 10¹² | 9.2 × 10¹¹ | 8.9 × 10¹¹ | 9.8 × 10¹¹ | 1.0 × 10¹² | 1.1 × 10¹² |
| Concentration by ddPCR | | | | | | |
| (GC/mL) | | | | | | |
| Free DNA (%) by SYBR | NT | Qualitative | Qualitative | NT | 1.7 | 2.0 |
| Gold^{a} | | | | | | |
| Size Distribution | 26.1 | 28.5 | 28.2 | 27.9 | 28.5 | 28.3 |
| (DLS Cumulant | | | | | | |
| Diameter in nm) | | | | | | |
| pH | 7.3 | NT | NT | NT | 7.3 | NT |
| Appearance | Clear, | Clear, | Clear, | Clear, | Clear, | Clear, |
| | Colorless, | Colorless, | Colorless, | Colorless, | Colorless, | Colorless, |
| | Particle | Particle | Particle | Particle | Particle | Particle |
| | Free | Free | Free | Free | Free | Free |

NT = Not tested
^{a}The free DNA method was initially established as a semiquantitative relative method and results were reported relative to the ≤−60° C. stability sample for time points up to 9 months. The method was updated to provide absolute % free DNA at time points after 9 months.

TABLE 27
SHORT-TERM STABILITY AT 2-8° C. FOR CONSTRUCT II FDP IN
MODIFIED DPBS WITH SUCROSE AT 2.1 × 10¹¹ GC/ML

| | Time point (months) | | | | | | | |
|---|---|---|---|---|---|---|---|---|
| Test | 0 | 1 | 2 | 3 | 6 | 9 | 12 | 18 |
| In Vitro | 113 | 115 | 100 | 100 | 77 | 90 | 64 | 68 |
| Potency (%) | | | | | | | | |
| Vector | 2.1 × 10¹¹ | 2.2 × 10¹¹ | 2.2 × 10¹¹ | 2.1 × 10¹¹ | 2.0 × 10¹¹ | 2.1 × 10¹¹ | 2.2 × 10¹¹ | 2.4 × 10¹¹ |
| Genome | | | | | | | | |
| Concentration | | | | | | | | |
| by ddPCR | | | | | | | | |
| (GC/mL) | | | | | | | | |
| Free DNA | NT | Qualitative | Qualitative | Qualitative | Qualitative | NT | 1.6 | 2.3 |
| (%) by | | | | | | | | |
| SYBR Gold | | | | | | | | |
| ^{a} | | | | | | | | |
| Size | 21.62 ^{b} | 26.9 | 27.5 | 27.0 | 26.6 | 28.8 | 28.7 | 28.6 |
| Distribution | | | | | | | | |
| (DLS | | | | | | | | |
| Cumulant | | | | | | | | |
| Diameter in | | | | | | | | |
| nm) | | | | | | | | |
| pH | 7.3 | NT | NT | NT | 7.4 | NT | 7.2 | NT |
| Appearance | Clear, | Clear, | Clear, | Clear, | Clear, | Clear, | Clear, | Clear, |
| | Colorless, | Colorless, | Colorless, | Colorless, | Colorless, | Colorless, | Colorless, | Colorless, |
| | Particle | Particle | Particle | Particle | Particle | Particle | Particle | Particle |
| | Free | Free | Free | Free | Free | Free | Free | Free |

NT = Not tested
^{a} The free DNA method was initially established as a semiquantitative relative method and results were reported relative to the ≤−60° C. stability sample for time points up to 9 months. The method was updated to provide absolute % free DNA at time points after 9 months.
^{b} At initial time point, DLS raw data collection at lower concentration shows matrix interference contribution from sucrose excipient skewing apparent size to lower value.

### 5.15 Example 15: Room Temperature Short-Term Stability

There are short periods where the drug product may be exposed to room temperature during manufacturing, labelling, dose preparation and delivery.

Controlled room temperature short-term development stability data of Construct II at 3.0×10¹³ GC/mL in modified dPBS with sucrose at about 22° C. (20.7-23.7° C.) is shown in Table 28. There was no trend in vector genome concentration, appearance, purity by SDS-CGE, size distribution by DLS, subvisible particles by HIAC, free DNA by SYBR gold, pH or osmolality. There was a decreasing trend in potency at controlled room temperature. A statistical analysis was performed using the nonlinear regression function in Prism 8 software (GraphPad LLC, San Diego, CA). The potency data was best fit to a linear regression model. The best-fit slope was −0.8958% per day at controlled room temperature. The potency trend at room temperature is shown in FIG. 51. The solid black line shows the regression fit to the data and the dotted lines show the 95% CI. Typically, up to 3 days total cumulative exposure is required for manufacturing and delivery of the drug product. This exposure corresponds to an expected average 2.7% decrease in potency, which is acceptable from a product stability perspective.

TABLE 28
SHORT-TERM STABILITY AT CONTROLLED ROOM TEMPERATURE FOR CONSTRUCT II FDP
LOT 200320-314-DL7 IN MODIFIED DPBS WITH SUCROSE AT 3.0 × 10¹³ GC/ML

| | Time point (days) | | | | | | | |
|---|---|---|---|---|---|---|---|---|
| Test | 0 | 1 | 3 | 7 | 10 | 15 | 24 | 32 |
| In Vitro | 113 | 107 | 103 | 102 | 92 | 86 | 90 | 80 |
| Potency (%) | | | | | | | | |
| Vector | 3.09 × 10¹³ | 3.18 × 10¹³ | 3.70 × 10¹³ | 3.26 × 10¹³ | 3.20 × 10¹³ | 3.25 × 10¹³ | 3.49 × 10¹³ | 2.85 × 10¹³ |
| Genome | | | | | | | | |
| Concentration | | | | | | | | |
| by ddPCR | | | | | | | | |
| (GC/mL) | | | | | | | | |
| Appearance | Clear, | Clear, | Clear, | Clear, | Clear, | Clear, | Clear, | Clear, |
| | Colorless, | Colorless, | Colorless, | Colorless, | Colorless, | Colorless, | Colorless, | Colorless, |
| | Particle | Particle | Particle | Particle | Particle | Particle | Particle | Particle |
| | Free | Free | Free | Free | Free | Free | Free | Free |
| Viral Capsid | 100 | NT | NT | NT | 100 | 100 | NT | 100 |
| Purity by | | | | | | | | |
| SDS-CGE | | | | | | | | |
| (%) | | | | | | | | |
| Size | Peak 1: | Peak 1: | Peak 1: | Peak 1: | Peak 1: | Peak 1: | Peak 1: | Peak 1: |
| Distribution | 29.6 | 29.8 | 30.5 | 29.6 | 30.0 | 29.3 | 30.0 | 29.6 |
| by DLS | 14.1 | 15.3 | 18.8 | 15.4 | 16.4 | 10.8 | 14.0 | 12.0 |
| Diameter | 100 | 100 | 100 | 100 | 100 | 100 | 100 | 100 |
| (nm) | 100 | 100 | 100 | 100 | 100 | 100 | 100 | 100 |
| % PD | | | | | | | | |
| % Mass | | | | | | | | |
| % Intensity | | | | | | | | |
| Subvisible | ≥2 μm = 22 | NT | NT | ≥2 μm = 22 | NT | ≥2 μm = 17 | NT | NT |
| particles by | ≥10 μm = 2 | | | ≥10 μm = 3 | | ≥10 μm = 0 | | |
| HIAC | ≥25 μm = 0 | | | ≥25 μm = 0 | | ≥25 μm = 0 | | |
| | ≥50 μm = 0 | | | ≥50 μm = 0 | | ≥50 μm = 0 | | |
| Free DNA | 1.0 | 1.2 | 1.3 | 1.4 | 1.5 | 1.6 | 1.0 | 0.7 |
| (%) by | | | | | | | | |
| SYBR Gold | | | | | | | | |
| pH | 7.5 | NT | NT | NT | NT | 7.5 | NT | 7.5 |
| Osmolality | 347 | NT | NT | NT | NT | 347 | NT | 346 |
| (mOsm/kg) | | | | | | | | |

NT = Not tested

### 5.16 Example 16: Formulation Composition Robustness Evaluated at Accelerated High Frozen Temperature Excursion Conditions (−15° C., −7° C., and −20° C. ‘Auto-Defrost’ Freezer)

The potency of several formulation variations were studied to determine their relative stability to formulation B when held at temperatures higher than the intended storage temperature excursion range of ≤−20° C.

The first row in Table 29 shows results for formulation B as a control. The results show excursions of 6 months at −15° C., −7° C. and a −20° C. ‘auto-defrost’ freezer (varying every 4 hours from −20° C. up to about −6° C.). The potency was maintained at acceptable levels for all conditions after 6 months demonstrating that formulation B has robust stability.

Formulation D is a variation of formulation B with significantly higher sucrose and lower salt. Formulation E is a variation of formulation B with moderately higher sucrose and lower salt. Both these formulations had similar potency to formulation B after 6 months at −15° C., demonstrating that formulation B has a robust stability design-space. Higher sucrose and lower salt than already in formulation B did not improve stability of formulation B indicating that the composition within this wide range is acceptable for stability.

Formulation F is formulation B modified with 5 mM of added TRIS buffer in an attempt to minimize or cancel out pH fluctuations. This formulation was studied based on the hypothesis that since the pH of phosphate decreases when frozen and the pH of TRIS increases a combination of both may be more stable. This formulation also had had similar potency to formulation B after 6 months at −15° C.

Formulation G is a new formulation with TRIS buffer instead of phosphate buffer, sodium sulfate substituted for NaCl at lower levels, higher levels of sucrose, and higher poloxamer 188. This formulation has similar stability to formulation B after 6 months at −7° C.

These data demonstrate that the level of sucrose added in formulation B (≥4%) is the key cryoprotective excipient. This level of sucrose is sufficient to mitigate pH shifts and salt crystallization to minimize potency loss. The sucrose is cryoprotective with either sodium chloride or with sodium sulfate-based salt formulations and either salt is suitable for the formulation. The formulation with lower salt by substituting sodium sulfate for sodium chloride did not have improved stability. Further stability improvement with lower salt or with higher sucrose is not observed demonstrating that 4% is a robust level of sucrose to provide cryoprotective properties with 100 mM sodium chloride. The substitution of phosphate with TRIS buffer did not improve stability and either buffer could be used in a stable sucrose and salt-based formulation. Higher levels of poloxamer 188 also did not improve stability.

TABLE 29
FORMULATION COMPOSITION ROBUSTNESS EVALUATED AT ACCELERATED
HIGH FROZEN TEMPERATURE EXCURSION CONDITIONS (−15°
C., −7° C., AND −20° C. ‘AUTO-DEFROST’ FREEZER)

| | In Vitro Potency (%) | | | |
|---|---|---|---|---|
| | | | | 6 months in |
| | Initial | 6 months | 6 months | −20° C. ‘auto- |
| Formulation | Time 0 | at -15° C. | at -7° C. | defrost’ |
| Formulation B - control for comparison | 136 | 93 | 93 | 86 |
| Modified dPBS with 4% sucrose, 0.001% | | | | |
| P188, pH 7.4 | | | | |
| Formulation D | 163 | 95 | Not | Not |
| (Modified dPBS, 14% Sucrose, 60 mM | | | tested | tested |
| NaCl, 0.001% P188, pH 7.4) | | | | |
| Formulation E | 138 | 92 | Not | Not |
| Modified dPBS, 6% Sucrose, 80 mM | | | tested | tested |
| NaCl, 0.001% P188, pH 7.4 | | | | |
| Formulation F | 143 | 84 | Not | Not |
| Modified dPBS, 5 mM Tris, 6% Sucrose, | | | tested | tested |
| 80 mM NaCl, 0.001% P188, pH 7.4 | | | | |
| Formulation G | 143 | Not | 93 | Not |
| 5 mM TRIS, 30 mM Na₂SO4, 9% | | tested | | tested |
| sucrose, 0.005% P188, pH 7.0, | | | | |

### 5.17 Example 17: Comparison of Formulation a and Formulation B in Release Characterization

This example is an updated version of Example 2 above.

This example shows the comparison of Formulation A and Formulation B in release characterization.

5.17.1 Release and Characterization Analytical Methods and Specification

This section shows comparison of lot release results from the certificate of analysis for one lot of final drug product (FDP) in the Formulation A and one lot of FDP in the new ‘modified DPBS with 4% Sucrose and 0.001% Poloxamer 188, pH 7.4’ (formulation B). In addition to the release tests the routine characterization test result for dynamic light scattering (DLS) may be compared. The panel of proposed tests to and the acceptance criteria to support comparability are shown in Table 30.

TABLE 30
ACCEPTANCE CRITERIA FOR RELEASE AND CHARACTERIZATION METHODS
MAY BE USED TO PERFORM ANALYTICAL COMPARABILITY OF THE FDP

| | Analytical | |
|---|---|---|
| Test | Method | Acceptance Criteria |
| Appearance | Visual Inspection | Colorless, clear to opalescent solution, free of |
| | | foreign particulates |
| pH | USP<791> | 6.9-7.9 |
| Osmolality | USP<785> | Meets lot release specification.¹ |
| Vector Genome | Transgene ddPCR | Meets lot release specification.² |
| Concentration (Content) | | |
| Vector Genome | Transgene ddPCR | Confirmed as Construct II Genome |
| Concentration (Identity) | | |
| Capsid Identity | ELISA | Confirmed as AAV8 Serotype |
| (Identity) | | |
| In Vitro Potency | HEK293 | 50-200% Relative Potency |
| (Potency) | Transduction/Anti- | |
| | VEGF ELISA | |
| Viral Capsid Purity | SDS-CGE | ≥90% Purity (% Purity: Sum of VP1, |
| (Purity) | | VP2, and VP3) |
| Particulate Matter | USP<787> HIAC | ≤50 particles/mL |
| (Impurity) | | (Size ≥10 μm) |
| | | ≤ 5 particles/mL |
| | | (Size ≥25 μm) |
| | | ≤ 2 particles/mL |
| | | (Size ≥50 μm) |
| Endotoxin (Safety) | USP<85> | ≤0.5 EU/mL |
| Sterility (Safety) | USP<71> | No Growth |
| Size Distribution | Dynamic Light | Main Peak diameter consistent |
| (Purity) | Scattering | with intact AAV |

5.17.2 Background on FDP Formulation Change

The ‘modified DPBS with 4% Sucrose and 0.00100 Poloxamer 188, pH 7.4’ (formulation B) was developed to improve the long-term frozen storage stability and robustness of the FDP stability to freeze/thaw cycles. The formulation change involved addition of 40% w/v of the cryoprotective excipient sucrose and a reduction in the sodium chloride level from 137 mM to 100 mM to maintain appropriate tonicity. The other formulation excipients and levels were identical. The generation of FDP in the ‘modified DPBS with 4% Sucrose and 0.00100 Poloxamer 188, pH 7.4’ FDP formulation was achieved by addition of a more concentrated sucrose spike solution to the BDS in DPBS (formulation A) to adjust the composition to the final composition. The composition may also be achieved using tangential flow filtration buffer exchange.

5.17.3 Assessment of Changes to FDP Formulation Process

Spiking, mixing, and filtration steps involved similar process steps, handling, and contact surfaces for both processes. The spiking process resulted in a dilution of 1.37-fold. Then there was a subsequent dilution to target concentration.

5.17.4 Assessment of Impacts of FDP Formulation Change to Clinical Safety and Efficacy

FDP formulation B might have no difference in safety or efficacy when compared to the DPBS formulation A. The product quality profile and release specifications were the same, except for the general attribute, osmolality.

The osmolality for the DPBS formulation A was 240-340 mOsm/kg and for the formulation B the osmolality was 295-395 mOsm/kg. These were due to the adjustment of the level of sucrose and sodium chloride in the formulation B. There might be no impact of on the resorption time for blebs with these slightly higher osmolality values based on the literature (see, e.g., Negi and Marmour, 1984 Invest Ophthalmol Vis Sci. 25(5):616-20).

5.17.5 Assessment of the Impact of Changing from Formulation a to Formulation B to Analytical Release Methods

Assessment of analytical methods indicated they are fit-for-purpose. Minor modifications to their procedures can accommodate the new FDP (Table 31).

TABLE 31
ASSESSMENT OF NEW FDP COMPOSITION IMPACT
TO ANALYTICAL RELEASE METHODS

| | Analytical | |
|---|---|---|
| Test | Method | Assessment of Impact of FDP Change |
| Appearance | Visual Inspection | No impact. New FDP formulation (formulation B) is |
| | | expected to be a colorless, clear to opalescent |
| | | solution, free of foreign particulates. |
| pH | USP<791> | No impact. New FDP formulation has same buffering |
| | | species and levels. |
| Osmolality | USP<785> | No impact. Freezing-point depression method gave |
| | | expected result for new FDP. |
| Vector Genome | Transgene ddPCR | No Impact. Development samples were on-target and |
| Concentration | | met assay suitability criteria for new FDP. |
| (Content) | | |
| Vector Genome | Transgene ddPCR | No Impact. |
| Concentration | | |
| (Identity) | | |
| Capsid Identity | ELISA | No impact. |
| (Identity) | | |
| In Vitro Potency | HEK293 | No Impact. Development samples were on-target and |
| (Potency) | Transduction/Anti- | met assay suitability criteria for new FDP. |
| | VEGF ELISA | |
| Viral Capsid | SDS-CGE | No impact. |
| Purity (Purity) | | |
| Particulate | USP<787> HIAC | No impact. Light obscuration depends on clarity of |
| Matter | | solution which is similar for new FDP. |
| (Impurity) | | |
| Endotoxin | USP<85> | No impact. Samples tested met assay suitability |
| (Safety) | | criteria for new FDP. |
| Sterility (Safety) | USP<71> | No impact. New FDP buffer was shown not to inhibit |
| | | growth. |
| Size | Dynamic Light | Change in solution viscosity can be adjusted in |
| Distribution | Scattering | method settings. Development data shows a slight |
| (Purity) | (data from routine | increase (1 to 3 nm) in apparent size related to the |
| | characterization | impact of sucrose on the hydration and therefore |
| | testing performed | hydrodynamic behavior of the AAV capsids. |
| | either at release or | |
| | T0 stability) | |

### 5.18 Example 18: Comparison of Formulation a and Formulation B in Stability

This example shows the comparison of Formulation A and Formulation B in their stability. This example is an updated version of Example 3 above.

The new Formulation B protects against disruption of capsids and release of small amounts of free DNA upon freeze/thaw cycles and temperature stress. Long-term stability studies currently 24 months demonstrated that the in-vitro relative potency and other quality attributes are maintained at −80° C. (≤−60° C.) and ≤−20° C. in the FDP formulation B.

The available freeze/thaw data, temperature stress data, and long-term stability data indicated similar or improved stability in the new formulation.

FDP lots in the new FDP formulation B can be set down on long-term stability at −80° C. (≤−60° C.) and ≤−20° C. and the stability trends data can be monitored as part of the stability program to ensure that the expiration date for the new FDP is compliant with regulations.

5.18.1 Freeze/Thaw Study for Construct II in DPBS and in the New FDP Formulation B

A study was conducted to assess the impact of freeze/thaw cycles on Construct II in DPBS and in the new FDP formulation B. Freeze/thaw rates were selected to bracket the expected rates that could occur during manufacturing for bottles of BDS or in the supply chain or clinic for vials of DP. Multiple cycles were applied to stress the samples beyond what might occur in the clinic.

The results of the study demonstrated that Formulation B was more robust than Formulation A when exposed to up to five freeze/thaw cycles from ≤−60° C. to 25° C. with either slow (0.12° C./min or over about 11 hours) and/or fast (1° C./min or over about 1 hour). All permutations of slow and fast rates were assessed for freezing and thawing respectively (i.e. FF/FT=fast freeze/fast thaw; FF/ST=fast freeze/slow thaw; SF/FT=slow freeze/fast thaw; SF/ST=slow freeze/slow thaw).

Freezing and thawing rates can impact the stability of biologics (Cao et al., 2003, Biotechnol. Bioeng. 82(6):684-90)). Crystallization of water during freezing can result in concentration of excipients which can impact the stability of biologics. Phase separation or pH shifts may also occur with an impact the stability of biologics. Fast freezing can lead to smaller ice crystals and a larger ice-water interface area which could impart interfacial stresses. Fast freezing could also entrap air bubbles in the ice leading to air-water interfacial stress during thawing. Slow thawing can result in re-crystallization of ice which can impact the stability of biologics in solution due to interfacial stress.

In this study, samples were analyzed by in vitro relative potency, size-exclusion chromatography purity (SEC), free DNA levels by fluorescent dye, and size distribution by dynamic light scattering. Phase changes of the formulations upon freezing and thawing were assessed by calorimetry.

There was little differentiation in results for different rates of fast and slow rates of freezing and thawing in this study. An overall summary of the freeze-thaw studies results is provided in Table 32. A representative example of a temperature profile (Fast Freeze/Slow Thaw) applied in this study is shown in FIG. 9 (other permutations of profiles not shown). Potency results were similar to the control for all permutations of fast and slow rates of freezing and thawing. There was no change in the size distribution within method variability upon freeze/thaw cycling of either Formulation A or Formulation B (see FIG. 12). The cumulants DLS diameter in formulation B of about 28 nm was slightly higher than in DPBS formulation A at 27 nm. This very slight apparent size increase is related to the impact of sucrose on the hydration and therefore hydrodynamic behavior of the AAV capsids. For Construct II in the DPBS formulation A, there was an increase in free DNA by dye fluorescence for all the freeze-thaw stress conditions (from 1.7% up to 6.9%). The free DNA quantitation by SEC was generally consistent with the dye fluorescence. By SEC the first pre-peak contains free DNA, the second small pre-peak contains aggregates, and the post-peak contains buffer species (see FIG. 10 and FIG. 11). The slightly lower free DNA by SEC might be due to very small or very large DNA fragments either co-eluting with the main peak or with the buffer, or not entering the column. The increases were of similar magnitude and not clearly differentiated for the different permutations of fast or very slow freeze-thaw rates. No increase in free DNA or aggregates were observed for Formulation B.

A small exotherm was observed in Formulation A at about −41° C. due to crystallization of amorphous sodium chloride that had not crystallized fully during cooling (FIG. 13). A eutectic melt with an unresolved low temperature shoulder was also observed with a peak at −22.1° C. for Formulation A. The same eutectic and re-crystallization events have been reported in the literature for sodium chloride (Milton et al., 2007, Journal of Pharmaceutical Sciences, 96(7)). The lack of a eutectic melt for formulation B (FIG. 14) was consistent with the inhibition of crystallization. Maintenance of an amorphous viscous state by the cryoprotective sucrose excipient in formulation B might provide protection against capsid disruption and release of free DNA upon freeze/thaw cycles. Therefore, the change to formulation B resulted in similar or improved stability compared to formulation A.

TABLE 32
IMPACT OF FREEZE-THAW RATES ON CONSTRUCT II
IN DPBS AND IN THE NEW FDP FORMULATION B

| | Construct II in DPBS with 0.001% | | | | Construct II in ‘modified DPBS with 4% | | | |
|---|---|---|---|---|---|---|---|---|
| | poloxamer 188, pH 7.4 | | | | Sucrose and 0.001% Poloxamer 188, pH 7.4’ | | | |
| | (Formulation A) | | | | (Formulation B) | | | |
| | | SE- | DLS | In vitro | | SE- | DLS | In vitro |
| Freeze | Free | HPLC^{b} | Cumulants | Relative | Free | HPLC^{b} | Cumulants | Relative |
| and Thaw | DNA | Pre-peak | Diameter | Potency | DNA | Pre-peak | Diameter | Potency |
| Rates^{c} | (%)^{a} | (%) | (nm) | (%) | (%)¹ | (%) | (nm) | (%) |
| Control | 1.7 | 2.1 | 26.8 | 88 | 2.5 | 2.3 | 28.0 | 87 |
| 5 × FF/FT | 6.9 | 4.5 | 28.4 | 88 | 2.7 | 1.6 | 28.5 | 89 |
| 5 × FF/ST | 5.9 | 4.5 | 26.7 | 87 | 2.5 | 1.2 | 27.9 | 88 |
| 5 × SF/FT | 6.6 | 3.6 | 26.7 | 88 | 2.6 | 1.1 | 27.7 | 92 |
| 5 × SF/ST | 6.8 | 3.4 | 26.9 | 86 | 2.7 | 1.5 | 28.1 | 88 |

d. Percent free DNA is based on the measured level compared to the total calculated from GC/mL (OD 260 nm).
e. SEC results calculated based on the 260 nm wavelength channel.
f. The actual product temperature ‘fast’ rate was about an hour for freezing and 1.5 hours for thawing. The ‘slow’ rate was about 11 hours for both freezing and thawing.

5.18.2 Temperature Stress Stability of Construct II in Formulation a and the New FDP Formulation B

A temperature stress development stability study conducted at 1.0×10¹² GC/mL over 4 days at 37° C. was used to evaluate the relative stability of formulation A and formulation B. Assays used to assess stability included in vitro relative potency (IVRP), vector genome concentration (VGC by ddPCR), free DNA by dye fluorescence, dynamic light scattering, appearance, and pH. Results for formulation A are shown in Table 33 and results for formulation B are shown in Table 34. A comparable decrease in potency within the assay variability of about 14 to 16% per day was observed for both formulations (see FIG. 15). There was also a small decrease in vector genome copies that was comparable for both formulation A and formulation B. The cumulants DLS diameter in Formulation B of about 29-34 nm was slightly higher than in DPBS formulation A at 28-30 nm. This very slight (1 to 3 nm) apparent size increase was related to the impact of sucrose on the hydration and therefore hydrodynamic behavior of the AAV capsids. There was no trend in DLS diameter within method variability for either Formulation A or Formulation B during the stress study. Formulation B was slightly more stable with respect to capsid disruption and had lower levels of free DNA. Free DNA increased from about 1 to 300 for Formulation A and remained below 100 for Formulation B (see FIG. 16). Overall the temperature stress stability of Formulation A and Formulation B were similar, with Formulation B being slightly more stable with respect to capsid disruption and release of free DNA. Therefore the change to Formulation B may result in similar or improved stability compared to Formulation A.

TABLE 33
TEMPERATURE STRESS STABILITY OF CONSTRUCT II IN FORMULATION
A 1.0 × 10¹² GC/ML HELD AT 37° C.

| | Time Point | | | |
|---|---|---|---|---|
| Test | 0 days | 1 day | 2 days | 4 days |
| IVRP (%) | 128 | 111 | 96 | 75 |
| VGC (GC/mL) | 1 × 10¹² | 9.7 × 10¹¹ | 9.5 × 10¹¹ | 8.6 × 10¹¹ |
| Free DNA (%) | 1.27 | 2.13 | 2.30 | 3.12 |
| DLS Diameter (nm) | 28.3 | 28.7 | 28.4 | 30.2 |
| pH | 7.4 | 7.4 | 7.4 | 7.6 |
| Appearance | Clear, | Clear, | Clear, | Clear, |
| | colorless, | colorless, | colorless, | colorless, |
| | particle free | particle free | particle free | particle free |

TABLE 34
TEMPERATURE STRESS STABILITY OF CONSTRUCT II IN FORMULATION
B 1.0 × 10¹² GC/ML HELD AT 37° C.

| | Time Point | | | |
|---|---|---|---|---|
| Test | 0 days | 1 day | 2 days | 4 days |
| IVRP (%) | 143 | 121 | 101 | 83 |
| VGC (GC/mL) | 1 × 10¹² | 1 × 10¹² | 9.2 × 10¹¹ | 8.8 × 10¹¹ |
| Free DNA (%) | 0.59 | 0.83 | 0.84 | 0.83 |
| DLS Diameter (nm) | 28.7 | 34.2 | 32.8 | 29.5 |
| pH | 7.2 | 7.4 | 7.3 | 7.3 |
| Appearance | Clear, | Clear, | Clear, | Clear, |
| | colorless, | colorless, | colorless, | colorless, |
| | particle free | particle free | particle free | particle free |

5.18.3 Long-Term Stability of Construct II in the New FDP Formulation B

Long-term development stability studies demonstrated that the in-vitro relative potency and other quality attributes were maintained at −80° C. (≤−60° C.) for 24 months and-20° C. for 18 months in the FDP formulation B. The study at −20° C. was stopped at 18 months as this represents a substantial time duration at this temperature and is sufficient to demonstrate the robustness of the formulation B to long-term stability at this higher (warmer) frozen stability temperature. Extrapolation of the data based on principles outlined in the ICH Q1E guideline, Evaluation of Stability Data, indicates that a 12 months extrapolation is justifiable and that the formulation will be stable for at least 36 months at −80° C. (≤−60° C.) and is stable to the higher storage temperature of −20° C. for 30 months. The study was conducted at both 1.0×10¹² GC/mL and at 2.1×10¹¹ GC/mL. Assays used to assess stability included in vitro relative potency (IVRP), vector genome concentration (VGC by ddPCR), free DNA by dye fluorescence (relative to −80° C. for time points up until 9 months, and absolute percentage at 12 months), appearance, size exclusion chromatography (SEC) for 1.0×10¹² GC/mL only, dynamic light scattering (DLS), pH, and appearance.

There was no trend on stability for all results at −80° C. for 24 months and no trend at −20° C. for 18 months. All results are similar for the −80° C. and −20° C. within method variability and similar to the initial time point results. The long-term stability data for 1.0×10¹² GC/mL held at −80° C. (Table 35) and −20° C. (Table 36 and for 2.1×10¹¹ GC/mL held at-80° C. (Table 37) and −20° C. (Table 38) demonstrate that Construct II is stable in formulation B for at least 24 months based on real-time data, and expected to be stable for at least 36 months with extrapolation. IVRP potency trend graphs are shown in FIG. 52 and FIG. 53. The IVRP potency shows no consistent trend compared to method variability over the 24 months of long-term stability data.

TABLE 35
LONG-TERM STABILITY OF CONSTRUCT II IN FORMULATION B
AT 1.0 × 10¹² GC/ML AT −80° C.

| | Time Point | | | | | | |
|---|---|---|---|---|---|---|---|
| Test | 0 months | 3 months | 6 months | 9 months | 12 months | 18 months | 24 months |
| IVRP (%) | 82% | 91% | 90% | 112% | 110% | 101% | 102% |
| VGC | 1 × 10¹² | 9.4 × 10¹¹ | 9.6 × 10¹¹ | 9.8 × 10¹¹ | 9.7 × 10¹¹ | 1.2 × 10¹¹ | 9.62 × 10¹¹ |
| (GC/mL) | | | | | | | |
| Free DNA | 1.00 | 1.00 | 1.00 | NA | 1.1% | 1.4% | 1.7% |
| (relative | | | | | absolute) | | |
| to −80° C. | | | | | | | |
| control, or %) | | | | | | | |
| SEC (% | 99.1 | 98.9 | 99.1 | Not | 99.16 | 98.09 | pending |
| purity) | | | | tested | | | |
| DLS Diameter | NA | 28.4 | 28.1 | 27.9 | 28.7 | 28.2 | 27.8 |
| (nm) | | | | | | | |
| pH | 7.3 | Not | Not | Not | 7.3 | Not | Not |
| | | tested | tested | tested | | tested | tested |
| Appearance | Clear, | Clear, | Clear, | Clear, | Clear, | Clear, | Clear, |
| | colorless, | colorless, | colorless, | colorless, | colorless, | colorless, | colorless, |
| | particle | particle | particle | particle | particle | particle | particle |
| | free | free | free | free | free | free | free |

TABLE 36
LONG-TERM STABILITY OF CONSTRUCT II IN FORMULATION B
AT 1.0 × 10¹² GC/ML AT −20° C.

| | Time Point | | | | | |
|---|---|---|---|---|---|---|
| Test | 0 months | 3 months | 6 months | 9 months | 12 months | 18 months |
| IVRP (%) | 82% | 98% | 90% | 115% | 85% | 91% |
| VGC (GC/mL) | 1 × 10¹² | 9 × 10¹¹ | 9.6 × 10¹¹ | 9.9 × 10¹¹ | 9.5 × 10¹¹ | 1.1 × 10¹² |
| Free DNA (relative | 1.00 | 0.96 | 0.97 | Not tested | 1.5% | 1.5% |
| to −80° C. control, or | | | | | | |
| %) | | | | | | |
| SEC (% purity) | 99.1 | 98.8 | 99.3 | Not tested | 99.20 | 99.11 |
| DLS Diameter (nm) | NA | 29.8 | 28.2 | 28 | 29.1 | 28.5 |
| pH | 7.3 | Not tested | Not tested | Not tested | 7.3 | Not tested |
| Appearance | Clear, | Clear, | Clear, | Clear, | Clear, | Clear, |
| | colorless, | colorless, | colorless, | colorless, | colorless, | colorless, |
| | particle | particle | particle | particle | particle | particle |
| | free | free | free | free | free | free |

TABLE 37
LONG-TERM STABILITY OF CONSTRUCT II IN FORMULATION B
AT 2.1 × 10¹¹ GC/ML AT −80° C.

| | Time Point | | | | | | |
|---|---|---|---|---|---|---|---|
| Test | 0 months | 3 months | 6 months | 9 months | 12 months | 18 months | 24 months |
| IVRP (%) | 113% | 107% | 120% | 104% | 97% | 111% | 115% |
| VGC (GC/mL) | 2.1 × 10¹¹ | 2.1 × 10¹¹ | 2.2 × 10¹¹ | 2.3 × 10¹¹ | 2.3 × 10¹¹ | 2.7 × 10¹¹ | 2.14 × 10¹¹ |
| Free DNA | 1.00 | 1.00 | 1.00 | Not | 1.4% | 1.4 | 1.6 |
| (relative | | | | tested | | | |
| to −80° C. | | | | | | | |
| control, or %) | | | | | | | |
| DLS Diameter | NA | 25.3 | 26.0 | 28.9 | 28.1 | 29.1 | 28.8 |
| (nm) | | | | | | | |
| pH | 7.3 | Not | Not | Not | 7.2 | Not | Not |
| | | tested | tested | tested | | tested | tested |
| Appearance | Clear, | Clear, | Clear, | Clear, | Clear, | Clear, | Clear, |
| | colorless, | colorless, | colorless, | colorless, | colorless, | colorless, | colorless, |
| | particle | particle | particle | particle | particle | particle | particle |
| | free | free | free | free | free | free | free |

TABLE 38
LONG-TERM STABILITY OF CONSTRUCT II IN FORMULATION B
AT 2.1 × 10¹¹ GC/ML AT −20° C.

| | Time Point | | | | | |
|---|---|---|---|---|---|---|
| Test | 0 months | 3 months | 6 months | 9 months | 12 months | 18 months |
| IVRP (%) | 113% | 104% | 115% | 109% | 86% | 91% |
| VGC (GC/mL) | 2.1 × 10¹¹ | 2.1 × 10¹¹ | 2.1 × 10¹¹ | 2.2 × 10¹¹ | 2.3 × 10¹¹ | 2.4 × 10¹¹ |
| Free DNA (relative | 1.00 | 0.99 | 1.10 | Not tested | 1.8% | 1.9% |
| to −80° C. control) | | | | | | |
| DLS Diameter (nm) | NA | 26.1 | 25.8 | 28.9 | 30.3 | 29.5 |
| pH | 7.3 | Not tested | Not tested | Not tested | 7.4 | Not tested |
| Appearance | Clear, | Clear, | Clear, | Clear, | Clear, | Clear, |
| | colorless, | colorless, | colorless, | colorless, | colorless, | colorless, |
| | particle free | particle free | particle free | particle free | particle free | particle free |

### 5.19 Example 19: Non-Human Primate Study of Construct II

The purpose of this study was to evaluate the pharmacodynamics, biodistribution, immunogenicity, and toxicity of Construct 11, when administered as a single dose via suprachoroidal or subretinal injection to cynomolgus monkeys. After dosing, animals were observed postdose for at least 13 weeks (Day 92 of the dosing phase; terminal sacrifice). Male and female cynomolgus monkeys were assigned to five groups, and doses were administered as indicated in Table 39. Animals in Group 1 were administered two suprachoroidal injection into the left eye on Day 1 of the dosing phase at a volume of 50 μL/injection/left eye (total of 100 μL/left eye) and subretinal injection in the right eye once on Day 1 of the dosing phase at a volume of 100 μL/right eye (administered as a single 100 μL bleb/right eye). Animals in Groups 2, 3, and 4 were administered two suprachoroidal injections to each eye on Day 1 of the dosing phase at a volume of 50 μL/injection/eye (total of 100 μL/eye). Animals in Group 5 were dosed via subretinal injection to each eye once on Day 1 of the dosing phase at a volume of 100 μL/eye (administered as a single 100 μL bleb/eye). The vehicle control article was Placebo.

Transgene product expression, as assessed by anti VEGF Fab concentrations, was assessed in the aqueous humor and serum during the course of the study and in a terminal sample of vitreous humor using and Electrochemiluminescent (ECL) assay. The assay is designed to measure any Construct II, the expressed gene product. An ECL immunoassay implemented using the Meso Scale Discovery (MSD) platform has been designed to quantitate Construct II TP in monkey aqueous humor samples or vitreous humor based on the bridging method. Briefly, the assay begins with overnight incubation of the calibrators, QCs, and samples with both VEGF-Biotin and rabbit anti-Construct II TP antibody. This allows the Construct II TP to bridge the VEGF-Biotin and rabbit anti-Construct II TP. The following day, the mixture is transferred to a blocked MSD streptavidin plate where bridging complexes can bind to the streptavidin via VEGF-Biotin. After incubation, the plate is washed and SULFO-TAG goat antirabbit secondary antibody is added. After incubation, the plate is washed and tripropylamine (TPA) containing MSD read buffer is added. In the presence of TPA and electric current, SULFO-TAG produces a chemiluminescent signal that is proportional to the amount of Construct II TP present in the Aqueous humor.

For animals given Construct II by suprachoroidal injection, dose proportional increases in transgene product were observed for aqueous and serum (as mean Cₘₐₓ, AUC_{0-56d}, and AUC_{0-92d}) and vitreous (as concentration) from 3×10¹⁰ to 3×10¹² GC/eye. In aqueous humor, a general decline in anti-VEGF Fab levels after Day 42 was observed at doses ≥3×10¹¹ GC/eye in the suprachoroidally-treated dose groups only which may be associated with anti-transgene product antibodies. For both aqueous and vitreous humor, concentrations of anti-VEGF Fab (as mean Cₘₐₓ, AUC_{0-56d}, and AUC_{0-92d}) were significantly higher in the subretinal group when compared to the same dose delivered suprachoroidally. However, concentration of anti-VEGF Fab in the serum for animals dosed at 3×10¹¹ GC/eye subretinally was greater than for animals dosed at 3×10¹¹ GC/eye suprachoroidally.

TABLE 39
STUDY DESIGN

| | | | | | | | | | | Dose | |
|---|---|---|---|---|---|---|---|---|---|---|---|
| | | | | | | Dosing | | Dose Level | | Concentration | |
| | | | | | | Regimen | | (GC/eye) | | (GC/mL) | |
| | Sub- | No. of Animals | | Dosing Route | | Left | Right | Left | Right | Left | Right |
| Group | group | Males | Females | Left Eye | Right Eye | Eye | Eye | Eye | Eye | Eye | Eye |
| 1 | 2 | 2 | 1 | Supra- | Sub- | VCA | VCA | 0 | 0 | 0 | 0 |
| | | | | choroidal | retinal | | | | | | |
| 2 | 1 | 2 | 1 | Supra- | Supra- | TA | TA | 3 × 1010 | 3 × 1010 | 3 × 1011 | 3 × 1011 |
| | | | | choroidal | choroidal | | | | | | |
| 3 | 1 | 2 | 1 | Supra- | Supra- | TA | TA | 3 × 1011 | 3 × 1011 | 3 × 1012 | 3 × 1012 |
| | | | | choroidal | choroidal | | | | | | |
| 4 | 1 | 2 | 2 | Supra- | Supra- | TA | TA | 3 × 1012 | 3 × 1012 | 3 × 1013 | 3 × 1013 |
| | | | | choroidal | choroidal | | | | | | |
| 5 | 1 | NA | 2 | Sub- | Sub- | TA | TA | 3 × 1011 | 3 × 1011 | 3 × 1012 | 3 × 1012 |
| | | | | retinal | retinal | | | | | | |
| 5 | 2 | 1 | NA | Sub- | Sub- | TA | TA | 3 × 1011 | 3 × 1011 | 3 × 1012 | 3 × 1012 |
| | | | | retinal | retinal | | | | | | |

Group 1 was administered vehicle control article only. The left eye was dosed at a volume of 100 μL/left eye (administered as two injections of 50 μL/injection/left eye). The right eye was dosed at a volume of 100 μL/right eye (administered as a single 100 μL bleb/right eye). Dose levels for animals in Groups 2, 3, and 4 were based on a total dose volume of 100 μL/eye. Animals in Groups 2, 3, and 4 were administered two injections of 50 μL/injection/eye for a total of 100 μL/eye. Both eyes of each animal in Group 5 were dosed at a total volume of 100 μL/eye, administered as a single 100 μL bleb/eye.

5.19.1 Results

All concentration values of anti-VEGF Fab in the vehicle control group were below the lower limit of quantitation (<0.100 ng/mL).

Transgene product expression, as assessed by anti-VEGF Fab mean Cₘₐₓ, AUC_{0-56d}, and AUC_{0-92d} values, increased with the increase in Construct II dose level from 3×10¹⁰ to 3×10¹² GC/eye when dosed suprachoroidally. The increases in mean Cₘₐₓ, AUC_{0-56d}, and AUC_{0-92d} values, when dosed suprachoroidally, were generally dose proportional. Anti-VEGF Fab concentrations for animals dosed at 3×10¹¹ GC/eye subretinally were greater than for animals dosed at 3×10₁₁ GC/eye suprachoroidally with both administration routes reaching, and maintaining, near Cₘₐₓ levels throughout the study. A general decline in anti-VEGF Fab levels after Day 42 is observed in the higher suprachoroidally dosed groups.

Mean levels of anti-VEGF Fab in vitreous humor generally increased with the increase of Construct II dose levels. Subretinal dosing showed significantly increased anti-VEGF Fab levels when compared with the same dose delivered suprachoroidally. The mean aqueous humor to vitreous humor anti-VEGF Fab concentration ratios on Day 92 ranged from 0.114 to 0.348 when dosed suprachoroidally and 0.201 when dosed subretinally.

### 5.20 Example 20: A Phase 2, Open-Label Study to Explore the Pharmacodynamics of Two Doses in Two Formulations of Construct II Gene Therapy Administered Via Subretinal Delivery in Participants with Neovascular Age-Related Macular Degeneration

In this phase 2, open-label, multiple cohort study, approximately 60 participants (15 per cohort) who meet the inclusion/exclusion criteria will be enrolled in 4 sequential dose cohorts. A dose cohort will be comprised of 1 of 2 doses of Construct II in 1 of 2 formulations, in order to explore the pharmacodynamics of Construct II based on aqueous humor TP concentrations. Endpoints are set forth in Table 40 below.

TABLE 40
OBJECTIVES AND ENDPOINTS

| | | |
|---|---|---|
| Measure | Objectives | Endpoints |

Primary

| | | |
|---|---|---|
| Pharmaco- | To assess Construct II TP | Construct II TP concentrations in aqueous |
| dynamics | concentrations in aqueous | humor at Week 24 |
| | humor at Week 24 | |

Secondary

| | | |
|---|---|---|
| Pharmaco- | To assess Construct II TP | Construct II TP concentrations in aqueous |
| dynamics | concentrations in aqueous | humor at Week 12 |
| | humor at Week 12 | |
| Safety | To evaluate the safety and | Incidence and severity of ocular AEs |
| | tolerability of Construct II | Incidence and severity of nonocular AEs |
| | through Week 24 | |
| Efficacy | To evaluate the effect of | Mean change from baseline in BCVA to |
| | Construct II on BCVA | Week 24 based on the ETDRS score |
| | To evaluate the effect of | Mean change from baseline in CRT, as |
| | Construct II on CRT, as | measured by SD-OCT to Week 24 |
| | measured by SD-OCT | Mean change from baseline in CFT, as |
| | To evaluate the effect of | measured by SD-OCT to Week 24 |
| | Construct II on CFT, as | Mean number of supplemental anti- |
| | measured by SD-OCT | VEGF injections through Week 24 |
| | To assess the need for | Percentage change in annualized rate of |
| | supplemental anti-VEGF | supplemental anti-VEGF injections pre |
| | therapy over 24 weeks | and post Construct II |
| | | Proportion of participants who receive |
| | | supplemental anti-VEGF injections after |
| | | Week 4 through Week 24 |

Exploratory

| | | |
|---|---|---|
| Safety | To evaluate incidence of new | Incidence of new geographic atrophy |
| | areas of atrophy and changes | Progression of geographic atrophy over |
| | over time | 24 weeks |
| | To evaluate incidence of new | Incidence of new retinal pigmentation |
| | areas of retinal pigmentation | Progression of retinal pigmentation over |
| | and changes over time | 24 weeks |
| Efficacy | Time to first supplemental | Time to first supplemental anti-VEGF |
| | anti-VEGF | injection |
| Immuno- | To evaluate the | Immunogenicity measurements (serum |
| genicity | immunogenicity of Construct | AAV8: NAbs and TAbs) |
| | II | AAV8 detected by PCR in tears |
| | To determine vector shedding | |
| | in tears samples | |
| Biomarkers | To assess aqueous VEGF-A | VEGF-A concentrations (aqueous) at |
| | concentrations | Week 12 and Week 24 |

AAV8 = adeno-associated virus serotype 8;
AE = adverse event;
BCVA = best-corrected visual acuity;
CFT = central foveal thickness;
CRT = central retinal thickness;
ETDRS = Early Treatment Diabetic Retinopathy Study;
NAbs = neutralizing antibodies;
PCR = polymerase chain reaction;
SD-OCT = spectral domain-optical coherence tomography;
TAbs = total binding antibodies;
TP = transgene product;
VEGF = vascular endothelial growth factor

5.20.1 Inclusion Criteria

Participants are eligible to be included in the study only if all of the following criteria apply:
1. Males or females, aged ≥50 years and ≤89 years.
2. An ETDRS BCVA-letter score between ≤78 and ≥40 in the study eye at Screening Visit 1.
   If both eyes are eligible, the study eye must be the participant's worse-seeing eye, as determined by the investigator prior to randomization.
3. Must have a diagnosis of subfoveal CNV secondary to age-related macular degeneration (AMD) in the study eye, along with fluid within the parafovea (3-mm center of the macula, based on the ETDRS grid) at Screening Visit 1, as assessed by the CRC.
   CNV lesion characteristics: lesion size needs to be less than 10-disc areas (typical disc area=2.54 mm²).
4. Must be pseudophakic (at least 12 weeks postcataract surgery) in the study eye.
5. Must be willing and able to comply with all study procedures and be available for the duration of the study.
6. Women must be postmenopausal (defined as being at least 12 consecutive months without menses) or surgically sterilized (i.e., having a bilateral tubal ligation/bilateral salpingectomy, bilateral tubal occlusive procedure, hysterectomy, or bilateral oophorectomy). If not, women must have negative urine and serum pregnancy test results at Screening Visit 1 and be willing to have additional pregnancy tests during the study.
7. Women of childbearing potential (WOCBP) (and their male partners) must be willing to use a highly effective method of contraception and male participants engaged in a sexual relationship with a WOCBP must be willing to use condoms from Screening Visit 1 until 24 weeks after Construct II administration.
8. Must be willing and able to provide written, signed informed consent.
9. Based on the Screening Visit 2 SD-OCT, participants must have improvement in fluid of >50 m or >50% (see Response Criterion below) and have a CRT<400 m, as determined by the CRC. Note that, if the participant has disease other than fluid contributing to an increase in CRT (i.e., pigment epithelial detachment [PED] or subretinal hyperreflective material [SHRM]), the participant will be enrolled if they have<50 m of fluid (intraretinal or subretinal), as determined by the CRC.
   Response Criterion: Participants must have an improvement in inner retinal (parafovea 3 mm) fluid relative to Screening Visit 1 of >50 μm or 50%.

5.20.2 Exclusion Criteria

Participants are excluded from the study if any of the following criteria apply:
10. CNV or macular edema in the study eye secondary to any causes other than AMD.
11. Subfoveal fibrosis or atrophy as determined by the CRC.
12. Participants who required >9 anti-VEGF injections in the 12 months prior to Screening Visit 1.
13. Any condition in the investigator's opinion that could limit VA improvement in the study eye.
14. Active or history of retinal detachment or retinal tear in the study eye.
15. Advanced glaucoma in the study eye, defined as an IOP >23 mmHg not controlled by 2 IOP-lowering medications or any invasive procedure to treat glaucoma (e.g., shunt, tube, or minimally invasive glaucoma surgery [MIGS] devices; selective laser trabeculectomy, argon laser trabeculoplasty, and MIGS devices are permitted).
16. Any participant with nAMD diagnosed >4 years prior to Screening Visit 1.
17. Any condition in the study eye that, in the opinion of the investigator, may increase the risk to the participant, require either medical or surgical intervention during the course of the study to prevent or treat vision loss, or interfere with study procedures or assessments.
18. History of intraocular surgery in the study eye within 12 weeks prior to Screening Visit 1. Yttrium aluminum garnet (YAG) capsulotomy is permitted if performed >10 weeks prior to Screening Visit 1.
19. History of intravitreal therapy in the study eye, such as intravitreal steroid injection or investigational product, other than anti-VEGF therapy, in the 6 months prior to Screening Visit 1.
20. Presence of any implant in the study eye at Screening Visit 1 (excluding intraocular lens or MIGS devices).
21. History of malignancy or hematologic malignancy that may compromise the immune system requiring chemotherapy and/or radiation in the 5 years prior to Screening Visit 1. Localized basal cell carcinoma will be permitted.
22. Receipt of any investigational product within the 30 days of enrollment or 5 half-lives of the investigational product, whichever is longer.
23. Received gene therapy.
24. History of retinal toxicity caused by a therapy, or concomitant therapy with any drug that may affect VA or with known retinal toxicity, e.g., chloroquine or hydroxychloroquine.
25. Ocular or periocular infection in the study eye that may interfere with the surgical procedure.
26. Myocardial infarction, cerebrovascular accident, or transient ischemic attack within the past 6 months.
27. Uncontrolled hypertension (systolic blood pressure [BP]>180 mmHg, diastolic BP >100 mmHg) despite maximal medical treatment.
28. Any participant with the following laboratory values at Screening Visit 1 will be withdrawn from study:
   Aspartate aminotransferase (AST)/alanine aminotransferase (ALT) >2.5× upper limit of normal (ULN).
   Total bilirubin >1.5×ULN, unless the participant has a previously known history of Gilbert's syndrome and a fractionated bilirubin that shows conjugated bilirubin <35% of total bilirubin.
   Prothrombin time >1.5×ULN, unless the participant is anticoagulated.
   Participants who are anticoagulated will be monitored by local laboratories and managed per local practice to hold or bridge anticoagulant therapy for the study procedure; consultation with the Medical Monitor is also required.
   Hemoglobin <10 g/dL for male participants and <9 g/dL for female participants.
   Platelets <100×10³/L.
   Estimated glomerular filtration rate <30 mL/min/1.73 m2.
   Currently receiving insulin for diabetes.
29. Currently taking anticoagulation therapy for which holding anticoagulation therapy for Construct II administration is not indicated or considered to be unsafe in the opinion of the treating investigator (i.e., retinal surgeon), as well as the physician prescribing anticoagulation for the participant
30. Any concomitant treatment that, in the opinion of the investigator, may interfere with ocular surgical procedure or healing process.
31. Known hypersensitivity to ranibizumab or any of its components.
32. Has a serious, chronic, or unstable medical or psychological condition that, in the opinion of the investigator or Sponsor, may compromise the participant's safety, interpretation of results, or ability to complete all assessments and follow-up in the study.

5.20.3 Study Intervention(s) Administered

Eligible participants will be assigned by cohort to 1 of 2 doses and 1 of 2 formulations of Construct II as set forth in Table 41 below. Participants will be given Construct II on Day 1 via subretinal delivery in an operating room. During the study, participants will receive ranibizumab 0.5 mg, administered by intravitreal injection, at Screening Visit 1, at Week 2, and then as needed every −28 days starting at Week 4.

TABLE 41
STUDY INTERVENTION(S) ADMINISTERED

| | Construct II | Construct II | |
|---|---|---|---|
| Arm Name | Dose 1 | Dose 2 | Ranibizumab (LUCENTIS) |
| Type | Gene therapy (Construct II) | | Drug (run-in/supplemental treatment) |
| Dose | Solution | | Solution |
| Formulation | | | |
| Unit Dose | 3.2 × 10¹¹ GC/mL | 6.5 × 10¹¹ GC/mL | 10 mg/mL |
| Strength | (nominal concentration) | (nominal concentration) | |
| Dose Level(s) | 200 μL | 200 μL | 0.5 mg (0.05 mL of 10 mg/mL |
| | (6.4 × 10¹⁰ GC/eye, | (1.3 × 10¹¹ GC/eye, | solution) once at Screening Visit 1 |
| | one-time dose) | one-time dose) | (run-in), once at Week 2, and, |
| | | | every ~28 days, as needed for |
| | | | supplemental treatment, starting |
| | | | 4 weeks post |
| | | | Construct II administration |
| | | | per retreatment criteria. |
| Formulation | Formulation 1: Dulbecco's phosphate buffered | | Not Applicable |
| | saline (DPBS) with 0.001% poloxamer 188 | | |
| | Formulation 2: Modified DPBS with 4% sucrose | | |
| | and 0.001% poloxamer 188 | | |
| Route of | Subretinal delivery | | Intravitreal injection in the study eye |
| Administration | | | |

### EQUIVALENTS

Although the invention is described in detail with reference to specific embodiments thereof, it will be understood that variations which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims. Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference in their entireties.

What is claimed:

1. A pharmaceutical composition comprising:
(a) a recombinant adeno-associated virus (AAV),
(b) potassium chloride,
(c) potassium phosphate monobasic,
(d) sodium chloride,
(e) sodium phosphate dibasic anhydrous,
(f) sucrose, and
(e) poloxamer 188, polysorbate 20, or polysorbate 80.

2. The pharmaceutical composition of claim 1, wherein the recombinant AAV comprises components from one or more adeno-associated virus serotypes selected from the group consisting of AAV1, AAV2, AAV2tYF, AAV3, AAV4, AAV5, AAV6, AAV7, AAV8, AAV9, AAV10, AAV11, AAVrh10, AAV.rh20, AAV.rh39, AAV.Rh74, AAV.RHM4-1, AAV.hu37, AAV.Anc80, AAV.Anc80L65, rAAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16.

3. The pharmaceutical composition of any one of claims 1-2, wherein the recombinant AAV is AAV8.

4. The pharmaceutical composition of any one of claims 1-2, wherein the recombinant AAV is AAV9.

5. The pharmaceutical composition of any one of claims 1-4, wherein the pharmaceutical composition further comprises one or more amino acids.

6. The pharmaceutical composition of any one of claims 1-5, wherein the ionic strength of the pharmaceutical composition is in a range from about 60 mM to about 115 mM.

7. The pharmaceutical composition of claim 4, wherein the ionic strength of the pharmaceutical composition is in a range from about 30 mM to about 100 mM

8. The pharmaceutical composition of any one of claims 1-3, wherein the pharmaceutical composition comprises
(a) potassium chloride at a concentration of 0.2 g/L,
(b) potassium phosphate monobasic at a concentration of 0.2 g/L,
(c) sodium chloride at a concentration of 5.84 g/L, and
(d) sodium phosphate dibasic anhydrous at a concentration of 1.15 g/L.

9. The pharmaceutical composition of any one of claims 1-8, wherein the pharmaceutical composition comprises sucrose at a concentration in a range from 3% (weight/volume, 30 g/L) to 18% (weight/volume, 180 g/L).

10. The pharmaceutical composition of any one of claims 1-8, wherein the pharmaceutical composition comprises sucrose at a concentration of 4% (weight/volume, 40 g/L).

11. The pharmaceutical composition of any one of claims 1-10, wherein the pharmaceutical composition comprises poloxamer 188, polysorbate 20, or polysorbate 80; and wherein the poloxamer 188, polysorbate 20, or polysorbate 80 is at a concentration in a range from 0.0005% (weight/volume, 0.005 g/L) to 0.05% (weight/volume, 0.5 g/L).

12. The pharmaceutical composition of any one of claims 1-10, wherein the pharmaceutical composition comprises poloxamer 188, polysorbate 20, or polysorbate 80; and wherein the poloxamer 188, polysorbate 20, or polysorbate 80 is at a concentration of 0.001% (weight/volume, 0.01 g/L).

13. The pharmaceutical composition of any one of claims 1-12, wherein the pH of the pharmaceutical composition is in a range from about 6.0 to about 9.0.

14. The pharmaceutical composition of any one of claims 1-12, wherein the pH of the pharmaceutical composition is about 7.4.

15. The pharmaceutical composition of any one of claims 1-14, wherein the osmolality of the pharmaceutical composition is in a range from about 200 mOsm/L to about 660 mOsm/L.

16. The pharmaceutical composition of any one of claims 1-15, wherein the pharmaceutical composition is in a hydrophobically-coated glass vial.

17. The pharmaceutical composition of any one of claims 1-15, wherein the pharmaceutical composition is in a Cyclo Olefin Polymer (COP) vial.

18. The pharmaceutical composition of any one of claims 1-15, wherein the pharmaceutical composition is in a Daikyo Crystal Zenith® (CZ) vial.

19. The pharmaceutical composition of any one of claims 1-15, wherein the pharmaceutical composition is in a TopLyo coated vial.

20. The pharmaceutical composition of any one of claims 1-19, wherein the pharmaceutical composition consists of:
(a) the recombinant AAV,
(b) potassium chloride at a concentration of 0.2 g/L,
(c) potassium phosphate monobasic at a concentration of 0.2 g/L,
(d) sodium chloride at a concentration of 5.84 g/L,
(e) sodium phosphate dibasic anhydrous at a concentration of 1.15 g/L,
(f) sucrose at a concentration of 4% weight/volume (40 g/L),
(g) poloxamer 188, polysorbate 20, or polysorbate 80 at a concentration of 0.001% weight/volume (0.01 g/L), and
(h) water, and
wherein the recombinant AAV is AAV8.

21. The pharmaceutical composition of any one of claims 1-20, wherein the vector genome concentration (VGC) of the pharmaceutical composition is about 3×10⁹ GC/mL, about 1×10¹⁰ GC/mL, about 1.2×10¹⁰ GC/mL, about 1.6×10¹⁰ GC/mL, about 4×10¹⁰ GC/mL, about 6×10¹⁰ GC/mL, about 2×10¹¹ GC/mL, about 2.4×10¹¹ GC/mL, about 2.5×10¹¹ GC/mL, about 3×10¹¹ GC/mL, about 3.2×10¹¹ GC/mL, about 6.2×10¹¹ GC/mL, about 6.5×10¹¹ GC/mL, about 1×10¹² GC/mL, about 3×10¹² GC/mL, about 2×10¹³ GC/mL or about 3×10¹³ GC/mL

22. The pharmaceutical composition of any one of claims 1-21, wherein the recombinant AAV is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more stable to freeze/thaw cycles than the same recombinant AAV in a reference pharmaceutical composition.

23. The pharmaceutical composition of any one of claims 1-22, wherein the recombinant AAV is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more stable than the same recombinant AAV in a reference pharmaceutical composition when stored at −20° C. for a period of time, wherein the period of time is about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, or about 24 months.

24. The pharmaceutical composition of any one of claims 1-23, wherein the recombinant AAV is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more stable than the same recombinant AAV in a reference pharmaceutical composition when stored at −80° C. for a period of time, wherein the period of time is about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, or about 24 months.

25. The pharmaceutical composition of any one of claims 1-24, wherein the recombinant AAV is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more stable than the same recombinant AAV in a reference pharmaceutical composition when stored at room temperate for a period of time, wherein the period of time is about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, or about 24 months.

26. The pharmaceutical composition of any one of claims 1-25, wherein, the recombinant AAV is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more stable than the same recombinant AAV in a reference pharmaceutical composition when (i) stored at −80° C. for a first period of time; (ii) subsequently thawed; and (iii) after thawing, stored at 4° C. for a second period of time.

27. The pharmaceutical composition of claim 26, wherein the first period of time is about 6 months, about 7 months, about 8 months, about 9 months, about 10 months, about 11 months, about 12 months, about 15 months, about 18 months, or about 24 months.

28. The pharmaceutical composition of claim 26, wherein the second period of time is about 1 week, about 2 weeks, about 3 weeks, about 4 weeks, about 1 month, about 2 months.

29. The pharmaceutical composition of any one of claims 1-22, wherein the vector genome concentration of the recombinant AAV after being stored at −80° C. for a period of time is at least 70%, 75%, 80%, 85%, 90%, 95%, 98%, or 99% of the vector genome concentration of the recombinant AAV before being stored at −80° C. for said period of time.

30. The pharmaceutical composition of any one of claims 1-22, wherein the vector genome concentration of the recombinant AAV after being stored at −20° C. for a period of time is at least 70%, 75%, 80%, 85%, 90%, 95%, 98%, or 99% of the vector genome concentration of the recombinant AAV before being stored at −20° C. for said period of time.

31. The pharmaceutical composition of any one of claims 1-22, wherein the vector genome concentration of the recombinant AAV after being stored at 4° C. for a period of time is at least 70%, 75%, 80%, 85%, 90%, 95%, 98%, or 99% of the vector genome concentration of the recombinant AAV before being stored at 4° C. for said period of time.

32. The pharmaceutical composition of any one of claims 1-22, wherein in the vitro potency of the recombinant AAV after being stored at −80° C. for a period of time is at least 70%, 75%, 80%, 85%, 90%, 95%, 98%, or 99% of the in vitro potency of the recombinant AAV before being stored at −80° C. for said period of time.

33. The pharmaceutical composition of any one of claims 1-22, wherein the in vitro potency of the recombinant AAV after being stored at −20° C. for a period of time is at least 70%, 75%, 80%, 85%, 90%, 95%, 98%, or 99% of the in vitro potency of the recombinant AAV before being stored at −20° C. for said period of time.

34. The pharmaceutical composition of any one of claims 1-22, wherein the in vitro potency of the recombinant AAV after being stored at 4° C. for a period of time is at least 70%, 75%, 80%, 85%, 90%, 95%, 98%, or 99% of the in vitro potency of the recombinant AAV before being stored at 4° C. for said period of time.

35. The pharmaceutical composition of any one of claims 1-22, wherein the size distribution of the recombinant AAV after being stored at −80° C. for a period of time is at least 70%, 75%, 80%, 85%, 90%, 95%, 98%, or 99% identical to the size distribution of the recombinant AAV before being stored at −80° C. for said period of time.

36. The pharmaceutical composition of any one of claims 1-22, wherein the size distribution of the recombinant AAV after being stored at −20° C. for a period of time is at least 70%, 75%, 80%, 85%, 90%, 95%, 98%, or 99% identical to the size distribution of the recombinant AAV before being stored at −20° C. for said period of time.

37. The pharmaceutical composition of any one of claims 1-22, wherein the size distribution of the recombinant AAV after being stored at 4° C. for a period of time is at least 70%, 75%, 80%, 85%, 90%, 95%, 98%, or 99% identical to the size distribution of the recombinant AAV before being stored at 4° C. for said period of time.

38. The pharmaceutical composition of any one of claims 29 to 37, wherein the period of time is about 1 month, about 2 months, about 3 months, about 4 months, about 5 months, about 6 months, about 7 months, about 8 months, about 9 months, about 10 months, about 11 months, about 12 months, about 15 months, about 18 months, or about 24 months.

39. The pharmaceutical composition of any one of claims 1-21, wherein the stability of recombinant AAV is determined by the infectivity of recombinant AAV.

40. The pharmaceutical composition of any one of claims 1-21, wherein the stability of recombinant AAV is determined by the levels of aggregation of recombinant AAV.

41. The pharmaceutical composition of any one of claims 1-21, wherein the stability of recombinant AAV is determined by the levels of free DNA released by the recombinant AAV particles.

42. The pharmaceutical composition of any one of claims 1-41, wherein the pharmaceutical composition is a liquid composition.

43. The pharmaceutical composition of any one of claims 1-41, wherein the pharmaceutical composition is a frozen composition.

44. The pharmaceutical composition of any one of claims 1-41, wherein the pharmaceutical composition is a lyophilized composition or a reconstituted lyophilized composition.

45. The pharmaceutical composition of any one of claims 1-44, the pharmaceutical composition has a property that is suitable for intravenous administration, subcutaneous administration, intramuscular injection, suprachoroidal injection, subretinal injection via transvitreal approach, subretinal administration via the suprachoroidal space, or a posterior juxtascleral depot procedure.

46. The pharmaceutical composition of any one of claims 1-44, the pharmaceutical composition has a desired density that is suitable for intravenous administration, subcutaneous administration, intramuscular injection, suprachoroidal injection, subretinal injection via transvitreal approach, subretinal administration via the suprachoroidal space, or a posterior juxtascleral depot procedure.

47. The pharmaceutical composition of any one of claims 1-44, the pharmaceutical composition has a desired osmolality that is suitable for intravenous administration, subcutaneous administration, intramuscular injection, suprachoroidal injection, subretinal injection via transvitreal approach, subretinal administration via the suprachoroidal space, or a posterior juxtascleral depot procedure.

48. The pharmaceutical composition of claim 47, wherein the osmolality is 160-230 mOsm/kg H₂O.

49. The pharmaceutical composition of claim 47, wherein the osmolality is less than 600 mOsm/kg H₂O.

50. The pharmaceutical composition of any one of claims 1-44, the pharmaceutical composition has a desired viscosity that is suitable for intravenous administration, subcutaneous administration, intramuscular injection, suprachoroidal injection, subretinal injection via transvitreal approach, subretinal administration via the suprachoroidal space, or a posterior juxtascleral depot procedure.

51. The pharmaceutical composition of any one of claims 1-44, the pharmaceutical composition is suitable for administration to the eye.

52. The pharmaceutical composition of claim 45, the pharmaceutical composition is suitable for suprachoroidal injection, subretinal injection via transvitreal approach, subretinal administration via the suprachoroidal space, or a posterior juxtascleral depot procedure.

53. The pharmaceutical composition of any one of claims 1-52, wherein the pharmaceutical composition is capable of being stored at 4° C. for 1 weeks, about 2 weeks, about 3 weeks, about 4 weeks, about 1 month, about 2 months after having previously been stored at −80° C. for about 6 months, about 7 months, about 8 months, about 9 months, about 10 months, about 11 months, about 12 months, about 15 months, about 18 months, about 24 months.

54. A method of treating or preventing a disease in a subject, comprising administering to the subject the pharmaceutical composition of any one of claims 1-53.

55. A method of treating a subject diagnosed with nAMD (wet AMD), dry AMD, retinal vein occlusion (RVO), diabetic macular edema (DME), or diabetic retinopathy (DR) comprising administering to the subject the pharmaceutical composition of any one of any one of claims 1-53.

56. A method of treating a subject diagnosed with mucopolysaccharidosis type IVA (MPS IVA), mucopolysaccharidosis type I (MPS I), mucopolysaccharidosis type II (MPS II), familial hypercholesterolemia (FH), homozygous familial hypercholesterolemia (HoFH), coronary artery disease, cerebrovascular disease, Duchenne muscular dystrophy, Limb Girdle muscular dystrophy, Becker muscular dystrophy and sporadic inclusion body myositis, or kallikrein-related disease comprising administering to the subject the pharmaceutical composition of any one of claims 1-53.

57. The method of any one of claims 54-56, wherein the pharmaceutical composition is administered by intravenous administration, subcutaneous administration, intramuscular injection, suprachoroidal injection, subretinal injection via transvitreal approach, subretinal administration via the suprachoroidal space, or a posterior juxtascleral depot procedure.

58. The method of any one of claims 54-57, wherein the subject is a human subject.

59. A method of treating a subject diagnosed with nAMD (wet AMD), dry AMD, retinal vein occlusion (RVO), diabetic macular edema (DME), or diabetic retinopathy (DR), the method comprising preparing the pharmaceutical composition of any one of claims 1 to 53, storing the pharmaceutical composition at −80° C. for a first period of time; (ii) thawing the pharmaceutical composition; and (iii) after thawing, storing the pharmaceutical composition at 4° C. for a second period of time.

60. The method of claim 59, wherein the first period of time is about 6 months, about 7 months, about 8 months, about 9 months, about 10 months, about 11 months, about 12 months, about 15 months, about 18 months, or about 24 months.

61. The method of claim 59, wherein the second period of time is about 1 week, about 2 weeks, about 3 weeks, about 4 weeks, about 1 month, about 2 months.

62. A kit comprising one or more containers and instructions for use, wherein the one or more containers comprise the pharmaceutical composition of any one of claims 1-53.

63. The kit of claim 62, wherein at least one of the one or more containers is made from hydrophobically-coated glass vial.

64. The kit of claim 62, wherein at least one of the one or more containers is made from Daikyo Crystal Zenith® (CZ) vial.

65. The kit of claim 62, wherein at least one of the one or more containers is made from TopLyo coated vial.

66. The kit of claim 62, wherein at least one of the one or more containers is made from Cyclo Olefin Polymer (COP).

67. A pharmaceutical composition comprising:
(a) a Construct II encoding an anti-human vascular endothelial growth factor (hVEGF) antibody,
(b) potassium chloride,
(c) potassium phosphate monobasic,
(d) sodium chloride,
(e) sodium phosphate dibasic anhydrous,
(f) sucrose, and
(e) poloxamer 188, polysorbate 20, or polysorbate 80, and
wherein the anti-hVEGF antibody comprises a heavy chain comprising the amino acid sequence of SEQ ID NO:2 or SEQ ID NO:4, and a light chain comprising the amino acid sequence of SEQ ID NO:1 or SEQ ID NO:3.

68. The pharmaceutical composition of claim 67, wherein the ionic strength of the pharmaceutical composition is in a range from about 60 mM to about 100 mM.

69. The pharmaceutical composition of any one of claims 67-68, wherein the pharmaceutical composition comprises:
(a) potassium chloride at a concentration of 0.2 g/L,
(b) potassium phosphate monobasic at a concentration of 0.2 g/L,
(c) sodium chloride at a concentration of 5.84 g/L, and
(d) sodium phosphate dibasic anhydrous at a concentration of 1.15 g/L.

70. The pharmaceutical composition of any one of claims 67-69, wherein the pharmaceutical composition comprises sucrose at a concentration in a range from 3% (weight/volume, 30 g/L) to 18% (weight/volume, 180 g/L).

71. The pharmaceutical composition of any one of claims 67-69, wherein the pharmaceutical composition comprises sucrose at a concentration of 4% (weight/volume, 40 g/L).

72. The pharmaceutical composition of any one of claims 67-71, wherein the pharmaceutical composition comprises poloxamer 188, polysorbate 20, or polysorbate 80; and wherein the poloxamer 188, polysorbate 20, or polysorbate 80 is at a concentration in a range from 0.0005% (weight/volume, 0.005 g/L) to 0.05% (weight/volume, 0.5 g/L).

73. The pharmaceutical composition of any one of claims 67-71, wherein the pharmaceutical composition comprises poloxamer 188, polysorbate 20, or polysorbate 80 at a concentration of 0.001% (weight/volume, 0.01 g/L).

74. The pharmaceutical composition of any one of claims 67-73, wherein the pH of the pharmaceutical composition is in a range from about 6.0 to about 9.0.

75. The pharmaceutical composition of any one of claims 67-73, wherein the pH of the pharmaceutical composition is about 7.4.

76. The pharmaceutical composition of any one of claims 67-75, wherein the osmolality of the pharmaceutical composition is in a range from about 200 mOsm/L to about 660 mOsm/L. The pharmaceutical composition of any one of claims 67-76, wherein the Construct II comprises the capsid from AAV8.

77. The pharmaceutical composition of any one of claims 67-76, wherein the Construct II is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more stable to freeze/thaw cycles than the same Construct II in a reference pharmaceutical composition.

78. The pharmaceutical composition of any one of claims 67-77, wherein the Construct II is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more stable than the same Construct II in a reference pharmaceutical composition when stored at −20° C. for a period of time, wherein the period of time is about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, or about 24 months.

79. The pharmaceutical composition of any one of claims 67-77, wherein the Construct II is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more stable than the same Construct II in a reference pharmaceutical composition when stored at −80° C. for a period of time, wherein the period of time is about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, or about 24 months.

80. The pharmaceutical composition of any one of claims 67-77, wherein the Construct II is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more stable than the same Construct II in a reference pharmaceutical composition when stored at room temperate for a period of time, wherein the period of time is about 1 weeks, about 2 weeks, about 3 weeks, about 4 weeks, about 1 month, about 2 months, about 3 months, about 4 months, about 5 months, about 6 months, about 7 months, about 8 months, about 9 months, about 10 months, about 11 months, about 12 months, about 15 months, about 18 months, or about 24 months.

81. The pharmaceutical composition of any one of claims 67-77, wherein, the recombinant AAV is at least 2%, 5%, 7%, 10%, 12%, 15%, 17%, 20%, 25%, 30%, 35%, 40%, 45%, 50%, 100%, 2 times, 3 times, 5 times, 10 times, 100 times, or 1000 more stable than the same Construct II in a reference pharmaceutical composition when (i) stored at −80° C. for a first period of time; (ii) subsequently thawed; and (iii) after thawing, stored at 4° C. for a second period of time.

82. The pharmaceutical composition of claim 81, wherein the first period of time is about 6 months, about 7 months, about 8 months, about 9 months, about 10 months, about 11 months, about 12 months, about 15 months, about 18 months, or about 24 months.

83. The pharmaceutical composition of claim 81, wherein the second period of time is about 1 week, about 2 weeks, about 3 weeks, about 4 weeks, about 1 month, about 2 months.

84. The pharmaceutical composition of any one of claims 67-79, wherein the vector genome concentration of the Construct II after being stored at −80° C. for a period of time is at least 70%, 75%, 80%, 85%, 90%, 95%, 98%, or 99% of the vector genome concentration of the Construct II before being stored at −80° C. for said period of time.

85. The pharmaceutical composition of any one of claims 67-79, wherein the vector genome concentration of the Construct II after being stored at −20° C. for a period of time is at least 70%, 75%, 80%, 85%, 90%, 95%, 98%, or 99% of the vector genome concentration of the Construct II before being stored at −20° C. for said period of time.

86. The pharmaceutical composition of any one of claims 67-79, wherein the vector genome concentration of the Construct II after being stored at 4° C. for a period of time is at least 70%, 75%, 80%, 85%, 90%, 95%, 98%, or 99% of the vector genome concentration of the Construct II before being stored at 4° C. for said period of time.

87. The pharmaceutical composition of any one of claims 67-79, wherein the in vitro potency of the Construct II after being stored at −80° C. for a period of time is at least 70%, 75%, 80%, 85%, 90%, 95%, 98%, or 99% of the in vitro potency of the Construct II before being stored at −80° C. for said period of time.

88. The pharmaceutical composition of any one of claims 67-79, wherein the in vitro potency of the Construct II after being stored at −20° C. for a period of time is at least 70%, 75%, 80%, 85%, 90%, 95%, 98%, or 99% of the in vitro potency of the Construct II before being stored at −20° C. for said period of time.

89. The pharmaceutical composition of any one of claims 67-79, wherein the in vitro potency of the Construct II after being stored at 4° C. for a period of time is at least 70%, 75%, 80%, 85%, 90%, 95%, 98%, or 99% of the in vitro potency of the Construct II before being stored at 4° C. for said period of time.

90. The pharmaceutical composition of any one of claims 67-79, wherein the size distribution of the Construct II after being stored at −80° C. for a period of time is at least 70%, 75%, 80%, 85%, 90%, 95%, 98%, or 99% identical to the size distribution of the Construct II before being stored at −80° C. for said period of time.

91. The pharmaceutical composition of any one of claims 67-79, wherein the size distribution of the Construct II after being stored at −20° C. for a period of time is at least 70%, 75%, 80%, 85%, 90%, 95%, 98%, or 99% identical to the size distribution of the Construct II before being stored at −20° C. for said period of time.

92. The pharmaceutical composition of any one of claims 67-79, wherein the size distribution of the Construct II after being stored at 4° C. for a period of time is at least 70%, 75%, 80%, 85%, 90%, 95%, 98%, or 99% identical to the size distribution of the Construct II before being stored at 4° C. for said period of time.

93. The pharmaceutical composition of any one of claims 84 to 92, wherein the period of time is about 1 month, about 2 months, about 3 months, about 4 months, about 5 months, about 6 months, about 7 months, about 8 months, about 9 months, about 10 months, about 11 months, about 12 months, about 15 months, about 18 months, or about 24 months.

94. The pharmaceutical composition of any one of claims 77-83, wherein the stability of the Construct II is determined by the infectivity of recombinant AAV.

95. The pharmaceutical composition of any one of claims 77-83, wherein the stability of the Construct II is determined by the levels of aggregation of recombinant AAV.

96. The pharmaceutical composition of any one of claims 77-83, wherein the stability of the Construct II is determined by the levels of free DNA released by the recombinant AAV particles.

97. The pharmaceutical composition of any one of claims 67-96, wherein the pharmaceutical composition is in a hydrophobically-coated glass vial.

98. The pharmaceutical composition of any one of claims 67-96, wherein the pharmaceutical composition is in a Cyclo Olefin Polymer (COP) vial.

99. The pharmaceutical composition of any one of claims 67-96, wherein the pharmaceutical composition is in a Daikyo Crystal Zenith® (CZ) vial.

100. The pharmaceutical composition of any one of claims 67-96, wherein the pharmaceutical composition is in a TopLyo coated vial.

101. The pharmaceutical composition of any one of claims 67-100, wherein the pharmaceutical composition consists of:
(a) the Construct II encoding an anti-human vascular endothelial growth factor (hVEGF) antibody,
(b) potassium chloride at a concentration of 0.2 g/L,
(c) potassium phosphate monobasic at a concentration of 0.2 g/L,
(d) sodium chloride at a concentration of 5.84 g/L,
(e) sodium phosphate dibasic anhydrous at a concentration of 1.15 g/L,
(f) sucrose at a concentration of 4% weight/volume (40 g/L),
(g) poloxamer 188, polysorbate 20, or polysorbate 80 at a concentration of 0.001% weight/volume (0.01 g/L), and
(h) water, and
wherein the anti-hVEGF antibody comprises a heavy chain comprising the amino acid sequence of SEQ ID NO:2 or SEQ ID NO:4, and a light chain comprising the amino acid sequence of SEQ ID NO:1 or SEQ ID NO:3.

102. The pharmaceutical composition of claim 101, wherein the vector genome concentration (VGC) of the pharmaceutical composition is about 3×10⁹ GC/mL, about 1×10¹⁰ GC/mL, about 1.2×10¹⁰ GC/mL, about 1.6×10¹⁰ GC/mL, about 4×10¹⁰ GC/mL, about 6×10¹⁰ GC/mL, about 2×10¹¹ GC/mL, about 2.4×10¹¹ GC/mL, about 2.5×10¹¹ GC/mL, about 3×10¹¹ GC/mL, about 3.2×10¹¹ GC/mL, about 6.2×10¹¹ GC/mL, about 6.5×10¹¹ GC/mL, about 1×10¹² GC/mL, about 3×10¹² GC/mL, about 2×10¹³ GC/mL. or about 3×10¹³ GC/mL.

103. The pharmaceutical composition of any one of claims 67-102, the pharmaceutical composition has a property that is suitable for intravenous administration, subcutaneous administration, intramuscular injection, suprachoroidal injection, subretinal injection via transvitreal approach, subretinal administration via the suprachoroidal space, or a posterior juxtascleral depot procedure.

104. The pharmaceutical composition of any one of claims 67-102, the pharmaceutical composition has a desired density that is suitable for intravenous administration, subcutaneous administration, intramuscular injection, suprachoroidal injection, subretinal injection via transvitreal approach, subretinal administration via the suprachoroidal space, or a posterior juxtascleral depot procedure.

105. The pharmaceutical composition of any one of claims 67-102, the pharmaceutical composition has a desired osmolality that is suitable for intravenous administration, subcutaneous administration, intramuscular injection, suprachoroidal injection, subretinal injection via transvitreal approach, subretinal administration via the suprachoroidal space, or a posterior juxtascleral depot procedure.

106. The pharmaceutical composition of claim 105 wherein the osmolality is 160-230 mOsm/kg H₂O.

107. The pharmaceutical composition of claim 105, wherein the osmolality is less than 600 mOsm/kg H₂O.

108. The pharmaceutical composition of any one of claims 67-102, the pharmaceutical composition has a desired viscosity that is suitable for intravenous administration, subcutaneous administration, intramuscular injection, suprachoroidal injection, subretinal injection via transvitreal approach, subretinal administration via the suprachoroidal space, or a posterior juxtascleral depot procedure.

109. The pharmaceutical composition of any one of claims 67-108, wherein the pharmaceutical composition is a liquid composition.

110. The pharmaceutical composition of any one of claims 67-108, wherein the pharmaceutical composition is a frozen composition.

111. The pharmaceutical composition of any one of claims 67-108, wherein the pharmaceutical composition is a lyophilized composition or a reconstituted lyophilized composition.

112. The pharmaceutical composition of any one of claims 67-111, wherein the pharmaceutical composition is capable of being stored at 4° C. for 1 weeks, about 2 weeks, about 3 weeks, about 4 weeks, about 1 month, about 2 months after having previously been stored at −80° C. for about 6 months, about 7 months, about 8 months, about 9 months, about 10 months, about 11 months, about 12 months, about 15 months, about 18 months, about 24 months.

113. A method of treating a subject diagnosed with nAMD (wet AMD), dry AMD, retinal vein occlusion (RVO), diabetic macular edema (DME), or diabetic retinopathy (DR) comprising administering to the subject the pharmaceutical composition of any one of claims 67-111.

114. The method of claim 113, wherein the pharmaceutical composition is administered by suprachoroidal injection, subretinal injection via transvitreal approach, subretinal administration via the suprachoroidal space, or a posterior juxtascleral depot procedure.

115. The method of claim 113 or 114, wherein the subject is a human subject.

116. A method of treating a subject diagnosed with nAMD (wet AMD), dry AMD, retinal vein occlusion (RVO), diabetic macular edema (DME), or diabetic retinopathy (DR), the method comprising preparing the pharmaceutical composition of any one of claims 67-108, storing the pharmaceutical composition at −80° C. for a first period of time; (ii) thawing the pharmaceutical composition; and (iii) after thawing, storing the pharmaceutical composition at 4° C. for a second period of time.

117. The method of claim 119, wherein the first period of time is about 6 months, about 7 months, about 8 months, about 9 months, about 10 months, about 11 months, about 12 months, about 15 months, about 18 months, or about 24 months.

118. The method of claim 119, wherein the second period of time is about 1 week, about 2 weeks, about 3 weeks, about 4 weeks, about 1 month, about 2 months.

119. A kit comprising one or more containers and instructions for use, wherein the one or more containers comprise the pharmaceutical composition of any one of claims 67-102.

120. The kit of claim 119, wherein at least one of the one or more containers is made from Cyclo Olefin Polymer (COP).

121. A stable liquid pharmaceutical composition comprising:
(a) a recombinant adeno-associated virus (rAAV),
(b) a buffering agent comprising ionic salt and having an ionic strength between 60 mM and 150 mM,
(d) sucrose, and
(e) surfactant.

122. The composition of claim 121, wherein the rAAV comprises AAV1, AAV2, AAV2tYF, AAV3, AAV4, AAV5, AAV6, AAV7, AAV8, AAV9, AAV10, AAV11, AAVrh10, AAV.rh20, AAV.rh39, AAV.Rh74, AAV.RHM4-1, AAV.hu37, AAV.Anc80, AAV.Anc80L65, rAAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16.

123. The composition of claim 121 or 122, wherein the composition comprises 3-16% sucrose.

124. The composition of any one of claims 121-123, wherein the buffering agent maintains pH between about pH 6 to about pH 9 over the temperature range of −20° C. to room temperature.

125. The composition of any one of claims 121-124, wherein the composition comprises 4-6% sucrose.

126. The composition of any one of claims 121-125, wherein the buffering agent has an ionic strength no greater than about 150 mM, about 145 mM, about 140 mM, about 135 mM, about 130 mM, about 125 mM, about 120 mM, about 115 mM, or about 110 mM.

127. The composition of any one of claims 121-126, wherein the buffering agent has an ionic strength between about 60 mM and about 115 mM.

128. The composition of any one of claims 121-127, wherein the buffering agent has an ionic strength between about 60 mM and about 110 mM.

129. The composition of any one of claims 121-128, wherein the composition comprises between 60 mM and 100 mM NaCl.

130. The composition of any one of claims 121-129, wherein the composition comprises 4-6% sucrose.

131. The composition of any one of claims 121-130, wherein the composition is frozen to a temperature of about −20° C.

132. The composition of any one of claims 121-131, wherein the frozen composition maintains pH between about pH 6 to about pH 9.

133. The composition of any one of claims 121-132, wherein the buffering agent comprises one or more components selected from the group consisting of potassium phosphate monobasic, potassium phosphate, sodium chloride, sodium phosphate dibasic anhydrous, sodium phosphate hexahydrate, sodium phosphate monobasic monohydrate, sodium phosphate hexahydrate, sodium phosphate monobasic monohydrate, tromethamine, tris(hydroxymethyl)aminomethane hydrochloride (Tris-HCl), amino acid, histidine, histidine hydrochloride (histidine-HCl), sodium succinate, sodium citrate, sodium acetate, and (4-(2-hydroxyethyl)-1-piperazineethanesulfonic acid) (HEPES), sodium sulfate, magnesium sulfate, magnesium chloride 6-hydrate, calcium sulfate, potassium chloride, calcium chloride, and calcium citrate.

134. The composition of any one of claims 121-132, wherein the buffering agent comprises potassium chloride, potassium phosphate monobasic, sodium chloride, sodium phosphate dibasic anhydrous.

135. The composition of any one of claims 121-132, wherein the buffering agent comprises sodium chloride, and Tris hydrochloride.

136. The composition of any one of claims 121-133, wherein the surfactant is poloxamer 188, polysorbate 20, or polysorbate 80.

137. The composition of any one of claims 121-133, wherein the surfactant is poloxamer 188, polysorbate 20, or polysorbate 80; and wherein the poloxamer 188, polysorbate 20, or polysorbate 80 is at a concentration in a range from 0.0005% (weight/volume, 0.005 g/L) to 0.05% (weight/volume, 0.5 g/L).

138. The composition of any one of claims 121-133, wherein the surfactant is poloxamer 188, polysorbate 20, or polysorbate 80; and wherein the poloxamer 188, polysorbate 20, or polysorbate 80 is at a concentration of 0.001% (weight/volume, 0.01 g/L).

139. The composition of any one of claims 121-138, wherein the liquid pharmaceutical composition is further lyophilized.

140. The composition of any one of claims 121-138, wherein the liquid pharmaceutical composition is a reconstituted lyophilized powder.

141. The pharmaceutical composition of any one of claims 121-140, wherein the vector genome concentration of the recombinant AAV after being stored at −80° C. for a period of time is at least 70%, 75%, 80%, 85%, 90%, 95%, 98%, or 99% of the vector genome concentration of the recombinant AAV before being stored at −80° C. for said period of time.

142. The pharmaceutical composition of any one of claims 121-140, wherein the vector genome concentration of the recombinant AAV after being stored at −20° C. for a period of time is at least 70%, 75%, 80%, 85%, 90%, 95%, 98%, or 99% of the vector genome concentration of the recombinant AAV before being stored at −20° C. for said period of time.

143. The pharmaceutical composition of any one of claims 121-140, wherein the vector genome concentration of the recombinant AAV after being stored at 4° C. for a period of time is at least 70%, 75%, 80%, 85%, 90%, 95%, 98%, or 99% of the vector genome concentration of the recombinant AAV before being stored at 4° C. for said period of time.

144. The pharmaceutical composition of any one of claims 121-140, wherein the in vitro potency of the recombinant AAV after being stored at −80° C. for a period of time is at least 70%, 75%, 80%, 85%, 90%, 95%, 98%, or 99% of the in vitro potency of the recombinant AAV before being stored at −80° C. for said period of time.

145. The pharmaceutical composition of any one of claims 121-140, wherein the in vitro potency of the recombinant AAV after being stored at −20° C. for a period of time is at least 70%, 75%, 80%, 85%, 90%, 95%, 98%, or 99% of the in vitro potency of the recombinant AAV before being stored at −20° C. for said period of time.

146. The pharmaceutical composition of any one of claims 121-140, wherein the in vitro potency of the recombinant AAV after being stored at 4° C. for a period of time is at least 70%, 75%, 80%, 85%, 90%, 95%, 98%, or 99% of the in vitro potency of the recombinant AAV before being stored at 4° C. for said period of time.

147. The pharmaceutical composition of any one of claims 121-140, wherein the size distribution of the recombinant AAV after being stored at −80° C. for a period of time is at least 70%, 75%, 80%, 85%, 90%, 95%, 98%, or 99% identical to the size distribution of the recombinant AAV before being stored at −80° C. for said period of time.

148. The pharmaceutical composition of any one of claims 121-140, wherein the size distribution of the recombinant AAV after being stored at −20° C. for a period of time is at least 70%, 75%, 80%, 85%, 90%, 95%, 98%, or 99% identical to the size distribution of the recombinant AAV before being stored at −20° C. for said period of time.

149. The pharmaceutical composition of any one of claims 121-140, wherein the size distribution of the recombinant AAV after being stored at 4° C. for a period of time is at least 70%, 75%, 80%, 85%, 90%, 95%, 98%, or 99% identical to the size distribution of the recombinant AAV before being stored at 4° C. for said period of time.

150. The pharmaceutical composition of any one of claims 141-149, wherein the period of time is about 1 month, about 2 months, about 3 months, about 4 months, about 5 months, about 6 months, about 7 months, about 8 months, about 9 months, about 10 months, about 11 months, about 12 months, about 15 months, about 18 months, or about 24 months.

151. The pharmaceutical composition of any one of claims 121-140, wherein the pharmaceutical composition is capable of being stored at 4° C. for 1 weeks, about 2 weeks, about 3 weeks, about 4 weeks, about 1 month, about 2 months after having previously been stored at −80° C. for about 6 months, about 7 months, about 8 months, about 9 months, about 10 months, about 11 months, about 12 months, about 15 months, about 18 months, about 24 months.

152. A method of treating a disease of interest comprising administering to the subject the pharmaceutical composition of any one of claims 121-140, wherein the rAAV encodes a transgene that treats, or otherwise ameliorates, prevents or slows the progression of the disease of interest.

153. A method of treating a subject diagnosed with nAMD (wet AMD), dry AMD, retinal vein occlusion (RVO), diabetic macular edema (DME), or diabetic retinopathy (DR), the method comprising preparing the pharmaceutical composition of any one of claims 121-140, storing the pharmaceutical composition at −80° C. for a first period of time; (ii) thawing the pharmaceutical composition; and (iii) after thawing, storing the pharmaceutical composition at 4° C. for a second period of time.

154. The method composition of claim 153 wherein the first period of time is about 6 months, about 7 months, about 8 months, about 9 months, about 10 months, about 11 months, about 12 months, about 15 months, about 18 months, or about 24 months.

155. The method of claim 153, wherein the second period of time is about 1 week, about 2 weeks, about 3 weeks, about 4 weeks, about 1 month, about 2 months.

156. A kit comprising one or more containers and instructions for use, wherein the one or more containers comprise the pharmaceutical composition of any one of claims 121-140.

157. A stable liquid formulation comprising the pharmaceutical composition of any one of claims 1-53, 67-111 and 121-140.

158. A single unit dosage form comprising 3.2×10¹¹ GC/mL of Construct II, 0.2 g/L potassium chloride, 0.2 g/L potassium phosphate monobasic, 8.01 g/L sodium chloride, 1.15 g/L sodium phosphate dibasic anhydrous, pH 7.4 and 0.001% P188 in a volume of about 0.95 mL in a Cyclo Olefin Polymer (COP) vial.

159. A single unit dosage form comprising 3.2×10¹¹ GC/mL of Construct II, 0.2 g/L potassium chloride, 0.2 g/L potassium phosphate monobasic, 8.01 g/L sodium chloride, 1.15 g/L sodium phosphate dibasic anhydrous, pH 7.4 and 0.001% P188 in a volume of at least 0.8 mL in a COP vial.

160. A single unit dosage form comprising 3.2×10¹¹ GC/mL of Construct II, 0.2 g/L potassium chloride, 0.2 g/L potassium phosphate monobasic, 8.01 g/L sodium chloride, 1.15 g/L sodium phosphate dibasic anhydrous, pH 7.4, 4% sucrose and 0.001% P188 in a volume of about 0.95 mL in a COP vial.

161. A single unit dosage form comprising 3.2×10¹¹ GC/mL of Construct II, 0.2 g/L potassium chloride, 0.2 g/L potassium phosphate monobasic, 8.01 g/L sodium chloride, 1.15 g/L sodium phosphate dibasic anhydrous, pH 7.4, 4% sucrose and 0.001% P188 in a volume of at least 0.8 mL in a COP vial.

162. A single unit dosage form comprising 6.5×10¹¹ GC/mL of Construct II, 0.2 g/L potassium chloride, 0.2 g/L potassium phosphate monobasic, 8.01 g/L sodium chloride, 1.15 g/L sodium phosphate dibasic anhydrous, pH 7.4 and 0.001% P188 in a volume of about 0.95 mL in a COP vial.

163. A single unit dosage form comprising 6.5×10¹¹ GC/mL of Construct II, 0.2 g/L potassium chloride, 0.2 g/L potassium phosphate monobasic, 8.01 g/L sodium chloride, 1.15 g/L sodium phosphate dibasic anhydrous, pH 7.4, and 0.001% P188 in a volume of at least 0.8 mL in a COP vial.

164. A single unit dosage form comprising 6.5×10¹¹ GC/mL of Construct II, 0.2 g/L potassium chloride, 0.2 g/L potassium phosphate monobasic, 8.01 g/L sodium chloride, 1.15 g/L sodium phosphate dibasic anhydrous, pH 7.4, 4% sucrose and 0.001% P188 in a volume of about 0.95 mL in a COP vial.

165. A single unit dosage form comprising 6.5×10¹¹ GC/mL of Construct II, 0.2 g/L potassium chloride, 0.2 g/L potassium phosphate monobasic, 8.01 g/L sodium chloride, 1.15 g/L sodium phosphate dibasic anhydrous, pH 7.4, 4% sucrose and 0.001% P188 in a volume of at least 0.8 mL in a COP vial.

166. A single unit dosage form comprising 2.5×10¹² GC/mL of Construct II, 0.2 g/L potassium chloride, 0.2 g/L potassium phosphate monobasic, 8.01 g/L sodium chloride, 1.15 g/L sodium phosphate dibasic anhydrous, pH 7.4, and 0.001% P188 in a volume of about 0.6 mL in a COP vial.

167. A single unit dosage form comprising 2.5×10¹² GC/mL of Construct II, 0.2 g/L potassium chloride, 0.2 g/L potassium phosphate monobasic, 8.01 g/L sodium chloride, 1.15 g/L sodium phosphate dibasic anhydrous, pH 7.4, and 0.001% P188 in a volume of at least 0.5 mL in a COP vial.

168. A single unit dosage form comprising 3×10¹³ GC/mL of Construct II, 0.2 g/L potassium chloride, 0.2 g/L potassium phosphate monobasic, 8.01 g/L sodium chloride, 1.15 g/L sodium phosphate dibasic anhydrous, pH 7.4, 4% sucrose and 0.001% P188 in a volume of about 0.6 mL in a COP vial.

169. A single unit dosage form comprising 3×10¹³ GC/mL of Construct II, 0.2 g/L potassium chloride, 0.2 g/L potassium phosphate monobasic, 8.01 g/L sodium chloride, 1.15 g/L sodium phosphate dibasic anhydrous, pH 7.4, 4% sucrose and 0.001% P188 in a volume of at least 0.5 mL in a COP vial.

170. The single unit dosage form of any one of claims 158-169, which is capable of being stored at 4° C. for 1 weeks, about 2 weeks, about 3 weeks, about 4 weeks, about 1 month, about 2 months.

171. The single unit dosage form of any one of claims 158-169, which is capable of being stored at −80° C. for about 6 months, about 7 months, about 8 months, about 9 months, about 10 months, about 11 months, about 12 months, about 15 months, about 18 months, about 24 months.

172. The single unit dosage form of any one of claims 158-169, which is capable of being stored at 4° C. for 1 weeks, about 2 weeks, about 3 weeks, about 4 weeks, about 1 month, about 2 months after having previously been stored at −80° C. for about 6 months, about 7 months, about 8 months, about 9 months, about 10 months, about 11 months, about 12 months, about 15 months, about 18 months, about 24 months.

173. The single unit dosage form of any one of claims 158-169, wherein the vector genome concentration of the Construct II after being stored at −80° C. for a period of time is at least 70%, 75%, 80%, 85%, 90%, 95%, 98%, or 99% of the vector genome concentration of the Construct II before being stored at −80° C. for said period of time.

174. The single unit dosage form of any one of claims 158-169, wherein the vector genome concentration of the Construct II after being stored at −20° C. for a period of time is at least 70%, 75%, 80%, 85%, 90%, 95%, 98%, or 99% of the vector genome concentration of the Construct II before being stored at −20° C. for said period of time.

175. The single unit dosage form of any one of claims 158-169, wherein the vector genome concentration of the Construct II after being stored at 4° C. for a period of time is at least 70%, 75%, 80%, 85%, 90%, 95%, 98%, or 99% of the vector genome concentration of the Construct II before being stored at 4° C. for said period of time.

176. The single unit dosage form of any one of claims 158-169, wherein the in vitro potency of the Construct II after being stored at −80° C. for a period of time is at least 70%, 75%, 80%, 85%, 90%, 95%, 98%, or 99% of the in vitro potency of the Construct II before being stored at −80° C. for said period of time.

177. The single unit dosage form of any one of claims 158-169, wherein the in vitro potency of the Construct II after being stored at −20° C. for a period of time is at least 70%, 75%, 80%, 85%, 90%, 95%, 98%, or 99% of the in vitro potency of the Construct II before being stored at −20° C. for said period of time.

178. The single unit dosage form of any one of claims 158-169, wherein the in vitro potency of the Construct II after being stored at 4° C. for a period of time is at least 70%, 75%, 80%, 85%, 90%, 95%, 98%, or 99% of the in vitro potency of the Construct II before being stored at 4° C. for said period of time.

179. The single unit dosage form of any one of claims 158-169, wherein the size distribution of the Construct II after being stored at −80° C. for a period of time is at least 70%, 75%, 80%, 85%, 90%, 95%, 98%, or 99% identical to the size distribution of the Construct II before being stored at −80° C. for said period of time.

180. The single unit dosage form of any one of claims 158-169, wherein the size distribution of the Construct II after being stored at −20° C. for a period of time is at least 70%, 75%, 80%, 85%, 90%, 95%, 98%, or 99% identical to the size distribution of the Construct II before being stored at −20° C. for said period of time.

181. The single unit dosage form of any one of claims 158-169, wherein the size distribution of the Construct II after being stored at 4° C. for a period of time is at least 70%, 75%, 80%, 85%, 90%, 95%, 98%, or 99% identical to the size distribution of the Construct II before being stored at 4° C. for said period of time.

182. The single unit dosage form of any one of claims 173-181, wherein the period of time is about 1 month, about 2 months, about 3 months, about 4 months, about 5 months, about 6 months, about 7 months, about 8 months, about 9 months, about 10 months, about 11 months, about 12 months, about 15 months, about 18 months, or about 24 months.

183. A pre-filled syringe containing the single unit dosage form of any one of claims 158-183.

184. A kit comprising the pre-filled syringe of claim 183.
